(19) United States
(12) Patent Application Publication
(10) Pub. No.: US 2024/0334818 A1
(43) Pub. Date: Oct. 3, 2024

(54) AROMATIC AMINE COMPOUNDS, AND COMPOSITIONS AND ORGANIC ELECTRONIC DEVICES INCLUDING THE SAME

(71) Applicants: SHENZHEN CHINA STAR OPTOELECTRONICS SEMICONDUCTOR DISPLAY TECHNOLOGY CO., LTD., Shenzhen (CN); GUANGZHOU CHINARAY OPTOELECTRONIC MATERIALS LTD., Guangzhou (CN)

(72) Inventors: Ruifeng HE, Shenzhen (CN); Canjie WU, Shenzhen (CN); Kewei HE, Shenzhen (CN); Jingyao SONG, Shenzhen (CN)

(21) Appl. No.: 18/210,407

(22) Filed: Jun. 15, 2023

(30) Foreign Application Priority Data
Mar. 27, 2023 (CN) 202310349059.0

(51) Int. Cl. H10K 85/60; C07D 307/77; C07D 307/91; C07D 405/12; C07D 409/12; C07D 493/04; C07D 495/04; C09K 11/06

(52) U.S. Cl. CPC H10K 85/636; C07D 307/77; C07D 307/91; C07D 405/12; C07D 409/12; C07D 493/04; C07D 495/04; C09K 11/06; H10K 85/633; C07B 2200/05; C09K 2211/1018; H10K 50/181; H10K 85/615; H10K 85/622; H10K 85/657; H10K 85/6572; H10K 85/6574; H10K 85/6576

(57) ABSTRACT

An aromatic amine compound has a structure represented by formula (1). In the formula (1), Ar₁ and Ar₂ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof; Ar₃ is selected from a substituted or unsubstituted aromatic group with 6 to 30 ring atoms or a substituted or unsubstituted heteroaromatic group with 5 to 30 ring atoms; and R₁ is selected from substituted or unsubstituted methyl, substituted or unsubstituted ethyl, substituted or unsubstituted isopropyl, substituted or unsubstituted tertbutyl, substituted or unsubstituted cyclopentyl, substituted or unsubstituted cyclohexyl, or a substituted or unsubstituted adamantane group.

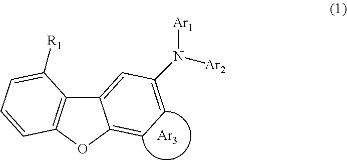

### CROSS-REFERENCE TO RELATED APPLICATION

The present disclosure claims priority to and the benefit of Chinese Patent Application No. 202310349059.0, filed on Mar. 27, 2023, the disclosure of which is incorporated herein by reference in its entirety.

### TECHNICAL FIELD

The disclosure relates to organic electroluminescent technology, in particular to aromatic amine compounds, and compositions and organic electronic devices including the same.

### BACKGROUND

Organic electroluminescent devices are structurally diverse, relatively inexpensive to manufacture, and have excellent optical and electrical properties. The organic electroluminescent devices such as organic-light emitting diodes (OLEDs) can be applied in optoelectronic devices, such as flat panel display devices and illumination devices. Moreover, the optoelectronic devices including the OLEDs have great potential for further development because of advantages of wide viewing angles, fast reaction time, low operating voltages, and thin thicknesses.

Further development of luminescent auxiliary materials is important to improve performances of the organic electroluminescent devices. At present, although a large number of the luminescent auxiliary materials have been developed, the organic electroluminescent devices containing the luminescent auxiliary materials still have problems of imbalanced charge transmission and insufficient lifespan. Therefore, how to design new luminescent auxiliary materials with better performances to regulate charge transmission balance, improve luminous efficiency, and prolong lifespan of the organic electroluminescent devices, are urgent problems for those skilled in the art.

### SUMMARY

In view of the above, the present disclosure provides an aromatic amine compound represented by formula (1):

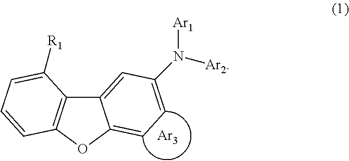

Ar₁ and Ar₂ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof.

Ar₃ is selected from a substituted or unsubstituted aromatic group with 6 to 30 ring atoms or a substituted or unsubstituted heteroaromatic group with 5 to 30 ring atoms.

R₁ is selected from substituted or unsubstituted methyl, substituted or unsubstituted ethyl, substituted or unsubstituted isopropyl, substituted or unsubstituted tertbutyl, substituted or unsubstituted cyclopentyl, substituted or unsubstituted cyclohexyl, or a substituted or unsubstituted adamantane group.

The present disclosure provides a composition. The composition includes the above-mentioned aromatic amine compound and an organic solvent.

Moreover, the present disclosure provides an organic electronic device. The organic electronic device includes a first electrode, a second electrode, and an organic functional layer disposed between the first electrode and the second electrode. The organic functional layer includes the above-mentioned aromatic amine compound.

### BRIEF DESCRIPTION OF THE DRAWINGS

FIG. 1 is a schematic structural diagram of an organic electronic device according to an embodiment of the present disclosure.

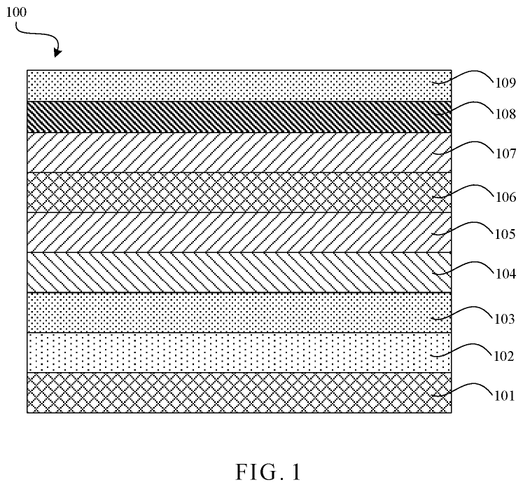

### DETAILED DESCRIPTION

The following provides a clear and complete description of the technical solution in the embodiments of the present disclosure, in conjunction with the accompanying drawings. It is apparent that the described embodiments are only some of the embodiments of the present disclosure, not all of them. Based on the embodiments of the present disclosure, other embodiments obtained by those skilled in the art without creative effort belong to a scope of the present disclosure. In addition, it should be understood that specific embodiments described herein are only used to explain and illustrate the present disclosure and are not used to limit the present disclosure. In the present disclosure, location terms used, such as “up” and “down”, generally refer to up and down in actual using or working state of devices, in particular drawing directions in the drawings, unless otherwise described.

In the description of the present disclosure, a term “include” refers to “include but not limited to”, and a term “more” refers to “two or more than two”. Various embodiments of the disclosure may exist in a form of a scope. It should be understood that description in a form of the scope is only for convenience and conciseness, and should not be understood as a rigid restriction on the scope of the disclosure. Therefore, it should be considered that the description of ranges has specifically disclosed all possible sub-ranges and single values within the sub-ranges. For example, it should be considered that the description of a range “from 1 to 6” has specifically disclosed a sub-range, such as from 1 to 3, from 1 to 4, from 1 to 5, from 2 to 4, from 2 to 6, from 3 to 6, or a single number within the sub-range, such as 1, 2, 3, 4, 5, or 6, which is applicable regardless of the ranges/scopes. In addition, whenever a numerical range is indicated in this article, it refers to a number (fraction or integer) including any reference within the range.

The present disclosure provides an aromatic amine compound, and a mixture, a composition, and an organic electronic device including the aromatic amine compound. In order to make the purpose(s), technical solutions, and effects of the present disclosure clear and definite, the following is a further detailed illustration of the present disclosure. It should be understood that the specific embodiments described here are only used to explain the present disclosure and are not intended to limit it.

In the present disclosure, “substituted” means that one or more hydrogen atoms in one substituted group are substituted by a substituent group.

In the present disclosure, a same substituent group at different substituent site may be independently selected from different groups. If a formula includes a plurality of R¹ groups, each of the R¹ groups may be independently selected from different groups. For example, in formula

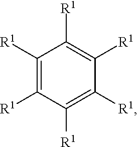

six R¹ groups of a benzene ring may be the same or different.

In the present disclosure, “substituted or unsubstituted” means that a defined group may be substituted or not be substituted. When the defined group is substituted, it can be understood that the defined group may be substituted by at least one substituent R. The substituent R is selected from, but not limited thereto: -D, a cyano group, an isocyano group, a nitro group, a halogen group, a C₁-C₃₀ alkyl group, a heterocyclic group with 3-20 ring atoms, an aromatic group with 6-20 ring atoms, a heteroaromatic group with 5-20 ring atoms, —NR′R″, a silane group, a carbonyl group, an alkoxycarbonyl group, an aryloxycarbonyl group, a carbamoyl group, a haloformyl group, a formyl group, an isocyanate group, a thiocyanate group, an isothiocyanate group, a hydroxyl group, and a trifluoromethyl group. The above-mentioned groups may further be substituted by acceptable substituent groups in the art. Understandably, R′ and R″ in the —NR′R″ are each independently selected from, but not limited thereto: -H, -D, a cyanogen group, an isocyano group, a nitro group, a halogen group, a C₁-C₁₀ alkyl group, a heterocyclic group with 3-20 ring atoms, an aromatic group with 6-20 ring atoms, and a heteroaromatic group with 5-20 ring atoms. In some embodiments, R′ and R″ are each independently selected from, but not limited thereto: -D, a cyano group, an isocyano group, a nitro group, a halogen group, a C₁-C₁₀ alkyl group, a heterocyclic group with 3-10 ring atoms, an aromatic group with 6-20 ring atoms, a heteroaromatic group with 5-20 ring atoms, a silane group, a carbonyl group, an alkoxycarbonyl group, an aryloxycarbonyl group, a carbamoyl group, a haloformyl group, a formyl group, an isocyanate group, a thiocyanate group, an isothiocyanate group, a hydroxyl group, and a trifluoromethyl group, and the above-mentioned groups may further be substituted by acceptable substituent groups in the art.

In the present disclosure, “a ring atom number” refers to a number of atoms constituting a ring of structural compounds (such as a monocyclic compound, a fused ring compound, a cross-linked compound, a carbon ring compound, and a heterocyclic compound) obtained by atomic bonding. In a ring substituted by a substituent group, atoms contained in the substituent group is not included in the atoms forming the ring. The same applies to the “number of ring atoms” described below unless otherwise specified. For example, a ring atom number of a benzene ring is 6, a ring atom number of a naphthalene ring is 10, and a ring atom number of a thiophene group is 5. Furthermore, unless otherwise specified, the ring atom is carbon atom, and the “heteroatom” is selected from N, S, or O.

In the present disclosure, “an aryl group or an aromatic group” refers to an aromatic hydrocarbon group derived from a basis of an aromatic ring compound removing an H. The aromatic ring compound may be an aromatic group with a single ring, a fused ring aromatic group, or a polycyclic aromatic group. For a polycyclic ring type, at least one ring is an aromatic ring system. For example, in some embodiments, “a substituted or unsubstituted aryl group with 6 to 40 ring atoms” refers to an aryl group containing 6 to 40 ring atoms. In some embodiments, “the substituted or unsubstituted aryl group with 6 to 30 ring atoms” refers to a substituted or unsubstituted aryl group containing 6 to 30 ring atoms. In some embodiments, “the substituted or unsubstituted aryl group with 6 to 18 ring atoms” refers to a substituted or unsubstituted aryl group containing 6 to 18 ring atoms. In some embodiments, “the substituted or unsubstituted aryl group with 6 to 14 ring atoms” refers to a substituted or unsubstituted aryl group containing 6 to 14 ring atoms, and the aryl group is optionally further substituted. Suitable examples include, but are not limited thereto: a phenyl group, a biphenyl group, a triphenyl group, a naphthyl group, an anthracyl group, a phenanthryl group, a fluoranthenyl group, a triphenylene group, a pyrenyl group, a perylene group, a tetraphenyl group, a fluorenyl group, a diphenyl group, an acenaphthenyl group, and derivatives thereof. Understandably, multiple aryl groups may further be disconnected by short non-aromatic units (for example, a non hydrogenium atoms contenting less than 10%, such as C, N, or O). In particular, an acenaphthene group, a fluorene group, a 9,9-diarylfluorene group, a triarylamine group, and a diaryl ether system may be further included in a definition of the aryl group.

In the present disclosure, “a heteroaryl group or a heteroaromatic group” refers a basis of an aryl group with at least one carbon atom substituted by a non-carbon atom, and the non-carbon atom may be N, O, S, or the like. For example, in some embodiments, “a substituted or unsubstituted heteroaryl group with 5 to 40 ring atoms” refers to a heteroaryl group containing 5 to 40 ring atoms. In some embodiments, “the substituted or unsubstituted heteroaryl group with 6 to 30 ring atoms” refers to a substituted or unsubstituted heteroaryl group containing 6 to 30 ring atoms. In some embodiments, “the substituted or unsubstituted heteroaryl group with 6 to 18 ring atoms” refers to a substituted or unsubstituted heteroaryl group containing 6 to 18 ring atoms. In some embodiments, “the substituted or unsubstituted heteroaryl group with 6 to 14 ring atoms” refers to a substituted or unsubstituted heteroaryl group containing 6 to 14 ring atoms, and the heteroaryl group are optionally further substituted. Suitable examples include, but are not limited thereto: a thiophene group, a furan group, a pyrrolyl group, a diazo group, a triazole group, an imidazolyl group, a pyridinyl group, a bipyridyl group, a pyrimidinyl group, a triazinyl group, an acridine group, a pyridazinyl group, a pyrazinyl group, a quinolinyl group, an isoquinolinyl group, a quinazolinyl group, a quinoxalinyl group, a phthalazinyl group, a pyridino pyrimidinyl group, a pyridino pyrazinyl group, a benzo thienyl group, a benzofuranyl group, an indolyl group, a pyrrolo imidazolyl group, a pyrrolo pyrrolyl group, a thiophenopyrrolyl group, a thiophenothiophenyl group, a furanopyrrolyl group, a furanofuranyl group, a thiophenofuranyl group, a benzoisoxazolyl group, a benzoisothiazolyl group, a benzimidazolyl group, an o-diaznaphthyl group, a phenanthryl group, a pyridinyl group, a quinazolinketone group, a dibenzothiophenyl group, a dibenzofuranyl group, a carbazolyl group, and derivatives thereof.

In the present disclosure, abbreviations of substituent groups are as follows: normal (n-), secondary (sec-), iso (i-), tertiary (tert-), ortho (o-), meta (m-), para (p-), methyl (Me), ethyl (Et), propyl (Pr), butyl (Bu), n-amyl (Am), hexyl (Hx), cyclohexyl (Cy).

In the present disclosure, “an alkyl group” may refer to a linear chain alkyl group, a branched chain alkyl group, or a cyclic alkyl group. A number of carbon atoms in the alkyl group may range from 1 to 50, 1 to 30, 1 to 20, 1 to 10, or 1 to 6. The term “a C₁₋₉ alkyl group” refers to the alkyl group with 1 to 9 carbon atoms, and “the C₁₋₉ alkyl group” may be a C₁ alkyl group, a C₂ alkyl group, a C₃ alkyl group, a C₄ alkyl group, a C₅ alkyl group, a C₆ alkyl group, a C₇ alkyl group, a C₈ alkyl group, or a C₉ alkyl group at each occurrence. Examples of the alkyl group include methyl, ethyl, n-propyl, isopropyl, n-butyl, sec-butyl, tert-butyl, isobutyl, 2-ethylbutyl, 3,3-dimethylbutyl, n-pentyl, isopentyl, neopentyl, tert-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butyl hexyl, cyclohexyl, 4-methylcyclohexyl 4-tert butylcyclohexyl, n-heptyl, 1-methylheptyl, 2,2-dimethylheptyl, 2-ethylheptyl, 2-butyl heptyl, n-octyl, tertoctyl, 2-ethyloctyl, 2-butyl octyl, 2-hexyloctyl, 3,7-dimethyloctyl, cyclooctyl, n-nonyl, n-decyl, adamantine, 2-ethyldecyl, 2-butyl decyl, 2-hexyldecyl, 2-octyldecyl, n-undecyl, n-dodecyl, 2-ethyldodecyl 2-butyl dodecyl, 2-hexyl dodecyl, 2-octyl dodecyl, n-tridecyl, n-tetradecyl, n-pentadecyl, n-hexadecyl, 2-ethyl hexadecyl, 2-butyl hexadecyl, 2-hexyl hexadecyl, 2-octyl hexadecyl, n-heptadecyl, n-octadecyl, n-octadecyl, n-eicosyl, 2-ethyl eicosyl, 2-butyl eicosyl, 2-hexyl eicosyl, 2-octyl eicosyl twenty one alkyl, twenty two alkyl, twenty three alkyl, twenty four alkyl, twenty five alkyl, twenty six alkyl, twenty seven alkyl, twenty eight alkyl, twenty nine alkyl, thirty alkyl, or the like.

In the present disclosure, “an amino group” refers to a derivative of the amine, has a feature of a group represented by formula —N(X)₂. X is independently selected from —H, a substituted or unsubstituted alkyl group, a substituted or unsubstituted cycloalkyl group, a substituted or unsubstituted heterocyclic group, or the like. Examples of amino groups includes —NH₂, —N(alkyl)₂, —NH (alkyl), —N(cycloalkyl)₂, —NH(cycloalkyl), —N(heterocyclic)₂, —NH (heterocyclic), —N(aryl)₂, —NH(aryl), —N(alkyl) (aryl), —N(alkyl) (heterocyclic), —N(cycloalkyl) (heterocyclic), —N(aryl) (heteroaryl), —N(alkyl) (heteroaryl), or the like.

In the present disclosure, the “*” connected to a single bond indicates a linking site, and the “* *” connected to a ring indicates a fused site.

In the present disclosure, when a linking site in a group is not specified, it means that any of connectable sites in the group may be selected as the linking site.

In the present disclosure, when a fused site in a group is not specified, it means that any of fusible sites in the group may be selected as the fused site. Preferably, two or more adjacent sites in the group are fused sites.

In the disclosure, a single bond connected to a substituent group and penetrated a corresponding ring indicates that the substituent group may be connected to any to any site of the ring. For example,

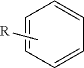

means that R may be connected substituent site of the benzene ring; and

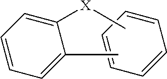

means that

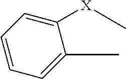

may be connected to any substituent site of the benzene ring to form a union ring.

The present disclosure provides an aromatic amine compound represented by formula (1):

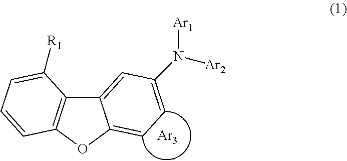

In formula (1), Ar₁ and Ar₂ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof. Ar₃ is selected from a substituted or unsubstituted aromatic group with 6 to 30 ring atoms or a substituted or unsubstituted heteroaromatic group with 5 to 30 ring atoms. R₁ is selected from substituted or unsubstituted methyl, substituted or unsubstituted ethyl, substituted or unsubstituted isopropyl, substituted or unsubstituted tertbutyl, substituted or unsubstituted cyclopentyl, substituted or unsubstituted cyclohexyl, or a substituted or unsubstituted adamantane group.

By fusing the alkyl group and the fused heterocycles containing oxygen in the aromatic amine compound, with the coordination of the aromatic amine, the present disclosure can effectively regulate the transmission of holes. When the aromatic amine compound is used as an electron barrier material in an electron barrier layer of an organic electronic device such as a red light OLED device, the charge transmission balance in the organic electronic device can be better regulated, improving the luminous efficiency and prolonging the lifespan of the organic electronic device.

In some embodiments, Ar₁ and Ar₂ are each independently selected from a substituted or unsubstituted aromatic group with 6 to 30 ring atoms or a substituted or unsubstituted heteroaromatic group with 5 to 30 ring atoms.

In some embodiments, Ar₁ and Ar₂ are each independently selected from a substituted or unsubstituted aromatic group with 6 to 20 ring atoms or a substituted or unsubstituted heteroaromatic group with 5 to 20 ring atoms.

In some embodiments, Ar₁ and Ar₂ are each independently selected from one of following substituted or unsubstituted groups:

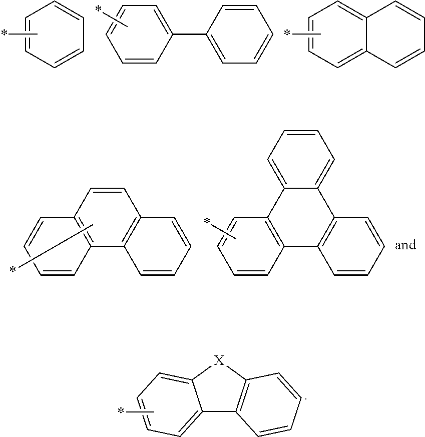

X is selected from NR₂, CR₃R₄, O, S, SiR₃R₄, or PR_{2.} R₂, R₃, and R₄ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof.

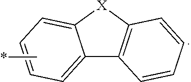

In some embodiments, at least one of Ar₁ and Ar₂ is X may be selected from NR₂, CR₃R₄, O, or S. R₂ is phenyl, R₃ and R₄ are each independently selected from methyl or phenyl.

In some embodiments, Ar₁ and Ar₂ are the same group.

In some embodiments, at least one of Ar₁ and Ar₂ is

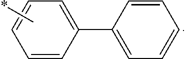

In some embodiments,

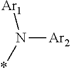

is selected from one of following structures:

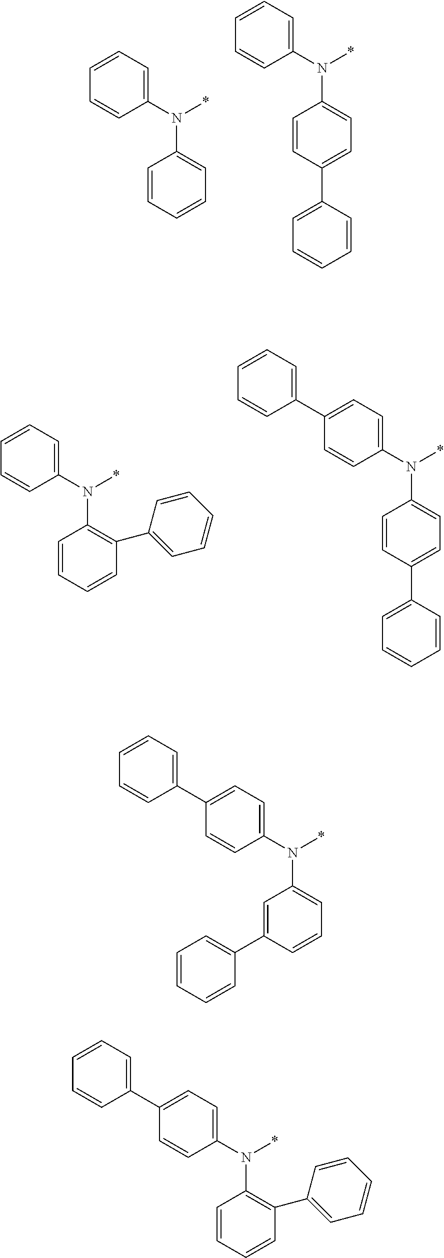 [SMILES: *N(c1ccccc1)c1ccccc1 ; *N(c1ccccc1)c1ccc(-c2ccccc2)cc1 ; *N(c1ccccc1)c1ccccc1-c1ccccc1 ; *N(c1ccc(-c2ccccc2)cc1)c1ccc(-c2ccccc2)cc1 ; *N(c1ccc(-c2ccccc2)cc1)c1cccc(-c2ccccc2)c1 ; *N(c1ccc(-c2ccccc2)cc1)c1ccccc1-c1ccccc1]

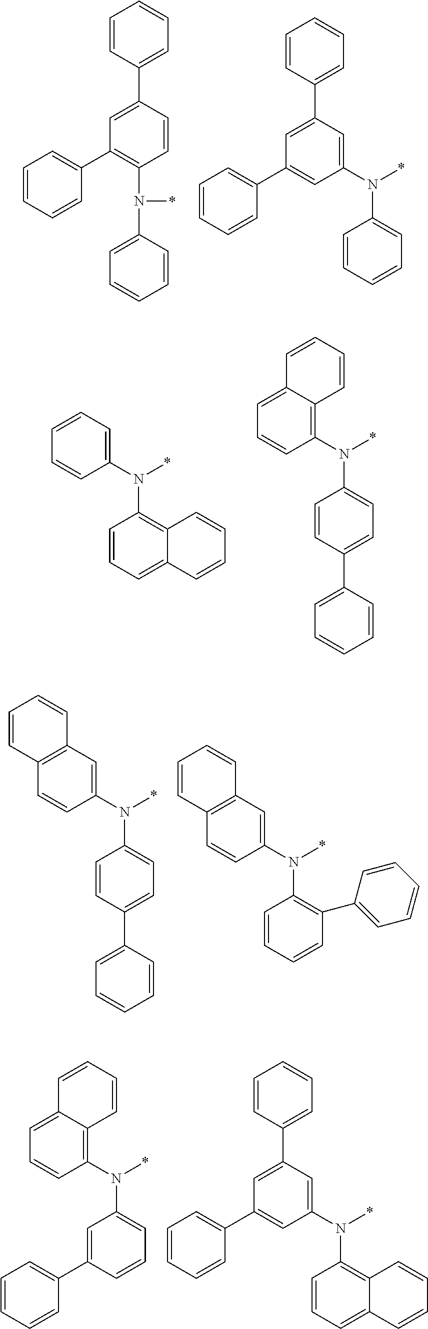

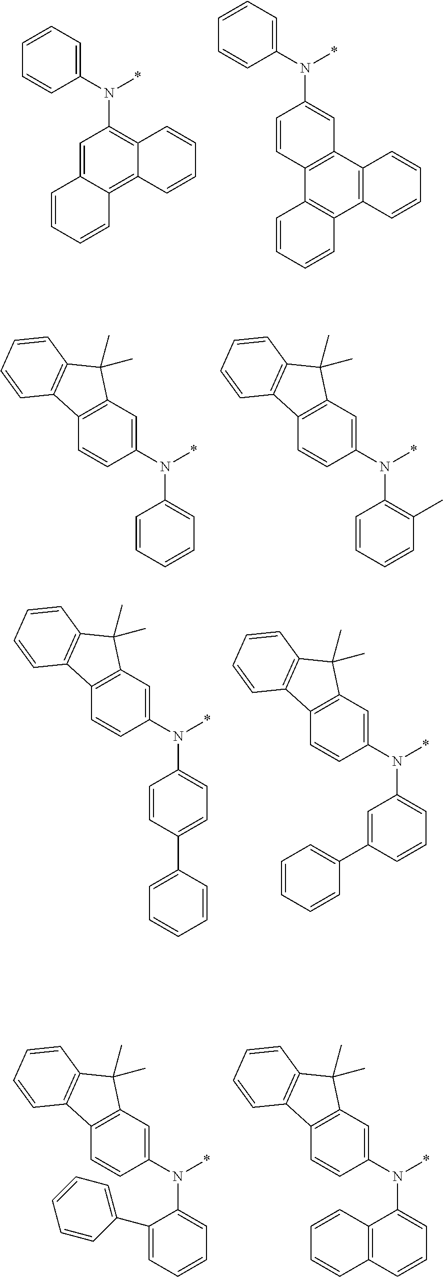

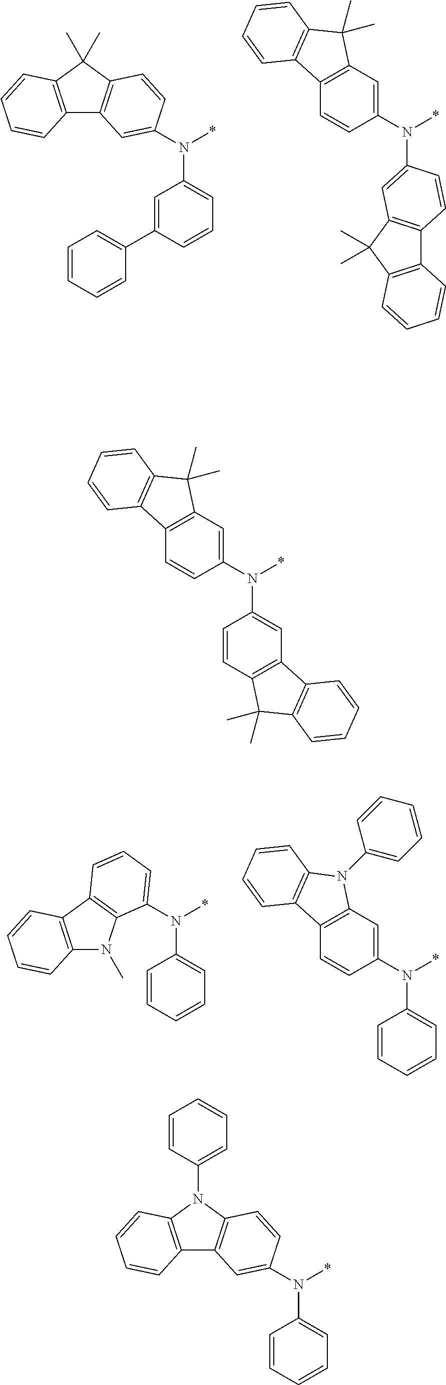

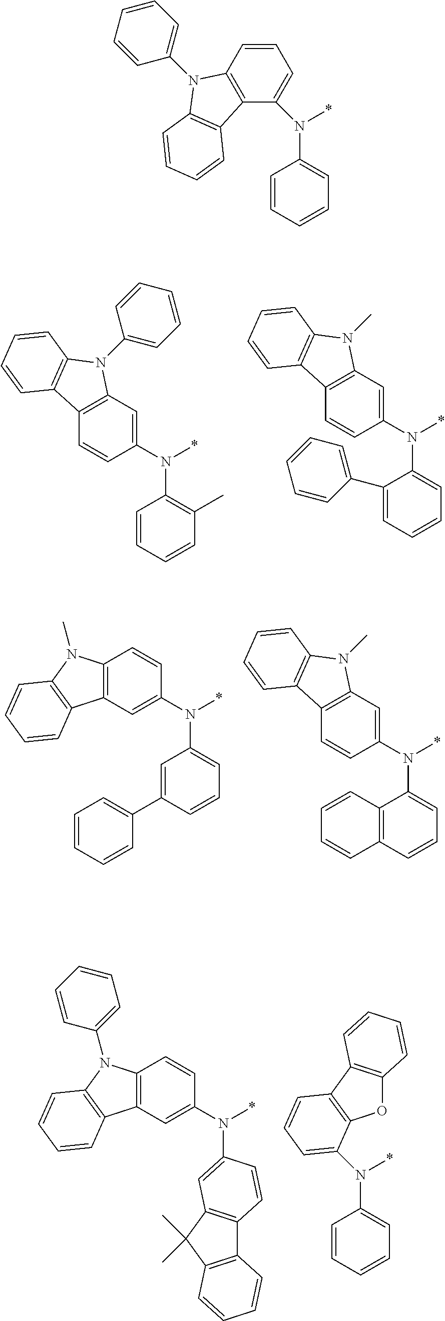

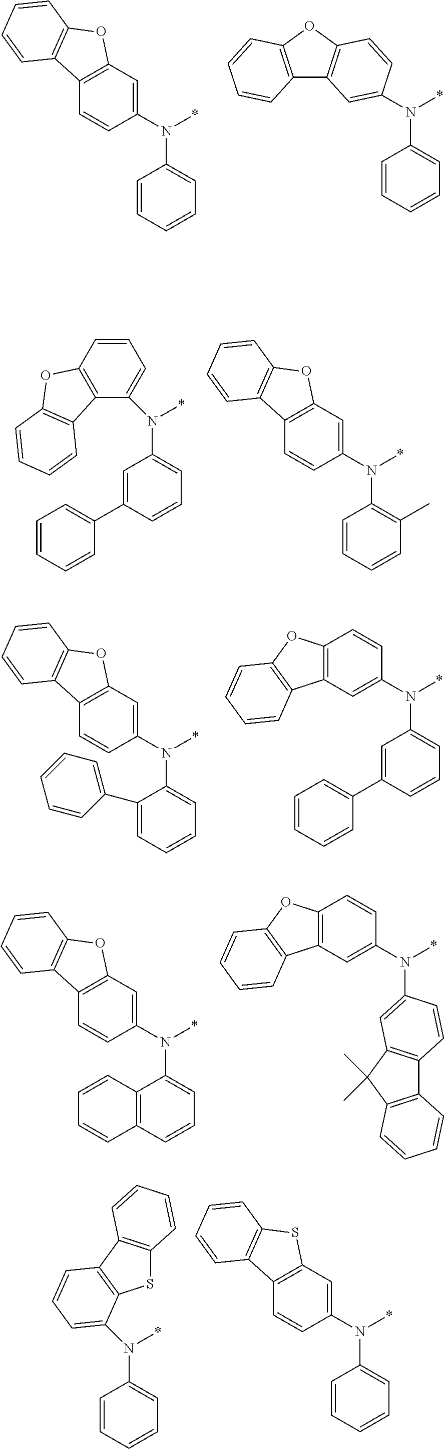

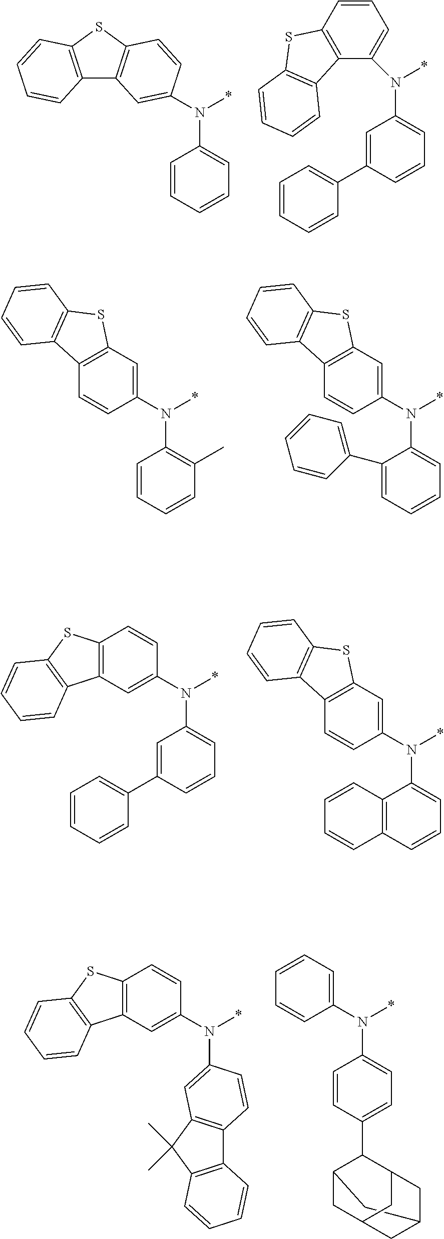

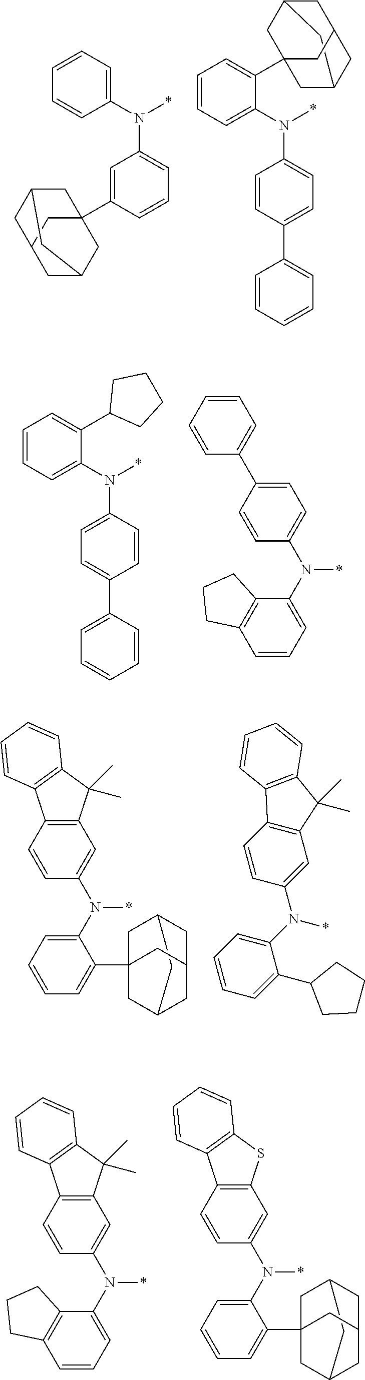

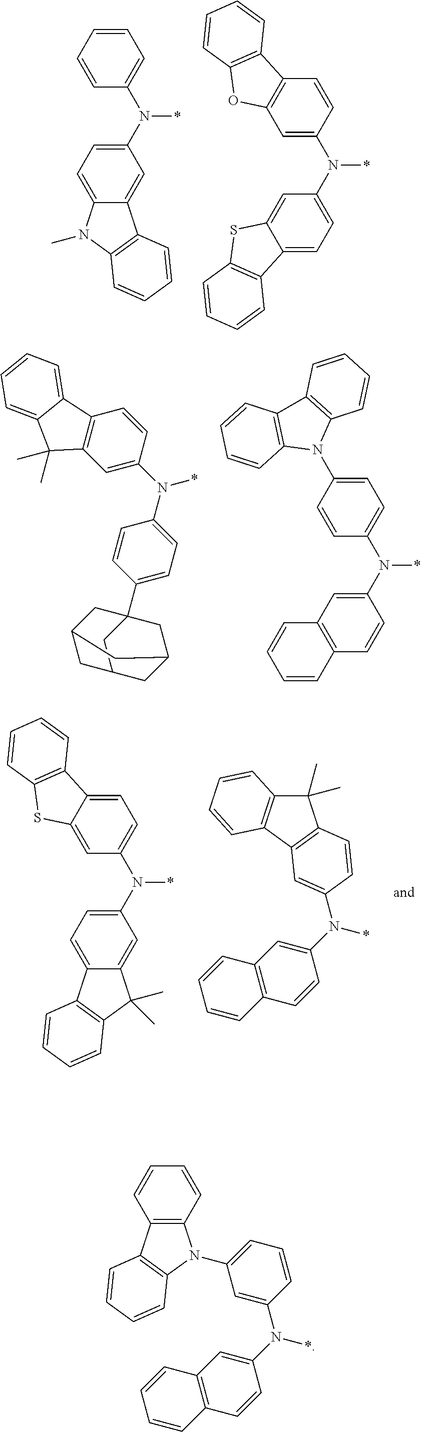

In some embodiments, Ar₃ is selected from a substituted or unsubstituted aromatic group with 6 to 20 ring atoms or a substituted or unsubstituted heteroaromatic group with 5 to 20 ring atoms.

In some embodiments, Ar₃ is selected from one of following groups:

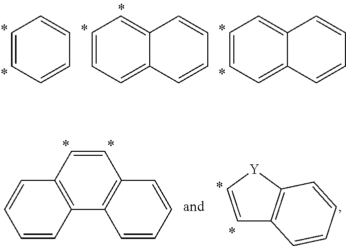

wherein Y is selected from NR₂, CR₃R₄, O, S, SiR₃R_{4,} or PR_{2.} R₂, R₃, and R₄ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof.

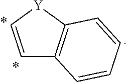

In some embodiments, Ar₃ is Y is selected from NR₂, CR₃R₄, O, or S. R₂-R₄ are each independently a substituted or unsubstituted C₁-C₂₀ alkyl group.

In some embodiments, Ar₃ is selected from one of following groups:

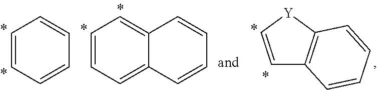

wherein Y is selected from O or S.

In some embodiments, R₁ is selected from one of following substituted or unsubstituted groups:

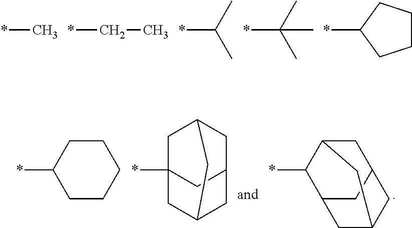

In some embodiments, when R₁ is selected from a substituted group mentioned above, R₁ is substituted by a deuterium atom.

In some embodiments, R₁ is selected from one of following substituted or unsubstituted groups:

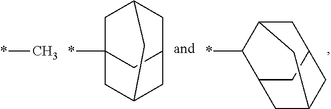

wherein R₁ is substituted by a deuterium atom when R₁ is one of substituted groups.

In some embodiments, at least one of Ar₁ and Ar₂ is substituted or unsubstituted

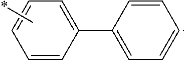

Ar₃ is phenyl. R₁ is substituted or unsubstituted methyl.

In some embodiments, R₁ is substituted methyl, and R₁ is substituted by a deuterium atom.

In some embodiments, the aromatic amine compound is selected from one of following structures:

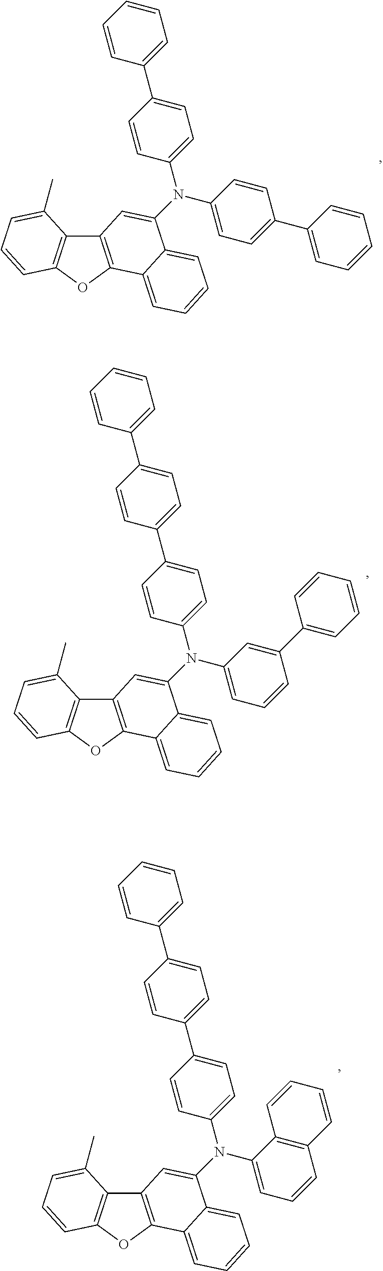 [SMILES: Cc1cccc2oc3c4ccccc4c(N(c4ccc(-c5ccccc5)cc4)c4ccc(-c5ccccc5)cc4)cc3c12 ; Cc1cccc2oc3c4ccccc4c(N(c4ccc(-c5ccc(-c6ccccc6)cc5)cc4)c4cccc(-c5ccccc5)c4)cc3c12 ; Cc1cccc2oc3c4ccccc4c(N(c4ccc(-c5ccc(-c6ccccc6)cc5)cc4)c4cccc5ccccc45)cc3c12]

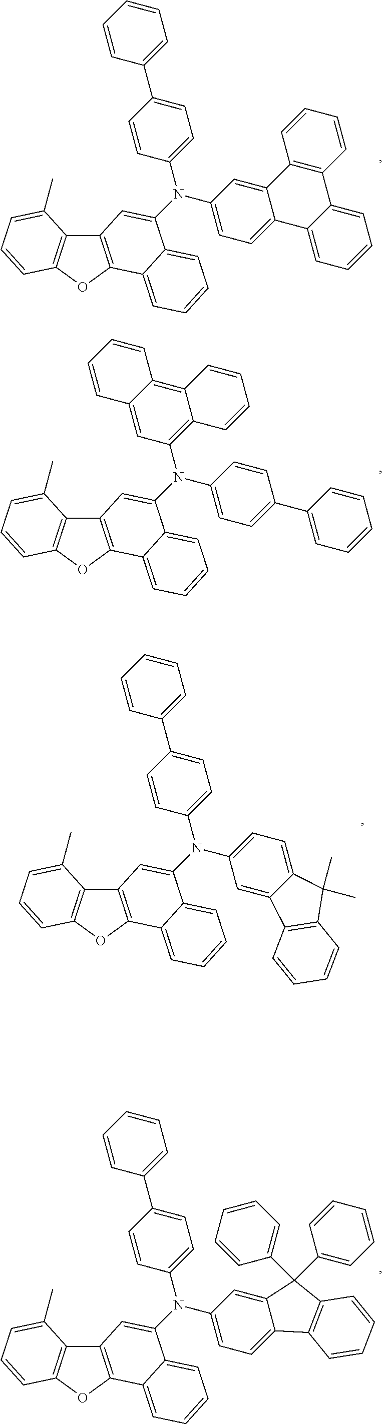

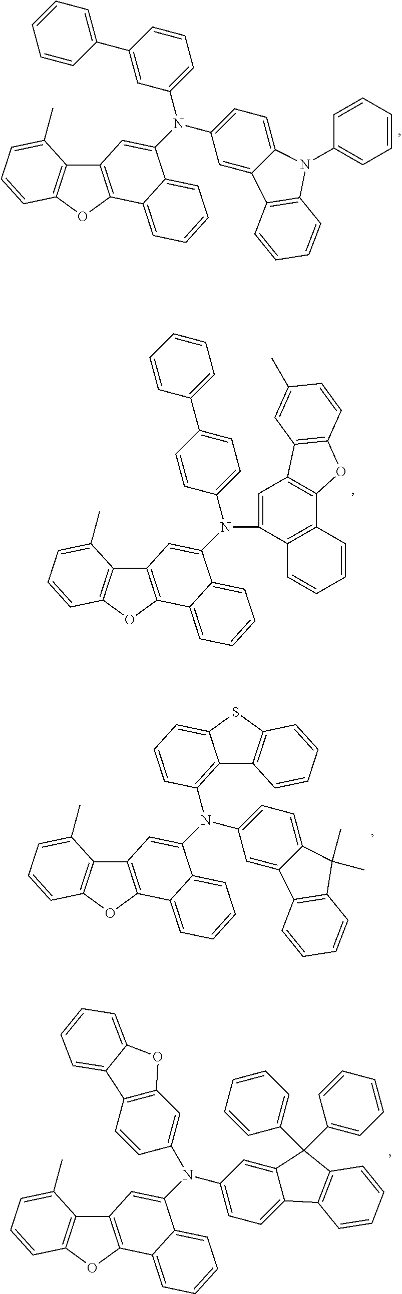 [SMILES: Cc1cccc2oc3c4ccccc4c(N(c4cccc(-c5ccccc5)c4)c4ccc5c(c4)c4ccccc4n5-c4ccccc4)cc3c12 ; Cc1ccc2oc3c4ccccc4c(N(c4ccc(-c5ccccc5)cc4)c4cc5c(oc6cccc(C)c65)c5ccccc45)cc3c2c1 ; Cc1cccc2oc3c4ccccc4c(N(c4ccc5c(c4)-c4ccccc4C5(C)C)c4cccc5sc6ccccc6c45)cc3c12 ; Cc1cccc2oc3c4ccccc4c(N(c4ccc5c(c4)C(c4ccccc4)(c4ccccc4)c4ccccc4-5)c4ccc5c(c4)oc4ccccc45)cc3c12]

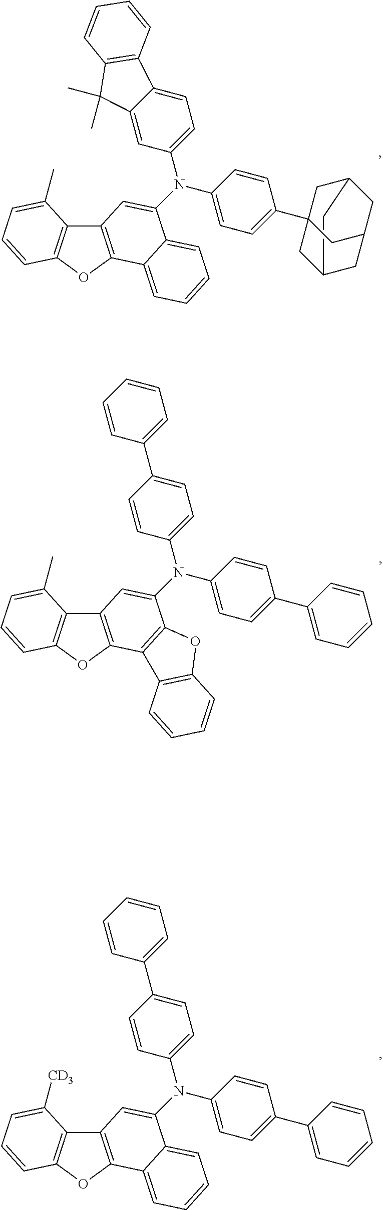 [SMILES: Cc1cccc2oc3c4ccccc4c(N(c4ccc(C56CC7CC(CC(C7)C5)C6)cc4)c4ccc5c(c4)C(C)(C)c4ccccc4-5)cc3c12 ; Cc1cccc2oc3c(cc(N(c4ccc(-c5ccccc5)cc4)c4ccc(-c5ccccc5)cc4)c4oc5ccccc5c43)c12 ; [2H]C([2H])([2H])c1cccc2oc3c4ccccc4c(N(c4ccc(-c5ccccc5)cc4)c4ccc(-c5ccccc5)cc4)cc3c12]

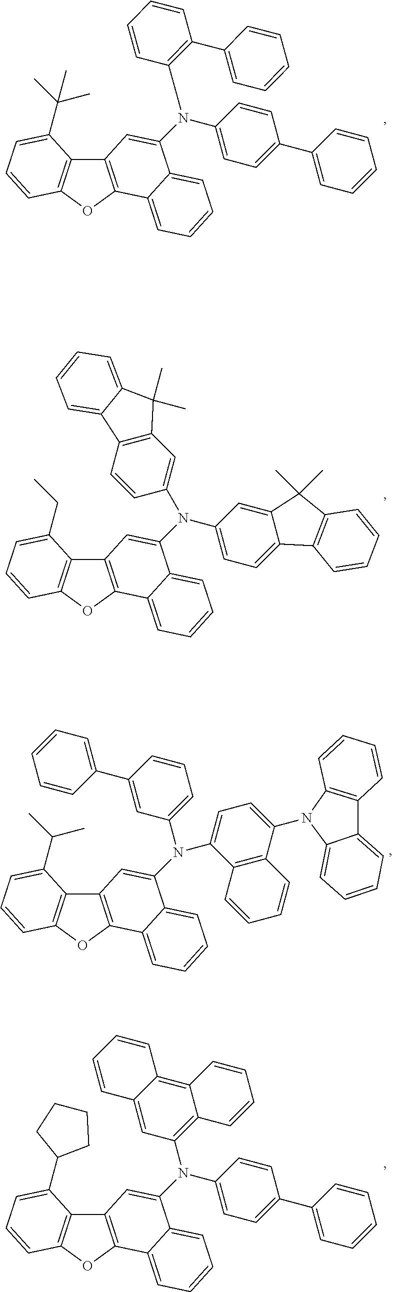 [SMILES: CC(C)(C)c1cccc2oc3c4ccccc4c(N(c4ccc(-c5ccccc5)cc4)c4ccccc4-c4ccccc4)cc3c12 ; CCc1cccc2oc3c4ccccc4c(N(c4ccc5c(c4)C(C)(C)c4ccccc4-5)c4ccc5c(c4)C(C)(C)c4ccccc4-5)cc3c12 ; CC(C)c1cccc2oc3c4ccccc4c(N(c4cccc(-c5ccccc5)c4)c4ccc(-n5c6ccccc6c6ccccc65)c5ccccc45)cc3c12 ; c1ccc(-c2ccc(N(c3cc4ccccc4c4ccccc34)c3cc4c(oc5cccc(C6CCCC6)c54)c4ccccc34)cc2)cc1]

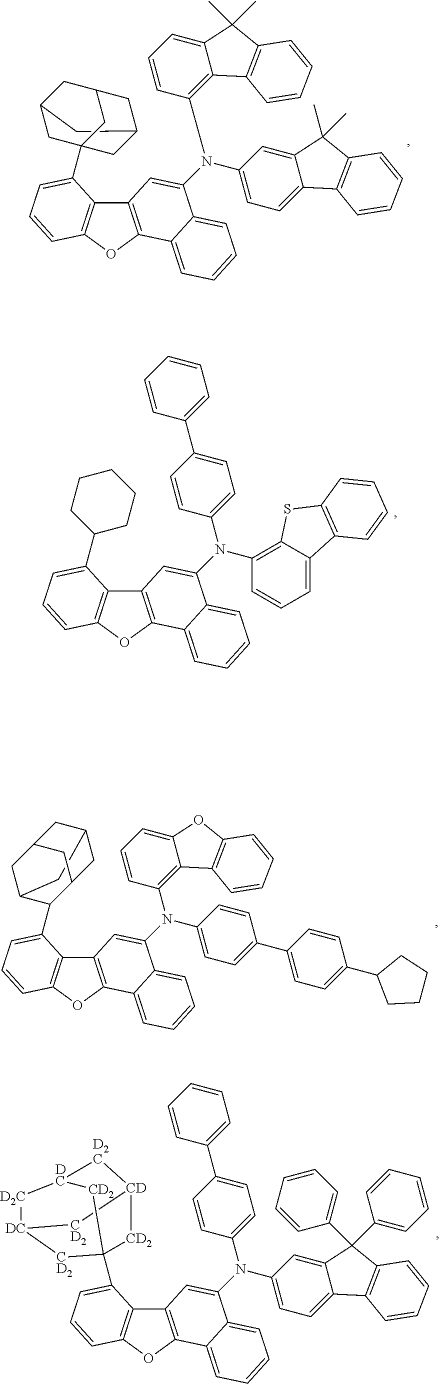

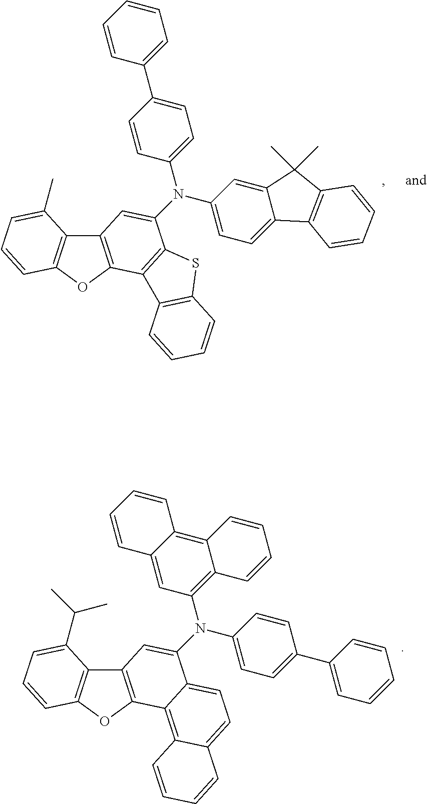 [SMILES: Cc1cccc2oc3c(cc(N(c4ccc(-c5ccccc5)cc4)c4ccc5c(c4)C(C)(C)c4ccccc4-5)c4sc5ccccc5c43)c12 ; CC(C)c1cccc2oc3c(cc(N(c4ccc(-c5ccccc5)cc4)c4cc5ccccc5c5ccccc45)c4ccc5ccccc5c43)c12]

In some embodiments, the aromatic amine compound is selected from but not limited to one of following structures:

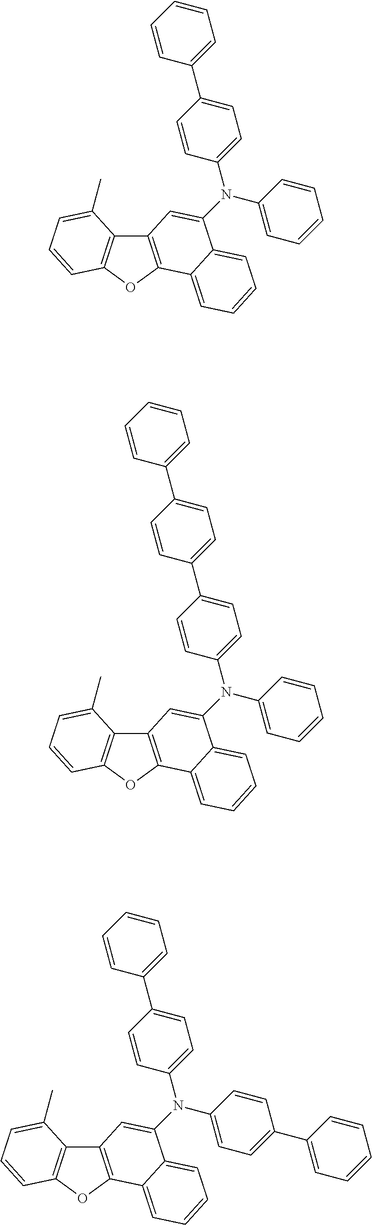 [SMILES: Cc1cccc2oc3c4ccccc4c(N(c4ccccc4)c4ccc(-c5ccccc5)cc4)cc3c12 ; Cc1cccc2oc3c4ccccc4c(N(c4ccccc4)c4ccc(-c5ccc(-c6ccccc6)cc5)cc4)cc3c12 ; Cc1cccc2oc3c4ccccc4c(N(c4ccc(-c5ccccc5)cc4)c4ccc(-c5ccccc5)cc4)cc3c12]

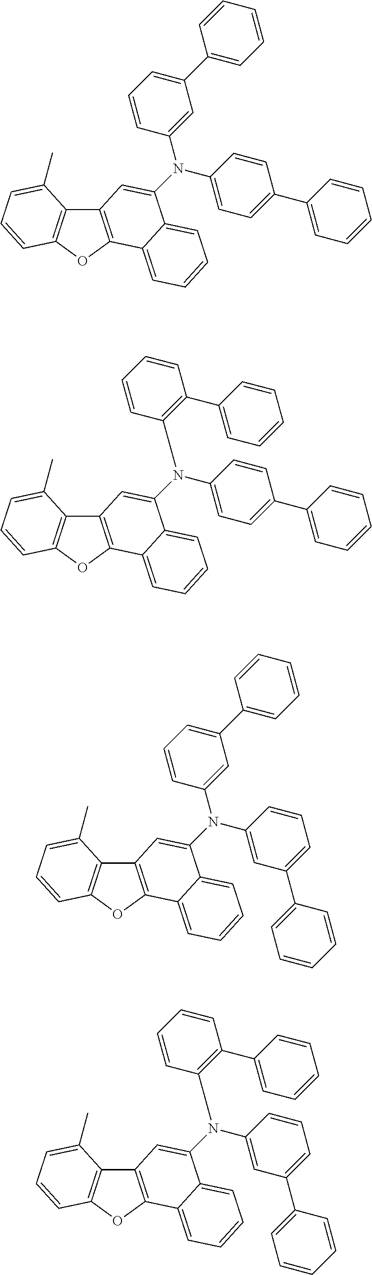 [SMILES: Cc1cccc2oc3c4ccccc4c(N(c4ccc(-c5ccccc5)cc4)c4cccc(-c5ccccc5)c4)cc3c12 ; Cc1cccc2oc3c4ccccc4c(N(c4ccc(-c5ccccc5)cc4)c4ccccc4-c4ccccc4)cc3c12 ; Cc1cccc2oc3c4ccccc4c(N(c4cccc(-c5ccccc5)c4)c4cccc(-c5ccccc5)c4)cc3c12 ; Cc1cccc2oc3c4ccccc4c(N(c4cccc(-c5ccccc5)c4)c4ccccc4-c4ccccc4)cc3c12]

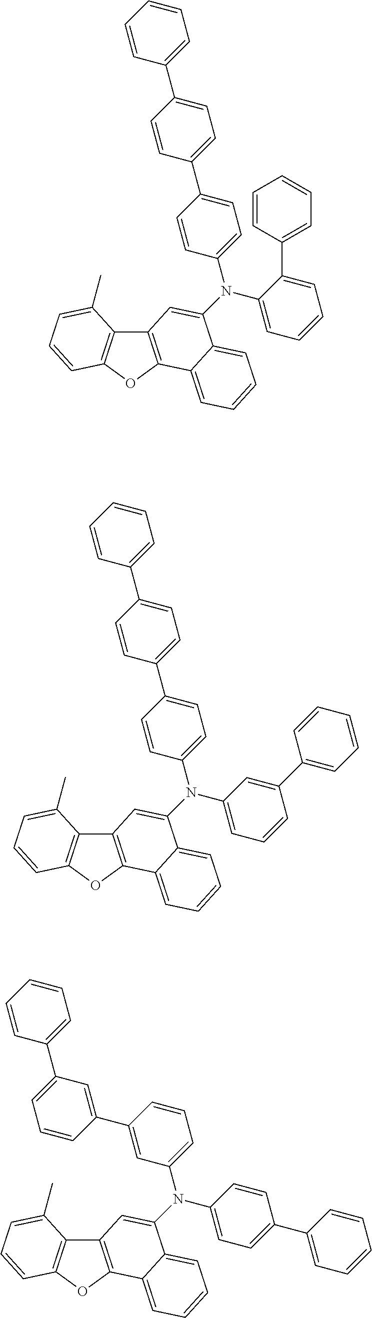 [SMILES: Cc1cccc2oc3c4ccccc4c(N(c4ccc(-c5ccc(-c6ccccc6)cc5)cc4)c4ccccc4-c4ccccc4)cc3c12 ; Cc1cccc2oc3c4ccccc4c(N(c4ccc(-c5ccc(-c6ccccc6)cc5)cc4)c4cccc(-c5ccccc5)c4)cc3c12 ; Cc1cccc2oc3c4ccccc4c(N(c4ccc(-c5ccccc5)cc4)c4cccc(-c5cccc(-c6ccccc6)c5)c4)cc3c12]

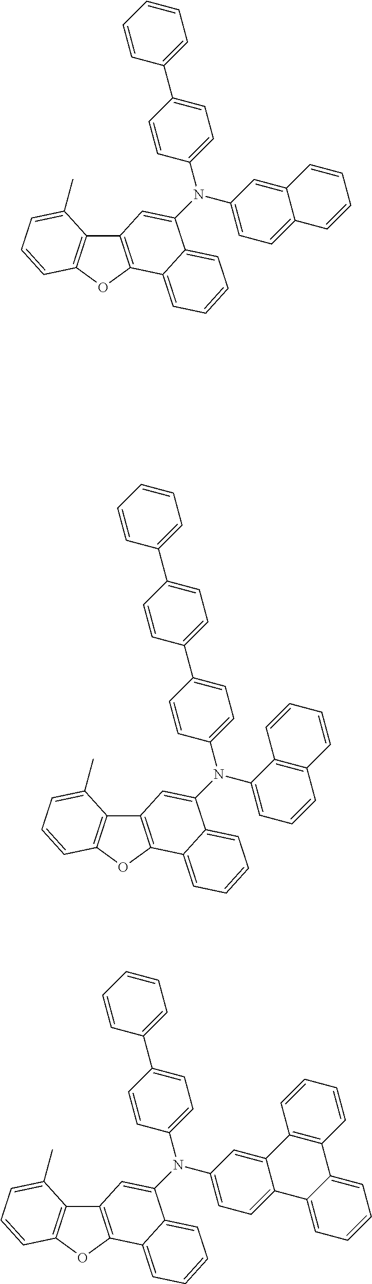 [SMILES: Cc1cccc2oc3c4ccccc4c(N(c4ccc(-c5ccccc5)cc4)c4ccc5ccccc5c4)cc3c12 ; Cc1cccc2oc3c4ccccc4c(N(c4ccc(-c5ccc(-c6ccccc6)cc5)cc4)c4cccc5ccccc45)cc3c12 ; Cc1cccc2oc3c4ccccc4c(N(c4ccc(-c5ccccc5)cc4)c4ccc5c6ccccc6c6ccccc6c5c4)cc3c12]

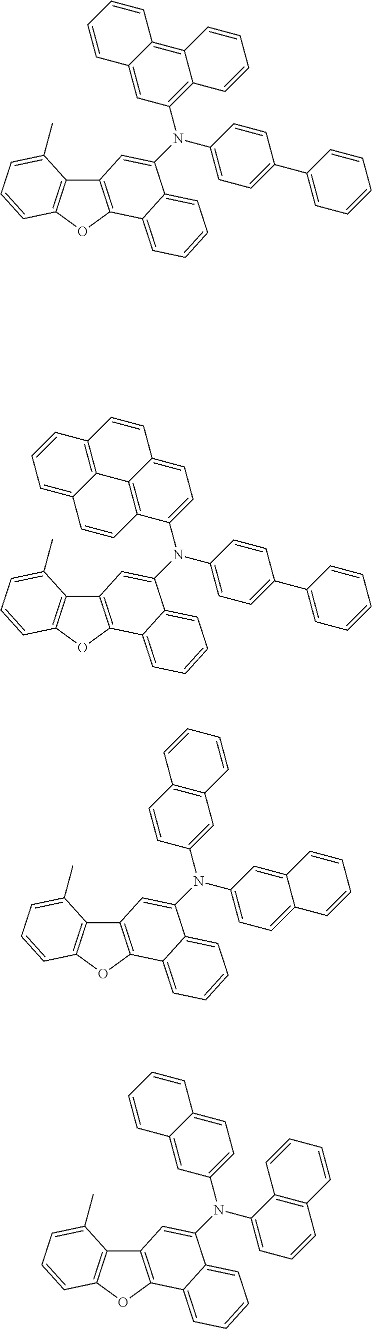 [SMILES: Cc1cccc2oc3c4ccccc4c(N(c4ccc(-c5ccccc5)cc4)c4cc5ccccc5c5ccccc45)cc3c12 ; Cc1cccc2oc3c4ccccc4c(N(c4ccc(-c5ccccc5)cc4)c4ccc5ccc6cccc7ccc4c5c67)cc3c12 ; Cc1cccc2oc3c4ccccc4c(N(c4ccc5ccccc5c4)c4ccc5ccccc5c4)cc3c12 ; Cc1cccc2oc3c4ccccc4c(N(c4ccc5ccccc5c4)c4cccc5ccccc45)cc3c12]

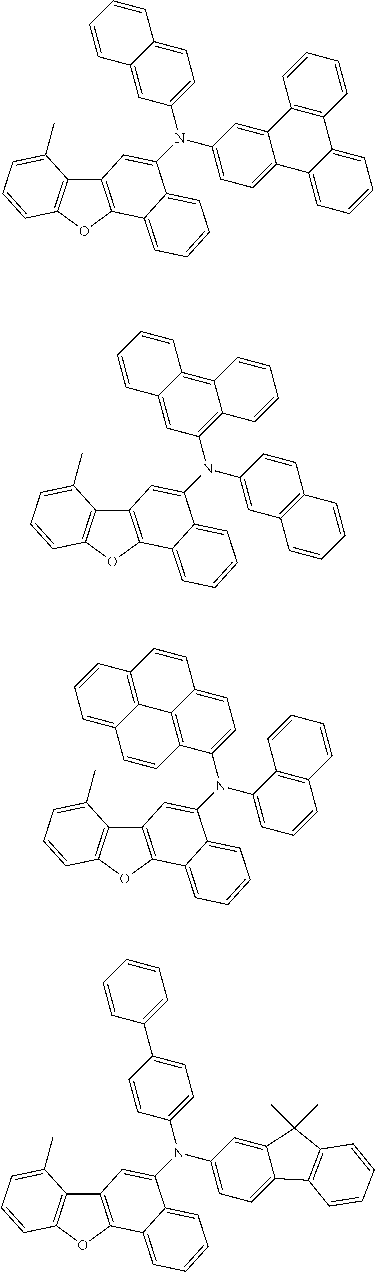 [SMILES: Cc1cccc2oc3c4ccccc4c(N(c4ccc5ccccc5c4)c4ccc5c6ccccc6c6ccccc6c5c4)cc3c12 ; Cc1cccc2oc3c4ccccc4c(N(c4ccc5ccccc5c4)c4cc5ccccc5c5ccccc45)cc3c12 ; Cc1cccc2oc3c4ccccc4c(N(c4cccc5ccccc45)c4ccc5ccc6cccc7ccc4c5c67)cc3c12 ; Cc1cccc2oc3c4ccccc4c(N(c4ccc(-c5ccccc5)cc4)c4ccc5c(c4)C(C)(C)c4ccccc4-5)cc3c12]

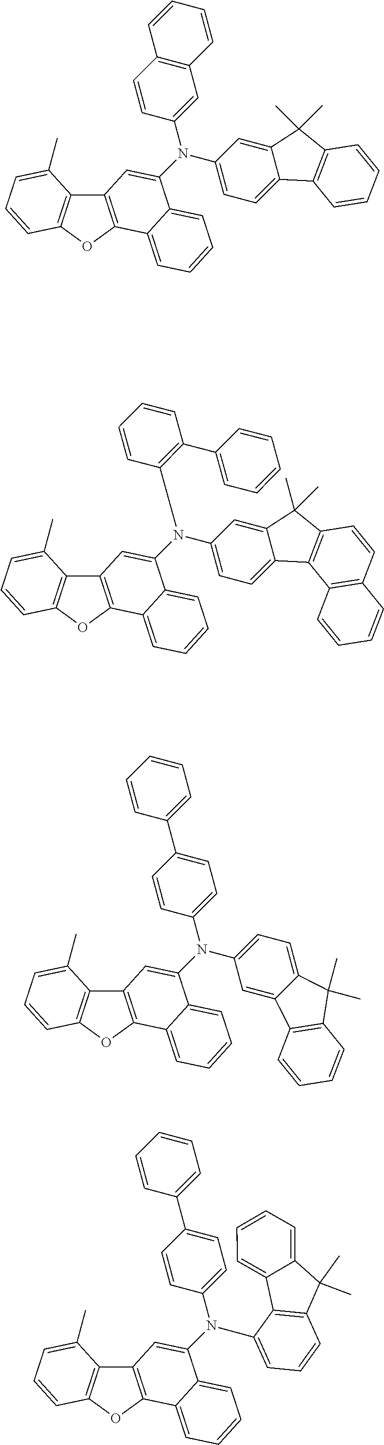 [SMILES: Cc1cccc2oc3c4ccccc4c(N(c4ccc5c(c4)C(C)(C)c4ccccc4-5)c4ccc5ccccc5c4)cc3c12 ; Cc1cccc2oc3c4ccccc4c(N(c4ccc5c(c4)C(C)(C)c4ccc6ccccc6c4-5)c4ccccc4-c4ccccc4)cc3c12 ; Cc1cccc2oc3c4ccccc4c(N(c4ccc(-c5ccccc5)cc4)c4ccc5c(c4)-c4ccccc4C5(C)C)cc3c12 ; Cc1cccc2oc3c4ccccc4c(N(c4ccc(-c5ccccc5)cc4)c4cccc5c4-c4ccccc4C5(C)C)cc3c12]

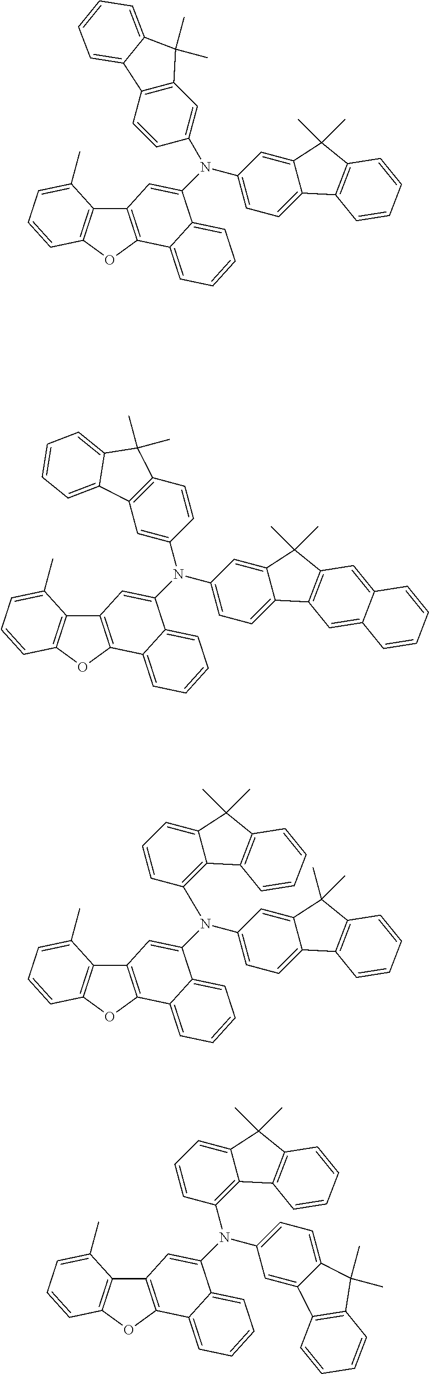 [SMILES: Cc1cccc2oc3c4ccccc4c(N(c4ccc5c(c4)C(C)(C)c4ccccc4-5)c4ccc5c(c4)C(C)(C)c4ccccc4-5)cc3c12 ; Cc1cccc2oc3c4ccccc4c(N(c4ccc5c(c4)-c4ccccc4C5(C)C)c4ccc5c(c4)C(C)(C)c4cc6ccccc6cc4-5)cc3c12 ; Cc1cccc2oc3c4ccccc4c(N(c4ccc5c(c4)C(C)(C)c4ccccc4-5)c4cccc5c4-c4ccccc4C5(C)C)cc3c12 ; Cc1cccc2oc3c4ccccc4c(N(c4ccc5c(c4)-c4ccccc4C5(C)C)c4cccc5c4-c4ccccc4C5(C)C)cc3c12]

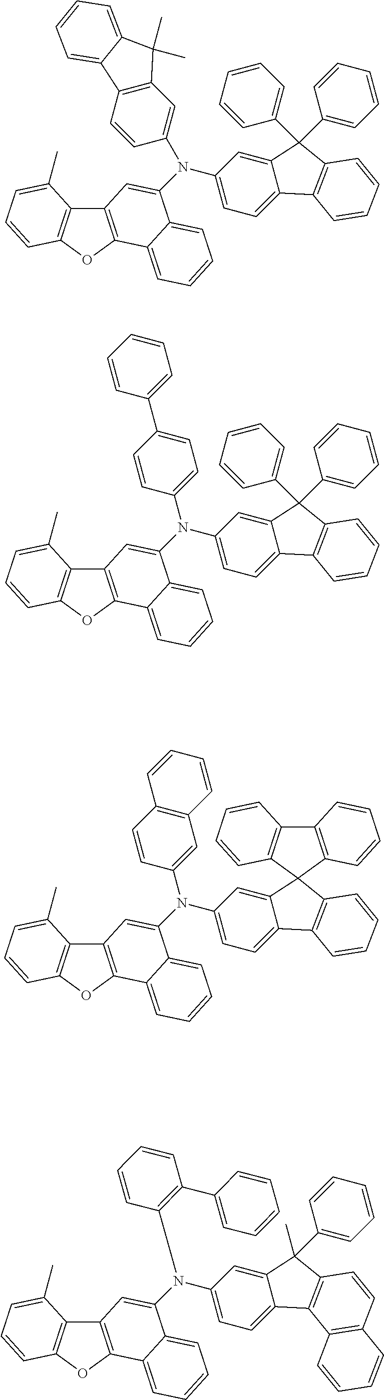 [SMILES: Cc1cccc2oc3c4ccccc4c(N(c4ccc5c(c4)C(C)(C)c4ccccc4-5)c4ccc5c(c4)C(c4ccccc4)(c4ccccc4)c4ccccc4-5)cc3c12 ; Cc1cccc2oc3c4ccccc4c(N(c4ccc(-c5ccccc5)cc4)c4ccc5c(c4)C(c4ccccc4)(c4ccccc4)c4ccccc4-5)cc3c12 ; Cc1cccc2oc3c4ccccc4c(N(c4ccc5c(c4)C4(c6ccccc6-c6ccccc64)c4ccccc4-5)c4ccc5ccccc5c4)cc3c12 ; Cc1cccc2oc3c4ccccc4c(N(c4ccc5c(c4)C(C)(c4ccccc4)c4ccc6ccccc6c4-5)c4ccccc4-c4ccccc4)cc3c12]

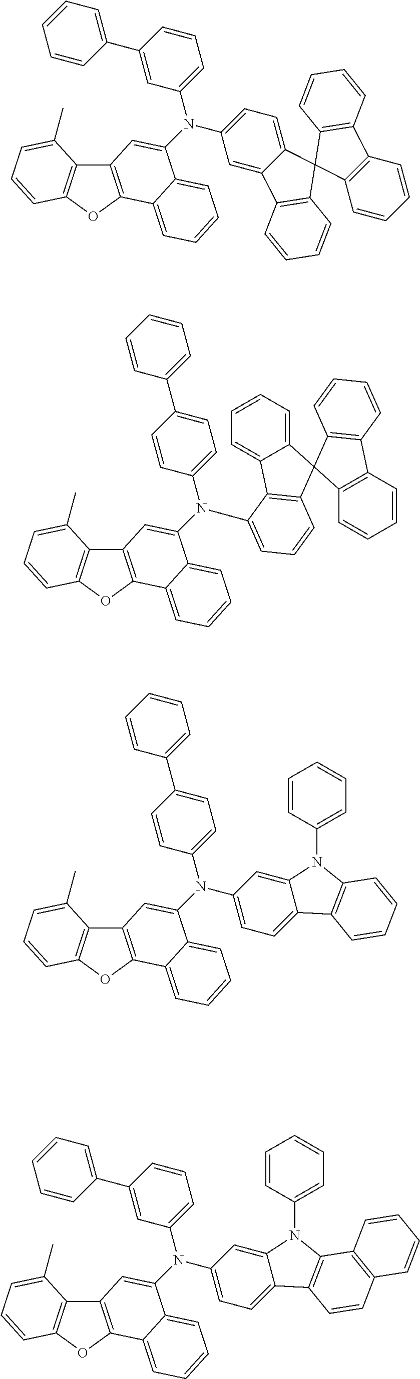 [SMILES: Cc1cccc2oc3c4ccccc4c(N(c4cccc(-c5ccccc5)c4)c4ccc5c(c4)-c4ccccc4C54c5ccccc5-c5ccccc54)cc3c12 ; Cc1cccc2oc3c4ccccc4c(N(c4ccc(-c5ccccc5)cc4)c4cccc5c4-c4ccccc4C54c5ccccc5-c5ccccc54)cc3c12 ; Cc1cccc2oc3c4ccccc4c(N(c4ccc(-c5ccccc5)cc4)c4ccc5c6ccccc6n(-c6ccccc6)c5c4)cc3c12 ; Cc1cccc2oc3c4ccccc4c(N(c4cccc(-c5ccccc5)c4)c4ccc5c6ccc7ccccc7c6n(-c6ccccc6)c5c4)cc3c12]

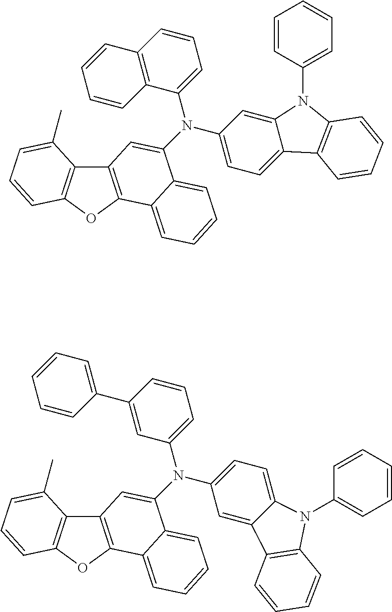 [SMILES: Cc1cccc2oc3c4ccccc4c(N(c4ccc5c6ccccc6n(-c6ccccc6)c5c4)c4cccc5ccccc45)cc3c12 ; Cc1cccc2oc3c4ccccc4c(N(c4cccc(-c5ccccc5)c4)c4ccc5c(c4)c4ccccc4n5-c4ccccc4)cc3c12]

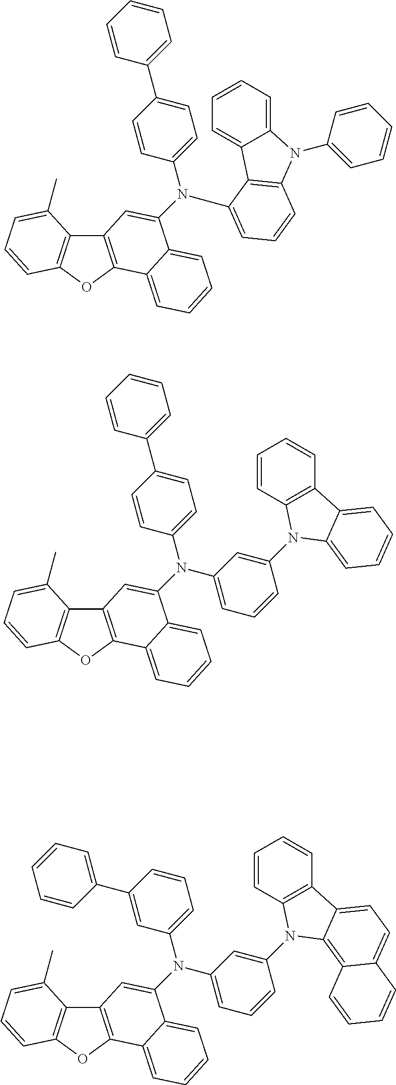 [SMILES: Cc1cccc2oc3c4ccccc4c(N(c4ccc(-c5ccccc5)cc4)c4cccc5c4c4ccccc4n5-c4ccccc4)cc3c12 ; Cc1cccc2oc3c4ccccc4c(N(c4ccc(-c5ccccc5)cc4)c4cccc(-n5c6ccccc6c6ccccc65)c4)cc3c12 ; Cc1cccc2oc3c4ccccc4c(N(c4cccc(-c5ccccc5)c4)c4cccc(-n5c6ccccc6c6ccc7ccccc7c65)c4)cc3c12]

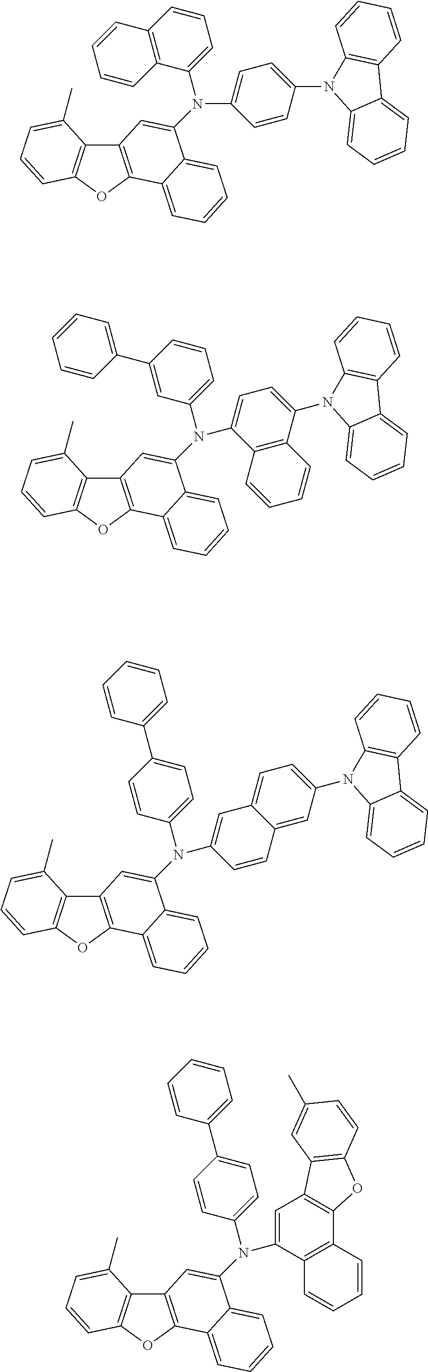 [SMILES: Cc1cccc2oc3c4ccccc4c(N(c4ccc(-n5c6ccccc6c6ccccc65)cc4)c4cccc5ccccc45)cc3c12 ; Cc1cccc2oc3c4ccccc4c(N(c4cccc(-c5ccccc5)c4)c4ccc(-n5c6ccccc6c6ccccc65)c5ccccc45)cc3c12 ; Cc1cccc2oc3c4ccccc4c(N(c4ccc(-c5ccccc5)cc4)c4ccc5cc(-n6c7ccccc7c7ccccc76)ccc5c4)cc3c12 ; Cc1ccc2oc3c4ccccc4c(N(c4ccc(-c5ccccc5)cc4)c4cc5c(oc6cccc(C)c65)c5ccccc45)cc3c2c1]

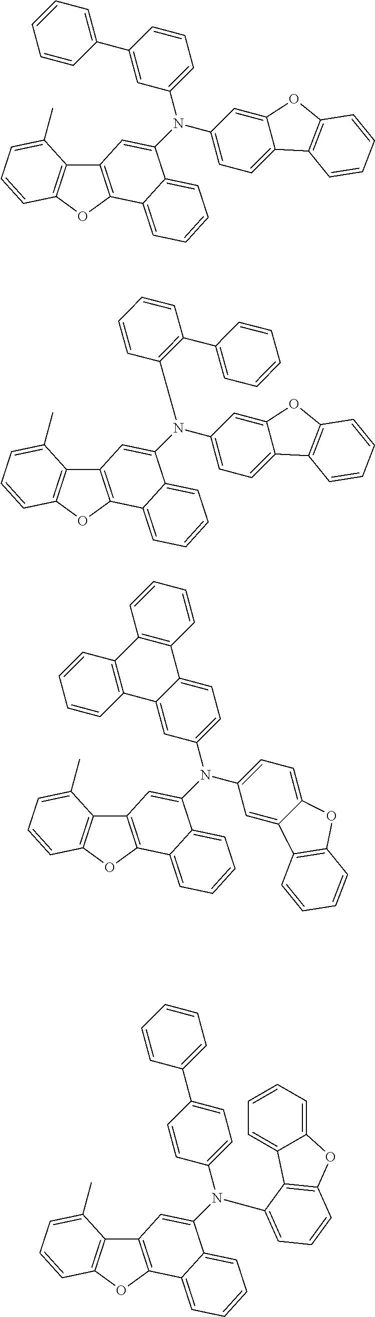

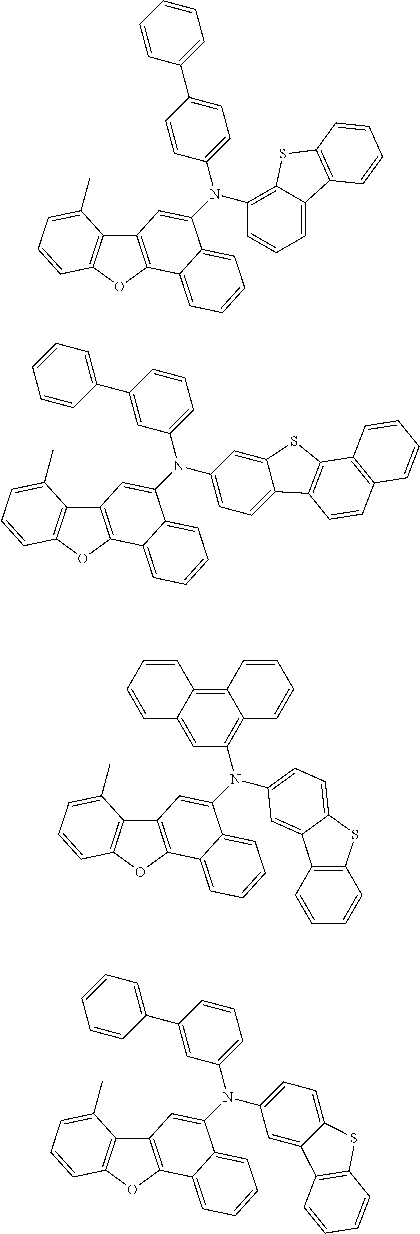 [SMILES: Cc1cccc2oc3c4ccccc4c(N(c4ccc(-c5ccccc5)cc4)c4cccc5c4sc4ccccc45)cc3c12 ; Cc1cccc2oc3c4ccccc4c(N(c4cccc(-c5ccccc5)c4)c4ccc5c(c4)sc4c6ccccc6ccc54)cc3c12 ; Cc1cccc2oc3c4ccccc4c(N(c4ccc5sc6ccccc6c5c4)c4cc5ccccc5c5ccccc45)cc3c12 ; Cc1cccc2oc3c4ccccc4c(N(c4cccc(-c5ccccc5)c4)c4ccc5sc6ccccc6c5c4)cc3c12]

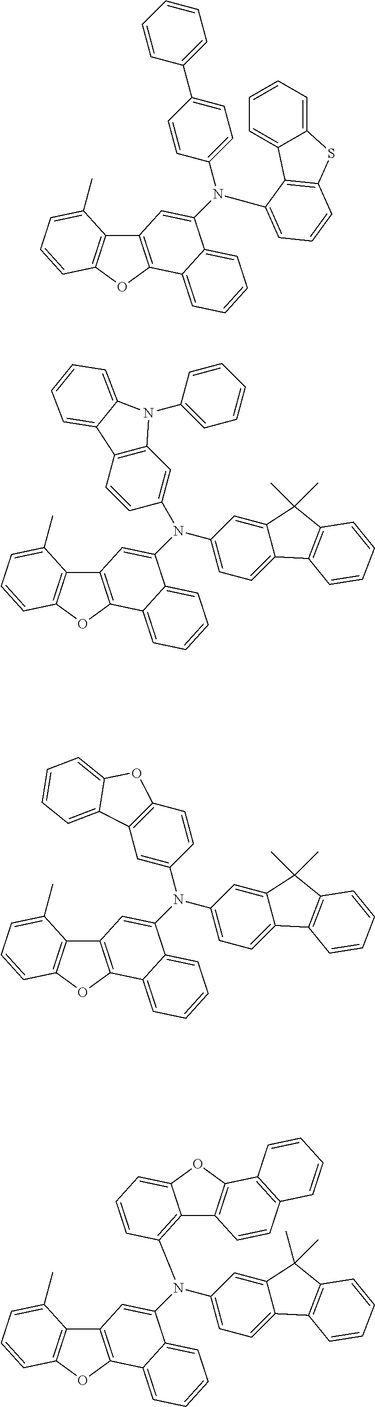 [SMILES: Cc1cccc2oc3c4ccccc4c(N(c4ccc(-c5ccccc5)cc4)c4cccc5sc6ccccc6c45)cc3c12 ; Cc1cccc2oc3c4ccccc4c(N(c4ccc5c(c4)C(C)(C)c4ccccc4-5)c4ccc5c6ccccc6n(-c6ccccc6)c5c4)cc3c12 ; Cc1cccc2oc3c4ccccc4c(N(c4ccc5c(c4)C(C)(C)c4ccccc4-5)c4ccc5oc6ccccc6c5c4)cc3c12 ; Cc1cccc2oc3c4ccccc4c(N(c4ccc5c(c4)C(C)(C)c4ccccc4-5)c4cccc5oc6c7ccccc7ccc6c45)cc3c12]

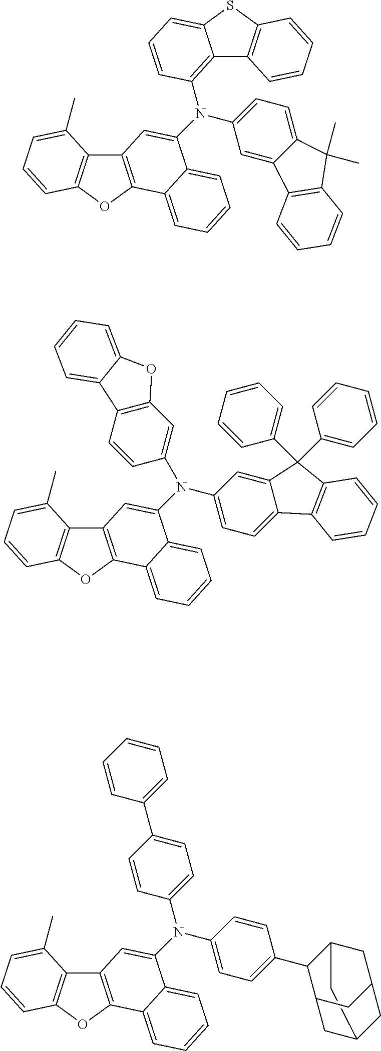 [SMILES: Cc1cccc2oc3c4ccccc4c(N(c4ccc5c(c4)-c4ccccc4C5(C)C)c4cccc5sc6ccccc6c45)cc3c12 ; Cc1cccc2oc3c4ccccc4c(N(c4ccc5c(c4)C(c4ccccc4)(c4ccccc4)c4ccccc4-5)c4ccc5c(c4)oc4ccccc45)cc3c12 ; Cc1cccc2oc3c4ccccc4c(N(c4ccc(-c5ccccc5)cc4)c4ccc(C5C6CC7CC(C6)CC5C7)cc4)cc3c12]

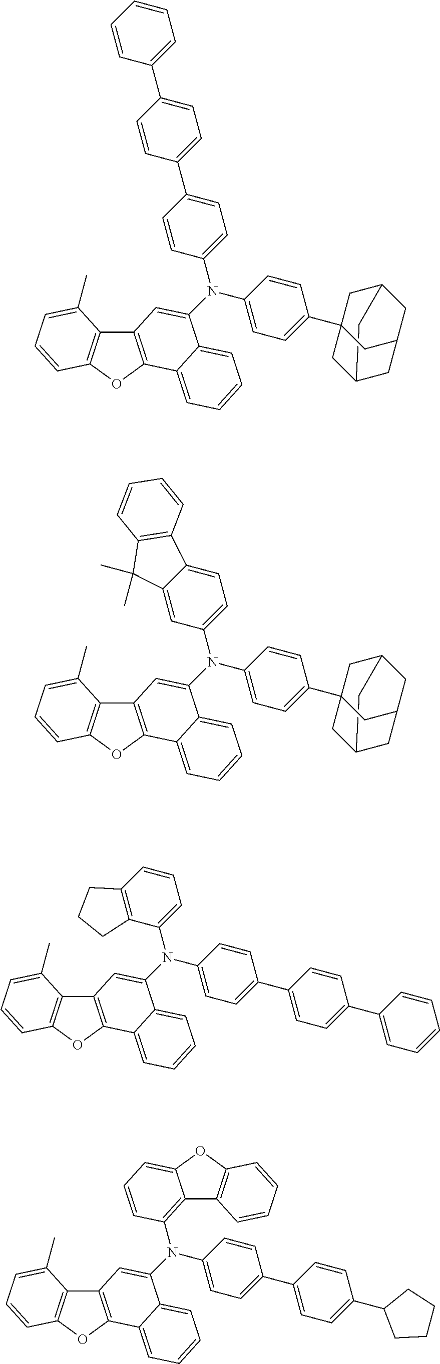 [SMILES: Cc1cccc2oc3c4ccccc4c(N(c4ccc(-c5ccc(-c6ccccc6)cc5)cc4)c4ccc(C56CC7CC(CC(C7)C5)C6)cc4)cc3c12 ; Cc1cccc2oc3c4ccccc4c(N(c4ccc(C56CC7CC(CC(C7)C5)C6)cc4)c4ccc5c(c4)C(C)(C)c4ccccc4-5)cc3c12 ; Cc1cccc2oc3c4ccccc4c(N(c4ccc(-c5ccc(-c6ccccc6)cc5)cc4)c4cccc5c4CCC5)cc3c12 ; Cc1cccc2oc3c4ccccc4c(N(c4ccc(-c5ccc(C6CCCC6)cc5)cc4)c4cccc5oc6ccccc6c45)cc3c12]

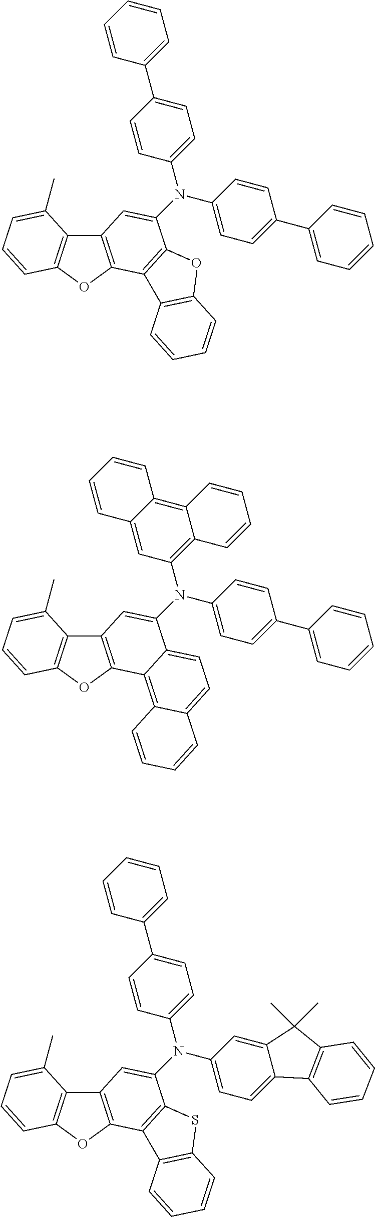 [SMILES: Cc1cccc2oc3c(cc(N(c4ccc(-c5ccccc5)cc4)c4ccc(-c5ccccc5)cc4)c4oc5ccccc5c43)c12 ; Cc1cccc2oc3c(cc(N(c4ccc(-c5ccccc5)cc4)c4cc5ccccc5c5ccccc45)c4ccc5ccccc5c43)c12 ; Cc1cccc2oc3c(cc(N(c4ccc(-c5ccccc5)cc4)c4ccc5c(c4)C(C)(C)c4ccccc4-5)c4sc5ccccc5c43)c12]

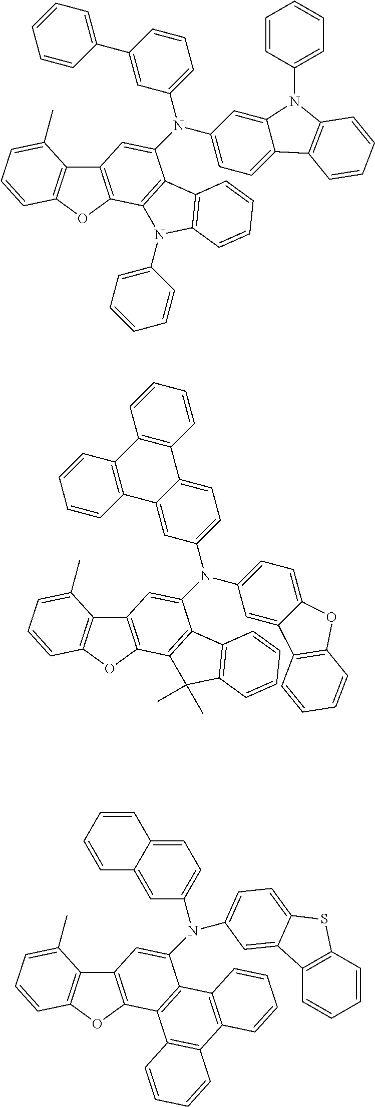 [SMILES: Cc1cccc2oc3c(cc(N(c4cccc(-c5ccccc5)c4)c4ccc5c6ccccc6n(-c6ccccc6)c5c4)c4c5ccccc5n(-c5ccccc5)c34)c12 ; Cc1cccc2oc3c4c(c(N(c5ccc6oc7ccccc7c6c5)c5ccc6c7ccccc7c7ccccc7c6c5)cc3c12)-c1ccccc1C4(C)C ; Cc1cccc2oc3c(cc(N(c4ccc5ccccc5c4)c4ccc5sc6ccccc6c5c4)c4c5ccccc5c5ccccc5c34)c12]

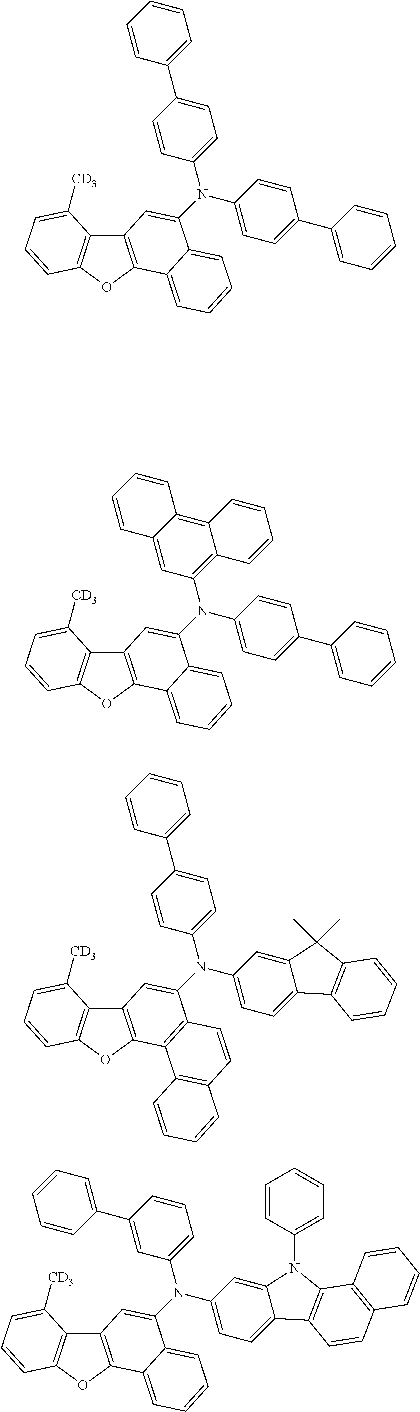 [SMILES: [2H]C([2H])([2H])c1cccc2oc3c4ccccc4c(N(c4ccc(-c5ccccc5)cc4)c4ccc(-c5ccccc5)cc4)cc3c12 ; [2H]C([2H])([2H])c1cccc2oc3c4ccccc4c(N(c4ccc(-c5ccccc5)cc4)c4cc5ccccc5c5ccccc45)cc3c12 ; [2H]C([2H])([2H])c1cccc2oc3c(cc(N(c4ccc(-c5ccccc5)cc4)c4ccc5c(c4)C(C)(C)c4ccccc4-5)c4ccc5ccccc5c43)c12 ; [2H]C([2H])([2H])c1cccc2oc3c4ccccc4c(N(c4cccc(-c5ccccc5)c4)c4ccc5c6ccc7ccccc7c6n(-c6ccccc6)c5c4)cc3c12]

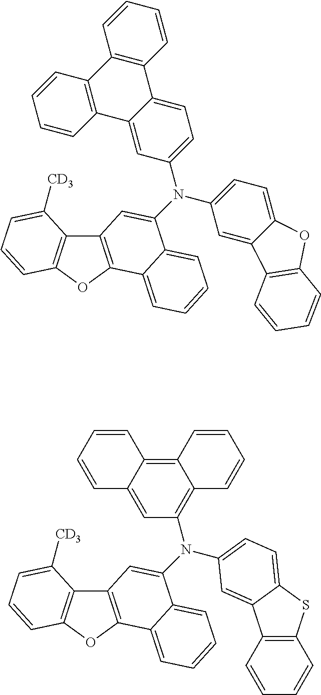 [SMILES: [2H]C([2H])([2H])c1cccc2oc3c4ccccc4c(N(c4ccc5oc6ccccc6c5c4)c4ccc5c6ccccc6c6ccccc6c5c4)cc3c12 ; [2H]C([2H])([2H])c1cccc2oc3c4ccccc4c(N(c4ccc5sc6ccccc6c5c4)c4cc5ccccc5c5ccccc45)cc3c12]

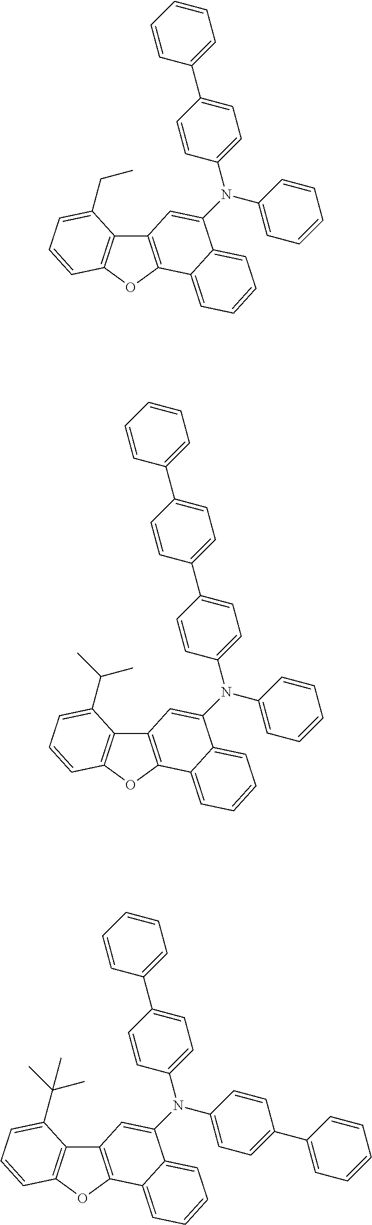 [SMILES: CCc1cccc2oc3c4ccccc4c(N(c4ccccc4)c4ccc(-c5ccccc5)cc4)cc3c12 ; CC(C)c1cccc2oc3c4ccccc4c(N(c4ccccc4)c4ccc(-c5ccc(-c6ccccc6)cc5)cc4)cc3c12 ; CC(C)(C)c1cccc2oc3c4ccccc4c(N(c4ccc(-c5ccccc5)cc4)c4ccc(-c5ccccc5)cc4)cc3c12]

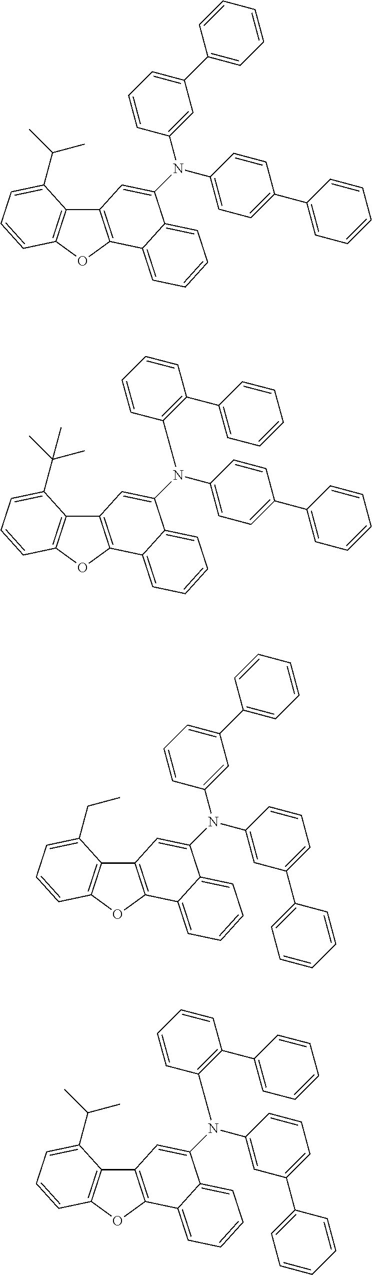 [SMILES: CC(C)c1cccc2oc3c4ccccc4c(N(c4ccc(-c5ccccc5)cc4)c4cccc(-c5ccccc5)c4)cc3c12 ; CC(C)(C)c1cccc2oc3c4ccccc4c(N(c4ccc(-c5ccccc5)cc4)c4ccccc4-c4ccccc4)cc3c12 ; CCc1cccc2oc3c4ccccc4c(N(c4cccc(-c5ccccc5)c4)c4cccc(-c5ccccc5)c4)cc3c12 ; CC(C)c1cccc2oc3c4ccccc4c(N(c4cccc(-c5ccccc5)c4)c4ccccc4-c4ccccc4)cc3c12]

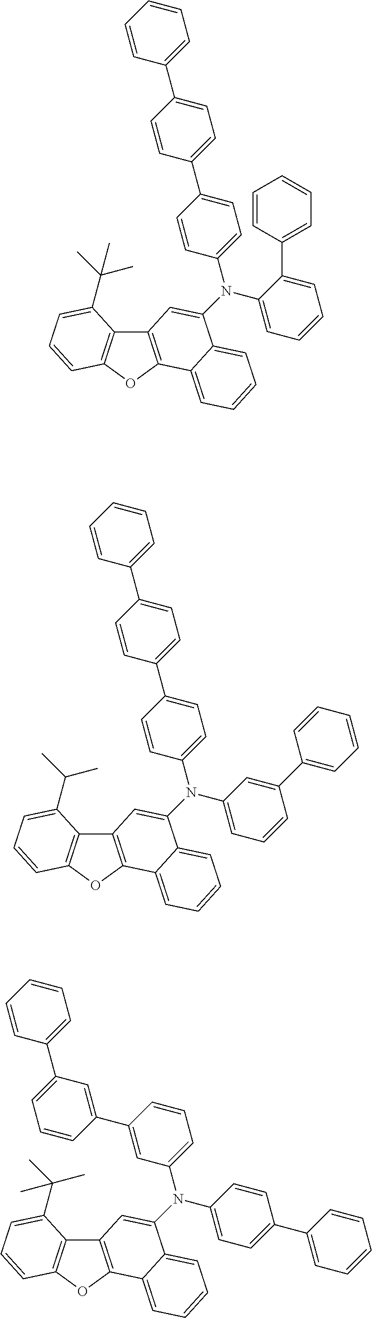 [SMILES: CC(C)(C)c1cccc2oc3c4ccccc4c(N(c4ccc(-c5ccc(-c6ccccc6)cc5)cc4)c4ccccc4-c4ccccc4)cc3c12 ; CC(C)c1cccc2oc3c4ccccc4c(N(c4ccc(-c5ccc(-c6ccccc6)cc5)cc4)c4cccc(-c5ccccc5)c4)cc3c12 ; CC(C)(C)c1cccc2oc3c4ccccc4c(N(c4ccc(-c5ccccc5)cc4)c4cccc(-c5cccc(-c6ccccc6)c5)c4)cc3c12]

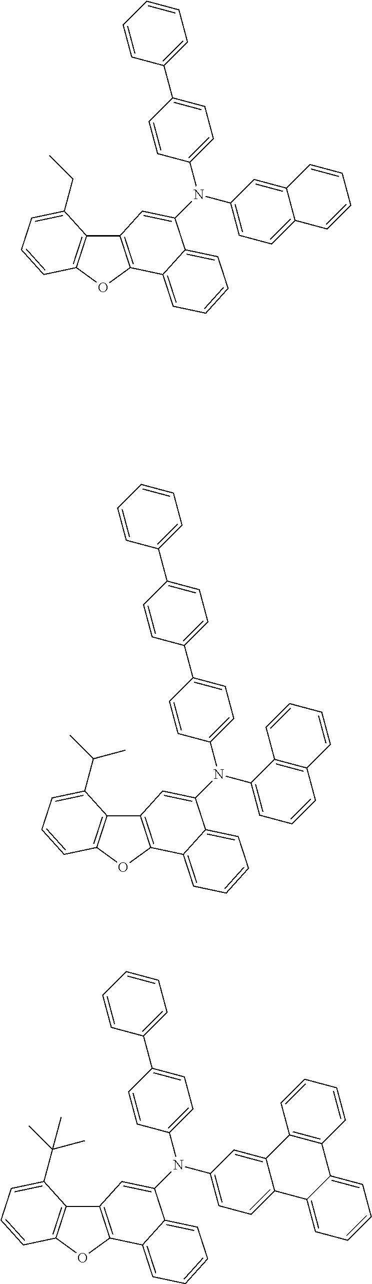 [SMILES: CCc1cccc2oc3c4ccccc4c(N(c4ccc(-c5ccccc5)cc4)c4ccc5ccccc5c4)cc3c12 ; CC(C)c1cccc2oc3c4ccccc4c(N(c4ccc(-c5ccc(-c6ccccc6)cc5)cc4)c4cccc5ccccc45)cc3c12 ; CC(C)(C)c1cccc2oc3c4ccccc4c(N(c4ccc(-c5ccccc5)cc4)c4ccc5c6ccccc6c6ccccc6c5c4)cc3c12]

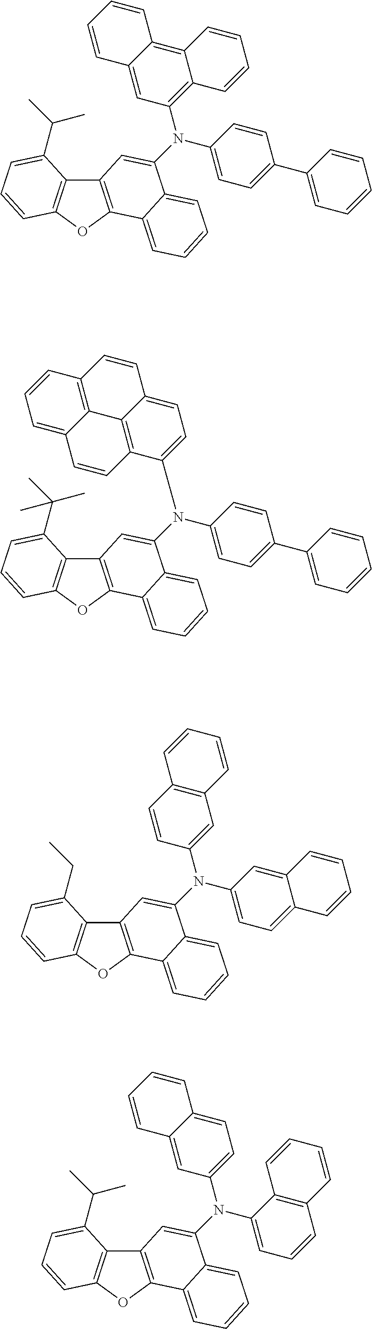 [SMILES: CC(C)c1cccc2oc3c4ccccc4c(N(c4ccc(-c5ccccc5)cc4)c4cc5ccccc5c5ccccc45)cc3c12 ; CC(C)(C)c1cccc2oc3c4ccccc4c(N(c4ccc(-c5ccccc5)cc4)c4ccc5ccc6cccc7ccc4c5c67)cc3c12 ; CCc1cccc2oc3c4ccccc4c(N(c4ccc5ccccc5c4)c4ccc5ccccc5c4)cc3c12 ; CC(C)c1cccc2oc3c4ccccc4c(N(c4ccc5ccccc5c4)c4cccc5ccccc45)cc3c12]

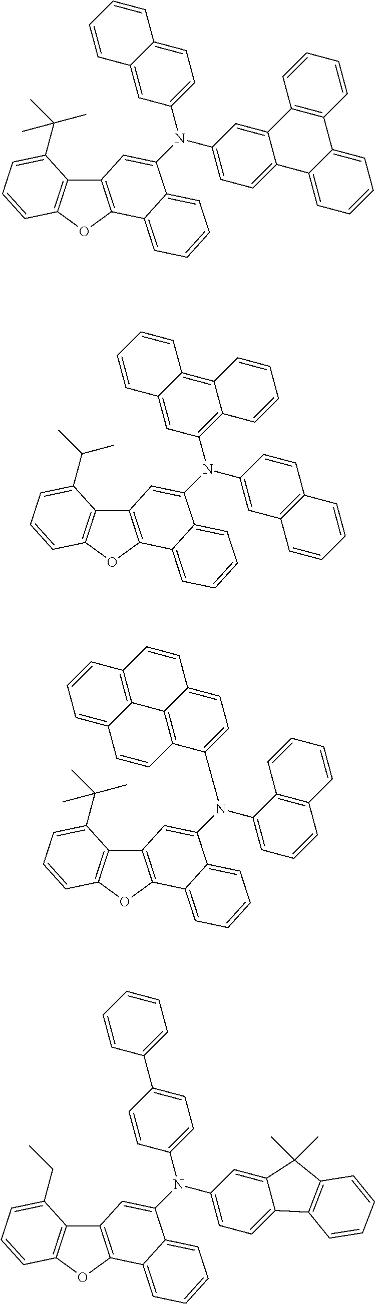 [SMILES: CC(C)(C)c1cccc2oc3c4ccccc4c(N(c4ccc5ccccc5c4)c4ccc5c6ccccc6c6ccccc6c5c4)cc3c12 ; CC(C)c1cccc2oc3c4ccccc4c(N(c4ccc5ccccc5c4)c4cc5ccccc5c5ccccc45)cc3c12 ; CC(C)(C)c1cccc2oc3c4ccccc4c(N(c4cccc5ccccc45)c4ccc5ccc6cccc7ccc4c5c67)cc3c12 ; CCc1cccc2oc3c4ccccc4c(N(c4ccc(-c5ccccc5)cc4)c4ccc5c(c4)C(C)(C)c4ccccc4-5)cc3c12]

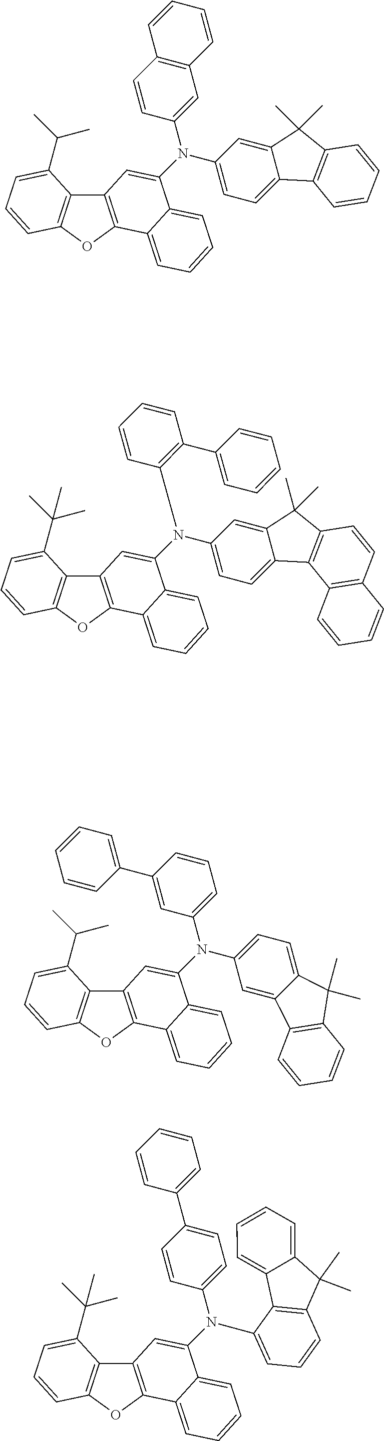 [SMILES: CC(C)c1cccc2oc3c4ccccc4c(N(c4ccc5c(c4)C(C)(C)c4ccccc4-5)c4ccc5ccccc5c4)cc3c12 ; CC(C)(C)c1cccc2oc3c4ccccc4c(N(c4ccc5c(c4)C(C)(C)c4ccc6ccccc6c4-5)c4ccccc4-c4ccccc4)cc3c12 ; CC(C)c1cccc2oc3c4ccccc4c(N(c4cccc(-c5ccccc5)c4)c4ccc5c(c4)-c4ccccc4C5(C)C)cc3c12 ; CC(C)(C)c1cccc2oc3c4ccccc4c(N(c4ccc(-c5ccccc5)cc4)c4cccc5c4-c4ccccc4C5(C)C)cc3c12]

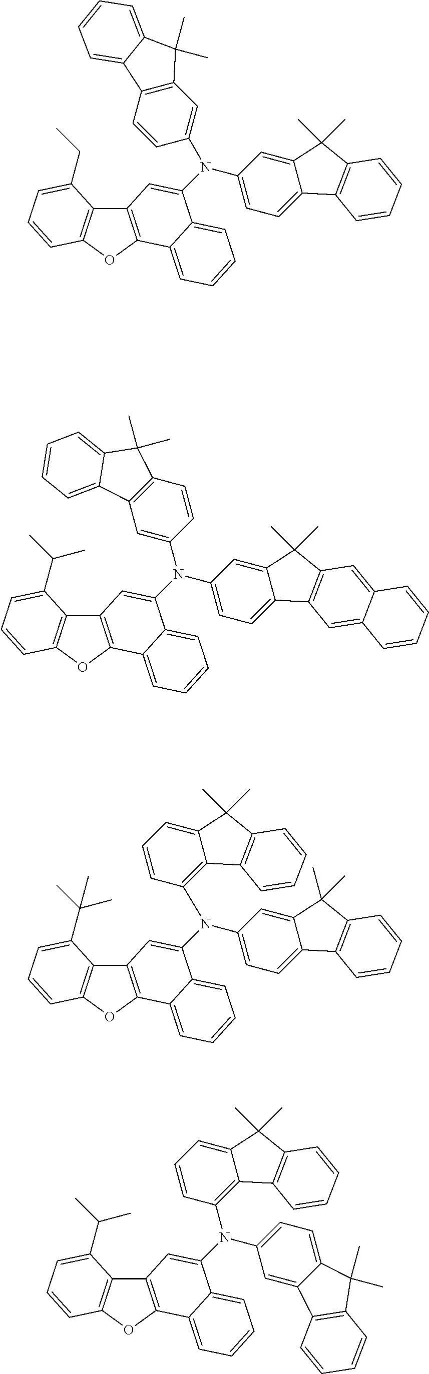 [SMILES: CCc1cccc2oc3c4ccccc4c(N(c4ccc5c(c4)C(C)(C)c4ccccc4-5)c4ccc5c(c4)C(C)(C)c4ccccc4-5)cc3c12 ; CC(C)c1cccc2oc3c4ccccc4c(N(c4ccc5c(c4)-c4ccccc4C5(C)C)c4ccc5c(c4)C(C)(C)c4cc6ccccc6cc4-5)cc3c12 ; CC(C)(C)c1cccc2oc3c4ccccc4c(N(c4ccc5c(c4)C(C)(C)c4ccccc4-5)c4cccc5c4-c4ccccc4C5(C)C)cc3c12 ; CC(C)c1cccc2oc3c4ccccc4c(N(c4ccc5c(c4)-c4ccccc4C5(C)C)c4cccc5c4-c4ccccc4C5(C)C)cc3c12]

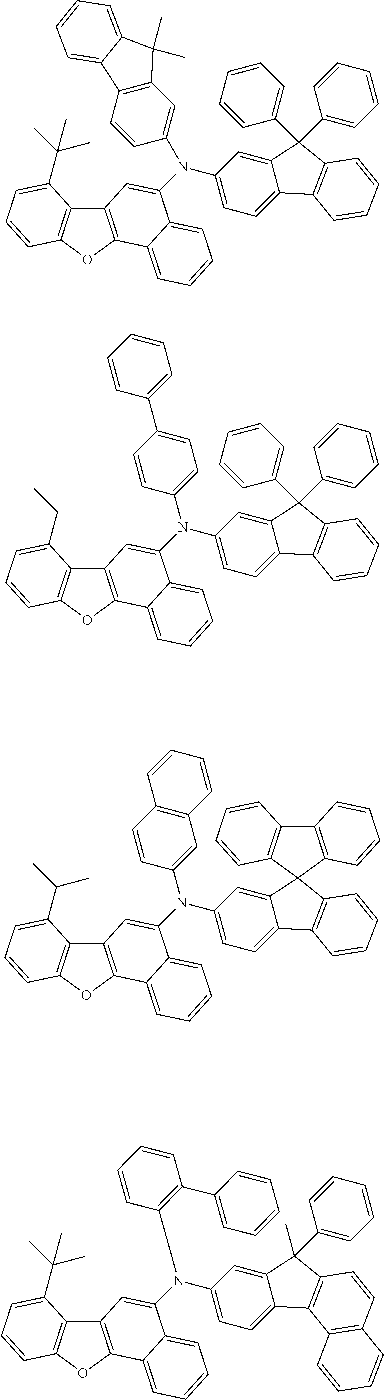 [SMILES: CC(C)(C)c1cccc2oc3c4ccccc4c(N(c4ccc5c(c4)C(C)(C)c4ccccc4-5)c4ccc5c(c4)C(c4ccccc4)(c4ccccc4)c4ccccc4-5)cc3c12 ; CCc1cccc2oc3c4ccccc4c(N(c4ccc(-c5ccccc5)cc4)c4ccc5c(c4)C(c4ccccc4)(c4ccccc4)c4ccccc4-5)cc3c12 ; CC(C)c1cccc2oc3c4ccccc4c(N(c4ccc5c(c4)C4(c6ccccc6-c6ccccc64)c4ccccc4-5)c4ccc5ccccc5c4)cc3c12 ; CC(C)(C)c1cccc2oc3c4ccccc4c(N(c4ccc5c(c4)C(C)(c4ccccc4)c4ccc6ccccc6c4-5)c4ccccc4-c4ccccc4)cc3c12]

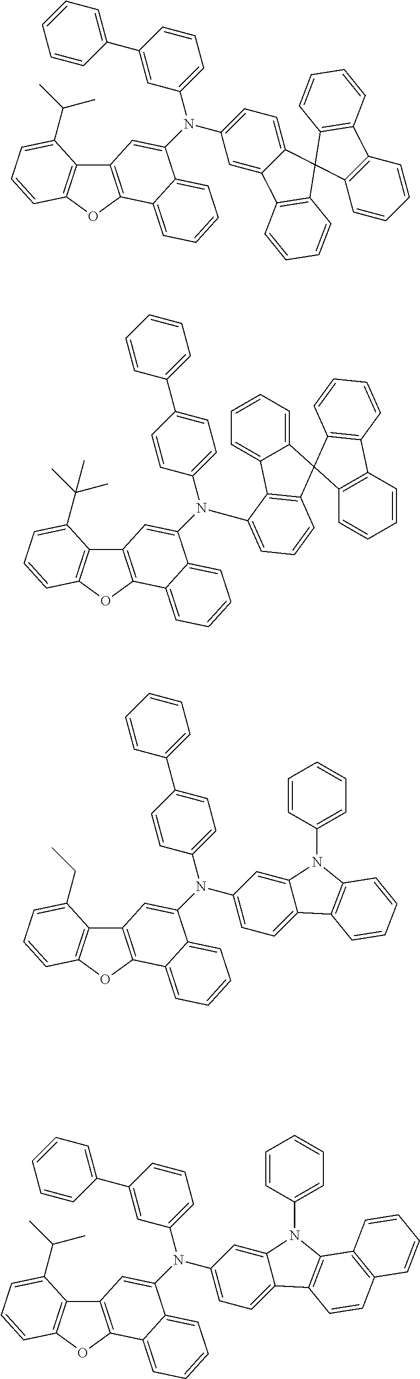 [SMILES: CC(C)c1cccc2oc3c4ccccc4c(N(c4cccc(-c5ccccc5)c4)c4ccc5c(c4)-c4ccccc4C54c5ccccc5-c5ccccc54)cc3c12 ; CC(C)(C)c1cccc2oc3c4ccccc4c(N(c4ccc(-c5ccccc5)cc4)c4cccc5c4-c4ccccc4C54c5ccccc5-c5ccccc54)cc3c12 ; CCc1cccc2oc3c4ccccc4c(N(c4ccc(-c5ccccc5)cc4)c4ccc5c6ccccc6n(-c6ccccc6)c5c4)cc3c12 ; CC(C)c1cccc2oc3c4ccccc4c(N(c4cccc(-c5ccccc5)c4)c4ccc5c6ccc7ccccc7c6n(-c6ccccc6)c5c4)cc3c12]

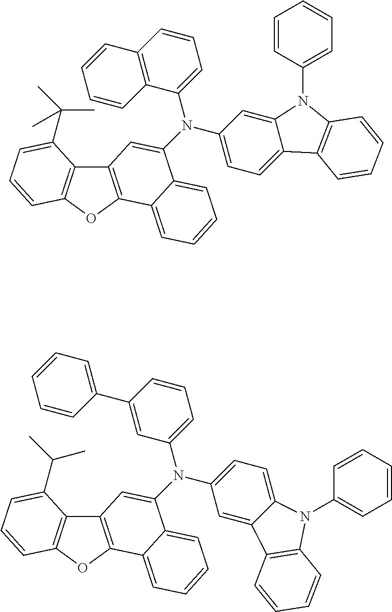 [SMILES: CC(C)(C)c1cccc2oc3c4ccccc4c(N(c4ccc5c6ccccc6n(-c6ccccc6)c5c4)c4cccc5ccccc45)cc3c12 ; CC(C)c1cccc2oc3c4ccccc4c(N(c4cccc(-c5ccccc5)c4)c4ccc5c(c4)c4ccccc4n5-c4ccccc4)cc3c12]

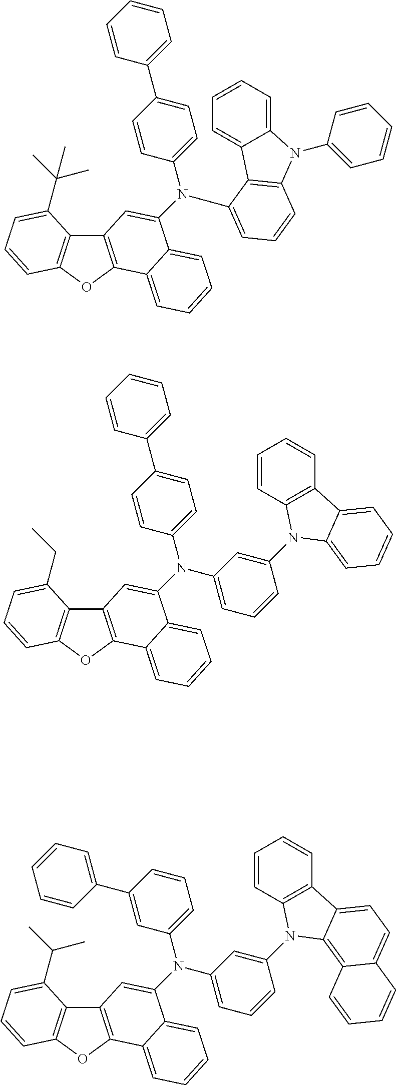 [SMILES: CC(C)(C)c1cccc2oc3c4ccccc4c(N(c4ccc(-c5ccccc5)cc4)c4cccc5c4c4ccccc4n5-c4ccccc4)cc3c12 ; CCc1cccc2oc3c4ccccc4c(N(c4ccc(-c5ccccc5)cc4)c4cccc(-n5c6ccccc6c6ccccc65)c4)cc3c12 ; CC(C)c1cccc2oc3c4ccccc4c(N(c4cccc(-c5ccccc5)c4)c4cccc(-n5c6ccccc6c6ccc7ccccc7c65)c4)cc3c12]

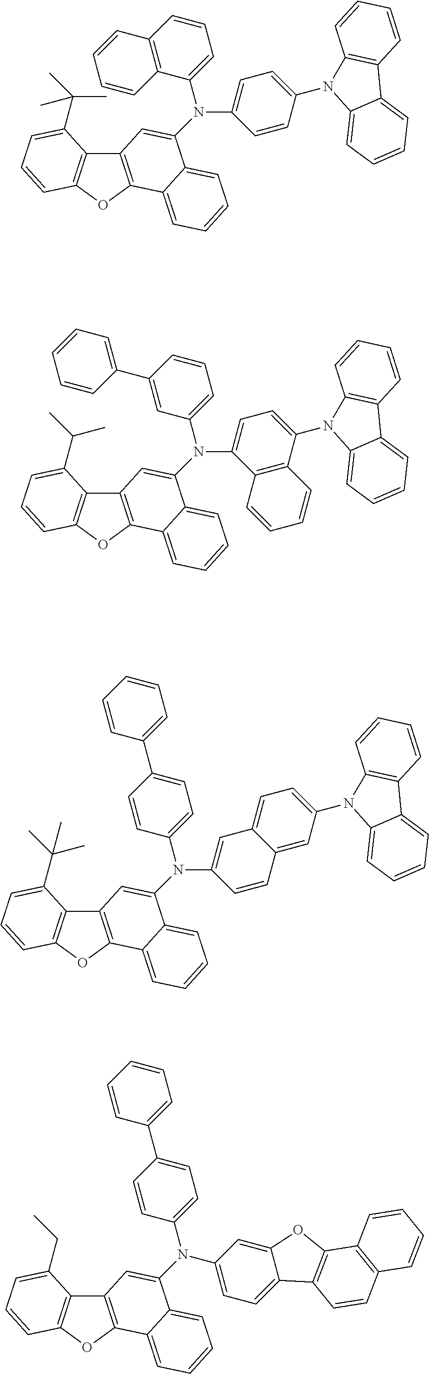 [SMILES: CC(C)(C)c1cccc2oc3c4ccccc4c(N(c4ccc(-n5c6ccccc6c6ccccc65)cc4)c4cccc5ccccc45)cc3c12 ; CC(C)c1cccc2oc3c4ccccc4c(N(c4cccc(-c5ccccc5)c4)c4ccc(-n5c6ccccc6c6ccccc65)c5ccccc45)cc3c12 ; CC(C)(C)c1cccc2oc3c4ccccc4c(N(c4ccc(-c5ccccc5)cc4)c4ccc5cc(-n6c7ccccc7c7ccccc76)ccc5c4)cc3c12 ; CCc1cccc2oc3c4ccccc4c(N(c4ccc(-c5ccccc5)cc4)c4ccc5c(c4)oc4c6ccccc6ccc54)cc3c12]

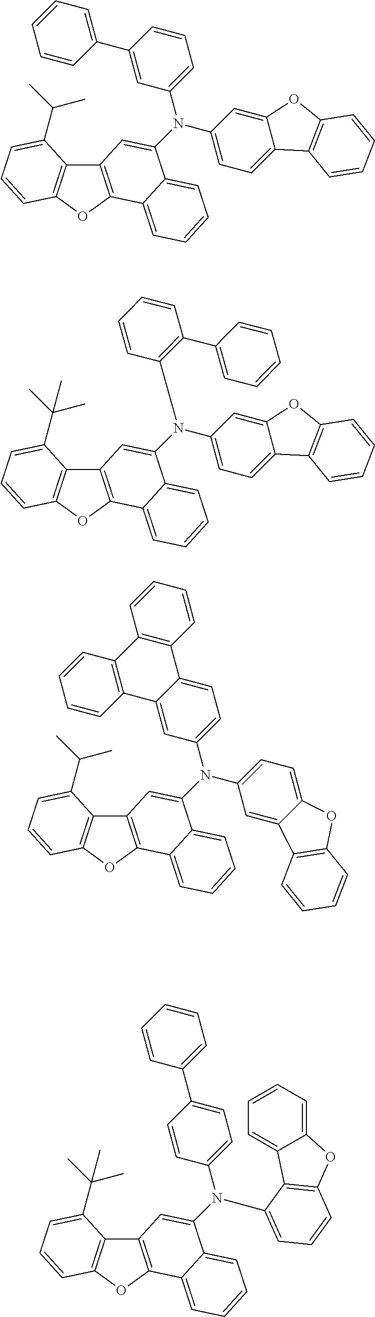

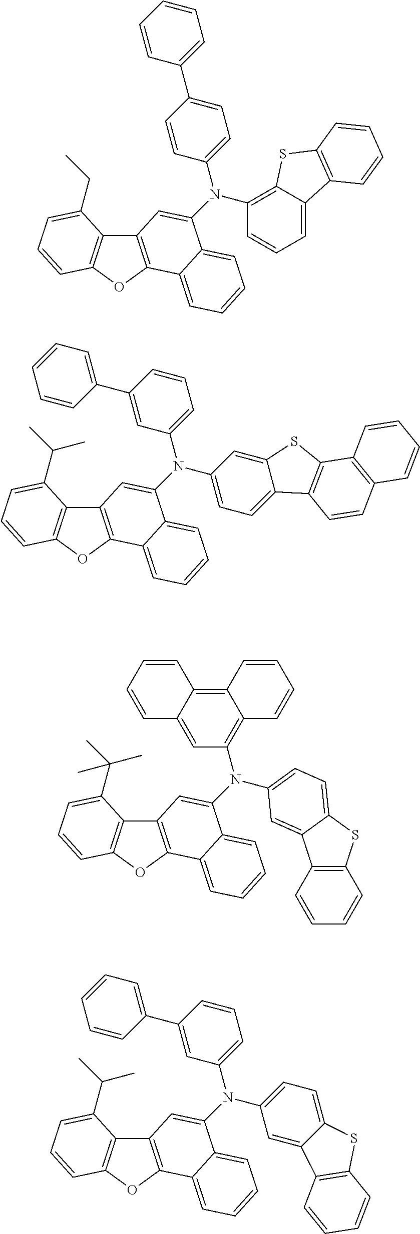 [SMILES: CCc1cccc2oc3c4ccccc4c(N(c4ccc(-c5ccccc5)cc4)c4cccc5c4sc4ccccc45)cc3c12 ; CC(C)c1cccc2oc3c4ccccc4c(N(c4cccc(-c5ccccc5)c4)c4ccc5c(c4)sc4c6ccccc6ccc54)cc3c12 ; CC(C)(C)c1cccc2oc3c4ccccc4c(N(c4ccc5sc6ccccc6c5c4)c4cc5ccccc5c5ccccc45)cc3c12 ; CC(C)c1cccc2oc3c4ccccc4c(N(c4cccc(-c5ccccc5)c4)c4ccc5sc6ccccc6c5c4)cc3c12]

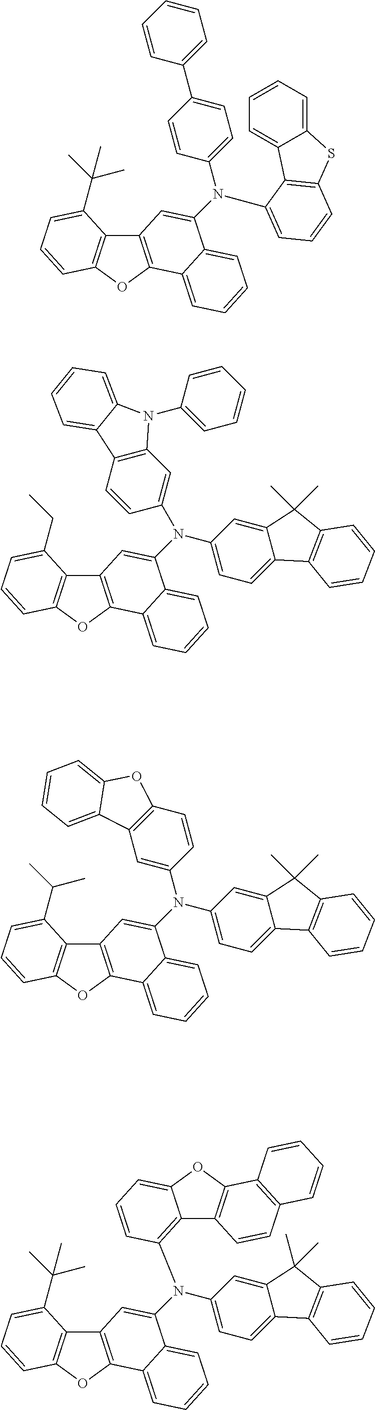 [SMILES: CC(C)(C)c1cccc2oc3c4ccccc4c(N(c4ccc(-c5ccccc5)cc4)c4cccc5sc6ccccc6c45)cc3c12 ; CCc1cccc2oc3c4ccccc4c(N(c4ccc5c(c4)C(C)(C)c4ccccc4-5)c4ccc5c6ccccc6n(-c6ccccc6)c5c4)cc3c12 ; CC(C)c1cccc2oc3c4ccccc4c(N(c4ccc5c(c4)C(C)(C)c4ccccc4-5)c4ccc5oc6ccccc6c5c4)cc3c12 ; CC(C)(C)c1cccc2oc3c4ccccc4c(N(c4ccc5c(c4)C(C)(C)c4ccccc4-5)c4cccc5oc6c7ccccc7ccc6c45)cc3c12]

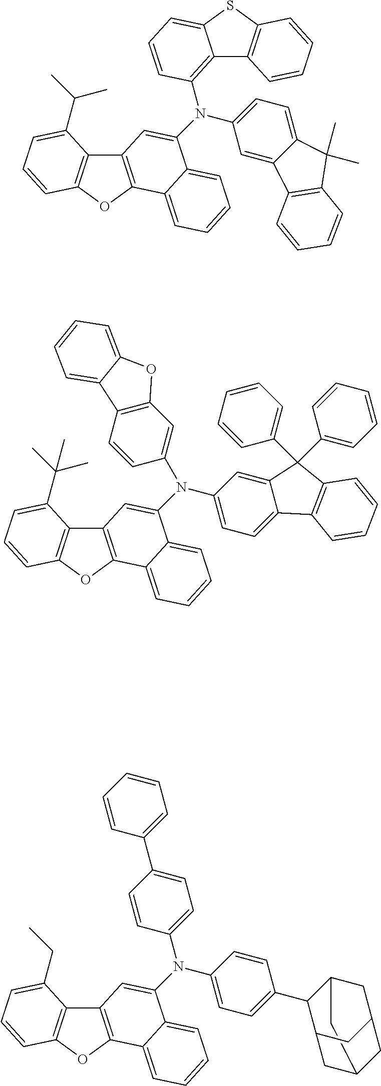 [SMILES: CC(C)c1cccc2oc3c4ccccc4c(N(c4ccc5c(c4)-c4ccccc4C5(C)C)c4cccc5sc6ccccc6c45)cc3c12 ; CC(C)(C)c1cccc2oc3c4ccccc4c(N(c4ccc5c(c4)C(c4ccccc4)(c4ccccc4)c4ccccc4-5)c4ccc5c(c4)oc4ccccc45)cc3c12 ; CCc1cccc2oc3c4ccccc4c(N(c4ccc(-c5ccccc5)cc4)c4ccc(C5C6CC7CC(C6)CC5C7)cc4)cc3c12]

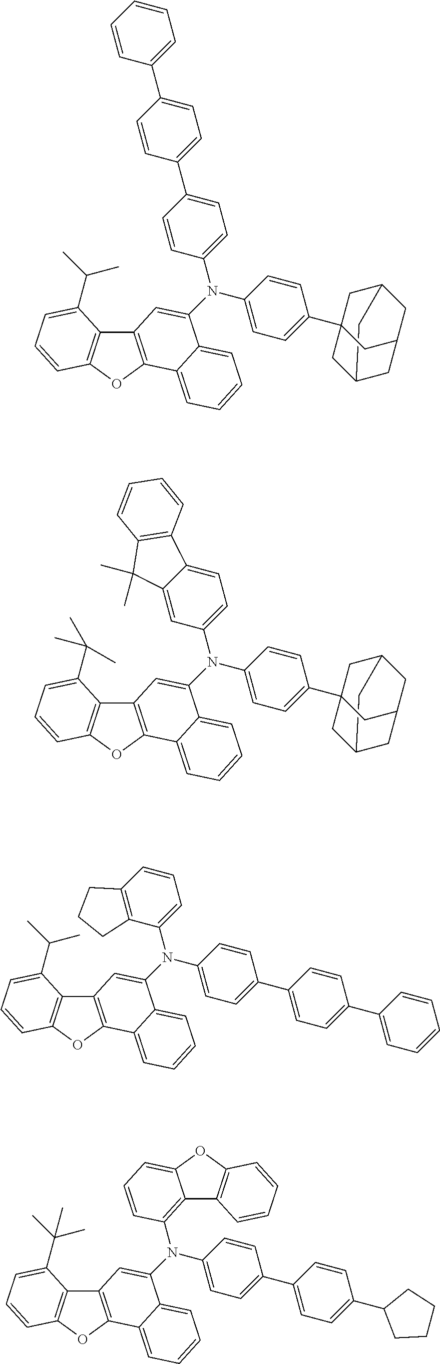 [SMILES: CC(C)c1cccc2oc3c4ccccc4c(N(c4ccc(-c5ccc(-c6ccccc6)cc5)cc4)c4ccc(C56CC7CC(CC(C7)C5)C6)cc4)cc3c12 ; CC(C)(C)c1cccc2oc3c4ccccc4c(N(c4ccc(C56CC7CC(CC(C7)C5)C6)cc4)c4ccc5c(c4)C(C)(C)c4ccccc4-5)cc3c12 ; CC(C)c1cccc2oc3c4ccccc4c(N(c4ccc(-c5ccc(-c6ccccc6)cc5)cc4)c4cccc5c4CCC5)cc3c12 ; CC(C)(C)c1cccc2oc3c4ccccc4c(N(c4ccc(-c5ccc(C6CCCC6)cc5)cc4)c4cccc5oc6ccccc6c45)cc3c12]

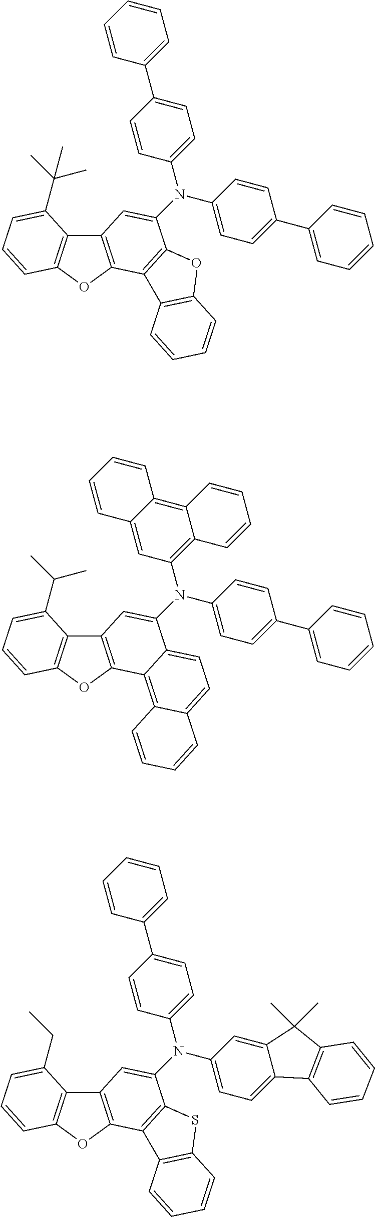 [SMILES: CC(C)(C)c1cccc2oc3c(cc(N(c4ccc(-c5ccccc5)cc4)c4ccc(-c5ccccc5)cc4)c4oc5ccccc5c43)c12 ; CC(C)c1cccc2oc3c(cc(N(c4ccc(-c5ccccc5)cc4)c4cc5ccccc5c5ccccc45)c4ccc5ccccc5c43)c12 ; CCc1cccc2oc3c(cc(N(c4ccc(-c5ccccc5)cc4)c4ccc5c(c4)C(C)(C)c4ccccc4-5)c4sc5ccccc5c43)c12]

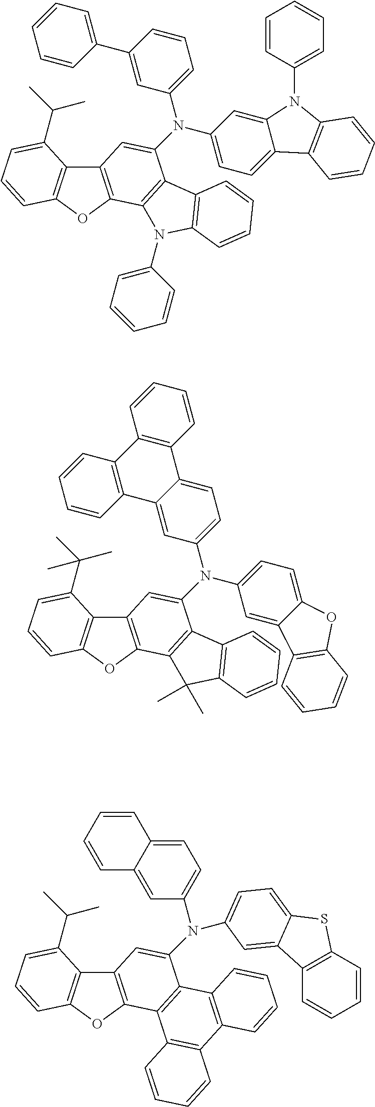 [SMILES: CC(C)c1cccc2oc3c(cc(N(c4cccc(-c5ccccc5)c4)c4ccc5c6ccccc6n(-c6ccccc6)c5c4)c4c5ccccc5n(-c5ccccc5)c34)c12 ; CC(C)(C)c1cccc2oc3c4c(c(N(c5ccc6oc7ccccc7c6c5)c5ccc6c7ccccc7c7ccccc7c6c5)cc3c12)-c1ccccc1C4(C)C ; CC(C)c1cccc2oc3c(cc(N(c4ccc5ccccc5c4)c4ccc5sc6ccccc6c5c4)c4c5ccccc5c5ccccc5c34)c12]

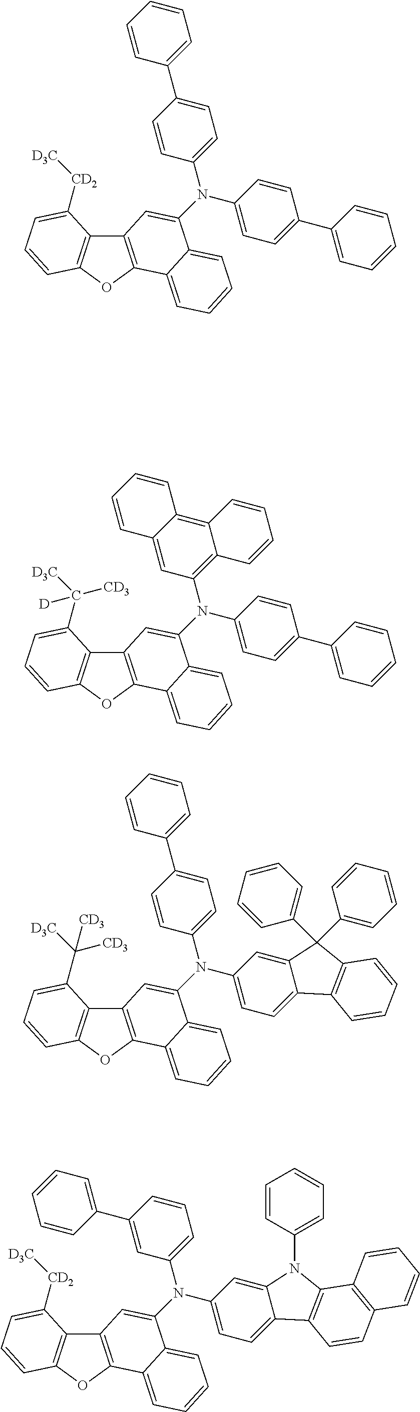

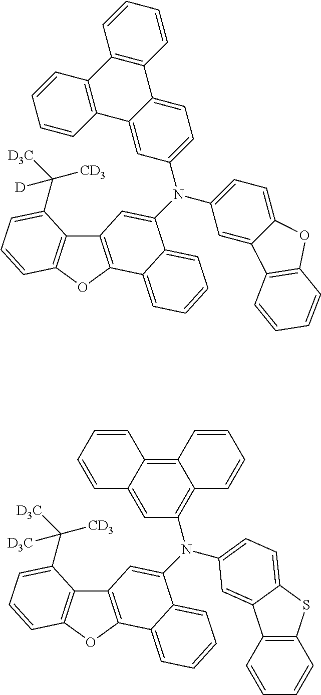 [SMILES: [2H]C([2H])([2H])C([2H])(c1cccc2oc3c4ccccc4c(N(c4ccc5oc6ccccc6c5c4)c4ccc5c6ccccc6c6ccccc6c5c4)cc3c12)C([2H])([2H])[2H] ; [2H]C([2H])([2H])C(c1cccc2oc3c4ccccc4c(N(c4ccc5sc6ccccc6c5c4)c4cc5ccccc5c5ccccc45)cc3c12)(C([2H])([2H])[2H])C([2H])([2H])[2H]]

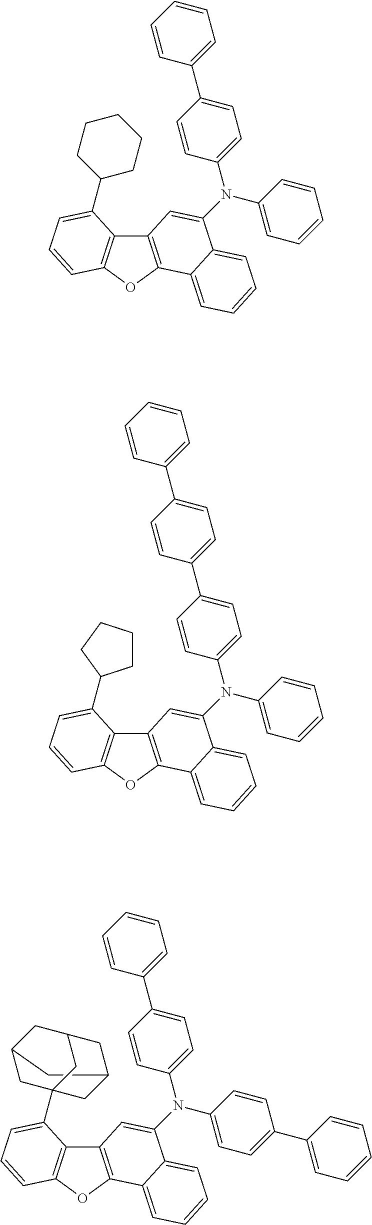 [SMILES: c1ccc(-c2ccc(N(c3ccccc3)c3cc4c(oc5cccc(C6CCCCC6)c54)c4ccccc34)cc2)cc1 ; c1ccc(-c2ccc(-c3ccc(N(c4ccccc4)c4cc5c(oc6cccc(C7CCCC7)c65)c5ccccc45)cc3)cc2)cc1 ; c1ccc(-c2ccc(N(c3ccc(-c4ccccc4)cc3)c3cc4c(oc5cccc(C67CC8CC(CC(C8)C6)C7)c54)c4ccccc34)cc2)cc1]

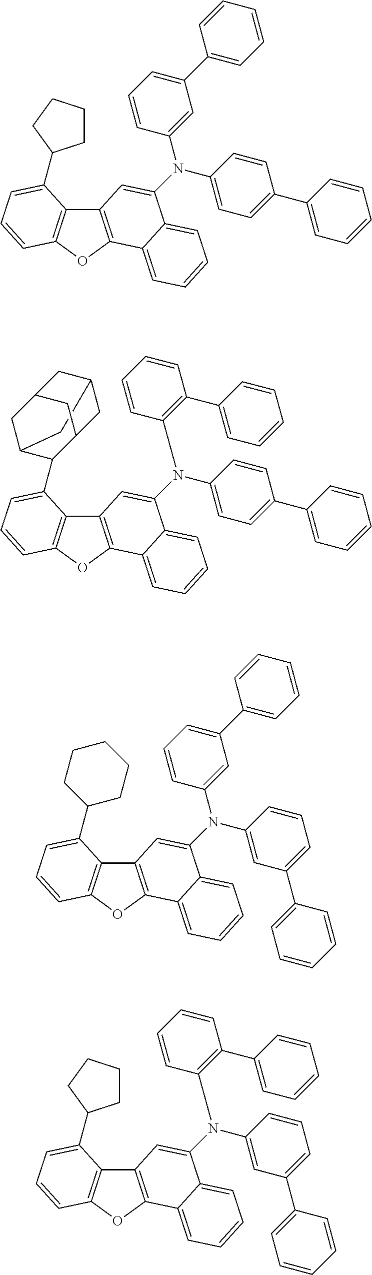 [SMILES: c1ccc(-c2ccc(N(c3cccc(-c4ccccc4)c3)c3cc4c(oc5cccc(C6CCCC6)c54)c4ccccc34)cc2)cc1 ; c1ccc(-c2ccc(N(c3ccccc3-c3ccccc3)c3cc4c(oc5cccc(C6C7CC8CC(C7)CC6C8)c54)c4ccccc34)cc2)cc1 ; c1ccc(-c2cccc(N(c3cccc(-c4ccccc4)c3)c3cc4c(oc5cccc(C6CCCCC6)c54)c4ccccc34)c2)cc1 ; c1ccc(-c2cccc(N(c3ccccc3-c3ccccc3)c3cc4c(oc5cccc(C6CCCC6)c54)c4ccccc34)c2)cc1]

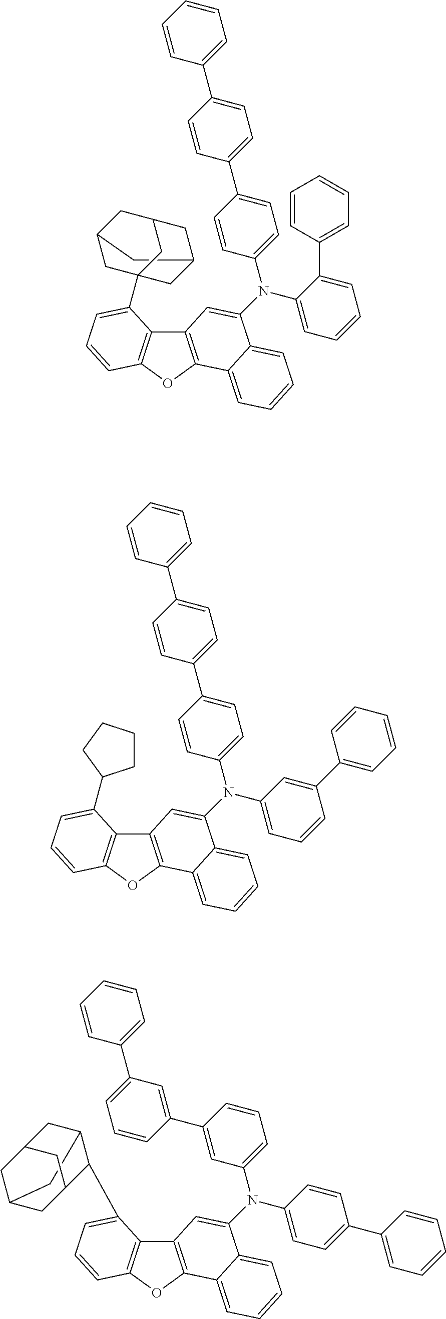 [SMILES: c1ccc(-c2ccc(-c3ccc(N(c4ccccc4-c4ccccc4)c4cc5c(oc6cccc(C78CC9CC(CC(C9)C7)C8)c65)c5ccccc45)cc3)cc2)cc1 ; c1ccc(-c2ccc(-c3ccc(N(c4cccc(-c5ccccc5)c4)c4cc5c(oc6cccc(C7CCCC7)c65)c5ccccc45)cc3)cc2)cc1 ; c1ccc(-c2ccc(N(c3cccc(-c4cccc(-c5ccccc5)c4)c3)c3cc4c(oc5cccc(C6C7CC8CC(C7)CC6C8)c54)c4ccccc34)cc2)cc1]

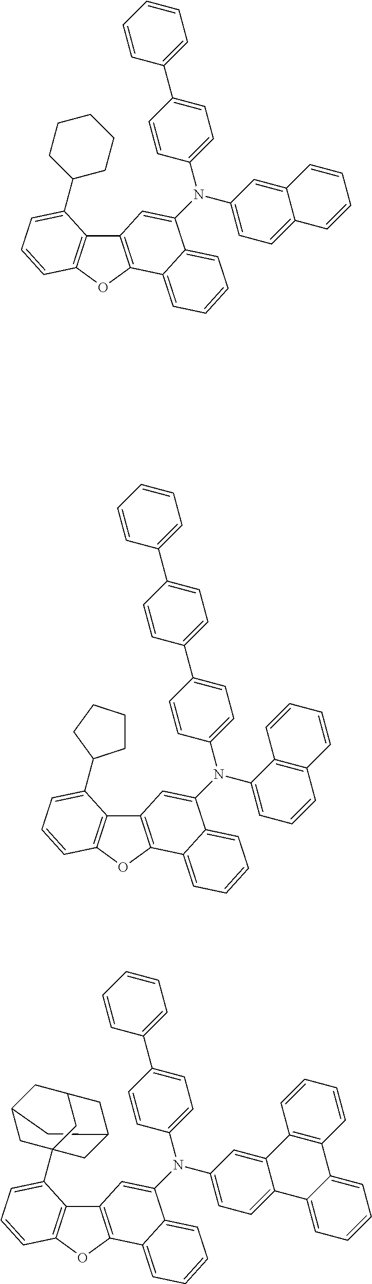 [SMILES: c1ccc(-c2ccc(N(c3ccc4ccccc4c3)c3cc4c(oc5cccc(C6CCCCC6)c54)c4ccccc34)cc2)cc1 ; c1ccc(-c2ccc(-c3ccc(N(c4cccc5ccccc45)c4cc5c(oc6cccc(C7CCCC7)c65)c5ccccc45)cc3)cc2)cc1 ; c1ccc(-c2ccc(N(c3ccc4c5ccccc5c5ccccc5c4c3)c3cc4c(oc5cccc(C67CC8CC(CC(C8)C6)C7)c54)c4ccccc34)cc2)cc1]

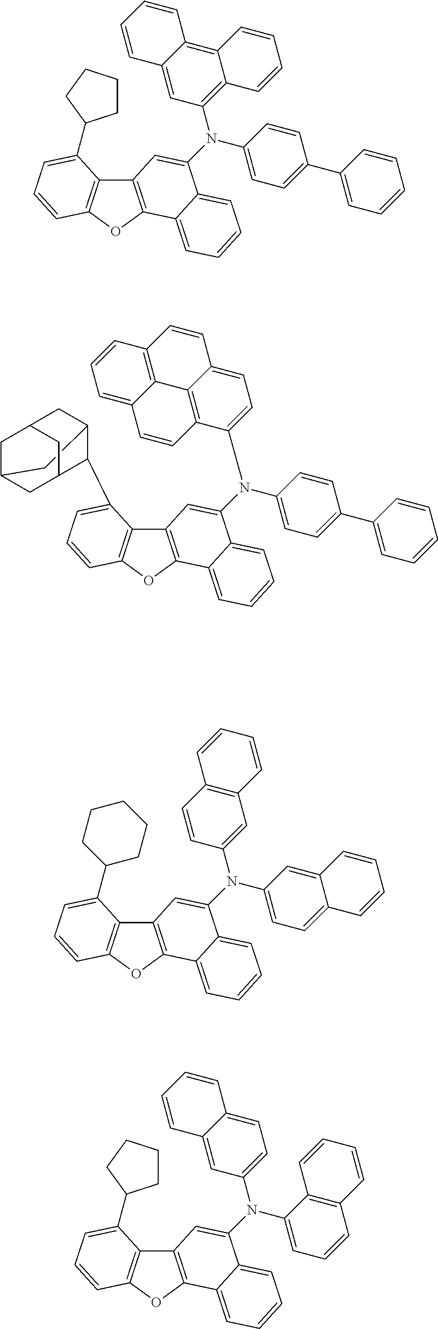 [SMILES: c1ccc(-c2ccc(N(c3cc4ccccc4c4ccccc34)c3cc4c(oc5cccc(C6CCCC6)c54)c4ccccc34)cc2)cc1 ; c1ccc(-c2ccc(N(c3cc4c(oc5cccc(C6C7CC8CC(C7)CC6C8)c54)c4ccccc34)c3ccc4ccc5cccc6ccc3c4c56)cc2)cc1 ; c1ccc2cc(N(c3ccc4ccccc4c3)c3cc4c(oc5cccc(C6CCCCC6)c54)c4ccccc34)ccc2c1 ; c1ccc2cc(N(c3cccc4ccccc34)c3cc4c(oc5cccc(C6CCCC6)c54)c4ccccc34)ccc2c1]

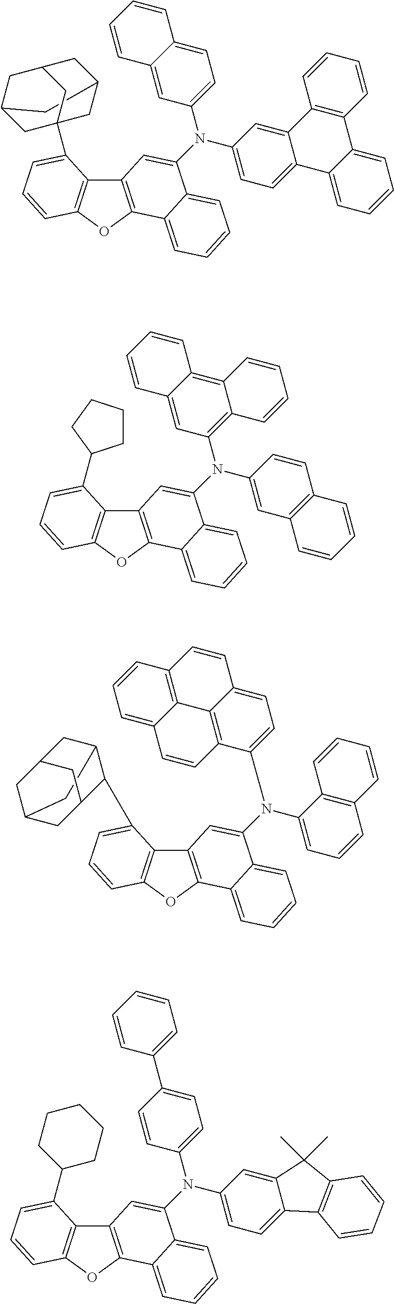 [SMILES: c1ccc2cc(N(c3ccc4c5ccccc5c5ccccc5c4c3)c3cc4c(oc5cccc(C67CC8CC(CC(C8)C6)C7)c54)c4ccccc34)ccc2c1 ; c1ccc2cc(N(c3cc4ccccc4c4ccccc34)c3cc4c(oc5cccc(C6CCCC6)c54)c4ccccc34)ccc2c1 ; c1ccc2c(N(c3cc4c(oc5cccc(C6C7CC8CC(C7)CC6C8)c54)c4ccccc34)c3ccc4ccc5cccc6ccc3c4c56)cccc2c1 ; CC1(C)c2ccccc2-c2ccc(N(c3ccc(-c4ccccc4)cc3)c3cc4c(oc5cccc(C6CCCCC6)c54)c4ccccc34)cc21]

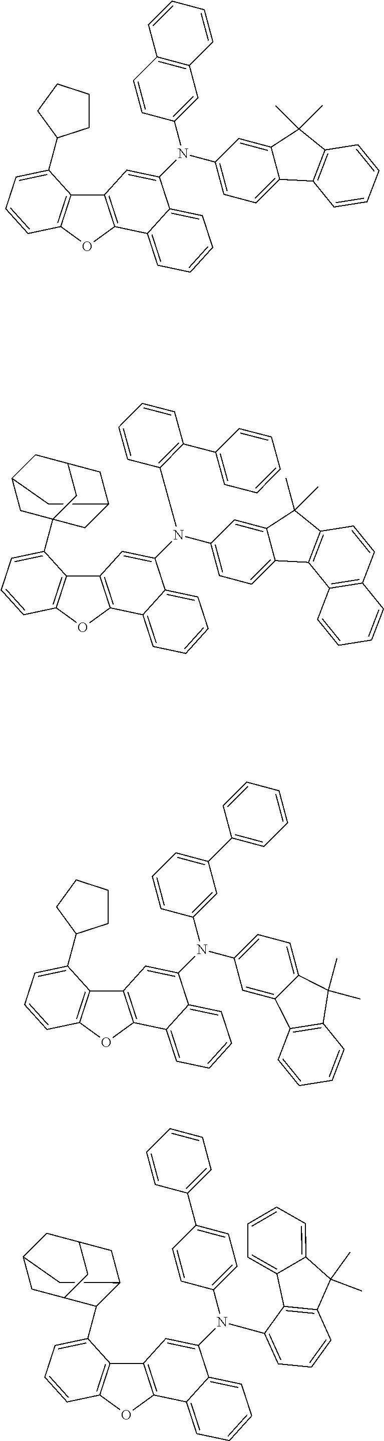 [SMILES: CC1(C)c2ccccc2-c2ccc(N(c3ccc4ccccc4c3)c3cc4c(oc5cccc(C6CCCC6)c54)c4ccccc34)cc21 ; CC1(C)c2cc(N(c3ccccc3-c3ccccc3)c3cc4c(oc5cccc(C67CC8CC(CC(C8)C6)C7)c54)c4ccccc34)ccc2-c2c1ccc1ccccc21 ; CC1(C)c2ccccc2-c2cc(N(c3cccc(-c4ccccc4)c3)c3cc4c(oc5cccc(C6CCCC6)c54)c4ccccc34)ccc21 ; CC1(C)c2ccccc2-c2c(N(c3ccc(-c4ccccc4)cc3)c3cc4c(oc5cccc(C6C7CC8CC(C7)CC6C8)c54)c4ccccc34)cccc21]

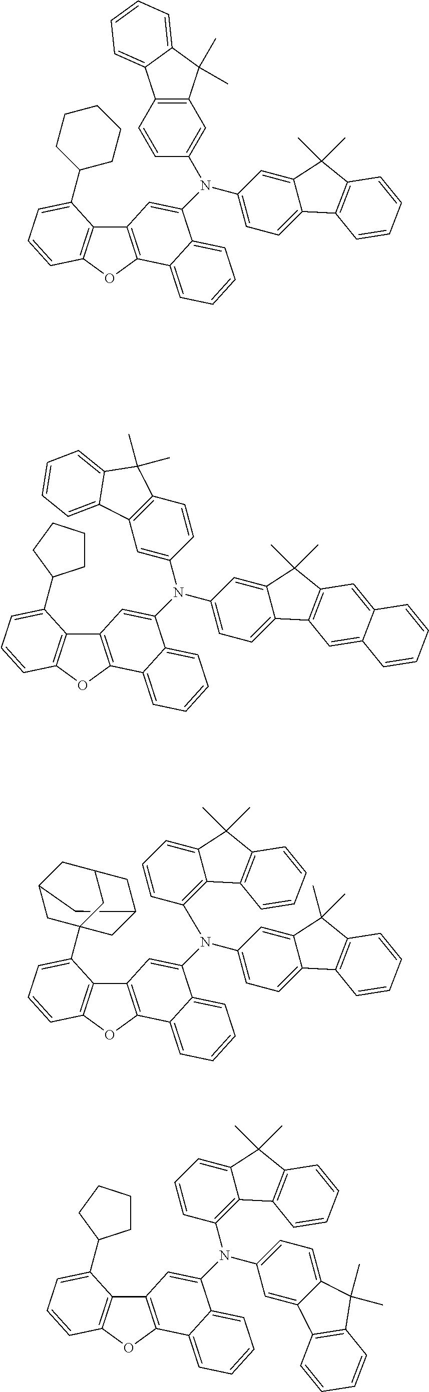 [SMILES: CC1(C)c2ccccc2-c2ccc(N(c3ccc4c(c3)C(C)(C)c3ccccc3-4)c3cc4c(oc5cccc(C6CCCCC6)c54)c4ccccc34)cc21 ; CC1(C)c2ccccc2-c2cc(N(c3ccc4c(c3)C(C)(C)c3cc5ccccc5cc3-4)c3cc4c(oc5cccc(C6CCCC6)c54)c4ccccc34)ccc21 ; CC1(C)c2ccccc2-c2ccc(N(c3cccc4c3-c3ccccc3C4(C)C)c3cc4c(oc5cccc(C67CC8CC(CC(C8)C6)C7)c54)c4ccccc34)cc21 ; CC1(C)c2ccccc2-c2cc(N(c3cccc4c3-c3ccccc3C4(C)C)c3cc4c(oc5cccc(C6CCCC6)c54)c4ccccc34)ccc21]

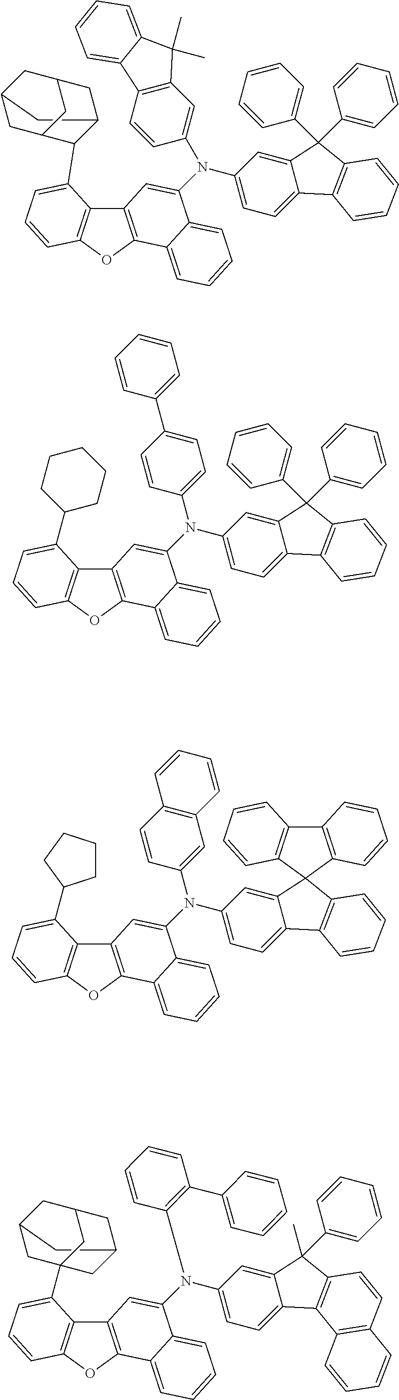 [SMILES: CC1(C)c2ccccc2-c2ccc(N(c3ccc4c(c3)C(c3ccccc3)(c3ccccc3)c3ccccc3-4)c3cc4c(oc5cccc(C6C7CC8CC(C7)CC6C8)c54)c4ccccc34)cc21 ; c1ccc(-c2ccc(N(c3ccc4c(c3)C(c3ccccc3)(c3ccccc3)c3ccccc3-4)c3cc4c(oc5cccc(C6CCCCC6)c54)c4ccccc34)cc2)cc1 ; c1ccc2c(c1)-c1ccccc1C21c2ccccc2-c2ccc(N(c3ccc4ccccc4c3)c3cc4c(oc5cccc(C6CCCC6)c54)c4ccccc34)cc21 ; CC1(c2ccccc2)c2cc(N(c3ccccc3-c3ccccc3)c3cc4c(oc5cccc(C67CC8CC(CC(C8)C6)C7)c54)c4ccccc34)ccc2-c2c1ccc1ccccc21]

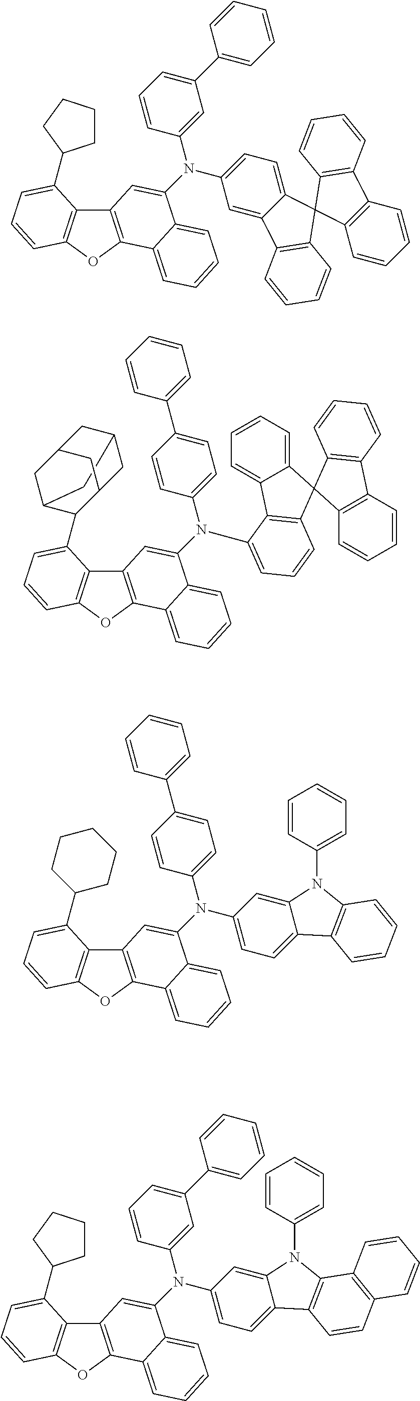 [SMILES: c1ccc(-c2cccc(N(c3ccc4c(c3)-c3ccccc3C43c4ccccc4-c4ccccc43)c3cc4c(oc5cccc(C6CCCC6)c54)c4ccccc34)c2)cc1 ; c1ccc(-c2ccc(N(c3cccc4c3-c3ccccc3C43c4ccccc4-c4ccccc43)c3cc4c(oc5cccc(C6C7CC8CC(C7)CC6C8)c54)c4ccccc34)cc2)cc1 ; c1ccc(-c2ccc(N(c3ccc4c5ccccc5n(-c5ccccc5)c4c3)c3cc4c(oc5cccc(C6CCCCC6)c54)c4ccccc34)cc2)cc1 ; c1ccc(-c2cccc(N(c3ccc4c5ccc6ccccc6c5n(-c5ccccc5)c4c3)c3cc4c(oc5cccc(C6CCCC6)c54)c4ccccc34)c2)cc1]

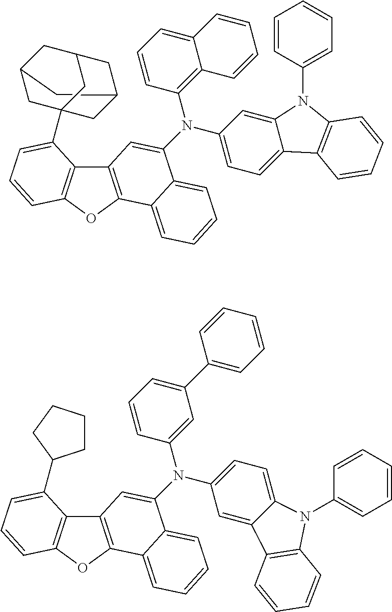 [SMILES: c1ccc(-n2c3ccccc3c3ccc(N(c4cccc5ccccc45)c4cc5c(oc6cccc(C78CC9CC(CC(C9)C7)C8)c65)c5ccccc45)cc32)cc1 ; c1ccc(-c2cccc(N(c3ccc4c(c3)c3ccccc3n4-c3ccccc3)c3cc4c(oc5cccc(C6CCCC6)c54)c4ccccc34)c2)cc1]

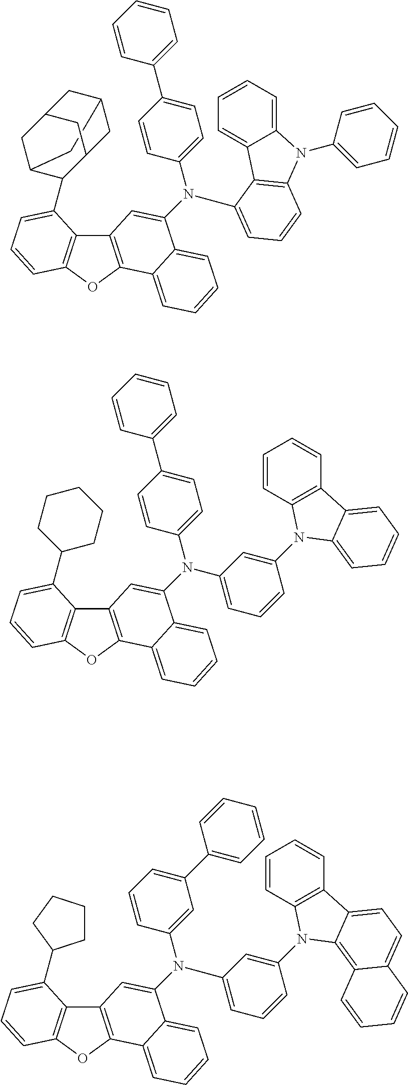 [SMILES: c1ccc(-c2ccc(N(c3cc4c(oc5cccc(C6C7CC8CC(C7)CC6C8)c54)c4ccccc34)c3cccc4c3c3ccccc3n4-c3ccccc3)cc2)cc1 ; c1ccc(-c2ccc(N(c3cccc(-n4c5ccccc5c5ccccc54)c3)c3cc4c(oc5cccc(C6CCCCC6)c54)c4ccccc34)cc2)cc1 ; c1ccc(-c2cccc(N(c3cccc(-n4c5ccccc5c5ccc6ccccc6c54)c3)c3cc4c(oc5cccc(C6CCCC6)c54)c4ccccc34)c2)cc1]

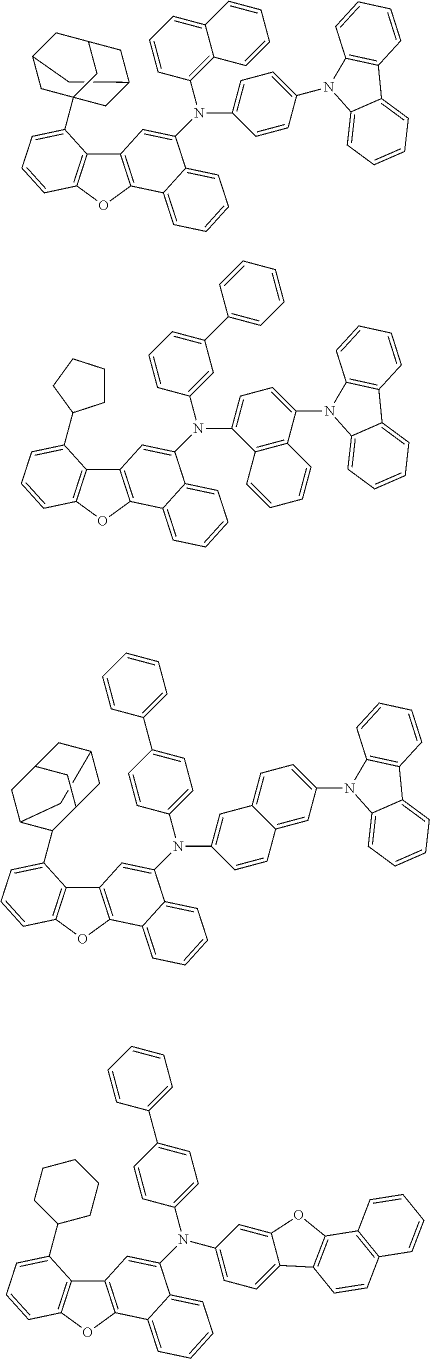 [SMILES: c1ccc2c(N(c3ccc(-n4c5ccccc5c5ccccc54)cc3)c3cc4c(oc5cccc(C67CC8CC(CC(C8)C6)C7)c54)c4ccccc34)cccc2c1 ; c1ccc(-c2cccc(N(c3ccc(-n4c5ccccc5c5ccccc54)c4ccccc34)c3cc4c(oc5cccc(C6CCCC6)c54)c4ccccc34)c2)cc1 ; c1ccc(-c2ccc(N(c3ccc4cc(-n5c6ccccc6c6ccccc65)ccc4c3)c3cc4c(oc5cccc(C6C7CC8CC(C7)CC6C8)c54)c4ccccc34)cc2)cc1 ; c1ccc(-c2ccc(N(c3ccc4c(c3)oc3c5ccccc5ccc43)c3cc4c(oc5cccc(C6CCCCC6)c54)c4ccccc34)cc2)cc1]

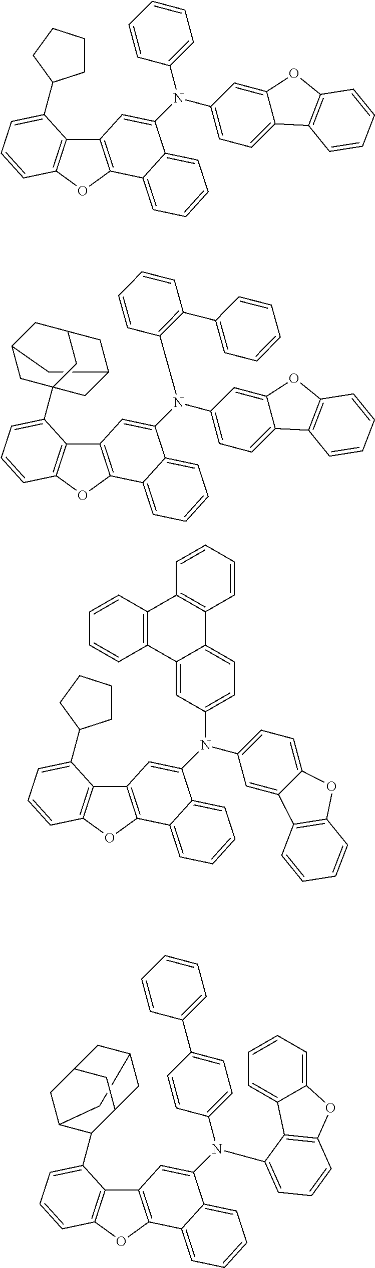

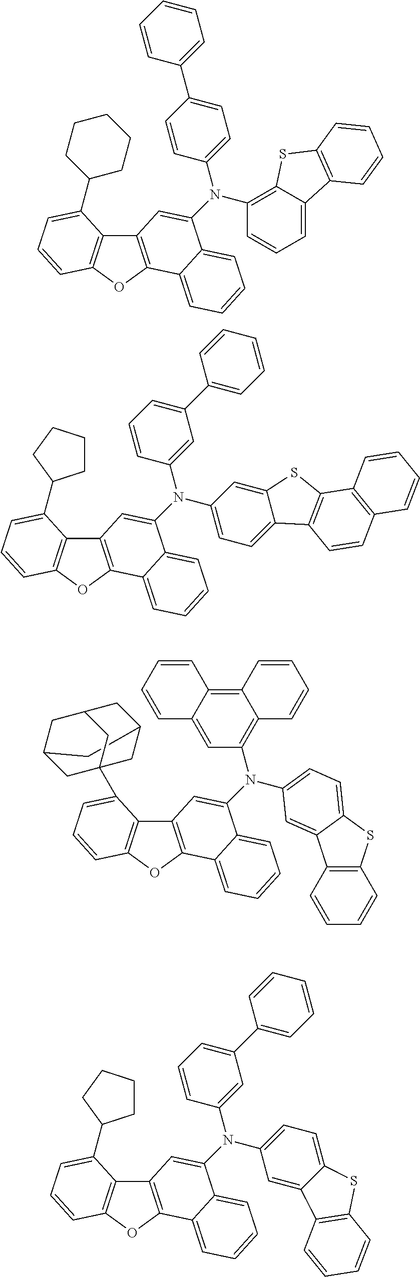

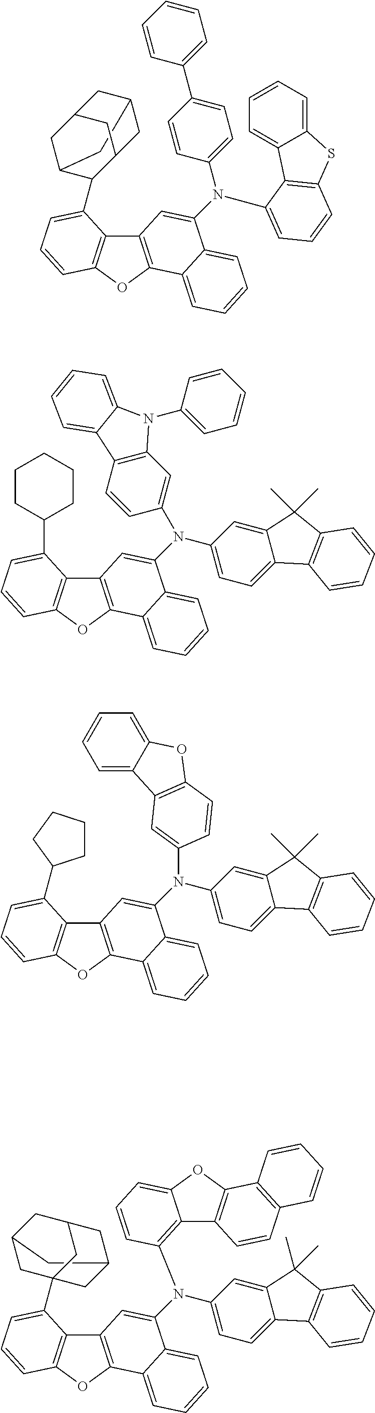 [SMILES: c1ccc(-c2ccc(N(c3cc4c(oc5cccc(C6C7CC8CC(C7)CC6C8)c54)c4ccccc34)c3cccc4sc5ccccc5c34)cc2)cc1 ; CC1(C)c2ccccc2-c2ccc(N(c3ccc4c5ccccc5n(-c5ccccc5)c4c3)c3cc4c(oc5cccc(C6CCCCC6)c54)c4ccccc34)cc21 ; CC1(C)c2ccccc2-c2ccc(N(c3ccc4oc5ccccc5c4c3)c3cc4c(oc5cccc(C6CCCC6)c54)c4ccccc34)cc21 ; CC1(C)c2ccccc2-c2ccc(N(c3cc4c(oc5cccc(C67CC8CC(CC(C8)C6)C7)c54)c4ccccc34)c3cccc4oc5c6ccccc6ccc5c34)cc21]

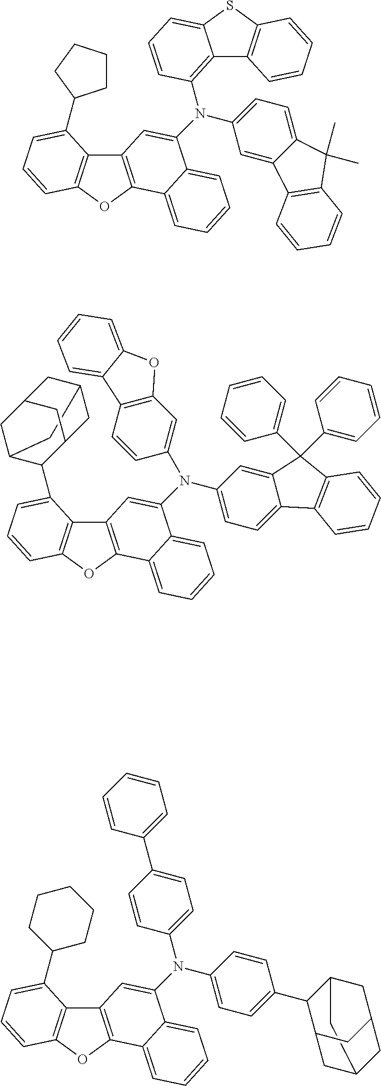 [SMILES: CC1(C)c2ccccc2-c2cc(N(c3cc4c(oc5cccc(C6CCCC6)c54)c4ccccc34)c3cccc4sc5ccccc5c34)ccc21 ; c1ccc(C2(c3ccccc3)c3ccccc3-c3ccc(N(c4ccc5c(c4)oc4ccccc45)c4cc5c(oc6cccc(C7C8CC9CC(C8)CC7C9)c65)c5ccccc45)cc32)cc1 ; c1ccc(-c2ccc(N(c3ccc(C4C5CC6CC(C5)CC4C6)cc3)c3cc4c(oc5cccc(C6CCCCC6)c54)c4ccccc34)cc2)cc1]

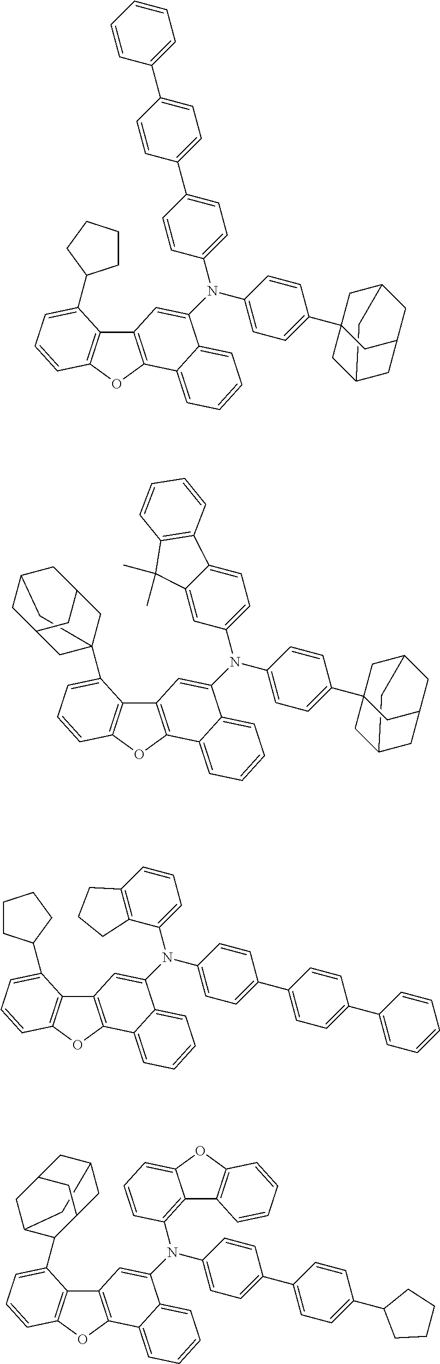 [SMILES: c1ccc(-c2ccc(-c3ccc(N(c4ccc(C56CC7CC(CC(C7)C5)C6)cc4)c4cc5c(oc6cccc(C7CCCC7)c65)c5ccccc45)cc3)cc2)cc1 ; CC1(C)c2ccccc2-c2ccc(N(c3ccc(C45CC6CC(CC(C6)C4)C5)cc3)c3cc4c(oc5cccc(C67CC8CC(CC(C8)C6)C7)c54)c4ccccc34)cc21 ; c1ccc(-c2ccc(-c3ccc(N(c4cccc5c4CCC5)c4cc5c(oc6cccc(C7CCCC7)c65)c5ccccc45)cc3)cc2)cc1 ; c1ccc2c(c1)oc1cccc(N(c3ccc(-c4ccc(C5CCCC5)cc4)cc3)c3cc4c(oc5cccc(C6C7CC8CC(C7)CC6C8)c54)c4ccccc34)c12]

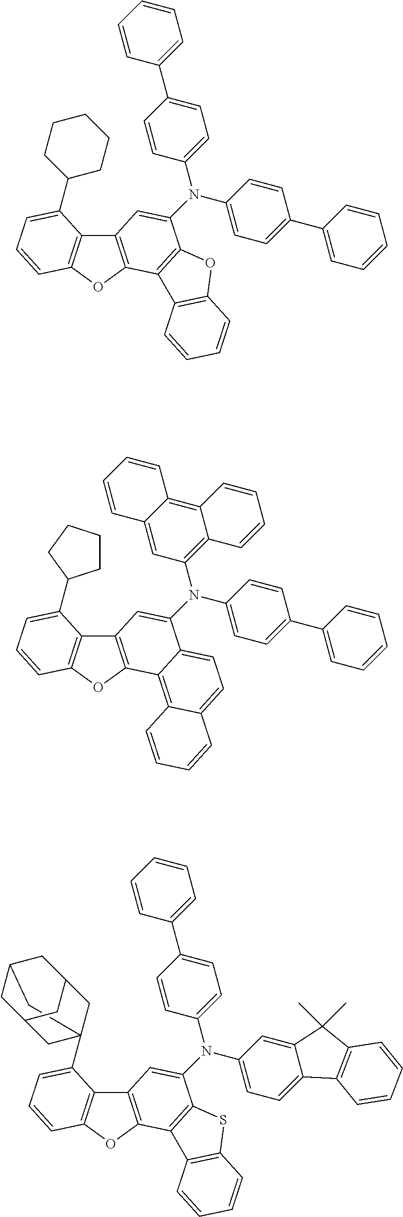 [SMILES: c1ccc(-c2ccc(N(c3ccc(-c4ccccc4)cc3)c3cc4c(oc5cccc(C6CCCCC6)c54)c4c3oc3ccccc34)cc2)cc1 ; c1ccc(-c2ccc(N(c3cc4ccccc4c4ccccc34)c3cc4c(oc5cccc(C6CCCC6)c54)c4c3ccc3ccccc34)cc2)cc1 ; CC1(C)c2ccccc2-c2ccc(N(c3ccc(-c4ccccc4)cc3)c3cc4c(oc5cccc(C67CC8CC(CC(C8)C6)C7)c54)c4c3sc3ccccc34)cc21]

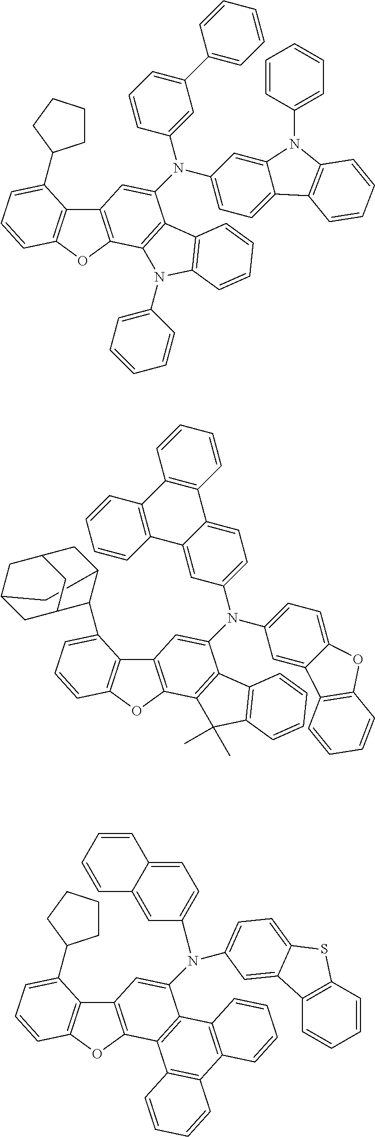 [SMILES: c1ccc(-c2cccc(N(c3ccc4c5ccccc5n(-c5ccccc5)c4c3)c3cc4c(oc5cccc(C6CCCC6)c54)c4c3c3ccccc3n4-c3ccccc3)c2)cc1 ; CC1(C)c2ccccc2-c2c(N(c3ccc4oc5ccccc5c4c3)c3ccc4c5ccccc5c5ccccc5c4c3)cc3c(oc4cccc(C5C6CC7CC(C6)CC5C7)c43)c21 ; c1ccc2cc(N(c3ccc4sc5ccccc5c4c3)c3cc4c(oc5cccc(C6CCCC6)c54)c4c5ccccc5c5ccccc5c34)ccc2c1]

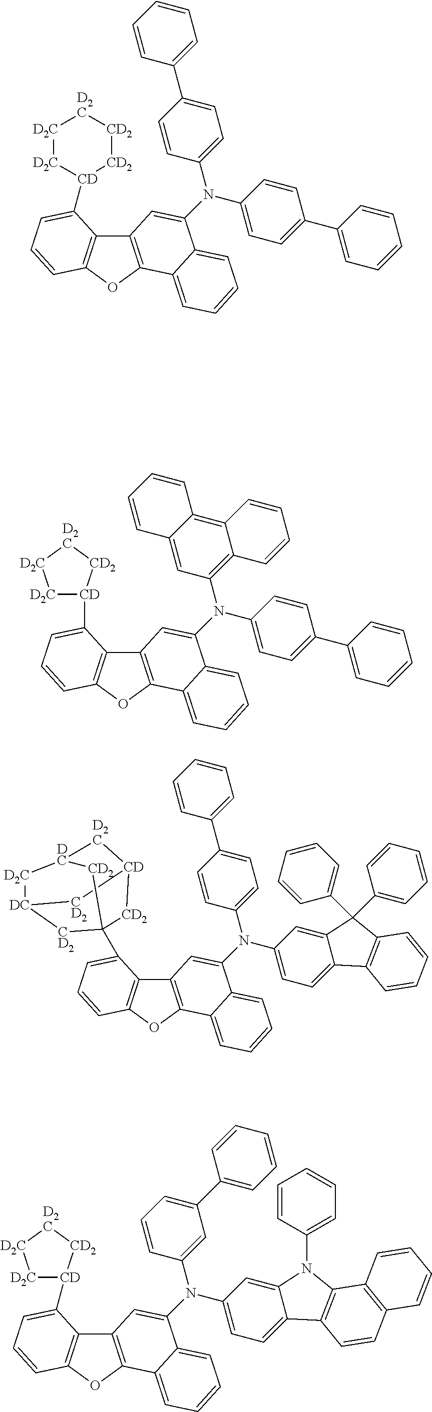

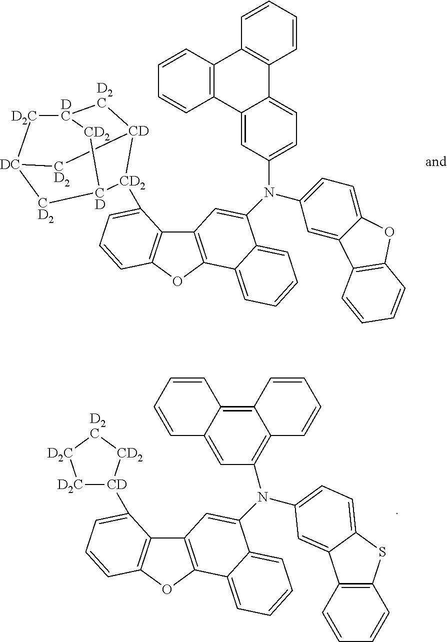

The aromatic amine compound according to the present disclosure may be used as an organic functional material in a functional layer of an organic electronic device, such as a functional layer of an OLED device. The functional layer includes but is not limited to a hole injection layer (HIL), hole transport layer (HTL), electron transport layer (ETL), electron injection layer (EIL), electron barrier layer (EBL), hole barrier layer (HBL), and a light-emitting layer (EML).

In some embodiments, the aromatic amine compound according to the present disclosure may be used in the electron barrier layer.

Furthermore, in some embodiments, the aromatic amine compound according to the present disclosure may be used in the electron barrier layer of a red light organic electronic device.

The present disclosure also provides a mixture. The mixture includes the aromatic amine compound and an organic functional material. The organic functional material may be selected from a hole injection material (HIM), a hole transport material (HTM), an electron transport material (ETM), an electron injection material (EIM), an electron barrier material (EBM), a hole barrier material (HBM), a light-emitting material, a host material, or an organic dye. Details of the above-mentioned organic functional material can refer to patent applications of WO2010135519A1, U.S. Pat. No. 20,090,134784A1, and WO2011110277A1. Therefore, all contents of the above-mentioned patent applications are incorporated into the present disclosure as a reference.

In some embodiments, the organic functional material may be the electron transport material and applied in the organic electronic device as a co-host.

The present disclosure also provides a composition. The composition includes the aromatic amine compound and an organic solvent. The organic solvent may be selected from aromatic, heteroaromatic, ester, aromatic ketone, aromatic ether, aliphatic ketone, aliphatic ether, alicyclic, olefin compound, borate ester, phosphate ester, or a mixture of two or more the above-mentioned solvents.

In some embodiments, the organic solvent may be selected from an aromatic-based solvent or an heteroaromatic-based solvent. The aromatic-based solvent and the heteroaromatic-based solvent suitable for the present disclosure include, but are not limited thereto: p-diisopropylbenzene, pentyl benzene, tetrahydronaphthalene, cyclohexylbenzene, chloronaphthalene, 1,4-dimethylnaphthalene, 3-isopropylbiphenyl, p-methylisopropyl benzene, dipentyl benzene, tripentyl benzene, pentyl toluene, o-diethylbenzene, m-diethylbenzene, p-diethylbenzene, 1,2,3,4-tetratoluene, 1,2,3,5-tetratoluene, 1,2,4,5-tetratoluene, butadiene benzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylcumene, 1-methylnaphthalene, 1,2,4-trichlorobenzene, 4,4-difluorodiphenylmethane, 1,2-dimethoxy-4-(1-propenyl) benzene, diphenylmethane, 2-phenylpyridine, 3-phenylpyridine, N-methyldiphenylamine, 4-isopropylbiphenyl, α, α-dichlorodiphenylmethane, 4-(3-phenylpropyl) pyridine, benzyl benzoate, 1,1-bis (3,4-dimethylphenyl) ethane, 2-isopropylnaphthalene, quinoline, isoquinoline, methyl 2-furanoate, and ethyl 2-furanoate.

In some embodiments, the organic solvent may be an aromatic ketone-based solvent. The aromatic ketone-based solvent suitable for the present disclosure includes, but is not limited thereto: 1-tetrahydronaphthalenone, 2-tetrahydronaphthalenone, 2-(phenyl epoxy) tetrahydronaphthalenone, 6-(methoxy) tetrahydronaphthalenone, acetophenone, phenylacetone, benzophenone, and derivatives of these compounds, such as 4-methylacetophenone, 3-methylacetophenone, 2-methylacetophenone, 4-methylphenylacetone, 3-methylphenylacetone, and 2-methylphenylacetone.

In some embodiments, the organic solvent may be an aromatic ether-based solvent. The aromatic ether-based solvent suitable for the present disclosure includes, but is not limited thereto: 3-phenoxytoluene, butoxybenzene, p-anisaldehyde dimethyl acetal, tetrahydro-2-phenoxy-2H-pyran, 1,2-dimethoxy-4-(1-propenyl) benzene, 1,4-benzodioxane, 1,3-dipropylbenzene, 2,5-dimethoxytoluene, 4-ethylbasic ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-p-propenylanisole, 1,2-dimethoxybenzene, 1-methoxynaphthalene, diphenyl ether, 2-phenoxymethyl ether, 2-phenoxytetrahydrofuran, and ethyl 2-naphthyl ether.

In some embodiments, the organic solvent may be an aliphatic ketone-based solvent. The aliphatic ketone-based solvent suitable for the present disclosure includes, but is not limited thereto: 2-nonone, 3-nonone, 5-nonone, 2-decanone, 2,5-hexanedione, 2,6,8-trimethyl-4-nonone, fenone, phorone, isophorone, and di-n-pentyl ketone.

In some embodiments, the organic solvent may be an aliphatic ether-based solvent. The aliphatic ether-based solvent suitable for the present disclosure includes, but is not limited thereto: amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, and tetraethylene glycol dimethyl ether.

In some embodiments, the organic solvent may be an ester-based solvent. The ester-based solvent suitable for the present disclosure includes, but is not limited octanoate, sebacate, stearate, benzoate, phenylacetate, cinnamate, oxalate, maleate, alkyl lactone, oleate, and the like. In some embodiments, the ester-based solvents may be selected from octyl octanoate, diethyl sebacate, diallyl phthalate, or isononyl isononanoate.

It can be understood that the organic solvent may be one organic solvent or as a mixture of two or more organic solvents.

In some embodiments, the composition according to the present disclosure may further include another organic solvent, other than the aromatic amine compound, the mixture, and the organic solvent as described above. Examples of another organic solvent includes, but is not limited thereto: methanol, ethanol, 2-methoxyethanol, dichloromethane, chloroform, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethyl sulfoxide, tetralin, naphthane, indene, and mixture thereof.

In some embodiments, the organic solvent suitable for the present disclosure is a solvent with Hansen solubility parameters within the following ranges: δd (dispersion force) of the organic solvent ranges from 17.0 MPa^{1/2} to 23.2 MPa^{1/2}, and further ranges from 18.5 MPa^{1/2} to 21.0 MPa^{1/2}; Sp (polarity force) of the organic solvent ranges from 0.2 MPa^{1/2} to 12.5 MPa^{1/2}, and further ranges from 2.0 MPa^{1/2} to 6.0 MPa^{1/2}; and 8h (hydrogen bonding force) of the organic solvent ranges from 0.9 MPa^{1/2} to 14.2 MPa^{1/2}, and further ranges from 2.0 MPa^{1/2} to 6.0 MPa^{1/2}.

In some embodiments, a boiling point of the organic solvent is greater than or equal to 150° C. In some embodiments, the boiling point is greater than or equal to 180° C. In some embodiments, the boiling point is greater than or equal to 200° C. In some embodiments, the boiling point is greater than or equal to 250° C. In some embodiments, the boiling point is greater than or equal to 275° C. or greater than or equal to 300° C. The boiling point within these ranges are beneficial to prevent nozzles of inkjet printing heads from clogging.

It can be understood that the organic solvent may be evaporated from a solvent system to form films including the aromatic amine compound.

In some embodiments, the composition may be a solution. In some embodiments, the composition may be a suspension. The solution or the suspension may also include an additive for adjusting viscosity, forming performance of films, and improving adhesion. The additive may be selected from but not limited to at least one of surfactant, lubricant, wetting agent, dispersant, hydrophobic agent, or adhesive.

In some embodiments, based on the total weight of the composition, a mass fraction of the aromatic amine compound or the mixture in the composition may range from 0.01 wt % to 10 wt %, 0.1 wt % to 15 wt %, 0.2 wt % to 5 wt %, or 0.25 wt % to 3 wt %.

The composition of the present disclosure may be used as a coating material or printing ink applying in preparation of organic electronic devices. In some embodiments, the composition may be used to prepare the organic electronic devices by a printing process or coating process. The printing process or the coating process includes but is not limited thereto: inkjet printing, intaglio printing, jet printing, letterpress printing, screen printing, dip coating, rotating coating, scraper coating, roller printing, rotary roller printing, lithographic printing, flexographic printing brush, rotary printing, spray coating, brush coating or pad printing, slit extrusion coating, or the like. In some embodiments, the printing process or the coating process may be intaglio printing, jet printing, or inkjet printing.

The present disclosure also provides an application of the aromatic amine compound, the mixture, or the composition in preparation of the organic electronic device. The organic electronic device includes a first electrode, a second electrode, and one or more organic functional layers disposed between the first electrode and the second electrode. The organic functional layer includes the aromatic amine compound and the mixture, or is prepared from the above-mentioned composition. In some specific embodiments, the organic electronic device includes a cathode, an anode, and one or more organic functional layers disposed between the cathode and the anode.

The organic electronic device may be, but not limited to, an organic light-emitting diode (OLED), an organic photovoltaic battery (OPV), an organic light-emitting battery (OLEEC), an organic field-effect tube (OFET), an organic light-emitting field-effect tube, an organic laser, an organic spin electron device, an organic sensor, an organic plasmon emission diode (OPED), or the like. In some embodiments, the organic electronic device may be an organic electroluminescent device, such as the OLED, or the organic light-emitting field-effect tube. In some embodiments, the organic electronic device may be the OLED.

The organic functional layer may be selected from a hole injection layer (HIL), a hole transport layer (HTL), a light-emitting layer (EML), an electron barrier layer (EBL), an electron injection layer (EIL), an electron transport layer (ETL), and a hole barrier layer (HBL). In some embodiments, the organic electronic device includes the cathode, the anode, the hole transport layer, the hole injection layer, the light-emitting layer, the electron barrier layer, the electron injection layer, or the electron injection layer.

In some embodiments, the organic functional layer includes the electron barrier layer, which includes the aromatic amine compound or the mixture as described above, or is prepared from the above-mentioned composition.

In some embodiments, the organic electronic device may be the OLED, which includes a substrate, an anode, an electron barrier layer, a light-emitting layer, and a cathode disposed sequentially.

The substrate may be a transparent substrate or an opaque substrate. The substrate may be rigid or elastic. Specifically, the substrate may be plastic, metal, a semiconductor wafer, or a glass. The substrate may have a smooth surface. For example, the substrate may be a substrate without surface defects. In some embodiments, the substrate may be flexible, and a material of the substrate may be selected from but not limited to a polymer film or plastic. A glass transition temperature Tg of the material of the substrate may be greater than 150° C., 200° C., 250° C., or 300° C. Examples of suitable flexible substrates include polyethylene terephthalate (PET) and polyethylene naphthalene-2,6-dicarboxylate (PEN).

The anode is an electrode used for injecting holes, and the holes in the anode may be easily injected into the hole injection layer, the hole transport layer, or the light-emitting layer. A material of the anode may include at least one of conductive metal, conductive metal oxide, and conductive polymer. In some embodiments, absolute value of a difference between work function of the anode and highest occupied molecular orbital (HOMO) energy level or valence band energy level of a light-emitting material of the light-emitting layer, or a p-type semiconductor material of the HIL, the HTL, or the EBL is less than 0.5 eV. In some embodiments, the above-mentioned absolute value is less than 0.3 eV. In some embodiments, the above-mentioned absolute value is less than 0.2 eV. Examples of the material of the anode includes but is not limited thereto: aluminum (Al), copper (Cu), aurum (Au), argentum (Ag), magnesium (Mg), ferrum (Fe), cobalt (Co), nickel (Ni), manganese (Mn), palladium (Pd), platinum (Pt), indium tin oxide (ITO), aluminum doped zinc oxide (AZO), or the like. The material of the anode may be applied to by any suitable technology, such as a suitable physical vapor deposition method including RF magnetron sputtering, vacuum thermal evaporation, electron beam (e-beam), or the like. In some embodiments, the anode is a patterned structure.

The electron barrier layer may be disposed between the anode and the light-emitting layer. A material of the electron barrier layer may include the aromatic amine compound according to the present disclosure.

The light-emitting layer may emit red light, green light, or blue light, and may be made of a phosphorescent material or a fluorescent material. The light-emitting material in the light-emitting layer may be a material that can receive holes and electrons from the hole transport layer and the electron transport layer, respectively, and combine them to emit light in a visible light region. In some embodiments, the light-emitting material may be a material with good quantum efficiency for fluorescence or phosphorescence.

Examples of host materials used for the light-emitting layer include a fused aromatic derivative or a heteroaromatic compound. Specifically, examples of the fused aromatic derivatives include but are not limited thereto: an anthracene derivative, a pyrene derivative, a naphthalene derivative, a pentacene derivative, a phenanthrene compound, a fluoranthene compound, a carbazole derivative, a dibenzofuran derivative, a ladder type furan compound, and a pyrimidine derivative.

The cathode is an electrode used for injecting electrons, and the electrons in the cathode may be easily injected into the electron injection layer, the electron transport layer, or the light-emitting layer. A material of the cathode may include at least one of conductive metal and conductive metal oxide. In some embodiments, absolute value of a difference between work function of the cathode and lowest unoccupied molecular orbital (LUMO) energy level or valence band energy level of the light-emitting material of the light-emitting layer, or a n-type semiconductor material of the EIL, the ETL, or the HBL is less than 0.5 eV. In some embodiments, the above-mentioned absolute value is less than 0.3 eV. In some embodiments, the above-mentioned absolute value is less than 0.2 eV. All materials that may be used in the cathode of organic electronic devices may be used as the material of the cathode of the organic electronic device according to the present disclosure. The material of the cathode includes, but is not limited thereto: Al, Au, Ag, calcium (Ca), barium (Ba), Mg, LiF/Al, MgAg alloy, BaF₂/Al, Cu, Fe, Co, Ni, Mn, Pd, Pt, ITO, or the like. The material of the cathode may be applied to by any suitable technology, such as a suitable physical vapor deposition method including RF magnetron sputtering, vacuum thermal evaporation, electron beam (e-beam), or the like.

In some embodiments, the OLED further includes a hole injection layer, a hole transport layer, an electron injection layer, and an electron transport layer.

The hole injection layer may be used for promoting an injection of holes from the anode to the light-emitting layer. A hole injection material in the hole injection layer may be used to receive holes injected from a positive electrode at low voltages. In some embodiments, HOMO energy level of the hole injection material is between work function of a positive electrode material and HOMO energy level of a peripheral organic material layer. The hole injection material includes, but is not limited thereto: metalloporphyrin, oligothiophene, an organic material based on arylamine, an organic material based on hexacyano hexaazabenzophenanthrene, an organic material based on quinacridone, an organic material based on perylene, anthraquinone, conductive polymer based on polyaniline, polythiophene, or the like.

The hole transport layer may be used for transmitting holes. A hole transport material in the hole transport layer may have high hole mobility, and is used to receive holes transmitted from the anode or the hole injection layer and transmit the holes to the light-emitting layer. The hole transport material may include, but is not limited thereto: an organic material based on aromatic amine, an organic material based on carbazole, conductive polymer, block copolymer with both conjugated and non-conjugated portions.

The electron injection layer may be used for injecting electrons. An electron injection material in the electron injection layer may have ability to transmit electrons, an effect of injecting electrons from a negative electrode, an excellent effect of injecting electrons into the light-emitting layer or the light-emitting material, and a function of preventing excitons generated by the light-emitting layer from transmitting to the hole injection layer. The electron injection material further has excellent ability to form thin films. The electron injection material includes, but is not limited thereto: 8-hydroxyquinoline lithium (LiQ), fluorenone, anthraquinone dimethyl, biphenylquinone, thian dioxide, azole, diazole, triazole, imidazole, perylene tetracarboxylic acid, fluorene methane, anthrone, derivatives of these compounds, metal complexes, nitrogen with 5-membered ring derivatives, or the like.

The electron transport layer may be used for transmitting electrons. An electron transport material in the electron transport layer has high electron mobility, and is used to receive electrons injected from a negative electrode and transmit the electrons to the light-emitting layer. The electron transport material may include, but is not limited thereto: an Al-based complex of 8-hydroxyquinoline, a complex containing Alq₃, an organic radical compound, an hydroxyflavone metal complex, 8-hydroxyquinoline lithium (LiQ), and a compound based on benzimidazole.

It can be understood that in some embodiments, the organic electronic device may also include the hole barrier layer disposed between the light-emitting layer and the electron transport layer. The hole barrier layer is a layer that blocks holes from reaching a negative electrode and may usually be formed under the same conditions as the hole injection layer. A hole barrier material in the hole barrier layer may include but is not limited thereto: a diazole derivative, a triazole derivative, a phenanthroline derivative, bromocresol purple sodium salt (BCP), an aluminum complex, or the like.

Referring to FIG., a specific embodiment of the present disclosure provides an organic electronic device 100, which includes a substrate 101, an anode 102, a hole injection layer 103, a hole transport layer 104, an electron barrier layer 105, a light-emitting layer 106, an electron transport layer 107, an electron injection layer 108, and a cathode 109.

In some embodiments, the organic electronic device according to the present disclosure may be a solution type organic electronic device, and one or more functional layers in the organic electronic device is prepared by a printing process. In some embodiments, the solution type organic electronic device may be a solution type OLED.

The present disclosure also provides an application of the organic electronic device in preparation of various electronic devices, and the electronic devices include but are not limited thereto: a display device, an illumination device, a light source, a sensor, or the like.

The following is specific examples to illustrate the present disclosure. The following examples are only some examples of the present disclosure and are not a limitation to the present disclosure.

The following is specific illustration of synthesis routes and synthesis methods of the aromatic amine compounds (hereinafter referred to as compounds M1-M24) of the present disclosure through specific examples. The following examples are only preferred examples of the present disclosure, but cannot be understood as limitations to the present disclosure.

### Example 1

Synthesis of Compound M1

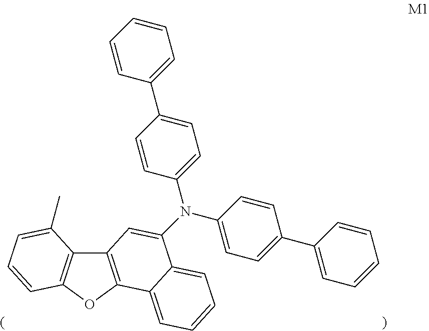 [SMILES: Cc1cccc2oc3c4ccccc4c(N(c4ccc(-c5ccccc5)cc4)c4ccc(-c5ccccc5)cc4)cc3c12]

Synthetic Route of the Compound M1 is as follows.

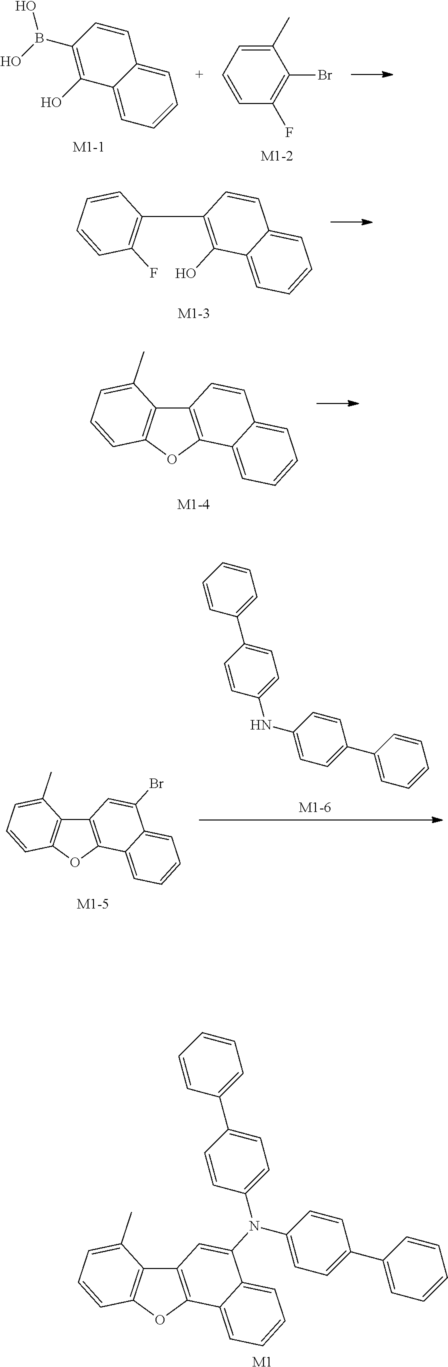

1) Synthesis of intermediate M1-3: in a nitrogen atmosphere, 18.8 g (100 mmol) of compound M1-1, 18.9 g (100 mmol) of compound M1-2, 3.31 g (3 mmol) of tetratriphenylphosphine palladium, 50 mL aqueous solution of potassium carbonate (27.6 g, 200 mmol), and 200 mL of toluene were added into a three necked-flask (500 mL). The solution was heated to a temperature of 60°° C., and stirred for 12 hours. After the reaction was completed, the solution was cooled to room temperature, rotationally evaporated to remove most of the solvent, dissolved with dichloromethane, and washed with water for three times. The organic solution was collected, and purified by column chromatography to obtain the intermediate M1-3 with yield of 72%.
2) Synthesis of intermediate M1-4: in a nitrogen atmosphere, 15.2 g (60 mmol) of intermediate M1-3, 32.6 g (100 mmol) of cesium carbonate, and 100 mL of dimethylformamide were added into a three necked-flask (300 mL). The solution was heated to 120° C., and stirred for 12 hours. After the reaction was completed, the solution was cooled to room temperature, and mixed with 300 mL of water and stirred to obtain the precipitate. The precipitate was filtered to obtain the filter residue. The filter residue was recrystallized with a mixed solution of ethyl acetate and ethanol, to obtain the intermediate M1-4 with yield of 78%.

13) Synthesis of intermediate M1-5: in a nitrogen atmosphere, 9.3 g (40 mmol) of intermediate M1-4 and 50 mL of chloroform were added into a three necked-flask (250 mL). The three neck-flask was putted in an ice bath where the solution was stirred. 50 mL of chloroform solution containing N-bromosuccinimide (7.2 g, 40 mmol) was slowly added into the solution. Naturally raise the reaction solution to room temperature. Then the reaction solution was stirred for 6 hours. After the reaction was completed, the reaction solution was mixed with 200 ml of water, extracted with dichloromethane, and washed with water for three times. The organic solution was collected, and purified by column chromatography to obtain the intermediate M1-5 with yield of 83%.
4) Synthesis of the compound M1: in a nitrogen atmosphere, 9.3 g (30 mmol) of compound M1-5, 9.7 g (30 mmol) of compound M1-6, 0.55 g (0.6 mmol) of Pd₂(dba)₃, 0.24 g (1.2 mmol) of tertbutyl phosphine, 4.1 g (45 mmol) of tert-butanol sodium, and 100 mL of anhydrous toluene solvent were added to a three necked-flask (300 mL). The solution was heated to 60° C., and stirred for 6 hours. After the reaction was completed, the solution was cooled to room temperature and quenched with water. The solution was filtered to evaporate most of the solvent, and then dissolved with dichloromethane, and washed with water for three times. The organic solution was collected, and purified by column chromatography to obtain the compound M1 with yield of 86%. A result of atmospheric solids analysis probe-mass spectrometry (ASAP-MS) of the compound M1 was as follows: MS (ASAP)=552

### Example 2

### Synthesis of Compound M2

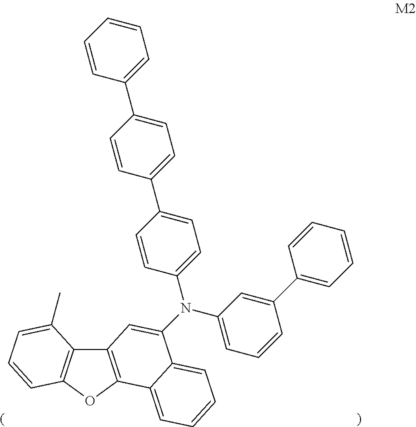 [SMILES: Cc1cccc2oc3c4ccccc4c(N(c4ccc(-c5ccc(-c6ccccc6)cc5)cc4)c4cccc(-c5ccccc5)c4)cc3c12]

Synthetic Route of the Compound M2 is as follows.

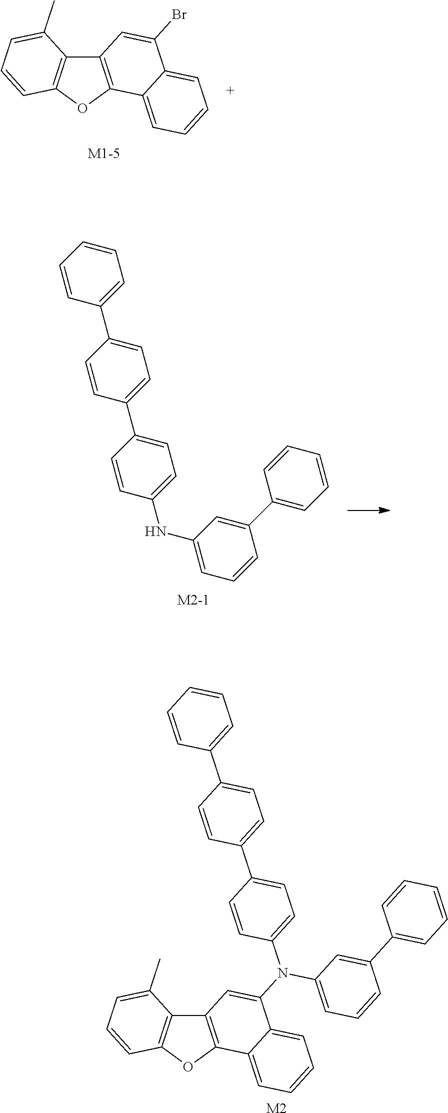

Synthesis of the compound M2: according to the synthesis method of the compound M1, compound M2-1 was substituted for the compound M1-6 to obtain the compound M2 with yield of 83%. MS (ASAP)=628.

### Example 3

### Synthesis of Compound M3

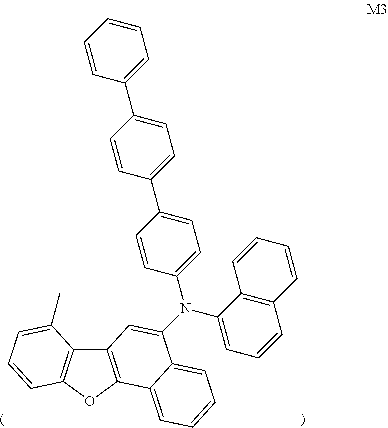 [SMILES: Cc1cccc2oc3c4ccccc4c(N(c4ccc(-c5ccc(-c6ccccc6)cc5)cc4)c4cccc5ccccc45)cc3c12]

Synthetic Route of the Compound M3 is as follows.

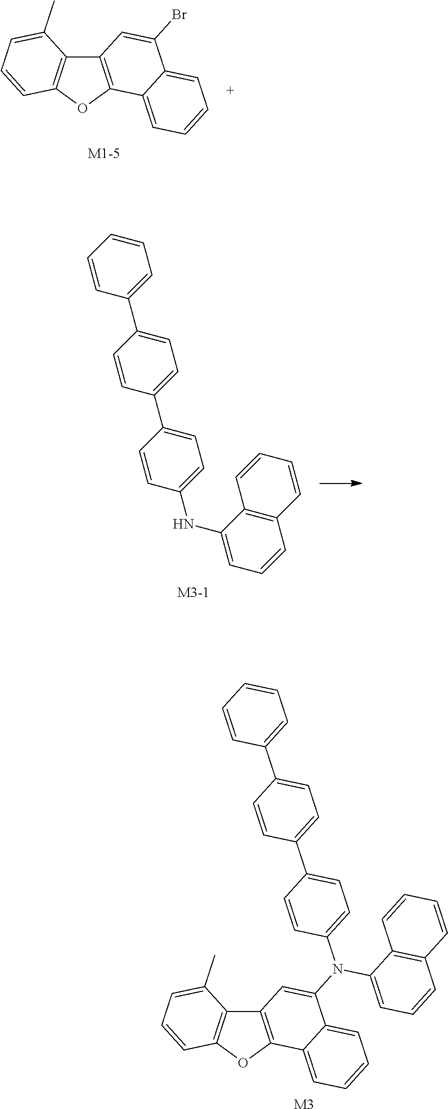

Synthesis of the compound M3: according to the synthesis method of the compound M1, compound M3-1 was substituted for the compound M1-6 to obtain the compound M3 with yield of 81%. MS (ASAP)=602.

### Example 4

### Synthesis of Compound M4

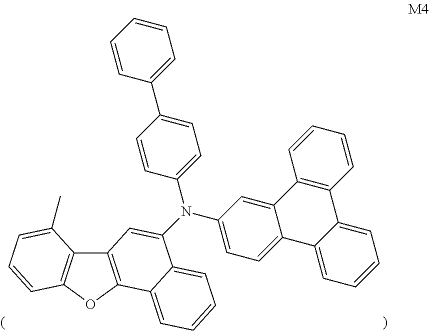 [SMILES: Cc1cccc2oc3c4ccccc4c(N(c4ccc(-c5ccccc5)cc4)c4ccc5c6ccccc6c6ccccc6c5c4)cc3c12]

Synthetic Route of the Compound M4 is as follows.

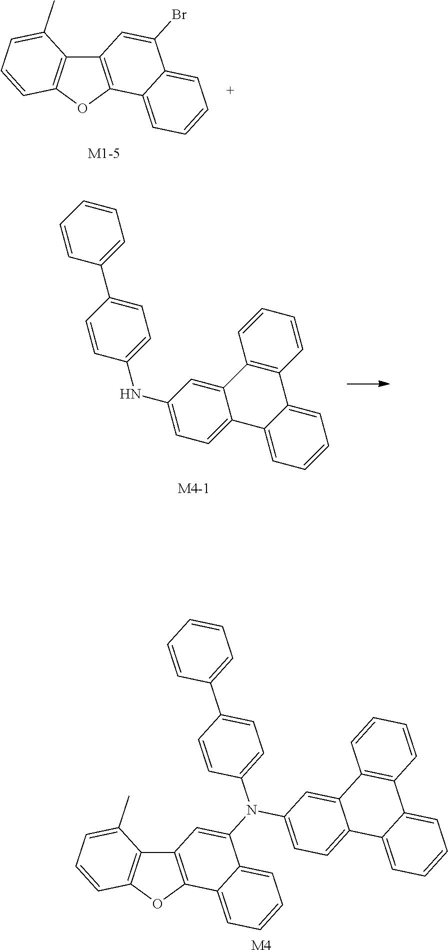

Synthesis of the compound M4: according to the synthesis method of the compound M1, compound M4-1 was substituted for the compound M1-6 to obtain the compound M4 with yield of 82%. MS (ASAP)=626.

### Example 5

### Synthesis of Compound M5

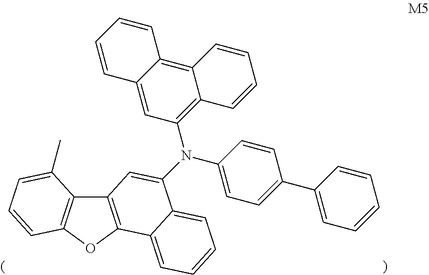 [SMILES: Cc1cccc2oc3c4ccccc4c(N(c4ccc(-c5ccccc5)cc4)c4cc5ccccc5c5ccccc45)cc3c12]

Synthetic Route of the Compound M5 is as follows.

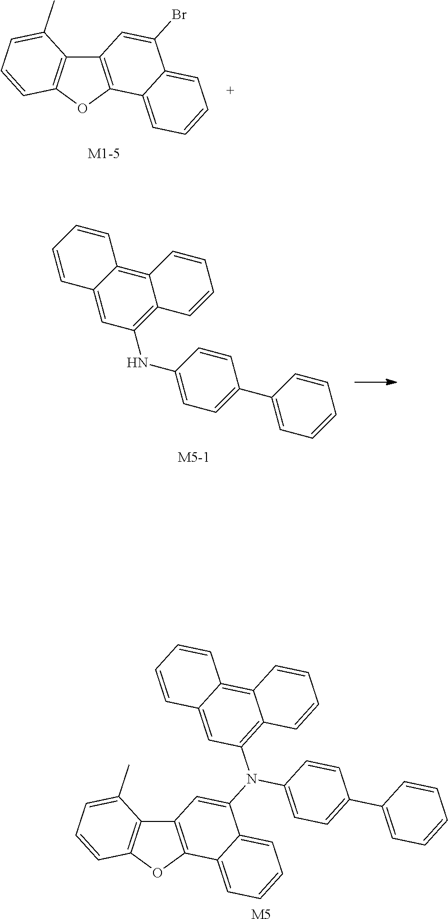

Synthesis of the compound M5: according to the synthesis method of the compound M1, compound M5-1 was substituted for the compound M1-6 to obtain the compound M5 with yield of 77%. MS (ASAP)=576.

### Example 6

### Synthesis of Compound M6

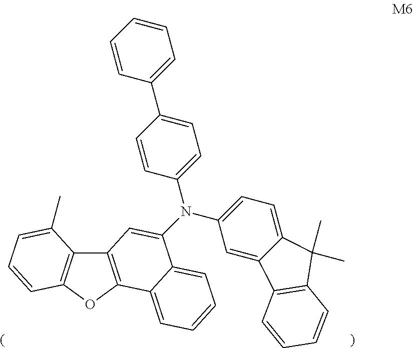 [SMILES: Cc1cccc2oc3c4ccccc4c(N(c4ccc(-c5ccccc5)cc4)c4ccc5c(c4)-c4ccccc4C5(C)C)cc3c12]

Synthetic Route of the Compound M6 is as follows.

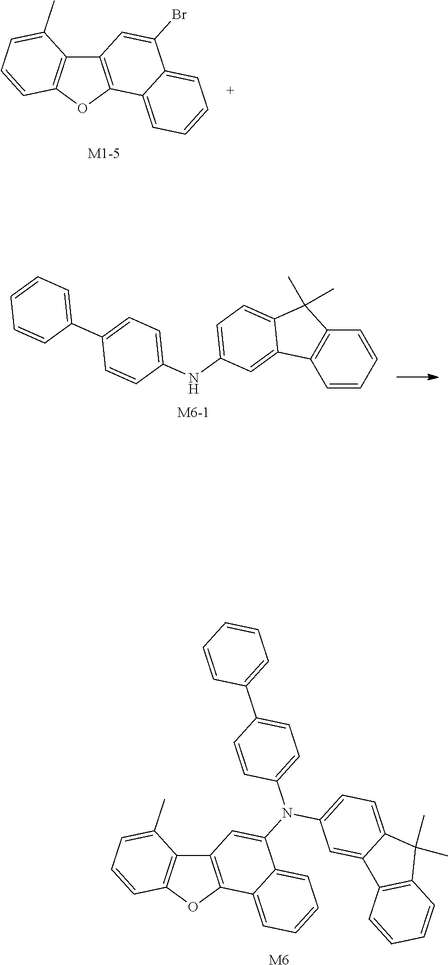

Synthesis of the compound M6: according to the synthesis method of the compound M1, compound M6-1 was substituted for the compound M1-6 to obtain the compound M6 with yield of 84%. MS (ASAP)=592.

### Example 7

### Synthesis of Compound M7

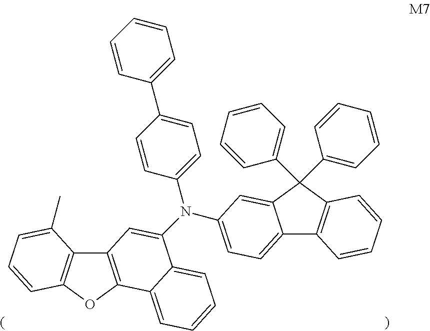 [SMILES: Cc1cccc2oc3c4ccccc4c(N(c4ccc(-c5ccccc5)cc4)c4ccc5c(c4)C(c4ccccc4)(c4ccccc4)c4ccccc4-5)cc3c12]

Synthetic Route of the Compound M7 is as follows.

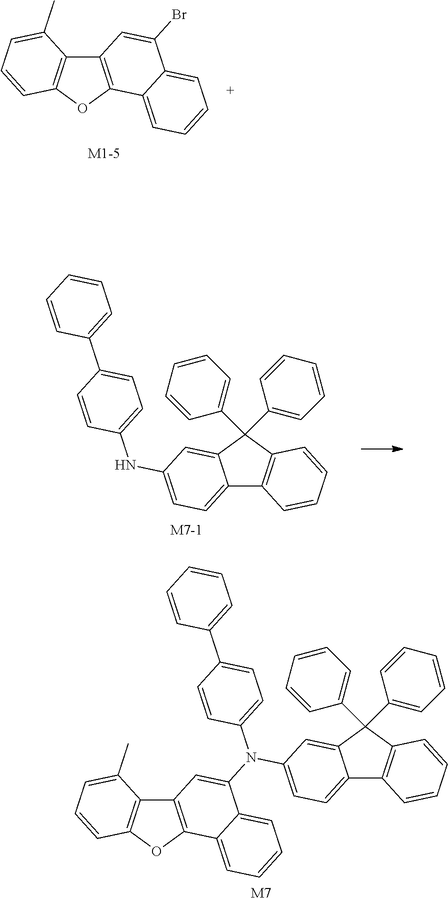

Synthesis of the compound M7: according to the synthesis method of the compound M1, compound M7-1 was substituted for the compound M1-6 to obtain the compound M7 with yield of 82%. MS (ASAP)=716.

### Example 8

### Synthesis of Compound M8

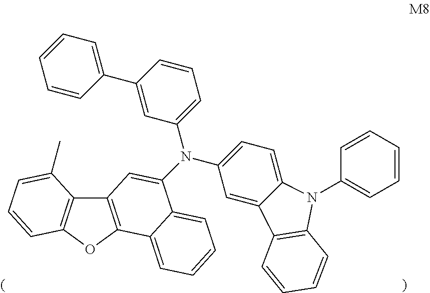 [SMILES: Cc1cccc2oc3c4ccccc4c(N(c4cccc(-c5ccccc5)c4)c4ccc5c(c4)c4ccccc4n5-c4ccccc4)cc3c12]

Synthetic Route of the Compound M8 is as follows.

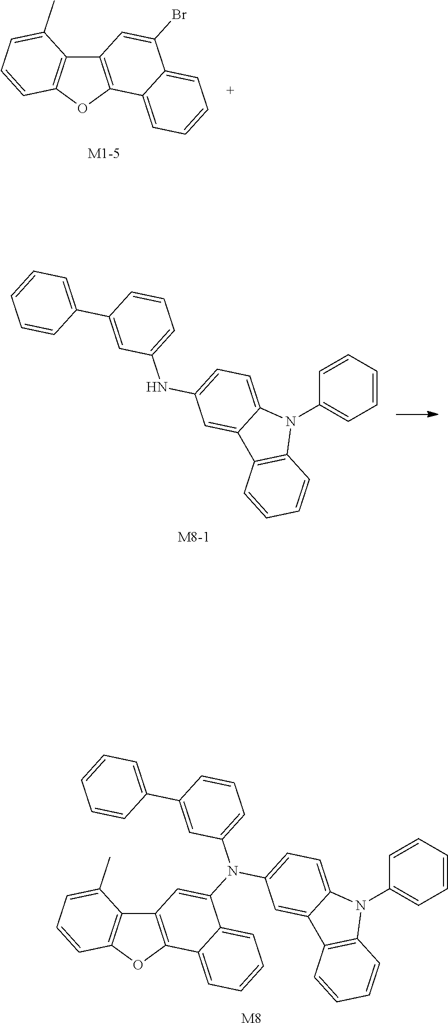

Synthesis of the compound M8: according to the synthesis method of the compound M1, compound M8-1 was substituted for the compound M1-6 to obtain the compound M8 with yield of 83%. MS (ASAP)=641.

### Example 9

### Synthesis of Compound M9

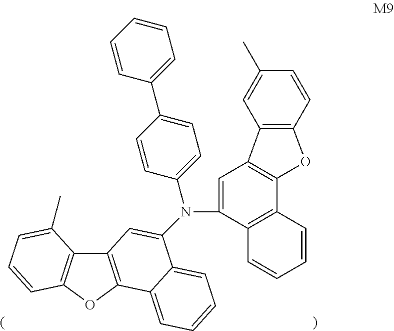 [SMILES: Cc1ccc2oc3c4ccccc4c(N(c4ccc(-c5ccccc5)cc4)c4cc5c(oc6cccc(C)c65)c5ccccc45)cc3c2c1]

Synthetic Route of the Compound M9 is as follows.

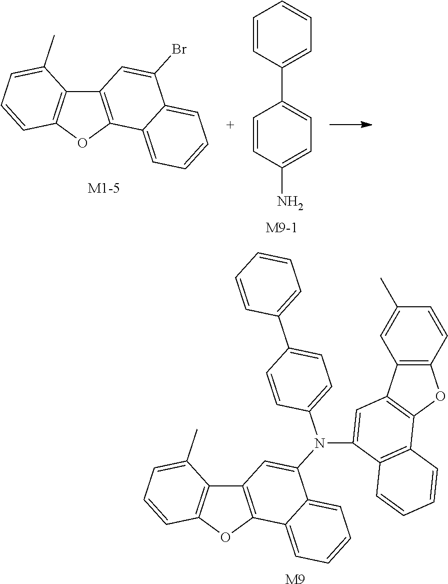

Synthesis of the compound M9: according to the synthesis method of the compound M1, compound M9-1 and double compound M1-5 were substituted for the compound M1-7 and the compound M1-5, respectively, to obtain the compound M9 with yield of 75%. MS (ASAP)=630.

### Example 10

### Synthesis of Compound M10

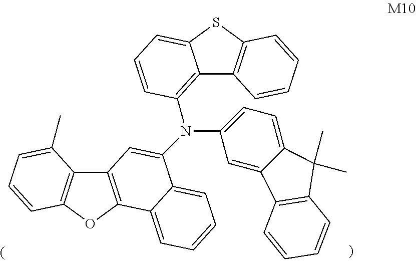 [SMILES: Cc1cccc2oc3c4ccccc4c(N(c4ccc5c(c4)-c4ccccc4C5(C)C)c4cccc5sc6ccccc6c45)cc3c12]

Synthetic Route of the Compound M10 is as follows.

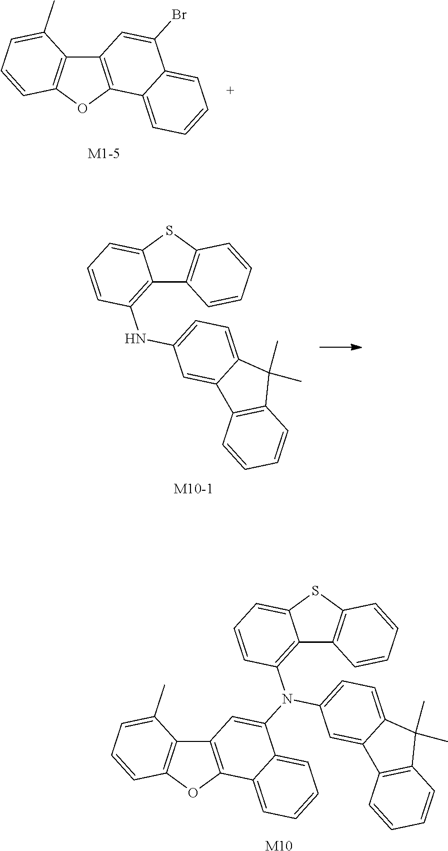

Synthesis of the compound M10: according to the synthesis method of the compound M1, compound M10-1 was substituted for the compound M1-6 to obtain the compound M10 with yield of 76%. MS (ASAP)=622.

### Example 11

### Synthesis of Compound M11

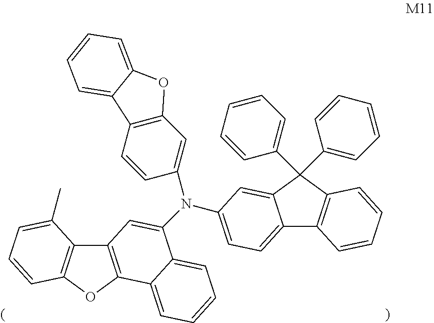 [SMILES: Cc1cccc2oc3c4ccccc4c(N(c4ccc5c(c4)C(c4ccccc4)(c4ccccc4)c4ccccc4-5)c4ccc5c(c4)oc4ccccc45)cc3c12]

Synthetic Route of the Compound M11 is as follows.

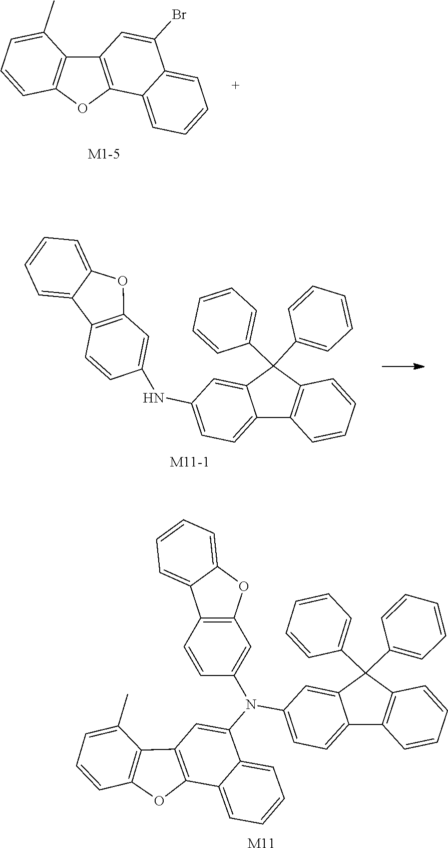

Synthesis of the compound M11: according to the synthesis method of the compound M1, compound M11-1 was substituted for the compound M1-6 to obtain the compound M11 with yield of 80%. MS (ASAP)=730.

### Example 12

### Synthesis of Compound M12

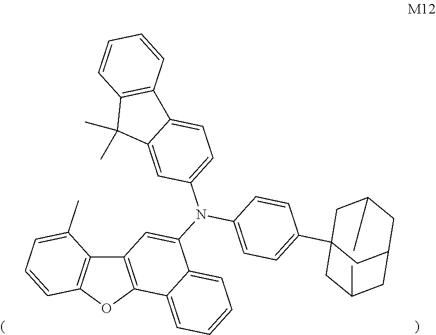 [SMILES: CCC1CC2CC(C)CC(c3ccc(N(c4ccc5c(c4)C(C)(C)c4ccccc4-5)c4cc5c(oc6cccc(C)c65)c5ccccc45)cc3)(C1)C2]

Synthetic Route of the Compound M12 is as follows.

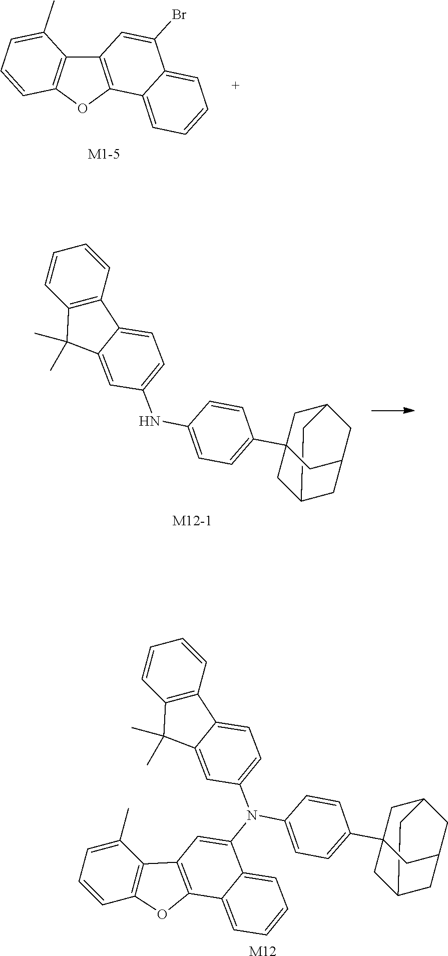

Synthesis of the compound M12: according to the synthesis method of the compound M1, compound M12-1 was substituted for the compound M1-6 to obtain the compound M12 with yield of 82%. MS (ASAP)=696.

### Example 13

### Synthesis of Compound M13

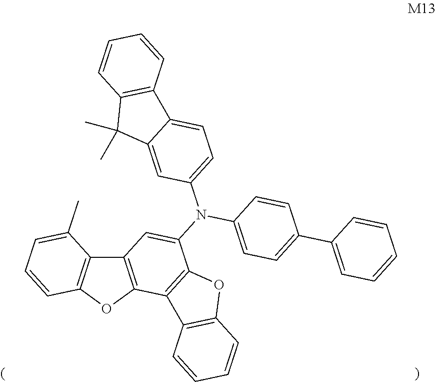 [SMILES: Cc1cccc2oc3c(cc(N(c4ccc(-c5ccccc5)cc4)c4ccc5c(c4)C(C)(C)c4ccccc4-5)c4oc5ccccc5c43)c12]

Synthetic Route of the Compound M13 is as follows:

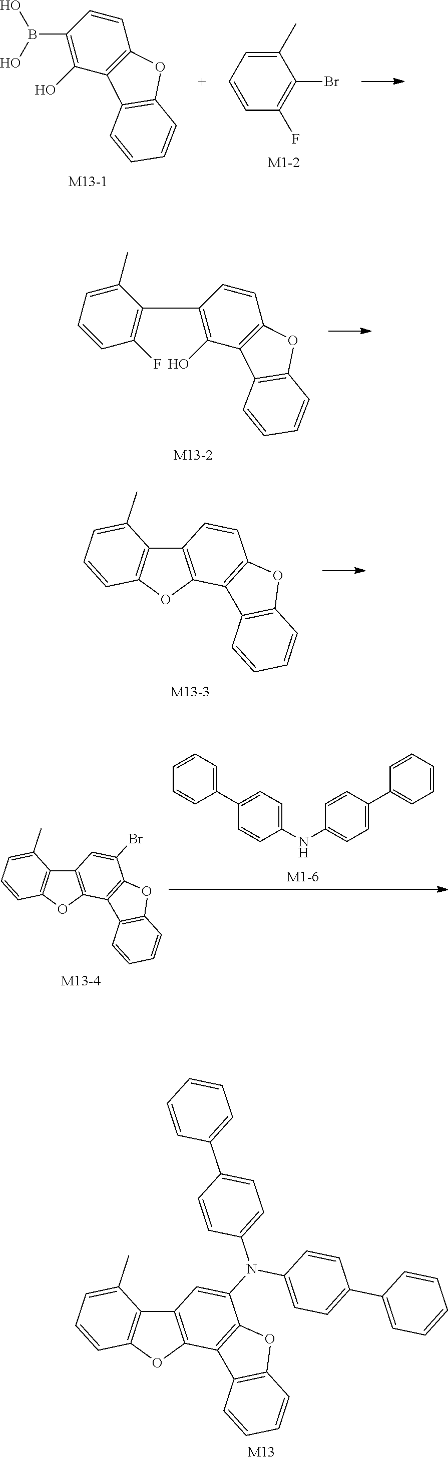

1) Synthesis of intermediate M13-2: according to the synthesis method of the compound M1-3, compound M13-1 was substituted for the compound M1-1 to obtain the intermediate M13-2 with yield of 68%.
2) Synthesis of intermediate M13-3: according to the synthesis method of the compound M1-4, compound M13-2 was substituted for the compound M1-3 to obtain the intermediate M13-3 with yield of 75%.
3) Synthesis of intermediate M13-4: according to the synthesis method of the compound M1-5, compound M13-3 was substituted for the compound M1-4 to obtain the intermediate M13-4 with yield of 82%.
4) Synthesis of the compound M13: according to the synthesis method of the compound M1, compound M13-4 was substituted for the compound M1-5 to obtain the compound M13 with yield of 85%. MS (ASAP)=592.

### Example 14

### Synthesis of Compound M14

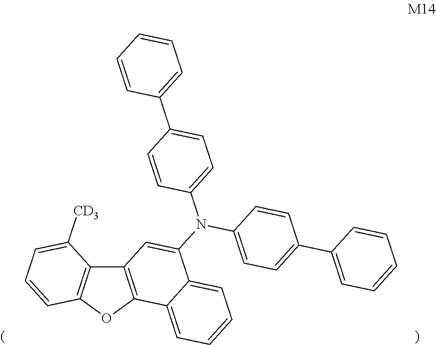 [SMILES: [2H]C([2H])([2H])c1cccc2oc3c4ccccc4c(N(c4ccc(-c5ccccc5)cc4)c4ccc(-c5ccccc5)cc4)cc3c12]

Synthetic Route of the Compound M14 is as follows.

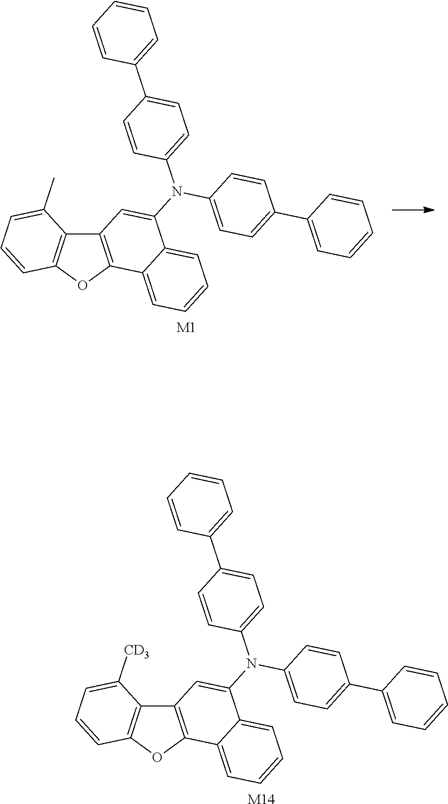

Synthesis of the compound M14: in a nitrogen atmosphere, 11 g (20 mmol) of the compound M1, 1.6 g (40 mmol) of sodium hydroxide, and 30 mL of deuterated dimethyl sulfoxide were added into a two necked-flask (150 mL). The solution was heated to 110° C., and stirred for 6 hours. After the reaction was completed, the solution was cooled to room temperature, quenched with water, and then dissolved with dichloromethane, and washed with water for three times. The organic solution was collected, and purified by column chromatography to obtain the compound M14 with yield of 92%. MS (ASAP)-555.

### Example 15

### Synthesis of Compound M15

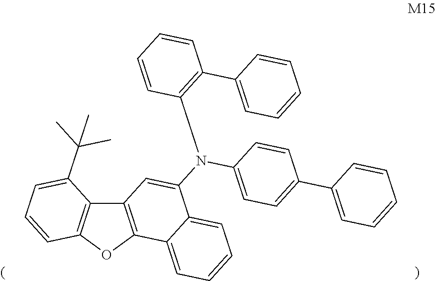 [SMILES: CC(C)(C)c1cccc2oc3c4ccccc4c(N(Cc4ccccc4-c4ccccc4)c4ccc(-c5ccccc5)cc4)cc3c12]

Synthetic Route of the Compound M15 is as follows.

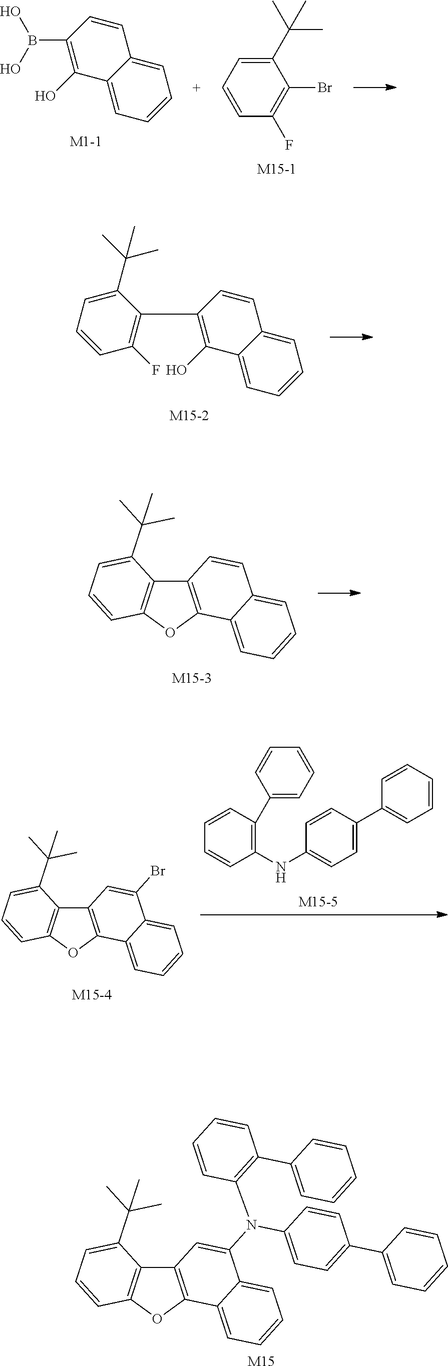

1) Synthesis of intermediate M15-2: according to the synthesis method of the compound M1-3, compound M15-1 was substituted for the compound M1-2 to obtain the intermediate M15-2 with yield of 70%.
2) Synthesis of intermediate M15-3: according to the synthesis method of the compound M1-4, compound M15-2 was substituted for the compound M1-3 to obtain the intermediate M15-3 with yield of 73%.
3) Synthesis of intermediate M15-4: according to the synthesis method of the compound M1-5, compound M15-3 was substituted for the compound M1-4 to obtain the intermediate M15-4 with yield of 81%.
4) Synthesis of the compound M15: according to the synthesis method of the compound M1, compound M15-4 and compound M15-5 were substituted for the compound M1-5 and the compound M1-6, respectively, to obtain the compound M15 with yield of 82%. MS (ASAP)=594.

### Example 16

### Synthesis of Compound M16

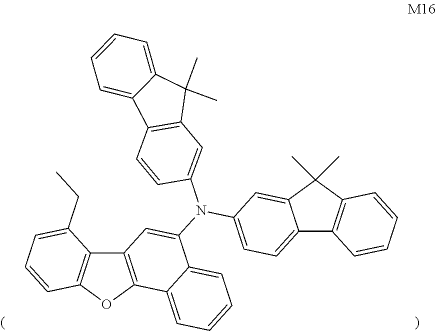 [SMILES: CCc1cccc2oc3c4ccccc4c(N(c4ccc5c(c4)C(C)(C)c4ccccc4-5)c4ccc5c(c4)C(C)(C)c4ccccc4-5)cc3c12]

Synthetic Route of the Compound M16 is as follows.

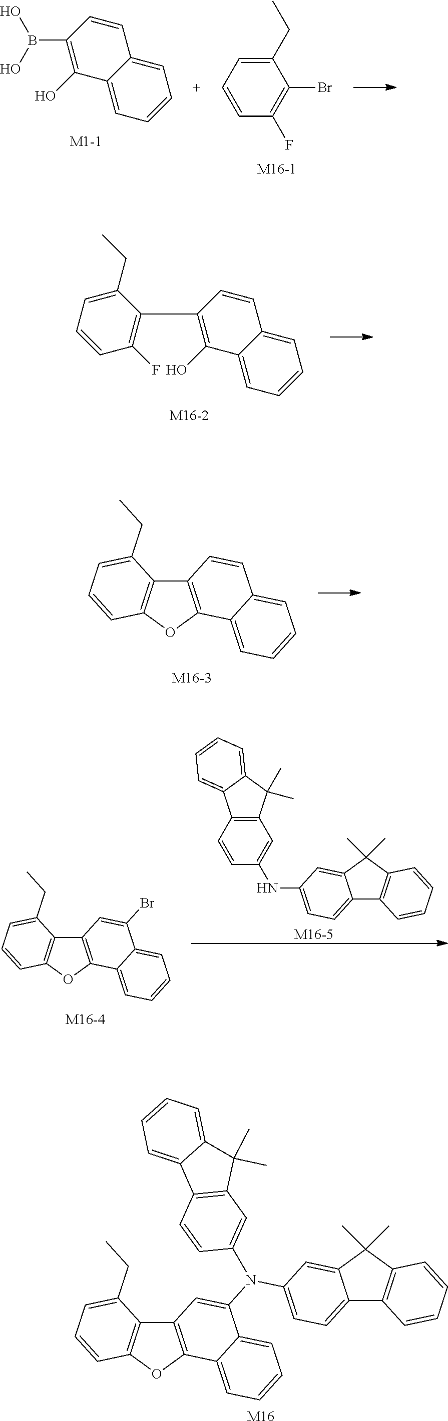

1) Synthesis of intermediate M16-2: according to the synthesis method of the compound M1-3, compound M16-1 was substituted for the compound M1-2 to obtain the intermediate M16-2 with yield of 72%.
2) Synthesis of intermediate M16-3: according to the synthesis method of the compound M1-4, compound M16-2 was substituted for the compound M1-3 to obtain the intermediate M16-3 with yield of 74%.
3) Synthesis of intermediate M16-4: according to the synthesis method of the compound M1-5, compound M16-3 was substituted for the compound M1-4 to obtain the intermediate M16-4 with yield of 82%.
4) Synthesis of the compound M16: according to the synthesis method of the compound M1, compound M16-4 and compound M16-5 were substituted for the compound M1-5 and the compound M1-6, respectively, to obtain the compound M16 with yield of 80%. MS (ASAP)-646.

### Example 17

### Synthesis of Compound M17

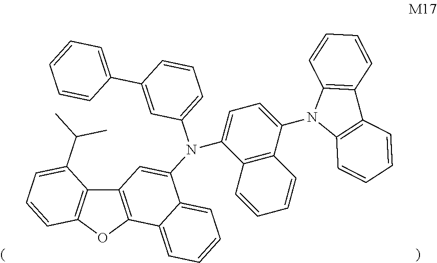 [SMILES: CC(C)c1cccc2oc3c4ccccc4c(N(c4cccc(-c5ccccc5)c4)c4ccc(-n5c6ccccc6c6ccccc65)c5ccccc45)cc3c12]

Synthetic Route of the Compound M17 is as follows.

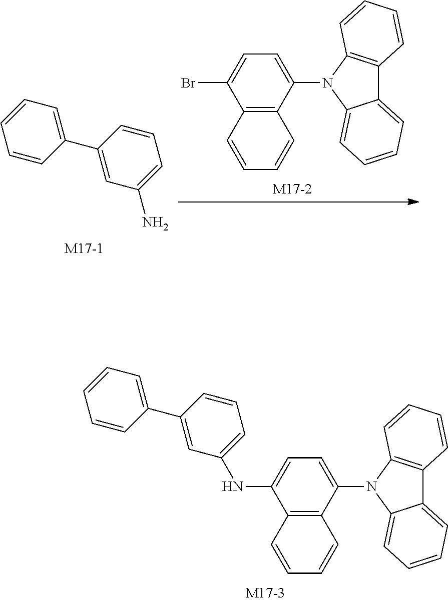 [SMILES: Nc1cccc(-c2ccccc2)c1 ; Brc1ccc(-n2c3ccccc3c3ccccc32)c2ccccc12 ; c1ccc(-c2cccc(Nc3ccc(-n4c5ccccc5c5ccccc54)c4ccccc34)c2)cc1]

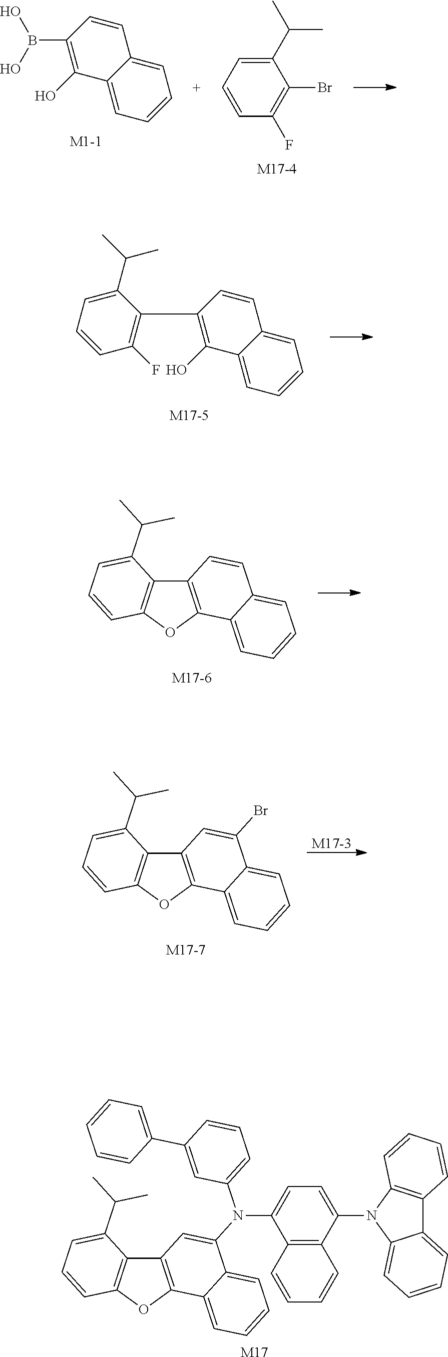

1) Synthesis of intermediate M17-3: according to the synthesis method of the compound M1, compound M17-2 and compound M17-1 were substituted for the compound M1-5 and the compound M1-6, respectively, to obtain the intermediate M17-3 with yield of 76%.
2) Synthesis of intermediate M17-5: according to the synthesis method of the compound M1-3, compound M17-4 was substituted for the compound M1-2 to obtain the intermediate M17-5 with yield of 70%.
3) Synthesis of intermediate M17-6: according to the synthesis method of the compound M1-4, compound M17-5 was substituted for the compound M1-3 to obtain the intermediate M17-6 with yield of 73%.
4) Synthesis of intermediate M17-7: according to the synthesis method of the compound M1-5, compound M17-6 was substituted for the compound M1-4 to obtain the intermediate M17-7 with yield of 81%.
5) Synthesis of the compound M17: according to the synthesis method of the compound M1, compound M17-7 and compound M17-3 were substituted for the compound M1-5 and the compound M1-6, respectively, to obtain the compound M17 with yield of 82%. MS (ASAP)-719.

### Example 18

### Synthesis of Compound M18

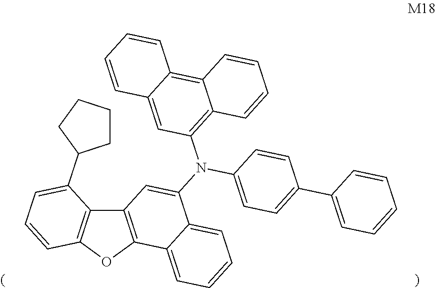 [SMILES: c1ccc(-c2ccc(N(c3cc4ccccc4c4ccccc34)c3cc4c(oc5cccc(C6CCCC6)c54)c4ccccc34)cc2)cc1]

Synthetic Route of the Compound M18 is as follows.

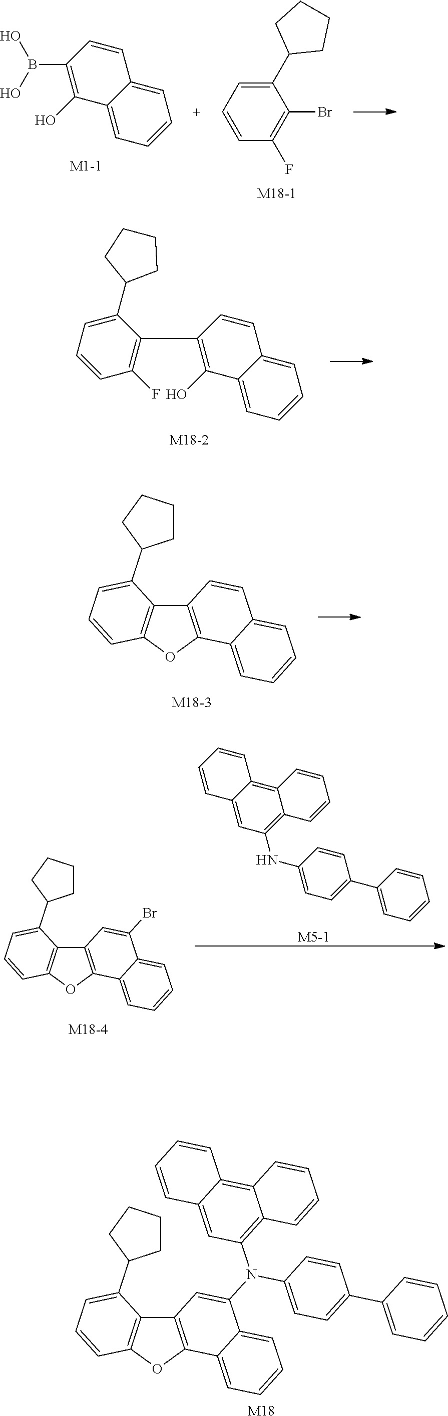

1) Synthesis of intermediate M18-2: according to the synthesis method of the compound M1-3, compound M18-1 was substituted for the compound M1-2 to obtain the intermediate M18-2 with yield of 72%.
2) Synthesis of intermediate M18-3: according to the synthesis method of the compound M1-4, compound M18-2 was substituted for the compound M1-3 to obtain the intermediate M18-3 with yield of 74%.
3) Synthesis of intermediate M18-4: according to the synthesis method of the compound M1-5, compound M18-3 was substituted for the compound M1-4 to obtain the intermediate M18-4 with yield of 82%.
4) Synthesis of the compound M18: according to the synthesis method of the compound M1, compound M18-4 and compound M5-1 were substituted for the compound M1-5 and the compound M1-6, respectively, to obtain the compound M18 with yield of 81%. MS (ASAP)=630.

### Example 19

### Synthesis of Compound M19

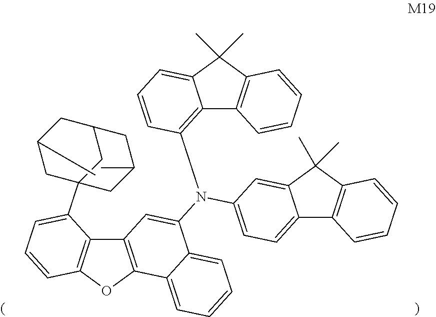 [SMILES: CCC1CC2CC(C)CC(c3cccc4oc5c6ccccc6c(N(Cc6cccc7c6-c6ccccc6C7(C)C)c6ccc7c(c6)C(C)(C)c6ccccc6-7)cc5c34)(C1)C2]

Synthetic Route of the Compound M19 is as follows.

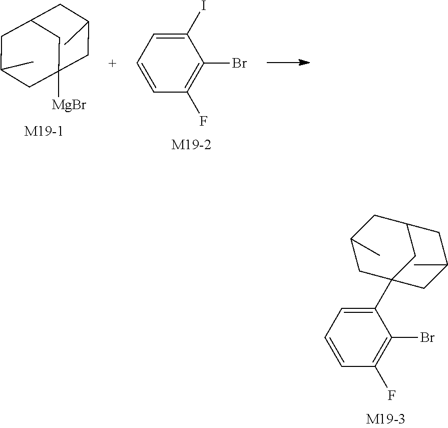

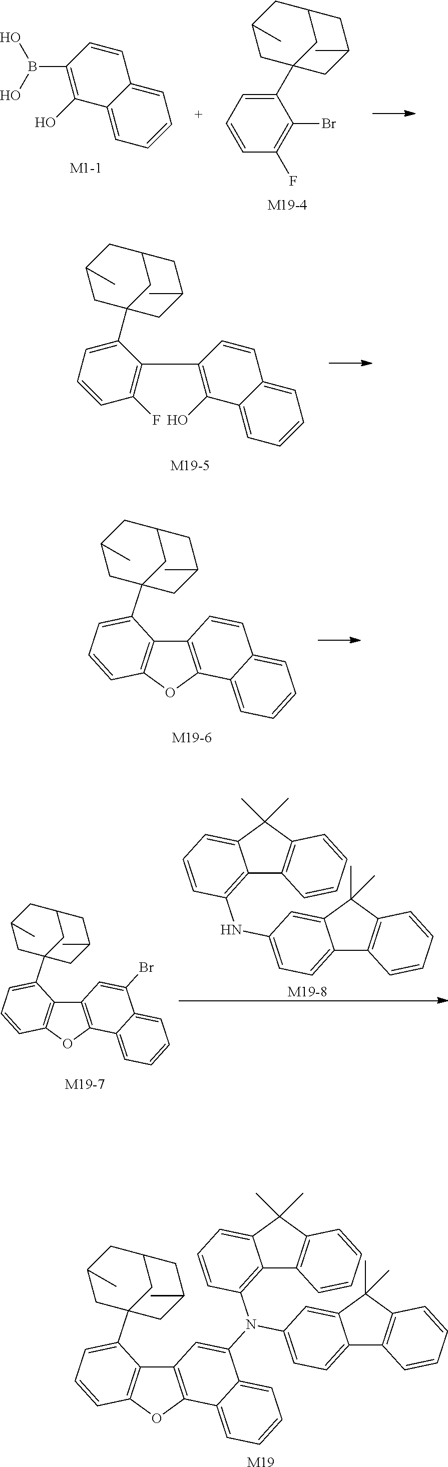

1) Synthesis of intermediate M19-3: in a nitrogen atmosphere, 30.1 g (100 mmol) of compound M19-2 and 150 mL of anhydrous ether were added into a three necked-flask (500 mL). The three neck-flask was putted in an ice bath where the solution was stirred. Ether solution including 100 mmol of compound M19-1 was slowly added into the solution. Then, the reaction solution was stirred for 6 hours. After the reaction was completed, the reaction solution was quenched with water, extracted with ethyl acetate, dissolved and washed with water for three times. The organic solution was collected, and purified by column chromatography to obtain the intermediate M19-3 with yield of 80%.
2) Synthesis of intermediate M19-5: according to the synthesis method of the compound M1-3, compound M19-4 was substituted for the compound M1-2 to obtain the intermediate M19-5 with yield of 72%.
3) Synthesis of intermediate M19-6: according to the synthesis method of the compound M1-4, compound M19-5 was substituted for the compound M1-3 to obtain the intermediate M19-6 with yield of 75%.
4) Synthesis of intermediate M19-7: according to the synthesis method of the compound M1-5, compound M19-6 was substituted for the compound M1-4 to obtain the intermediate M19-7 with yield of 83%.
5) Synthesis of the compound M19: according to the synthesis method of the compound M1, compound M19-7 and compound M19-8 were substituted for the compound M1-5 and the compound M1-6, respectively, to obtain the compound M19 with yield of 82%. MS (ASAP)=752.

### Example 20

### Synthesis of Compound M20

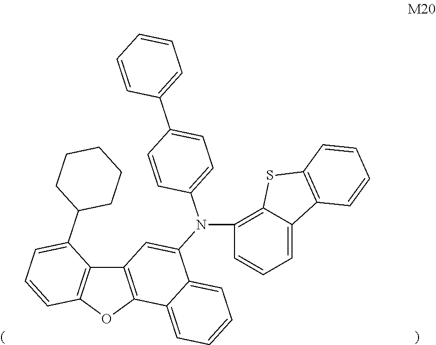 [SMILES: c1ccc(-c2ccc(N(c3cc4c(oc5cccc(C6CCCCC6)c54)c4ccccc34)c3cccc4c3sc3ccccc34)cc2)cc1]

Synthetic Route of the Compound M20 is as follows.

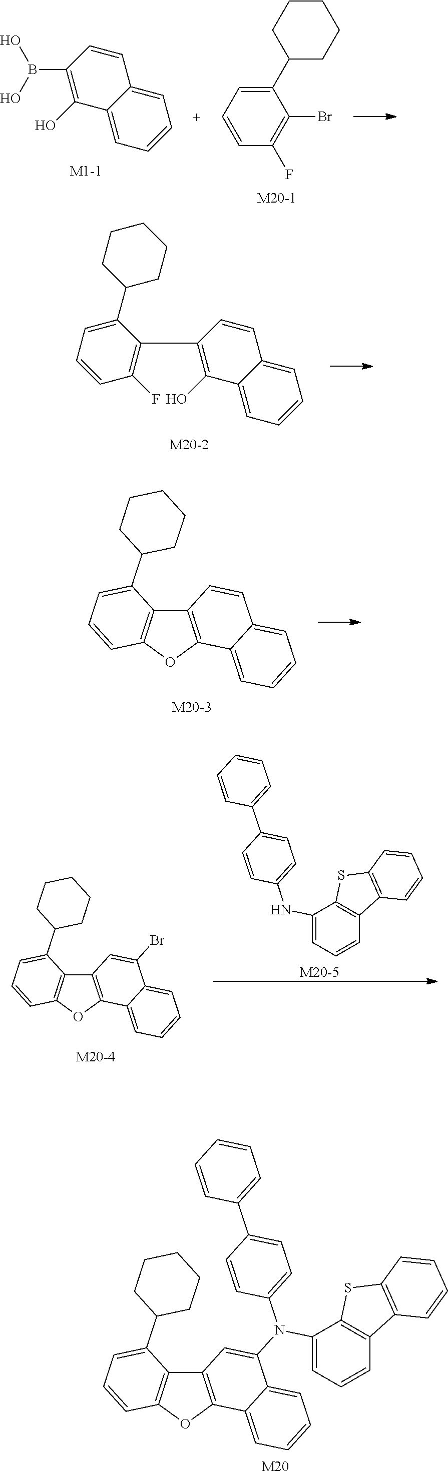

1) Synthesis of intermediate M20-2: according to the synthesis method of the compound M1-3, compound M20-1 was substituted for the compound M1-2 to obtain the intermediate M20-2 with yield of 73%.
2) Synthesis of intermediate M20-3: according to the synthesis method of the compound M1-4, compound M20-2 was substituted for the compound M1-3 to obtain the intermediate M20-3 with yield of 72%.
3) Synthesis of intermediate M20-4: according to the synthesis method of the compound M1-5, compound M20-3 was substituted for the compound M1-4 to obtain the intermediate M20-4 with yield of 81%.
4) Synthesis of the compound M20: according to the synthesis method of the compound M1, compound M20-4 and compound M20-5 were substituted for the compound M1-5 and the compound M1-6, respectively, to obtain the compound M20 with yield of 80%. MS (ASAP)=650.

### Example 21

### Synthesis of Compound M21

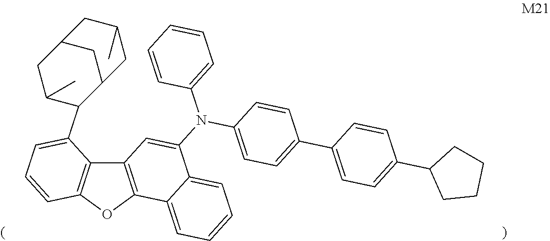 [SMILES: CC1CC2CC(C)C(c3cccc4oc5c6ccccc6c(N(c6ccccc6)c6ccc(-c7ccc(C8CCCC8)cc7)cc6)cc5c34)C(C1)C2]

Synthetic Route of the Compound M21 is as follows.

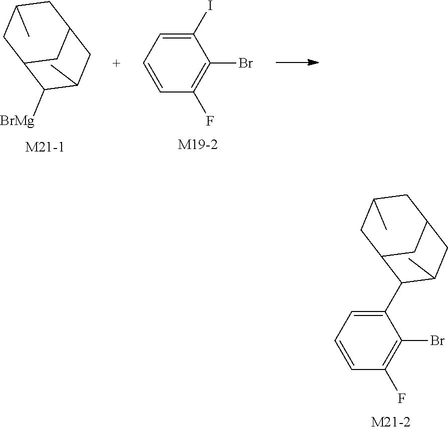

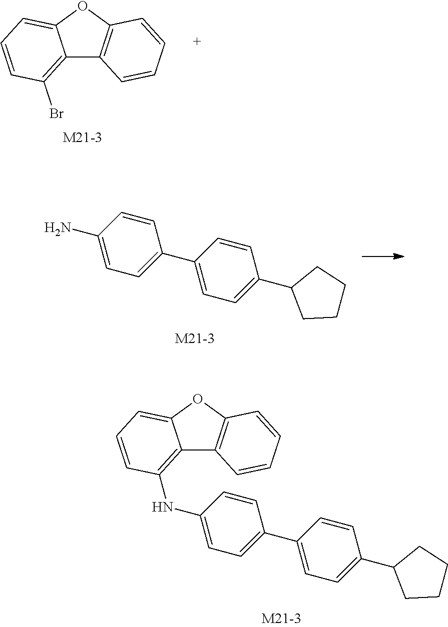 [SMILES: Brc1cccc2oc3ccccc3c12 ; Nc1ccc(-c2ccc(C3CCCC3)cc2)cc1 ; c1ccc2c(c1)oc1cccc(Nc3ccc(-c4ccc(C5CCCC5)cc4)cc3)c12]

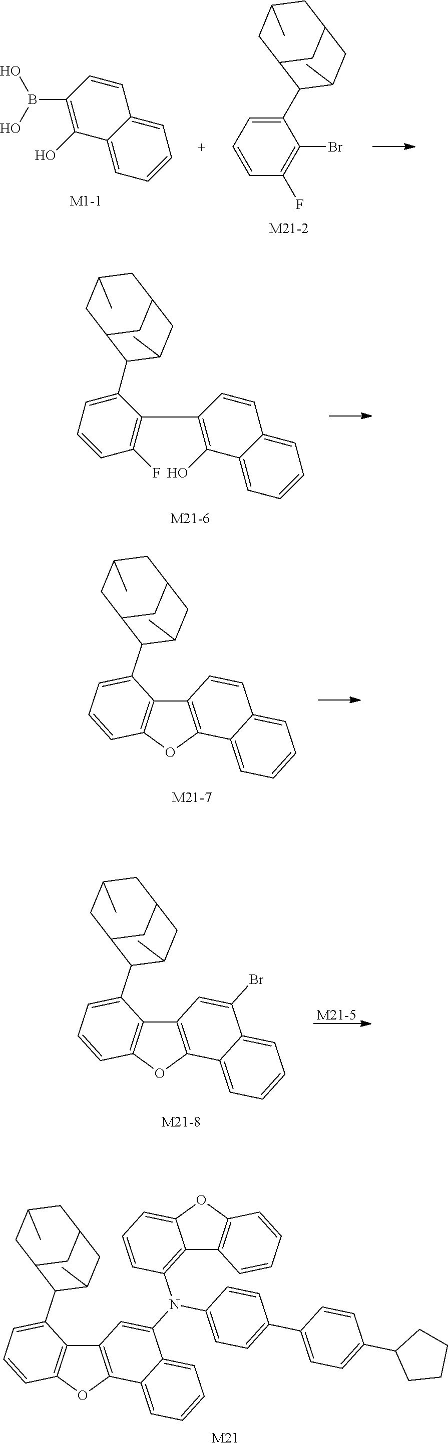

1) Synthesis of intermediate M21-2: according to the synthesis method of the compound M19-3, compound M21-1 was substituted for the compound M19-1 to obtain the intermediate M21-2 with yield of 71%.
2) Synthesis of intermediate M21-5: according to the synthesis method of the compound M1, compound M21-3 and compound M21-4 were substituted for the compound M1-5 and the compound M1-6, respectively, to obtain the intermediate M21-5 with yield of 76%.
3) Synthesis of intermediate M21-6: according to the synthesis method of the compound M1-3, compound M21-2 was substituted for the compound M1-2 to obtain the intermediate M21-6 with yield of 72%.
4) Synthesis of intermediate M21-7: according to the synthesis method of the compound M1-4, compound M21-6 was substituted for the compound M1-3 to obtain the intermediate M21-7 with yield of 73%.
5) Synthesis of intermediate M21-8: according to the synthesis method of the compound M1-5, compound M21-7 was substituted for the compound M1-4 to obtain the intermediate M21-8 with yield of 82%.
6) Synthesis of the compound M21: according to the synthesis method of the compound M1, compound M21-8 and compound M21-5 were substituted for the compound M1-5 and the compound M1-6, respectively, to obtain the compound M21 with yield of 81%. MS (ASAP)=754.

### Example 22

### Synthesis of Compound M22

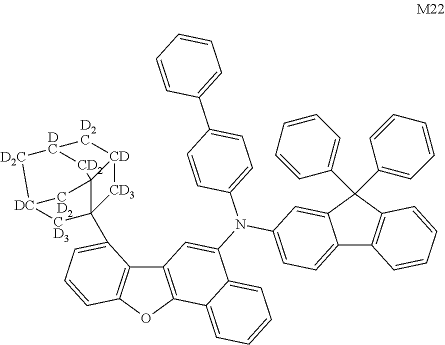

Synthetic Route of the Compound M22 is as follows.

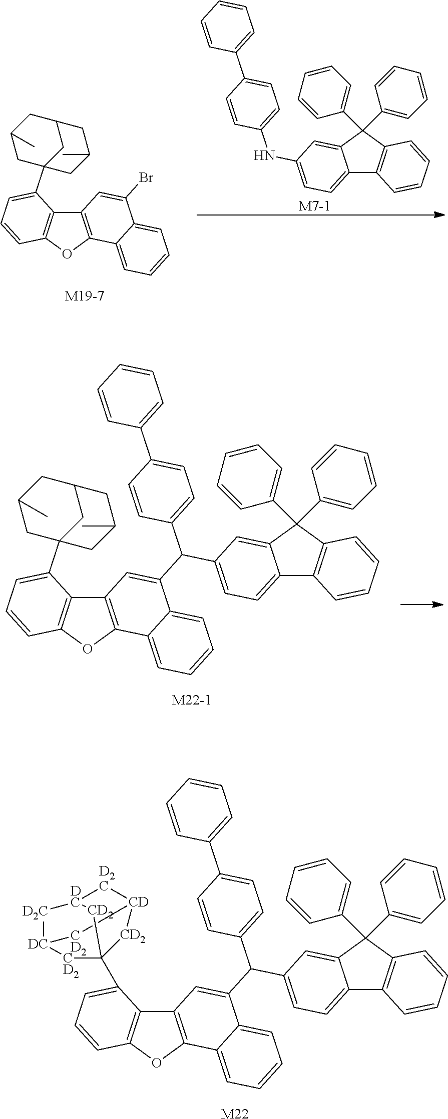

1) Synthesis of intermediate M22-1: according to the synthesis method of the compound M1, compound M19-7 and compound M7-1 were substituted for the compound M1-5 and the compound M1-6, respectively, to obtain the intermediate M22-1 with yield of 80%.
2) Synthesis of the compound M22: according to the synthesis method of the compound M14, compound M22-1 was substituted for the compound M1 to obtain the compound M22 with yield of 84%. MS (ASAP)=852.

### Example 23

### Synthesis of Compound M23

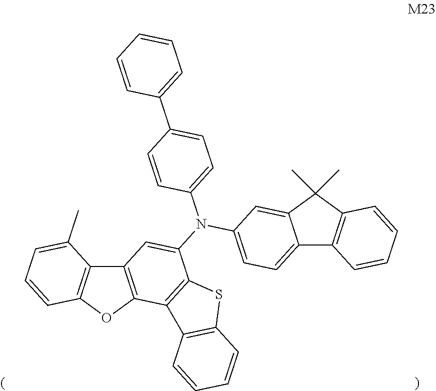 [SMILES: Cc1cccc2oc3c(cc(N(c4ccc(-c5ccccc5)cc4)c4ccc5c(c4)C(C)(C)c4ccccc4-5)c4sc5ccccc5c43)c12]

Synthetic Route of the Compound M23 is as follows.

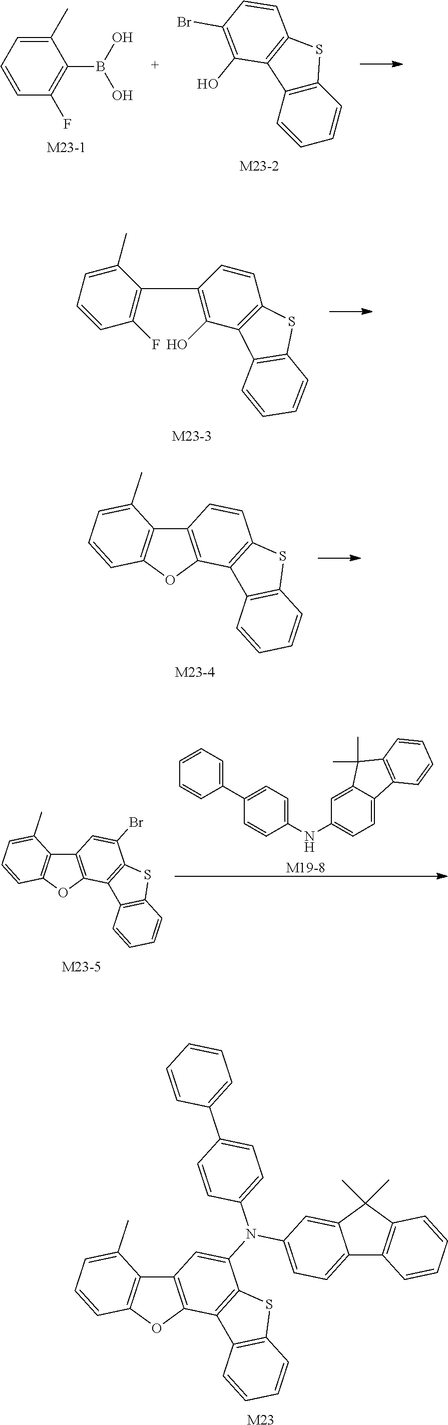

1) Synthesis of intermediate M23-3: according to the synthesis method of the compound M1-3, compound M23-1 and compound M23-2 were substituted for the compound M1-1 and the compound M1-2, respectively, to obtain the intermediate M23-3 with yield of 64%.
2) Synthesis of intermediate M23-4: according to the synthesis method of the compound M1-4, compound M23-3 was substituted for the compound M1-3 to obtain the intermediate M23-4 with yield of 72%.
3) Synthesis of intermediate M23-5: according to the synthesis method of the compound M1-5, compound M23-4 was substituted for the compound M1-4 to obtain the intermediate M23-5 with yield of 80%.
4) Synthesis of the compound M23: according to the synthesis method of the compound M1, compound M23-5 and compound M23-5 were substituted for the compound M1-5 and the compound M1-6, respectively, to obtain the compound M23 with yield of 83%. MS (ASAP)-648.

### Example 24

### Synthesis of Compound M24

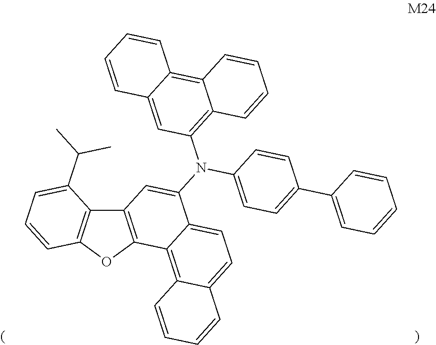 [SMILES: CC(C)c1cccc2oc3c(cc(N(c4ccc(-c5ccccc5)cc4)c4cc5ccccc5c5ccccc45)c4ccc5ccccc5c43)c12]

Synthetic Route of the Compound M24 is as follows.

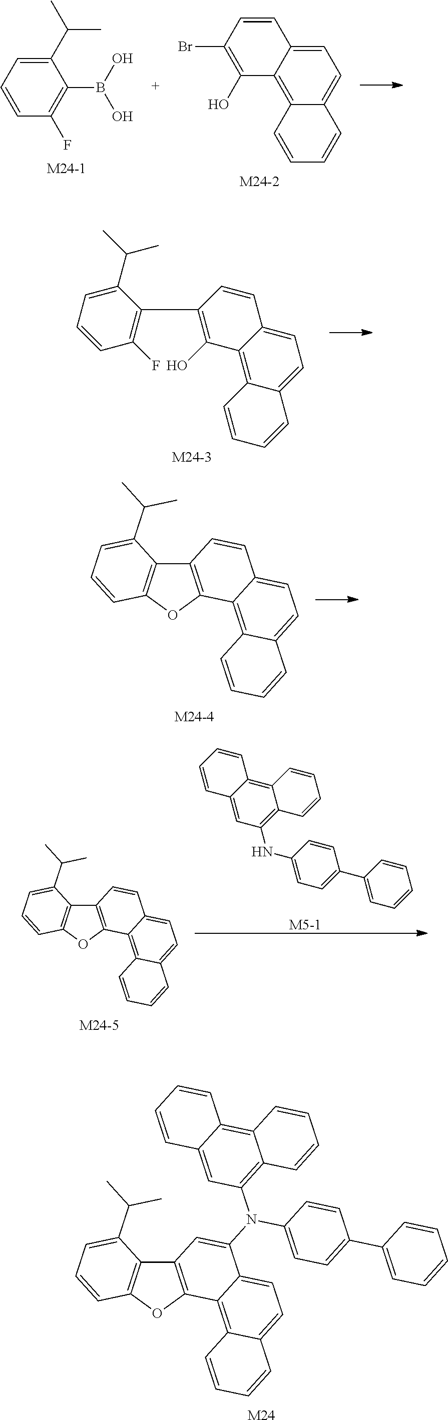

1) Synthesis of intermediate M24-3: according to the synthesis method of the compound M1-3, compound M24-1 and compound M24-2 were substituted for the compound M1-1 and the compound M1-2, respectively, to obtain the intermediate M24-3 with yield of 66%.
2) Synthesis of intermediate M24-4: according to the synthesis method of the compound M1-4, compound M24-3 was substituted for the compound M1-3 to obtain the intermediate M24-4 with yield of 74%.
3) Synthesis of intermediate M24-5: according to the synthesis method of the compound M1-5, compound M24-4 was substituted for the compound M1-4 to obtain the intermediate M24-5 with yield of 82%.
4) Synthesis of the compound M24: according to the synthesis method of the compound M1, compound M24-5 and compound M5-1 were substituted for the compound M1-5 and the compound M1-6, respectively, to obtain the compound M24 with yield of 82%. MS (ASAP)-654.

### Preparation and Characterization of OLED Devices

Taking the preparation of red light OLED devices for examples, the following is a detailed explanation of preparation methods of OLED devices including the compounds prepared in specific examples according to the present disclosure.

In the following preparation methods of the OLED devices, the material of the anode is ITO, the hole injection material is 2,3,6, 7,10, 11-hexacyano-1,4,5,8,9, 12-hexaazatriphenylene (HATCN), the hole transport material is HT, the host material of the light-emitting layer is RH, the doping material of the light-emitting layer is RD, the electron transport materials include ET and 8-hydroxyquinoline lithium (Liq), the electron injection material is Li, and the material of the cathode is Al. In addition, the compounds M1-M24 from the above-mentioned examples 1-24 are used as electron barrier materials to prepare corresponding OLED devices. The structures of HATCN, HT, RD, RH, ET, and Liq are as follows.

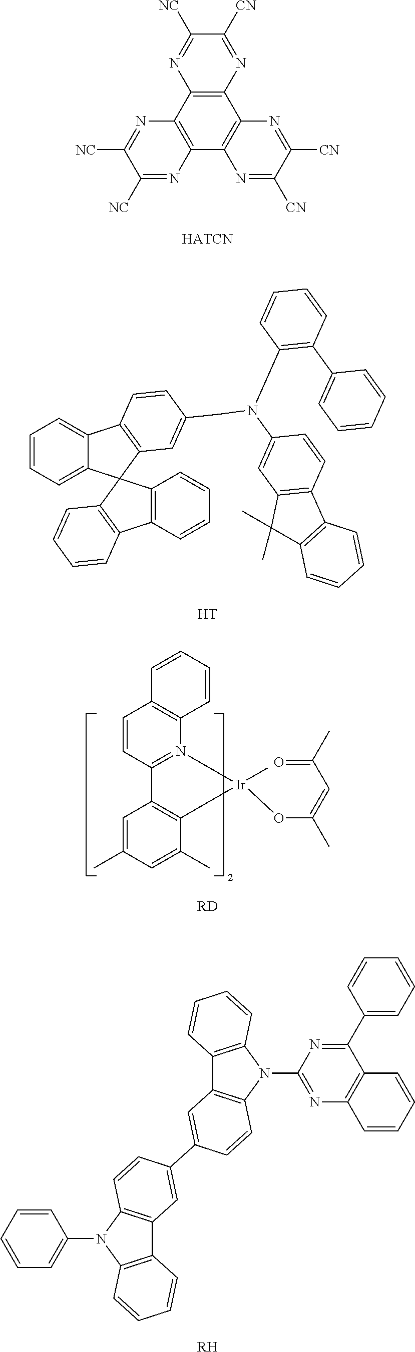

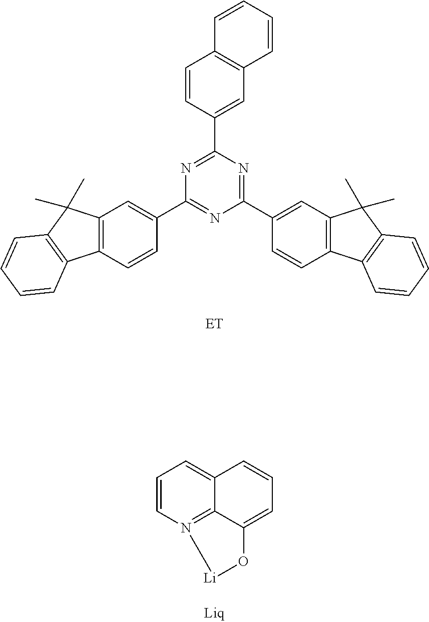

Taking the preparation method of the OLED device using the compound M1 as the electron barrier material for example, the prepared OLED device is referred as “OLED-1”. The preparation method of the OLED-1 includes the following steps.
Step (1), an ITO conductive glass substrate was provided, and cleaned with cleaning agents such as deionized water, acetone, isopropanol, or the like. Then, the cleaned substrate was placed into a plasma cleaner to improve the functionality of the substrate.
Step (2), the HATCN was evaporated on the ITO conductive glass substrate to obtained a hole injection layer with a thickness of 30 nm.
Step (3), the HT was evaporated on the hole injection layer to obtain a hole transport layer with a thickness of 60 nm.
Step (4), the compound M1 was evaporated on the hole transport layer to obtain an electron barrier layer with a thickness of 10 nm.
Step (5), the RH and the RD were evaporated on the electron barrier layer with a mass ratio of 3:100, to obtain a light-emitting layer with a thickness of 40 nm.
Step (6), the ET and the Liq were evaporated on the light-emitting layer with a mass ratio of 5:5, to obtain an electron transport layer with a thickness of 30 nm.
Step (7), the Liq was evaporated on the electron transport layer to obtain an electron injection layer with a thickness of 1 nm.
Step (8), the Al was evaporated on the electron injection layer to obtain a cathode with a thickness of 100 nm.

Furthermore, according to the preparation method of the OLED-1, the compounds M2-M24 were used as electron barrier materials in OLED devices, to prepare devices OLED-2 to OLED-24, respectively. It can be understood that in the preparation methods of the devices OLED-1 to OLED-24, all other experimental conditions are the same, other than the electron barrier materials.

Furthermore, according to the preparation method of the OLED-1, the comparative compounds Ref1-Ref3 were used as electron barrier materials in OLED devices, to prepare devices OLED-Ref1 to OLED-Ref3, respectively. It can be understood that in the preparation methods of the devices OLED-Ref1 to OLED-Ref3, all other experimental conditions are the same, other than the electron barrier materials.

The structures of the comparative compounds Ref1-Ref3 are as follows.

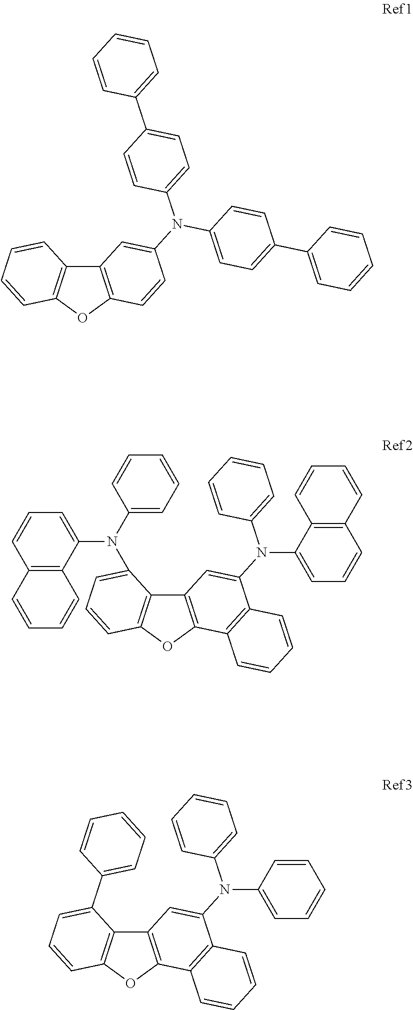 [SMILES: c1ccc(-c2ccc(N(c3ccc(-c4ccccc4)cc3)c3ccc4oc5ccccc5c4c3)cc2)cc1 ; c1ccc(N(c2cccc3ccccc23)c2cc3c(oc4cccc(N(c5ccccc5)c5cccc6ccccc56)c43)c3ccccc23)cc1 ; c1ccc(-c2cccc3oc4c5ccccc5c(N(c5ccccc5)c5ccccc5)cc4c23)cc1]

In the present disclosure, the current densities and the voltages of the devices OLED-1 to OLED-24 and OLED-Ref1 to OLED-Ref3 are tested, to obtain the current efficiency (CE@1knits) and the lifespan (LT90@1knits) of the above-mentioned devices, and the results are shown in table 1. The current efficiency is the relative value obtained at the brightness of 1000 nits and the current density of 10 mA/cm², and the lifetime LT90@1knits refers to a time when the brightness of the device delays from initial brightness of 1000 nits to 90% of the initial brightness under a constant current.

TABLE 1

| | Electron barrier | CE@1knits | LT90@1knits |
|---|---|---|---|
| Device | material | [cd/A] | [h] |
| OLED-1 | Compound 1 | 1.84 | 1.95 |
| OLED-2 | Compound 2 | 1.77 | 1.87 |
| OLED-3 | Compound 3 | 1.83 | 1.93 |
| OLED-4 | Compound 4 | 1.76 | 1.85 |
| OLED-5 | Compound 5 | 1.79 | 1.88 |
| OLED-6 | Compound 6 | 1.81 | 1.92 |
| OLED-7 | Compound 7 | 1.68 | 1.76 |
| OLED-8 | Compound 8 | 1.65 | 1.73 |
| OLED-9 | Compound 9 | 1.80 | 1.90 |
| OLED-10 | Compound 10 | 1.63 | 1.72 |
| OLED-11 | Compound 11 | 1.66 | 1.75 |
| OLED-12 | Compound 12 | 1.71 | 1.80 |
| OLED-13 | Compound 13 | 1.74 | 1.83 |
| OLED-14 | Compound 14 | 1.85 | 1.96 |
| OLED-15 | Compound 15 | 1.62 | 1.70 |
| OLED-16 | Compound 16 | 1.54 | 1.62 |
| OLED-17 | Compound 17 | 1.55 | 1.64 |
| OLED-18 | Compound 18 | 1.60 | 1.68 |
| OLED-19 | Compound 19 | 1.69 | 1.78 |
| OLED-20 | Compound 20 | 1.57 | 1.65 |
| OLED-21 | Compound 21 | 1.58 | 1.67 |
| OLED-22 | Compound 22 | 1.73 | 1.82 |
| OLED-23 | Compound 23 | 1.73 | 1.83 |
| OLED-24 | Compound 24 | 1.72 | 1.81 |
| OLED-Ref1 | Comparative | 1 | 1 |
| | compound Ref1 | | |
| OLED-Ref2 | Comparative | 1.06 | 1.08 |
| | compound Ref2 | | |
| OLED-Ref3 | Comparative | 1.08 | 1.10 |
| | compound Ref3 | | |

It can be seen from table 1 that the luminescent efficiency and the lifespan of the devices OLED-1 to OLED-24 prepared using the compounds M1-M24 in the examples of the present disclosure can be significantly improved, compared to the devices OLED-Ref1 to OLED-Ref3 prepared using the comparative compounds Ref1-Ref3. Therefore, it can be seen that by fusing the alkyl group and the fused heterocycle containing oxygen in the aromatic amine compound, with the coordination of the aromatic amine, the present disclosure can effectively regulate the transmission of the holes. When the aromatic amine compound is used as the electron barrier material in the electron barrier layer of the organic electronic device such as the red light OLED device, the charge transmission balance in the organic electronic device can be better regulated, improving the luminous efficiency and prolonging the lifespan of the organic electronic device.

The aromatic amine compound, the mixture, the composition, and the organic electronic device according to the embodiments of the present disclosure are described in detail. In this context, specific embodiments are adopted to illustrate a principle and implementation modes of the present disclosure. The description of the above-mentioned embodiments is only used to help understand methods and a core idea of the present disclosure. At the same time, for those skilled in the art, according to the idea of the present disclosure, there might be changes in specific implementation modes and a scope of the present disclosure, which falls within the scope of the protection of the present disclosure. In conclusion, contents of the specification should not be interpreted as a limitation of the present disclosure.

What is claimed is:

1. An aromatic amine compound represented by formula (1): 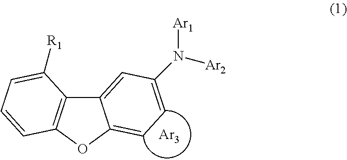
wherein Ar₁ and Ar₂ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof;
wherein Ar₃ is selected from a substituted or unsubstituted aromatic group with 6 to 30 ring atoms or a substituted or unsubstituted heteroaromatic group with 5 to 30 ring atoms; and
wherein R¹ is selected from substituted or unsubstituted methyl, substituted or unsubstituted ethyl, substituted or unsubstituted isopropyl, substituted or unsubstituted tertbutyl, substituted or unsubstituted cyclopentyl, substituted or unsubstituted cyclohexyl, or a substituted or unsubstituted adamantane group.

2. The aromatic amine compound of claim 1, wherein R₁ is selected from one of following substituted or unsubstituted groups: 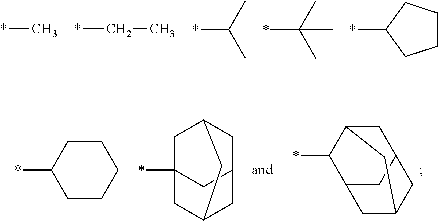
wherein * indicates a linking site.

3. The aromatic amine compound of claim 1, wherein R₁ is selected from one of following substituted or unsubstituted groups: 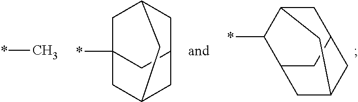
wherein R₁ is substituted by a deuterium atom when R₁ is one of substituted groups; and
wherein * indicates a linking site.

4. The aromatic amine compound of claim 1, wherein Ar₁ and Ar₂ are each independently selected from one of following substituted or unsubstituted groups: 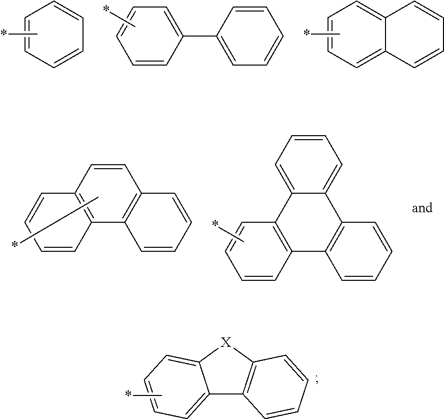
wherein X is selected from NR₂, CR₃R₄, O, S, SiR₃R₄, or PR₂; and wherein R₂, R₄, and R₄ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof; and
wherein * indicates a linking site.

5. The aromatic amine compound of claim 4, wherein X is selected from NR₂, CR₃R₄, O, or S; and wherein R₂ is phenyl, R₃ and R₄ are each independently selected from methyl or phenyl.

6. The aromatic amine compound of claim 1, wherein 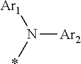
is selected from one of following structures: 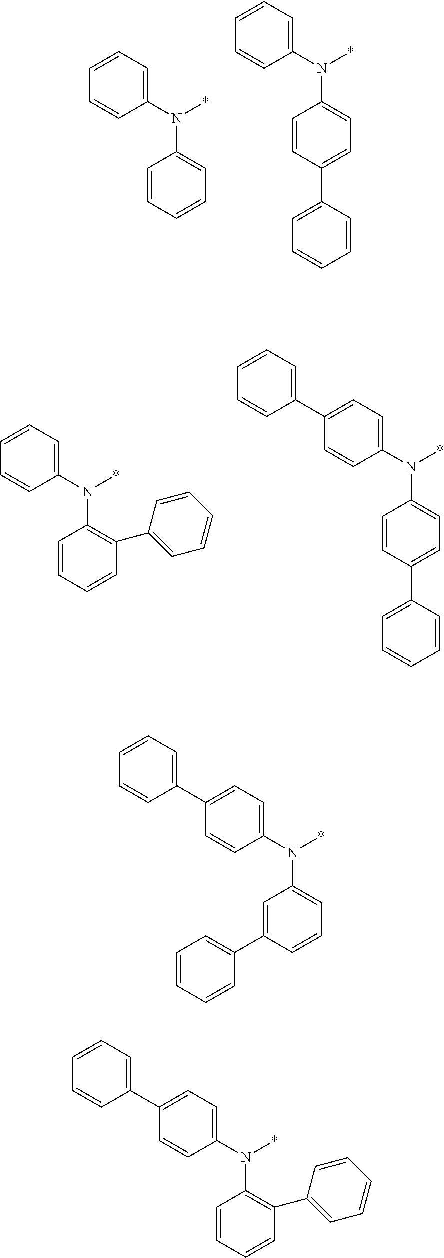 [SMILES: *N(c1ccccc1)c1ccccc1 ; *N(c1ccccc1)c1ccc(-c2ccccc2)cc1 ; *N(c1ccccc1)c1ccccc1-c1ccccc1 ; *N(c1ccc(-c2ccccc2)cc1)c1ccc(-c2ccccc2)cc1 ; *N(c1ccc(-c2ccccc2)cc1)c1cccc(-c2ccccc2)c1 ; *N(c1ccc(-c2ccccc2)cc1)c1ccccc1-c1ccccc1] 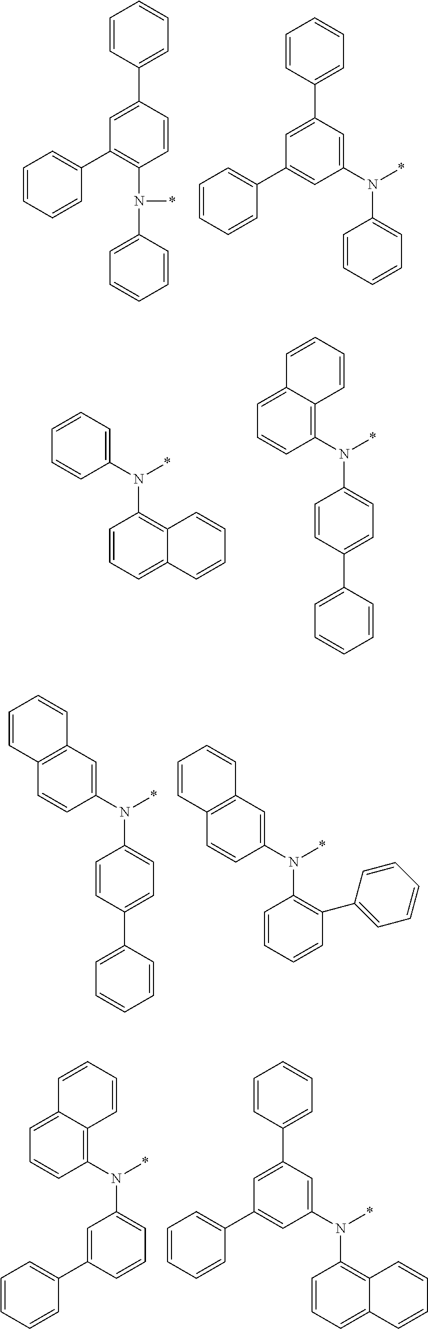 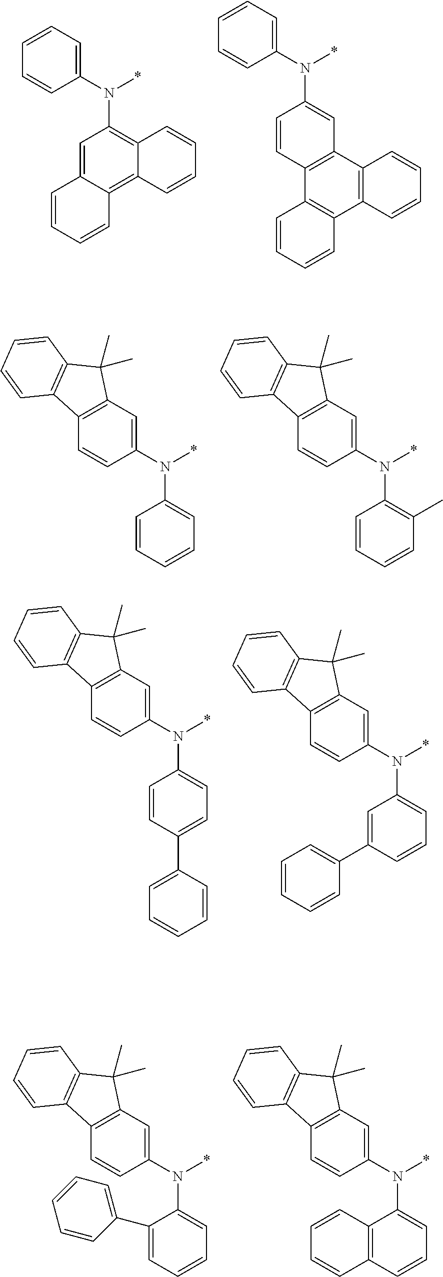 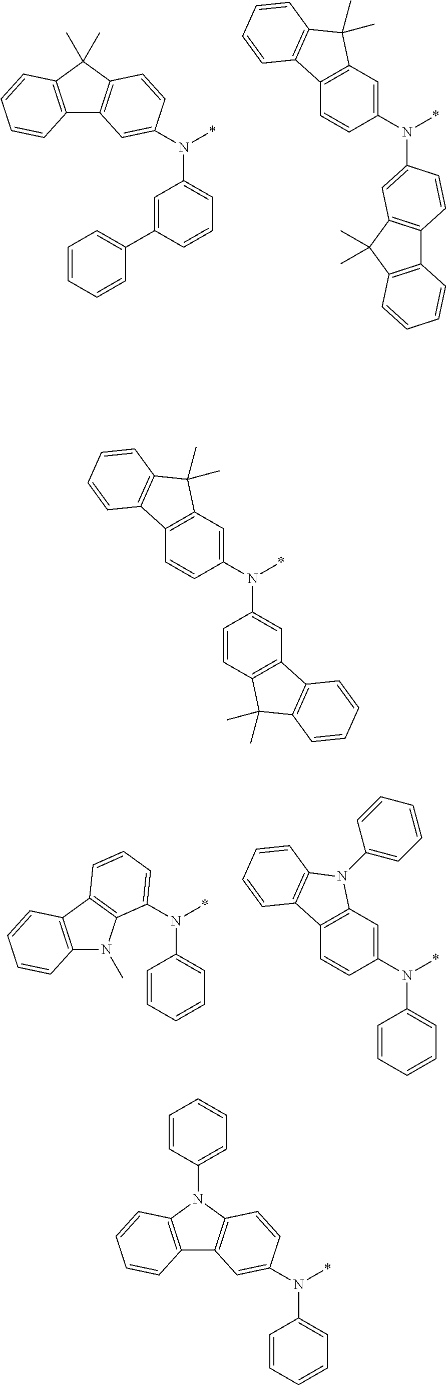 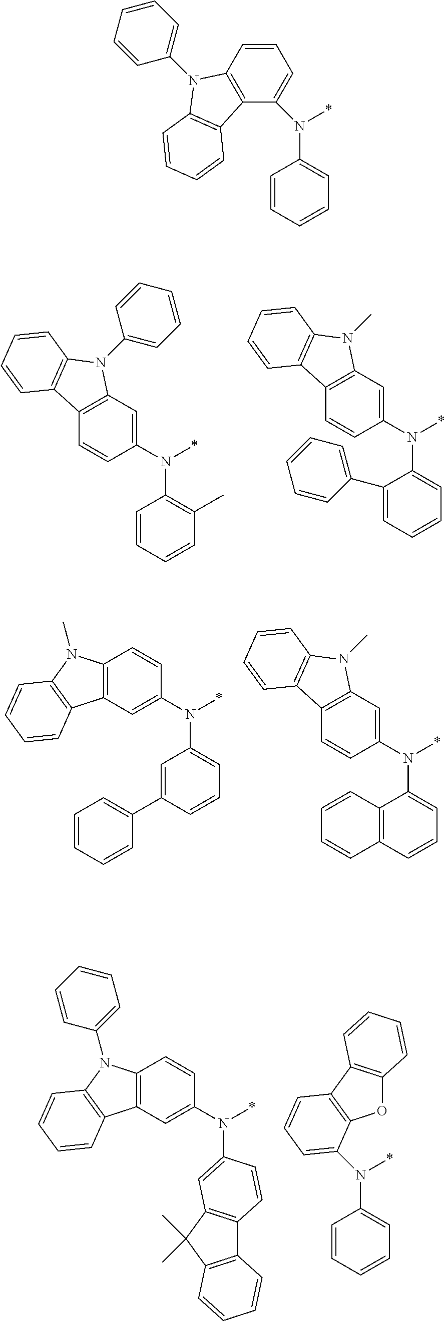 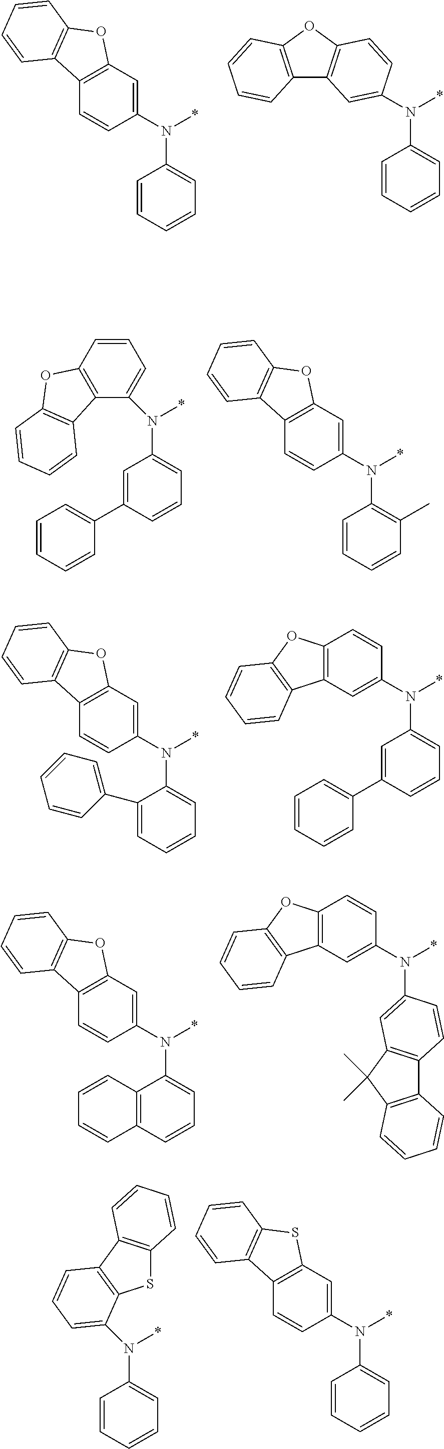 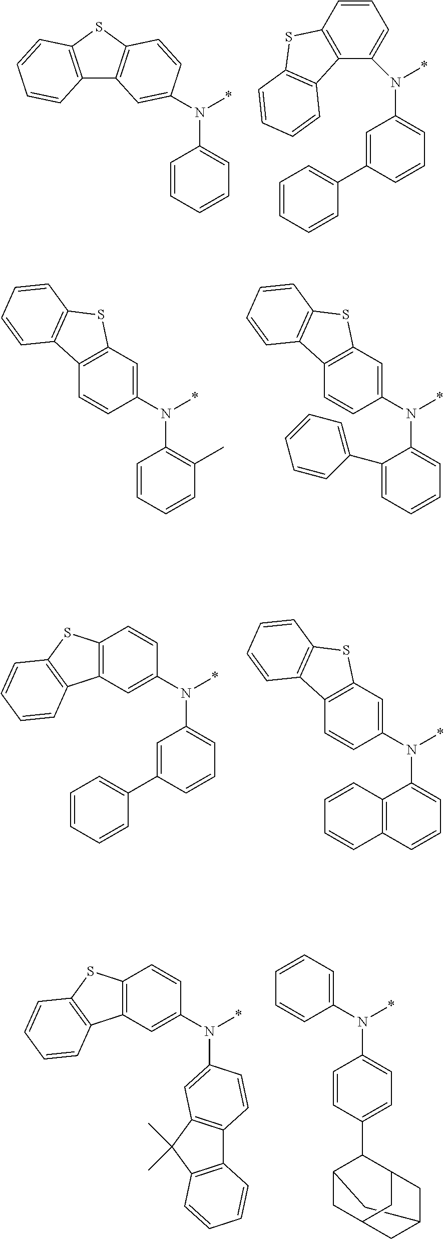 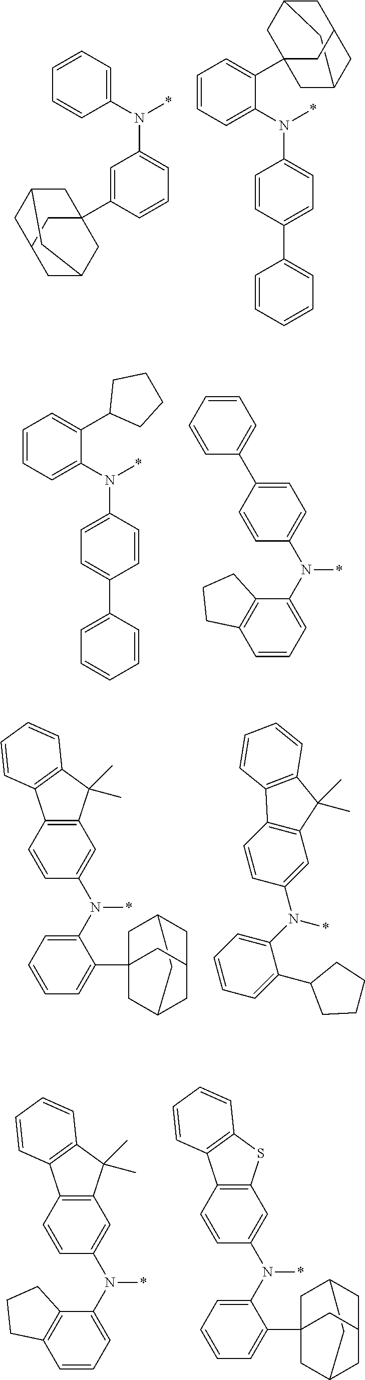 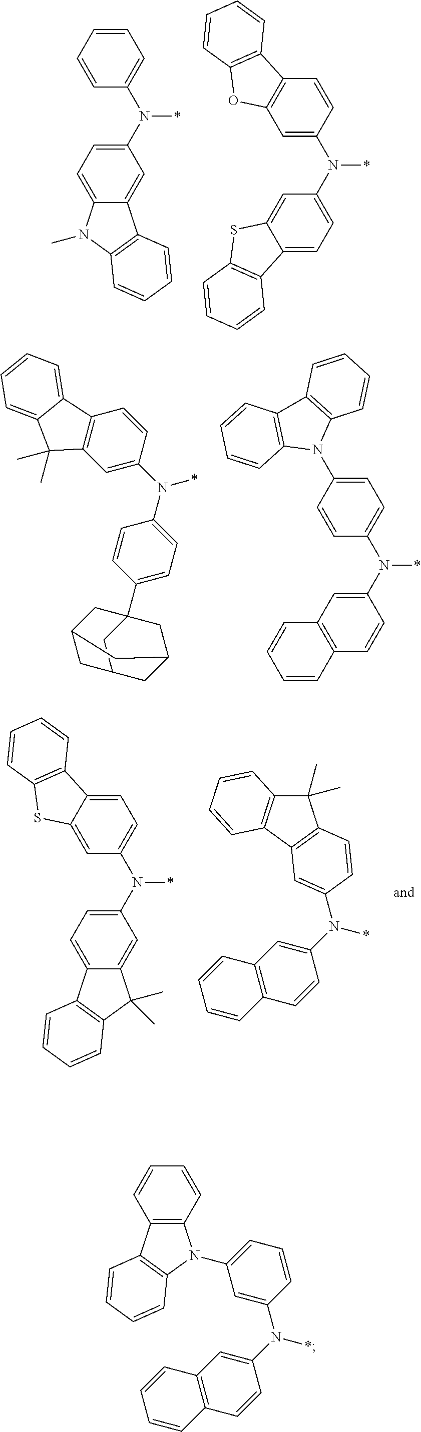
wherein * indicates a linking site.

7. The aromatic amine compound of claim 1, wherein Ar₃ is selected from one of following groups: 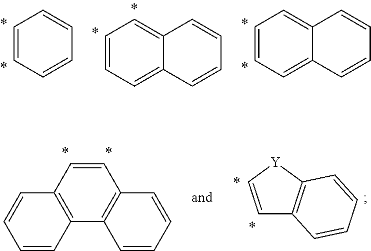
wherein Y is selected from NR₂, CR₃R₄, O, S, SiR₃R₄, or PR₂; and wherein R₂, R₃, and R₄ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof; and
wherein * indicates a fused site.

8. The aromatic amine compound of claim 1, wherein Ar₃ is selected from one of following groups: 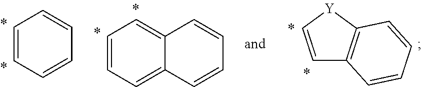
wherein Y is selected from O or S; and
wherein * * indicates a fused site.

9. The aromatic amine compound of claim 1, wherein at least one of Ar₁ and Ar₂ is substituted or unsubstituted 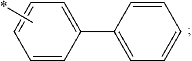
Ar₃ is phenyl; and R₁ is substituted or unsubstituted methyl; and
wherein * indicates a linking site.

10. The aromatic amine compound of claim 9, wherein R₁ is substituted methyl, and R₁ is substituted by a deuterium atom.

11. The aromatic amine compound of claim 1, wherein Ar₁ and Ar₂ are the same group.

12. The aromatic amine compound of claim 1, wherein at least one of Ar₁ and Ar₂ is 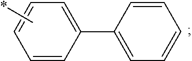
and
wherein * indicates a linking site.

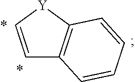

13. The aromatic amine compound of claim 1, wherein Ar₃ is wherein Y is selected from NR₂, CR₃R₄, O, or S; and wherein R₂, R₃, and R₄ are each independently a substituted or unsubstituted C₁-C₂₀ alkyl group; and
wherein * * indicates a fused site.

14. The aromatic amine compound of claim 1, wherein the aromatic amine compound is one selected from following structures:

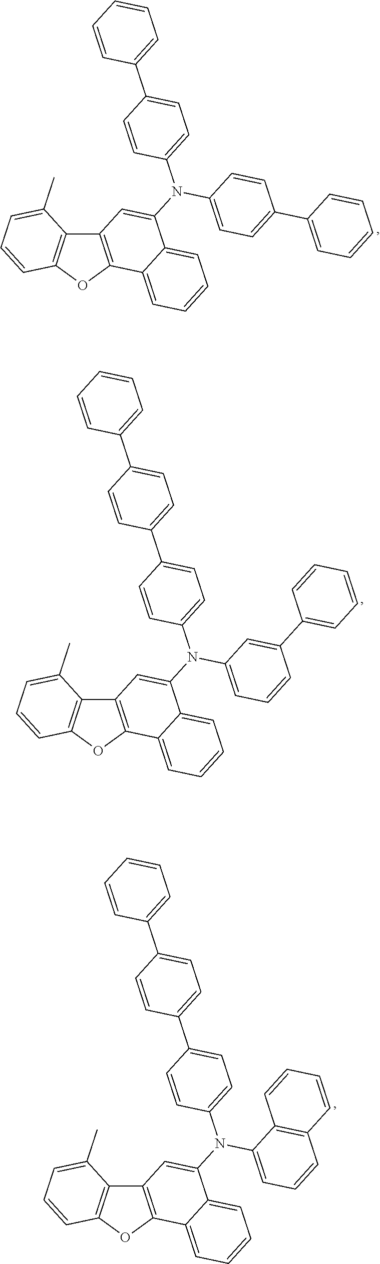 [SMILES: Cc1cccc2oc3c4ccccc4c(N(c4ccc(-c5ccccc5)cc4)c4ccc(-c5ccccc5)cc4)cc3c12 ; Cc1cccc2oc3c4ccccc4c(N(c4ccc(-c5ccc(-c6ccccc6)cc5)cc4)c4cccc(-c5ccccc5)c4)cc3c12 ; Cc1cccc2oc3c4ccccc4c(N(c4ccc(-c5ccc(-c6ccccc6)cc5)cc4)c4cccc5ccccc45)cc3c12]

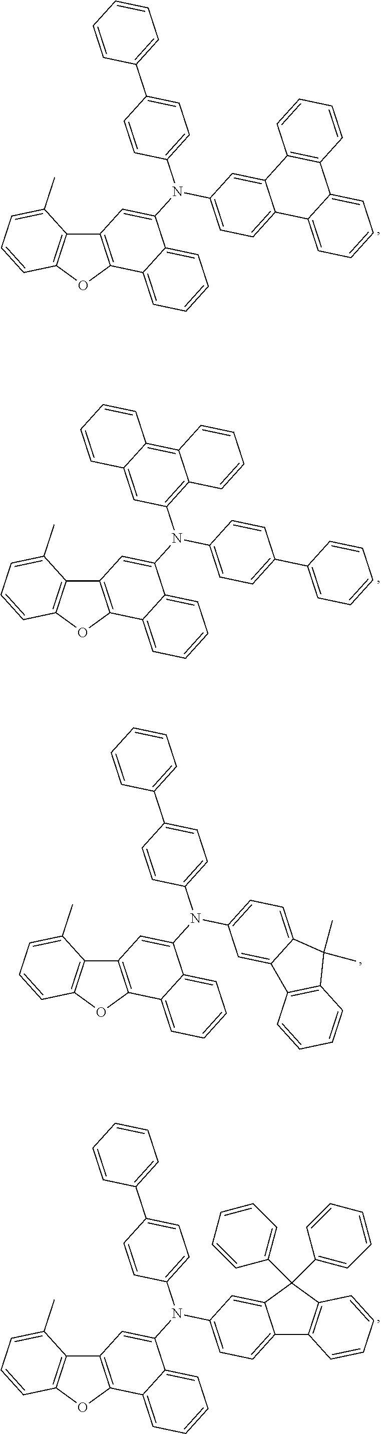 [SMILES: Cc1cccc2oc3c4ccccc4c(N(c4ccc(-c5ccccc5)cc4)c4ccc5c6ccccc6c6ccccc6c5c4)cc3c12 ; Cc1cccc2oc3c4ccccc4c(N(c4ccc(-c5ccccc5)cc4)c4cc5ccccc5c5ccccc45)cc3c12 ; Cc1cccc2oc3c4ccccc4c(N(c4ccc(-c5ccccc5)cc4)c4ccc5c(c4)-c4ccccc4C5(C)C)cc3c12 ; Cc1cccc2oc3c4ccccc4c(N(c4ccc(-c5ccccc5)cc4)c4ccc5c(c4)C(c4ccccc4)(c4ccccc4)c4ccccc4-5)cc3c12]

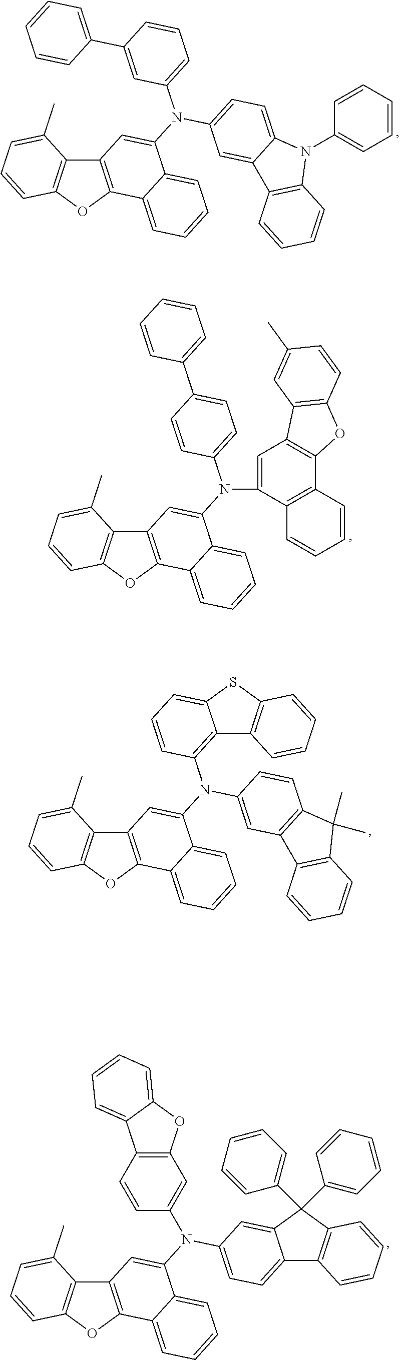 [SMILES: Cc1cccc2oc3c4ccccc4c(N(c4cccc(-c5ccccc5)c4)c4ccc5c(c4)c4ccccc4n5-c4ccccc4)cc3c12 ; Cc1ccc2oc3c4ccccc4c(N(c4ccc(-c5ccccc5)cc4)c4cc5c(oc6cccc(C)c65)c5ccccc45)cc3c2c1 ; Cc1cccc2oc3c4ccccc4c(N(c4ccc5c(c4)-c4ccccc4C5(C)C)c4cccc5sc6ccccc6c45)cc3c12 ; Cc1cccc2oc3c4ccccc4c(N(c4ccc5c(c4)C(c4ccccc4)(c4ccccc4)c4ccccc4-5)c4ccc5c(c4)oc4ccccc45)cc3c12]

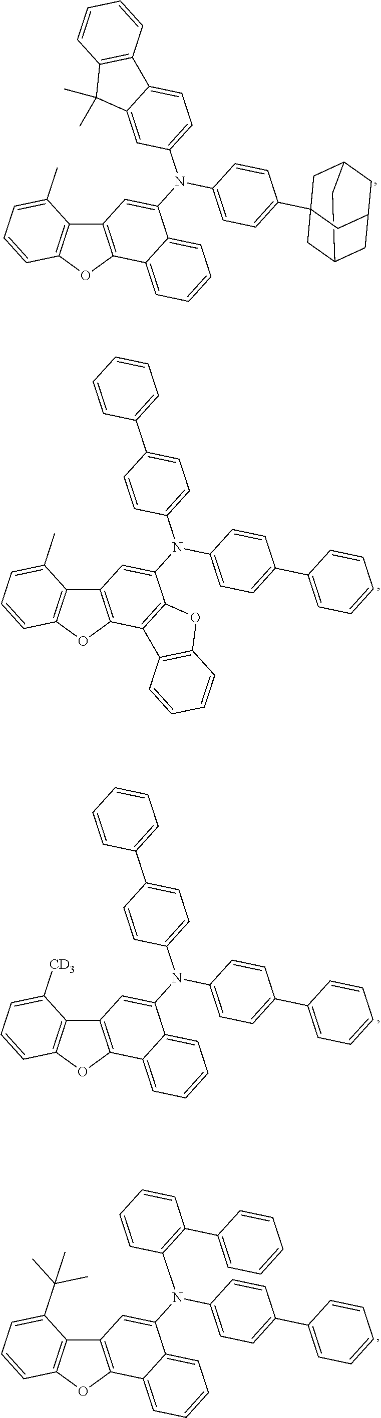 [SMILES: Cc1cccc2oc3c4ccccc4c(N(c4ccc(C56CC7CC(CC(C7)C5)C6)cc4)c4ccc5c(c4)C(C)(C)c4ccccc4-5)cc3c12 ; Cc1cccc2oc3c(cc(N(c4ccc(-c5ccccc5)cc4)c4ccc(-c5ccccc5)cc4)c4oc5ccccc5c43)c12 ; [2H]C([2H])([2H])c1cccc2oc3c4ccccc4c(N(c4ccc(-c5ccccc5)cc4)c4ccc(-c5ccccc5)cc4)cc3c12 ; CC(C)(C)c1cccc2oc3c4ccccc4c(N(c4ccc(-c5ccccc5)cc4)c4ccccc4-c4ccccc4)cc3c12]

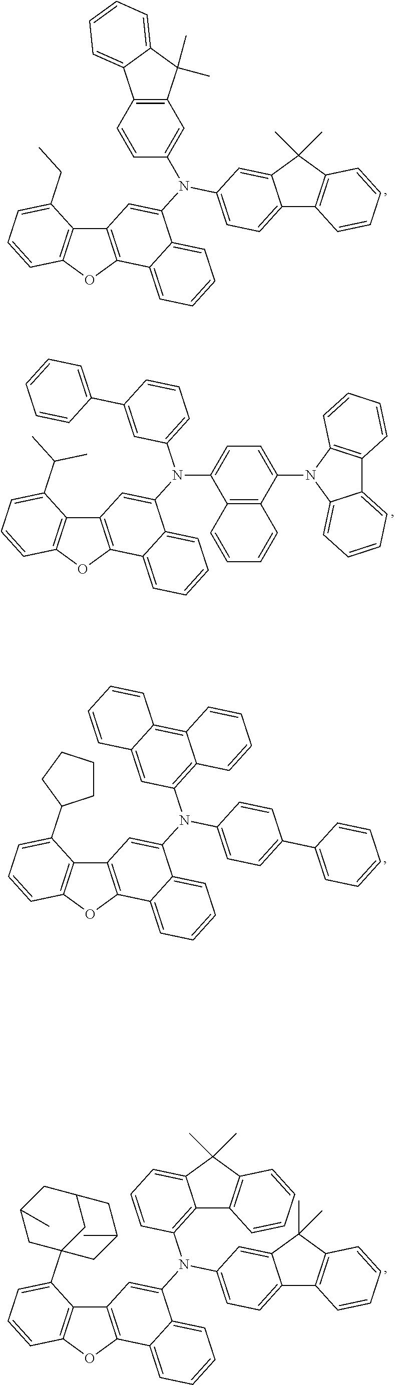 [SMILES: CCc1cccc2oc3c4ccccc4c(N(c4ccc5c(c4)C(C)(C)c4ccccc4-5)c4ccc5c(c4)C(C)(C)c4ccccc4-5)cc3c12 ; CC(C)c1cccc2oc3c4ccccc4c(N(c4cccc(-c5ccccc5)c4)c4ccc(-n5c6ccccc6c6ccccc65)c5ccccc45)cc3c12 ; c1ccc(-c2ccc(N(c3cc4ccccc4c4ccccc34)c3cc4c(oc5cccc(C6CCCC6)c54)c4ccccc34)cc2)cc1 ; CC1CC2CC(C)CC(c3cccc4oc5c6ccccc6c(N(c6ccc7c(c6)C(C)(C)c6ccccc6-7)c6cccc7c6-c6ccccc6C7(C)C)cc5c34)(C1)C2]

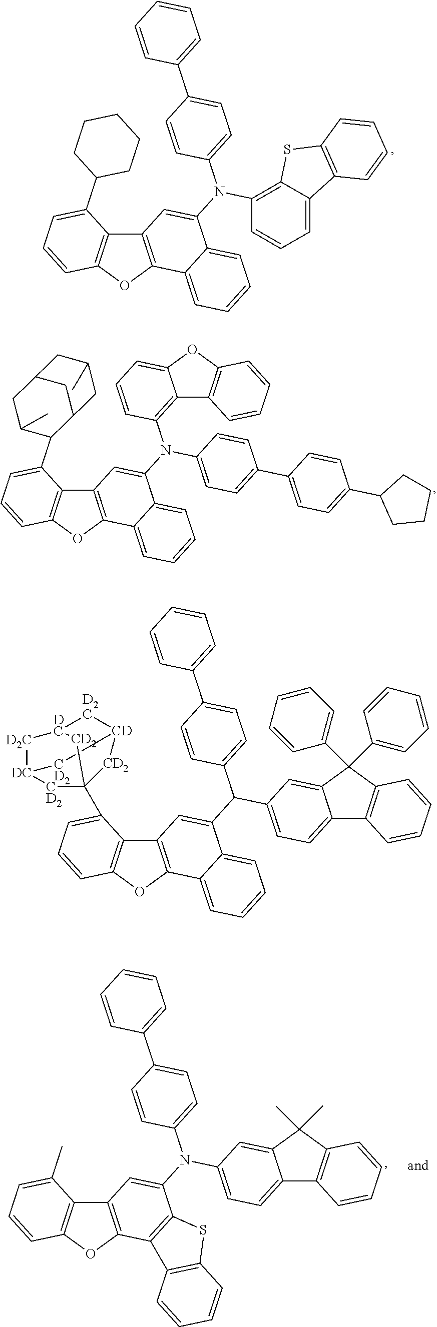

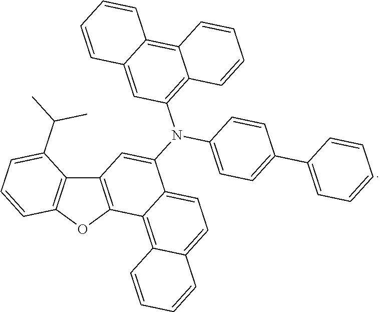 [SMILES: CC(C)c1cccc2oc3c(cc(N(c4ccc(-c5ccccc5)cc4)c4cc5ccccc5c5ccccc45)c4ccc5ccccc5c43)c12]

15. The aromatic amine compound of claim 1, wherein the aromatic amine compound is one selected from following structures:

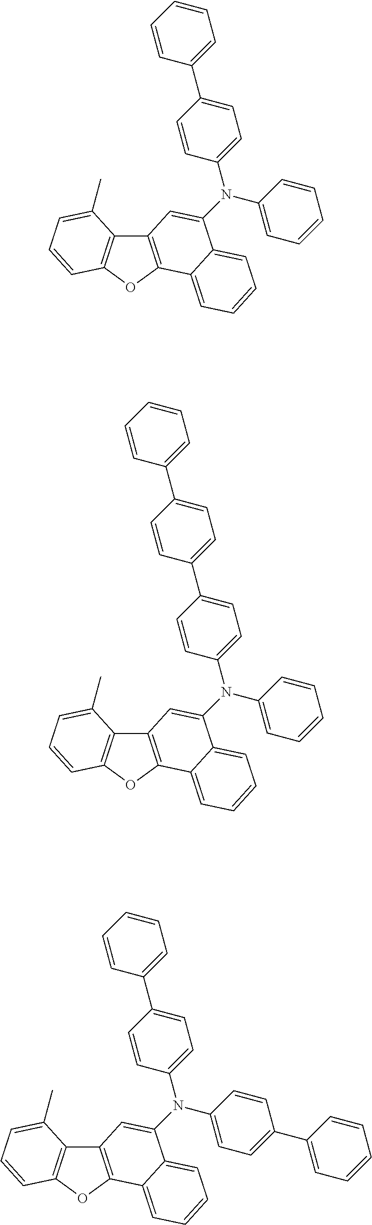 [SMILES: Cc1cccc2oc3c4ccccc4c(N(c4ccccc4)c4ccc(-c5ccccc5)cc4)cc3c12 ; Cc1cccc2oc3c4ccccc4c(N(c4ccccc4)c4ccc(-c5ccc(-c6ccccc6)cc5)cc4)cc3c12 ; Cc1cccc2oc3c4ccccc4c(N(c4ccc(-c5ccccc5)cc4)c4ccc(-c5ccccc5)cc4)cc3c12]

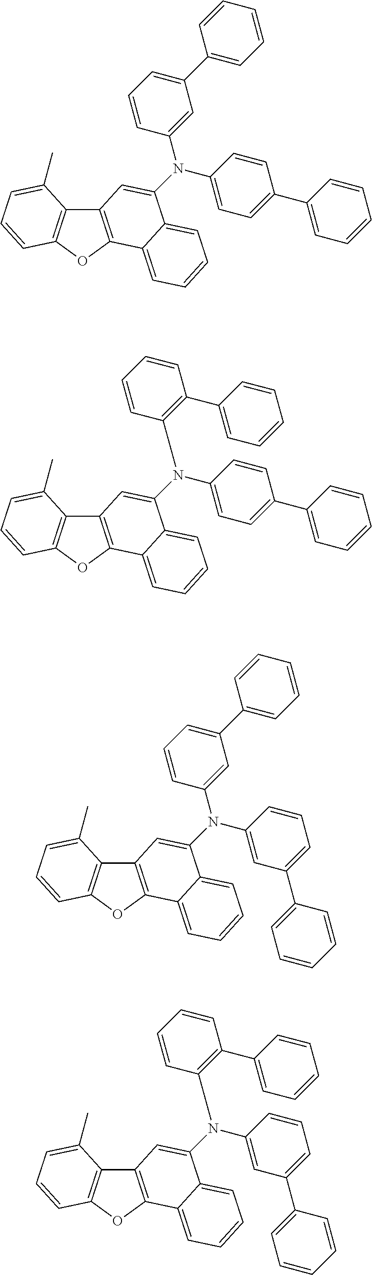 [SMILES: Cc1cccc2oc3c4ccccc4c(N(c4ccc(-c5ccccc5)cc4)c4cccc(-c5ccccc5)c4)cc3c12 ; Cc1cccc2oc3c4ccccc4c(N(c4ccc(-c5ccccc5)cc4)c4ccccc4-c4ccccc4)cc3c12 ; Cc1cccc2oc3c4ccccc4c(N(c4cccc(-c5ccccc5)c4)c4cccc(-c5ccccc5)c4)cc3c12 ; Cc1cccc2oc3c4ccccc4c(N(c4cccc(-c5ccccc5)c4)c4ccccc4-c4ccccc4)cc3c12]

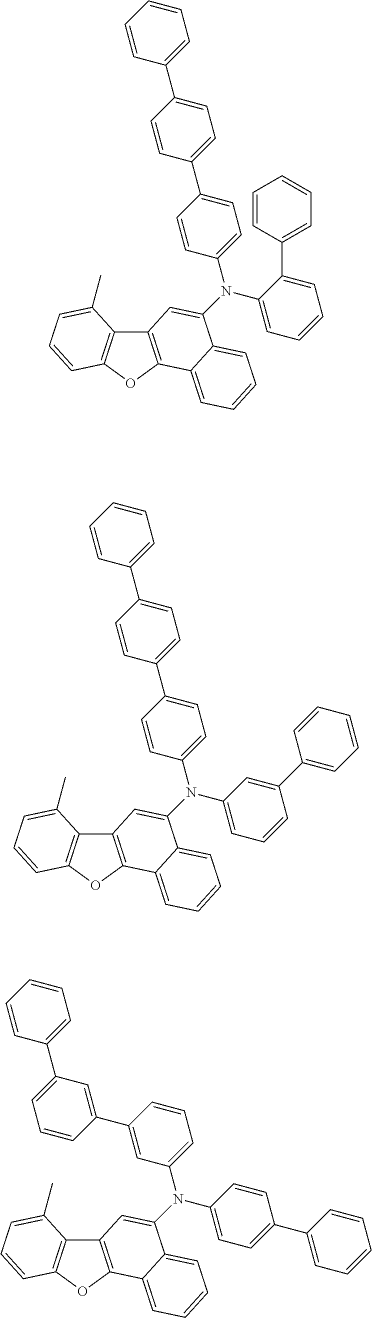 [SMILES: Cc1cccc2oc3c4ccccc4c(N(c4ccc(-c5ccc(-c6ccccc6)cc5)cc4)c4ccccc4-c4ccccc4)cc3c12 ; Cc1cccc2oc3c4ccccc4c(N(c4ccc(-c5ccc(-c6ccccc6)cc5)cc4)c4cccc(-c5ccccc5)c4)cc3c12 ; Cc1cccc2oc3c4ccccc4c(N(c4ccc(-c5ccccc5)cc4)c4cccc(-c5cccc(-c6ccccc6)c5)c4)cc3c12]

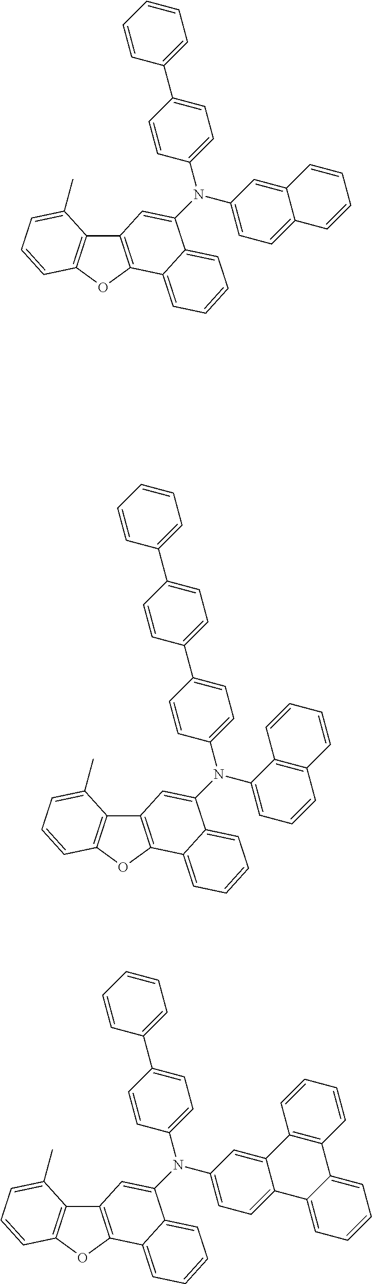 [SMILES: Cc1cccc2oc3c4ccccc4c(N(c4ccc(-c5ccccc5)cc4)c4ccc5ccccc5c4)cc3c12 ; Cc1cccc2oc3c4ccccc4c(N(c4ccc(-c5ccc(-c6ccccc6)cc5)cc4)c4cccc5ccccc45)cc3c12 ; Cc1cccc2oc3c4ccccc4c(N(c4ccc(-c5ccccc5)cc4)c4ccc5c6ccccc6c6ccccc6c5c4)cc3c12]

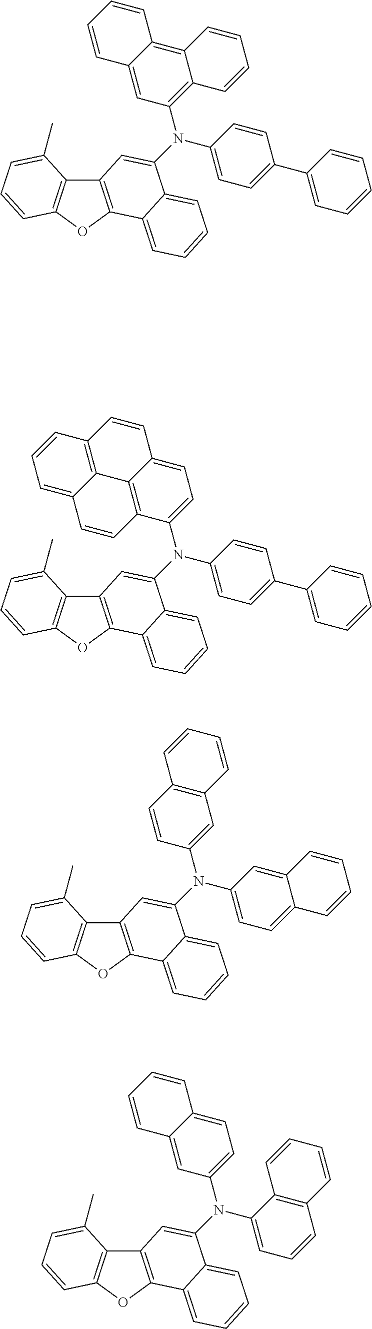 [SMILES: Cc1cccc2oc3c4ccccc4c(N(c4ccc(-c5ccccc5)cc4)c4cc5ccccc5c5ccccc45)cc3c12 ; Cc1cccc2oc3c4ccccc4c(N(c4ccc(-c5ccccc5)cc4)c4ccc5ccc6cccc7ccc4c5c67)cc3c12 ; Cc1cccc2oc3c4ccccc4c(N(c4ccc5ccccc5c4)c4ccc5ccccc5c4)cc3c12 ; Cc1cccc2oc3c4ccccc4c(N(c4ccc5ccccc5c4)c4cccc5ccccc45)cc3c12]

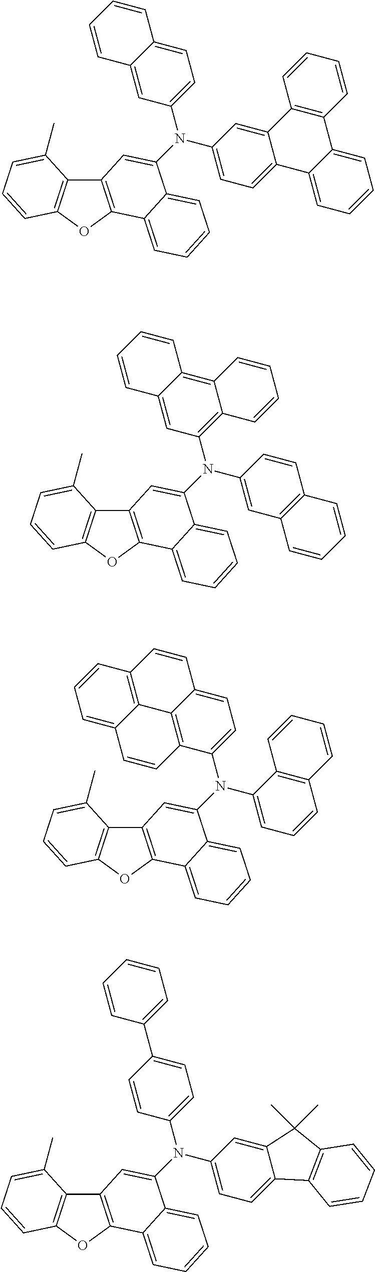 [SMILES: Cc1cccc2oc3c4ccccc4c(N(c4ccc5ccccc5c4)c4ccc5c6ccccc6c6ccccc6c5c4)cc3c12 ; Cc1cccc2oc3c4ccccc4c(N(c4ccc5ccccc5c4)c4cc5ccccc5c5ccccc45)cc3c12 ; Cc1cccc2oc3c4ccccc4c(N(c4cccc5ccccc45)c4ccc5ccc6cccc7ccc4c5c67)cc3c12 ; Cc1cccc2oc3c4ccccc4c(N(c4ccc(-c5ccccc5)cc4)c4ccc5c(c4)C(C)(C)c4ccccc4-5)cc3c12]

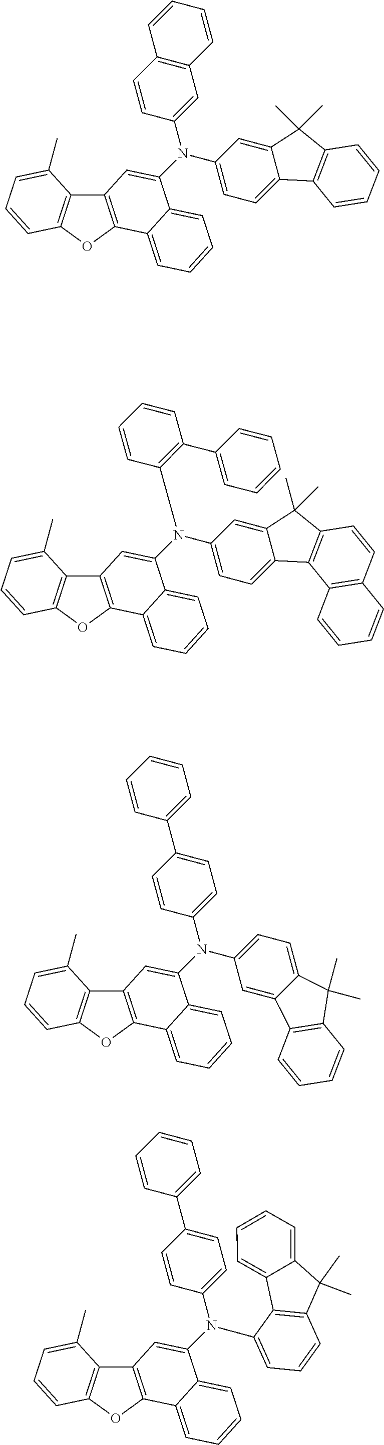 [SMILES: Cc1cccc2oc3c4ccccc4c(N(c4ccc5c(c4)C(C)(C)c4ccccc4-5)c4ccc5ccccc5c4)cc3c12 ; Cc1cccc2oc3c4ccccc4c(N(c4ccc5c(c4)C(C)(C)c4ccc6ccccc6c4-5)c4ccccc4-c4ccccc4)cc3c12 ; Cc1cccc2oc3c4ccccc4c(N(c4ccc(-c5ccccc5)cc4)c4ccc5c(c4)-c4ccccc4C5(C)C)cc3c12 ; Cc1cccc2oc3c4ccccc4c(N(c4ccc(-c5ccccc5)cc4)c4cccc5c4-c4ccccc4C5(C)C)cc3c12]

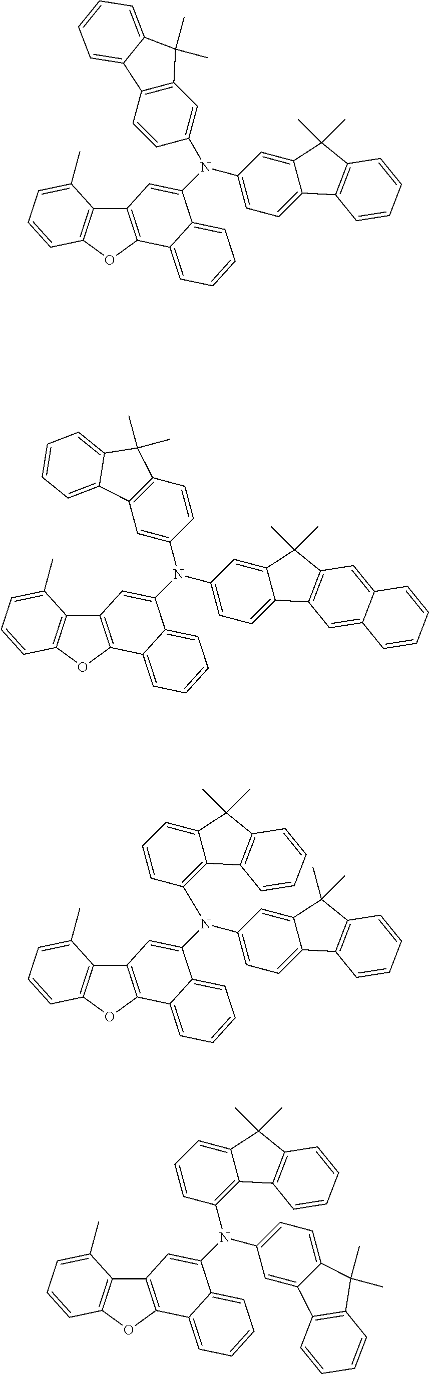 [SMILES: Cc1cccc2oc3c4ccccc4c(N(c4ccc5c(c4)C(C)(C)c4ccccc4-5)c4ccc5c(c4)C(C)(C)c4ccccc4-5)cc3c12 ; Cc1cccc2oc3c4ccccc4c(N(c4ccc5c(c4)-c4ccccc4C5(C)C)c4ccc5c(c4)C(C)(C)c4cc6ccccc6cc4-5)cc3c12 ; Cc1cccc2oc3c4ccccc4c(N(c4ccc5c(c4)C(C)(C)c4ccccc4-5)c4cccc5c4-c4ccccc4C5(C)C)cc3c12 ; Cc1cccc2oc3c4ccccc4c(N(c4ccc5c(c4)-c4ccccc4C5(C)C)c4cccc5c4-c4ccccc4C5(C)C)cc3c12]

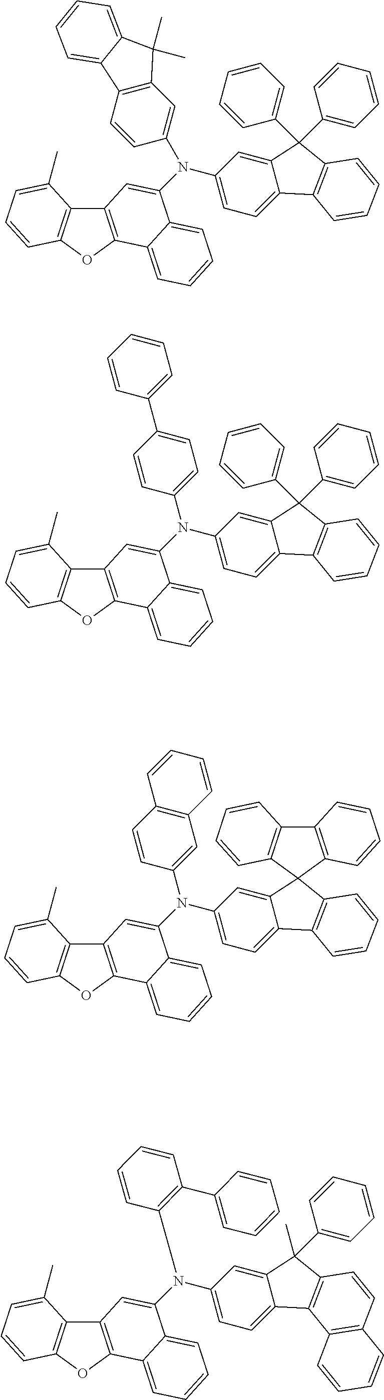 [SMILES: Cc1cccc2oc3c4ccccc4c(N(c4ccc5c(c4)C(C)(C)c4ccccc4-5)c4ccc5c(c4)C(c4ccccc4)(c4ccccc4)c4ccccc4-5)cc3c12 ; Cc1cccc2oc3c4ccccc4c(N(c4ccc(-c5ccccc5)cc4)c4ccc5c(c4)C(c4ccccc4)(c4ccccc4)c4ccccc4-5)cc3c12 ; Cc1cccc2oc3c4ccccc4c(N(c4ccc5c(c4)C4(c6ccccc6-c6ccccc64)c4ccccc4-5)c4ccc5ccccc5c4)cc3c12 ; Cc1cccc2oc3c4ccccc4c(N(c4ccc5c(c4)C(C)(c4ccccc4)c4ccc6ccccc6c4-5)c4ccccc4-c4ccccc4)cc3c12]

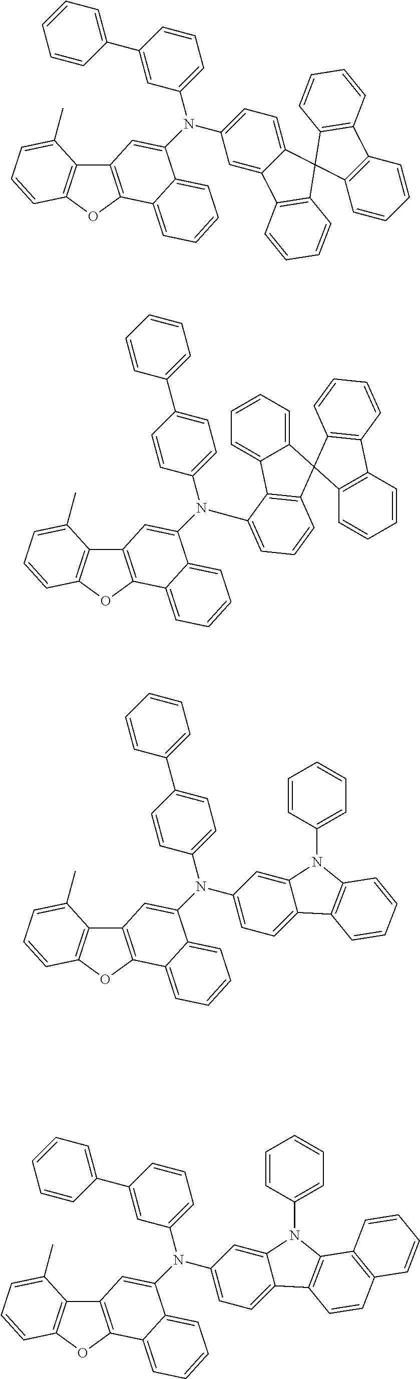 [SMILES: Cc1cccc2oc3c4ccccc4c(N(c4cccc(-c5ccccc5)c4)c4ccc5c(c4)-c4ccccc4C54c5ccccc5-c5ccccc54)cc3c12 ; Cc1cccc2oc3c4ccccc4c(N(c4ccc(-c5ccccc5)cc4)c4cccc5c4-c4ccccc4C54c5ccccc5-c5ccccc54)cc3c12 ; Cc1cccc2oc3c4ccccc4c(N(c4ccc(-c5ccccc5)cc4)c4ccc5c6ccccc6n(-c6ccccc6)c5c4)cc3c12 ; Cc1cccc2oc3c4ccccc4c(N(c4cccc(-c5ccccc5)c4)c4ccc5c6ccc7ccccc7c6n(-c6ccccc6)c5c4)cc3c12]

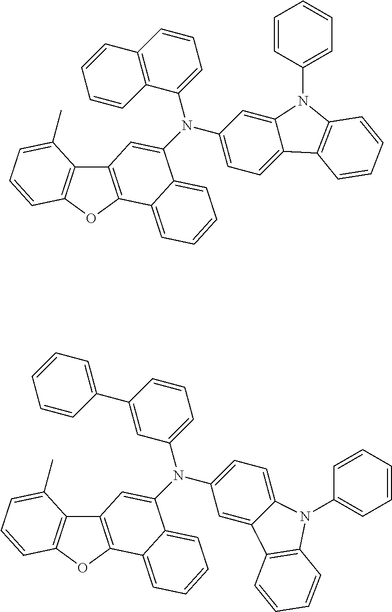 [SMILES: Cc1cccc2oc3c4ccccc4c(N(c4ccc5c6ccccc6n(-c6ccccc6)c5c4)c4cccc5ccccc45)cc3c12 ; Cc1cccc2oc3c4ccccc4c(N(c4cccc(-c5ccccc5)c4)c4ccc5c(c4)c4ccccc4n5-c4ccccc4)cc3c12]

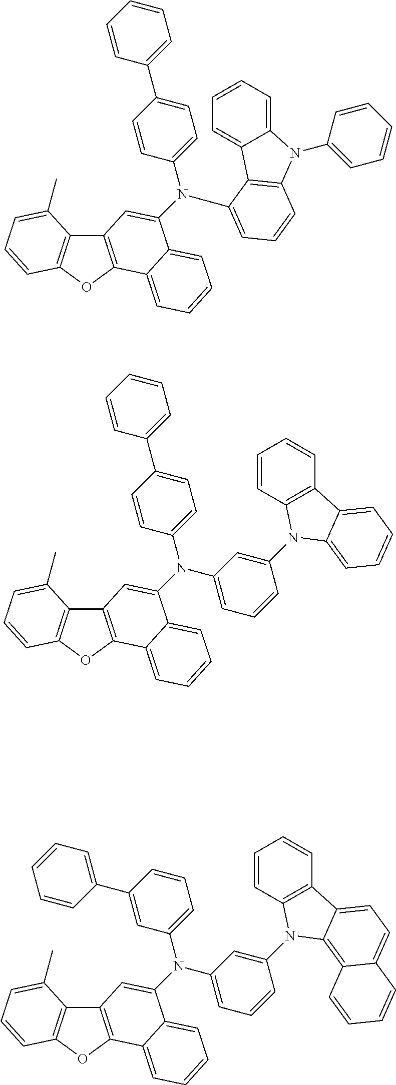 [SMILES: Cc1cccc2oc3c4ccccc4c(N(c4ccc(-c5ccccc5)cc4)c4cccc5c4c4ccccc4n5-c4ccccc4)cc3c12 ; Cc1cccc2oc3c4ccccc4c(N(c4ccc(-c5ccccc5)cc4)c4cccc(-n5c6ccccc6c6ccccc65)c4)cc3c12 ; Cc1cccc2oc3c4ccccc4c(N(c4cccc(-c5ccccc5)c4)c4cccc(-n5c6ccccc6c6ccc7ccccc7c65)c4)cc3c12]

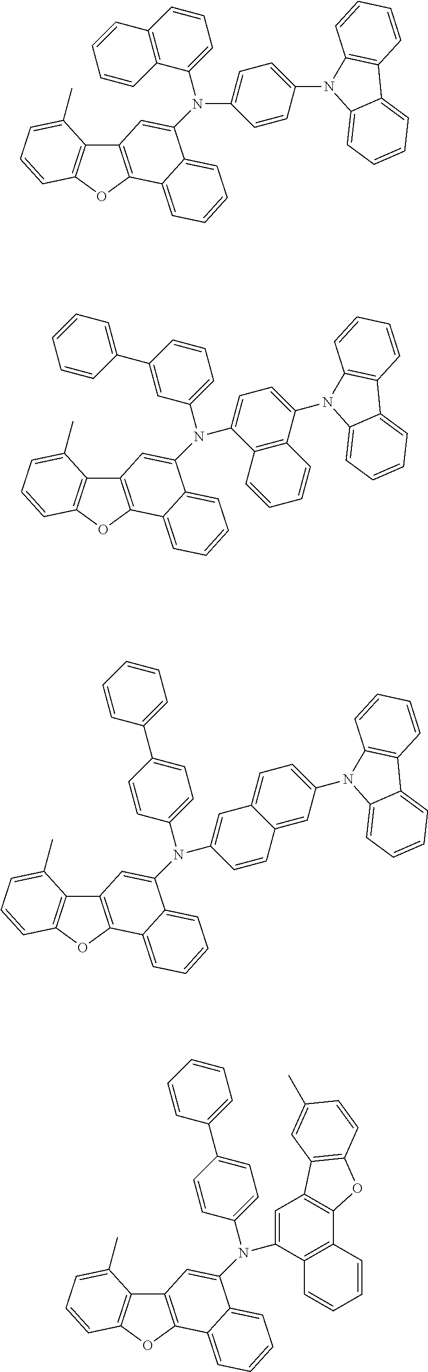 [SMILES: Cc1cccc2oc3c4ccccc4c(N(c4ccc(-n5c6ccccc6c6ccccc65)cc4)c4cccc5ccccc45)cc3c12 ; Cc1cccc2oc3c4ccccc4c(N(c4cccc(-c5ccccc5)c4)c4ccc(-n5c6ccccc6c6ccccc65)c5ccccc45)cc3c12 ; Cc1cccc2oc3c4ccccc4c(N(c4ccc(-c5ccccc5)cc4)c4ccc5cc(-n6c7ccccc7c7ccccc76)ccc5c4)cc3c12 ; Cc1ccc2oc3c4ccccc4c(N(c4ccc(-c5ccccc5)cc4)c4cc5c(oc6cccc(C)c65)c5ccccc45)cc3c2c1]

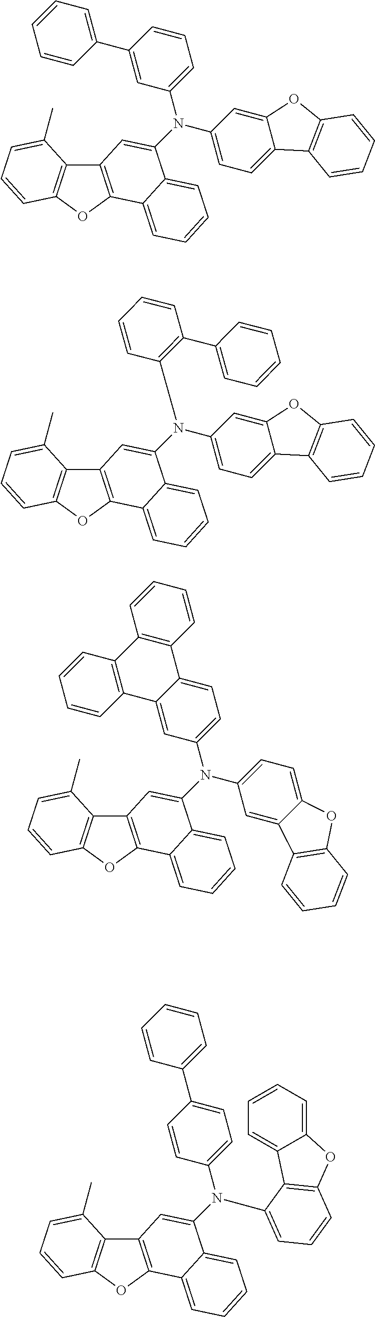

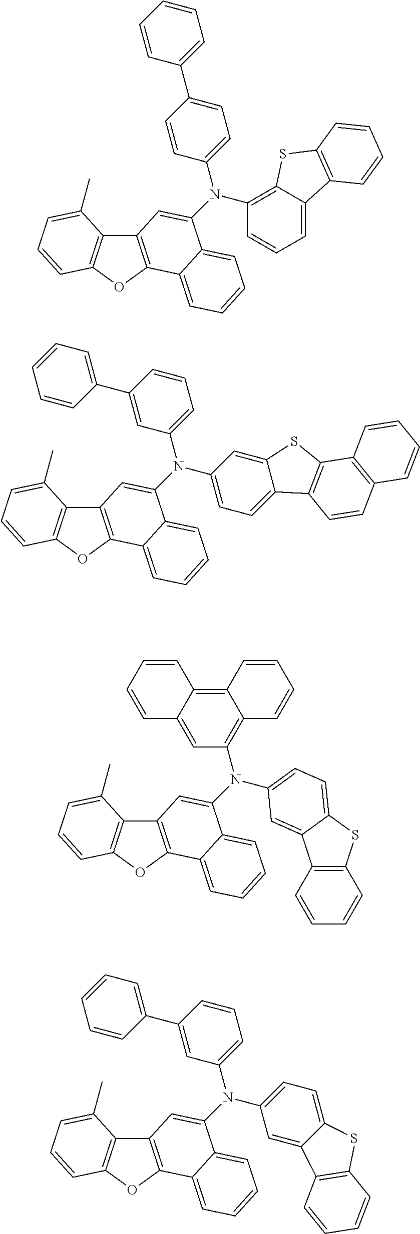 [SMILES: Cc1cccc2oc3c4ccccc4c(N(c4ccc(-c5ccccc5)cc4)c4cccc5c4sc4ccccc45)cc3c12 ; Cc1cccc2oc3c4ccccc4c(N(c4cccc(-c5ccccc5)c4)c4ccc5c(c4)sc4c6ccccc6ccc54)cc3c12 ; Cc1cccc2oc3c4ccccc4c(N(c4ccc5sc6ccccc6c5c4)c4cc5ccccc5c5ccccc45)cc3c12 ; Cc1cccc2oc3c4ccccc4c(N(c4cccc(-c5ccccc5)c4)c4ccc5sc6ccccc6c5c4)cc3c12]

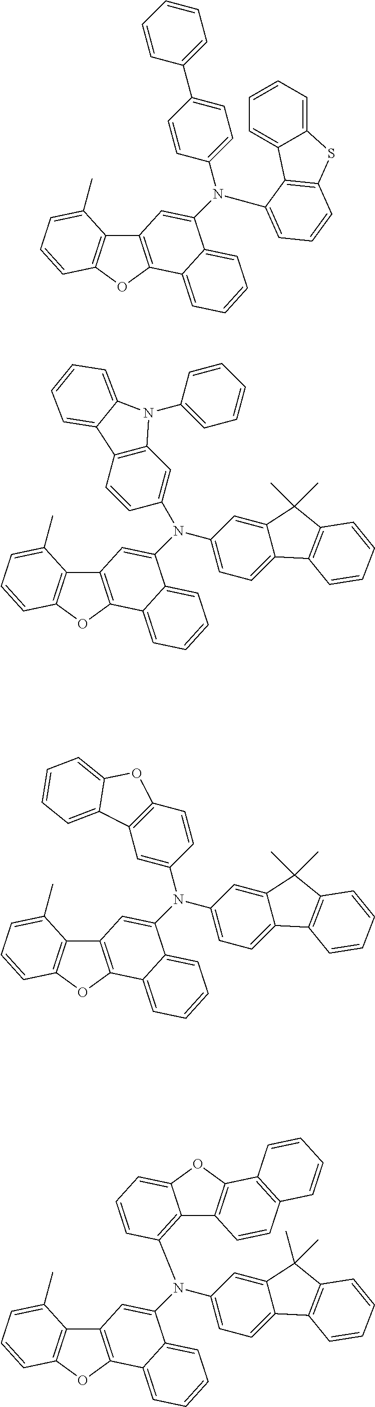 [SMILES: Cc1cccc2oc3c4ccccc4c(N(c4ccc(-c5ccccc5)cc4)c4cccc5sc6ccccc6c45)cc3c12 ; Cc1cccc2oc3c4ccccc4c(N(c4ccc5c(c4)C(C)(C)c4ccccc4-5)c4ccc5c6ccccc6n(-c6ccccc6)c5c4)cc3c12 ; Cc1cccc2oc3c4ccccc4c(N(c4ccc5c(c4)C(C)(C)c4ccccc4-5)c4ccc5oc6ccccc6c5c4)cc3c12 ; Cc1cccc2oc3c4ccccc4c(N(c4ccc5c(c4)C(C)(C)c4ccccc4-5)c4cccc5oc6c7ccccc7ccc6c45)cc3c12]

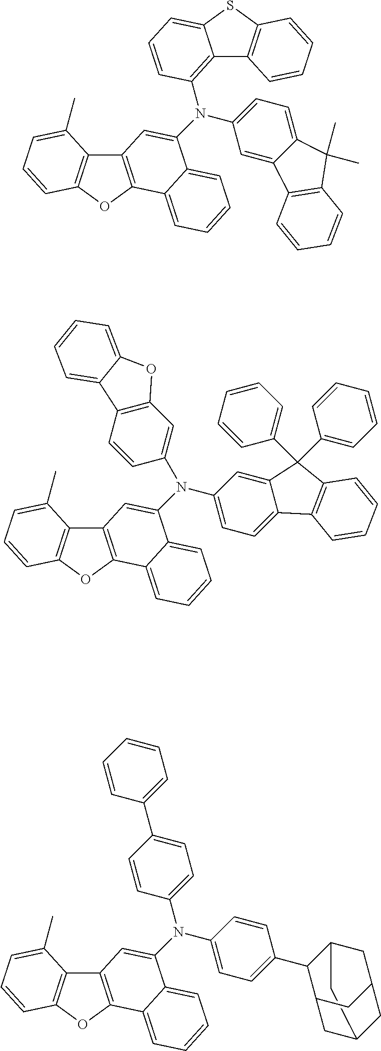 [SMILES: Cc1cccc2oc3c4ccccc4c(N(c4ccc5c(c4)-c4ccccc4C5(C)C)c4cccc5sc6ccccc6c45)cc3c12 ; Cc1cccc2oc3c4ccccc4c(N(c4ccc5c(c4)C(c4ccccc4)(c4ccccc4)c4ccccc4-5)c4ccc5c(c4)oc4ccccc45)cc3c12 ; Cc1cccc2oc3c4ccccc4c(N(c4ccc(-c5ccccc5)cc4)c4ccc(C5C6CC7CC(C6)CC5C7)cc4)cc3c12]

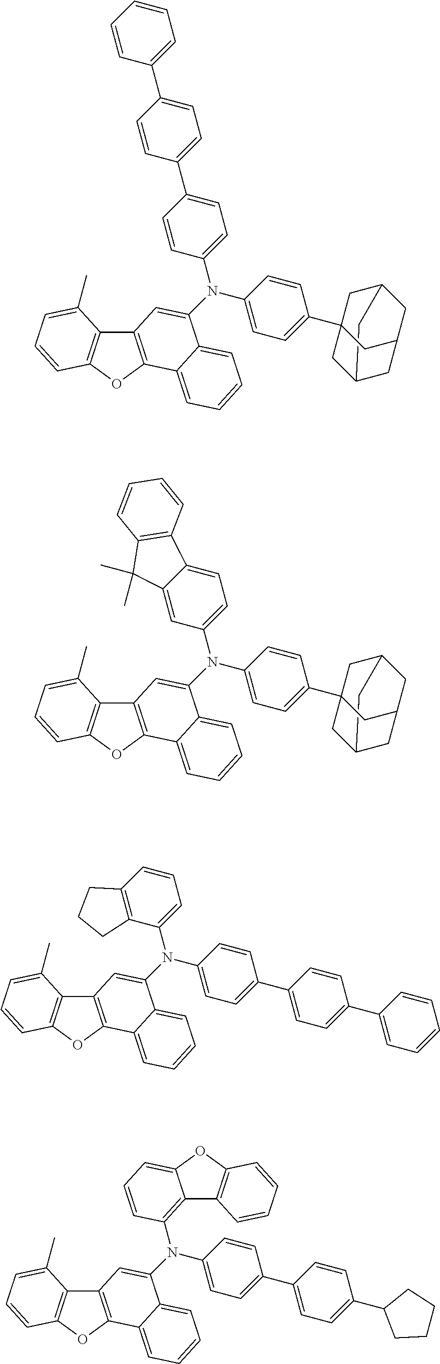 [SMILES: Cc1cccc2oc3c4ccccc4c(N(c4ccc(-c5ccc(-c6ccccc6)cc5)cc4)c4ccc(C56CC7CC(CC(C7)C5)C6)cc4)cc3c12 ; Cc1cccc2oc3c4ccccc4c(N(c4ccc(C56CC7CC(CC(C7)C5)C6)cc4)c4ccc5c(c4)C(C)(C)c4ccccc4-5)cc3c12 ; Cc1cccc2oc3c4ccccc4c(N(c4ccc(-c5ccc(-c6ccccc6)cc5)cc4)c4cccc5c4CCC5)cc3c12 ; Cc1cccc2oc3c4ccccc4c(N(c4ccc(-c5ccc(C6CCCC6)cc5)cc4)c4cccc5oc6ccccc6c45)cc3c12]

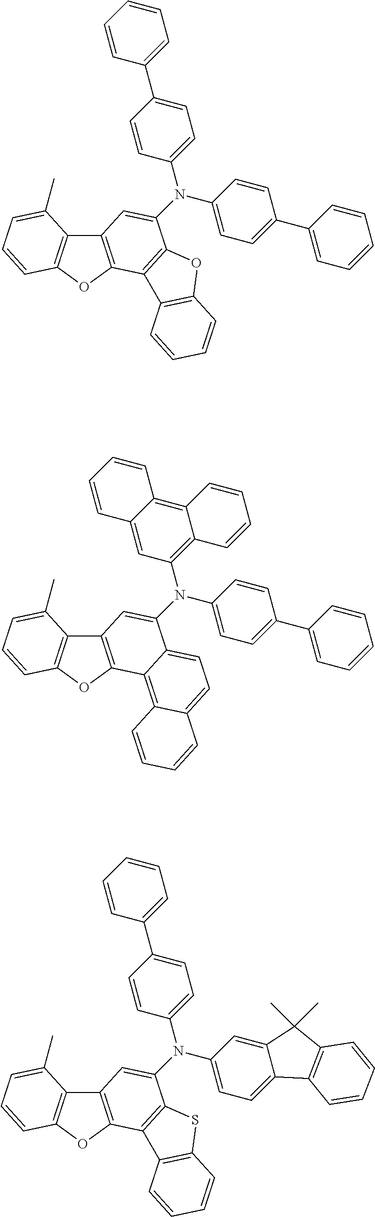 [SMILES: Cc1cccc2oc3c(cc(N(c4ccc(-c5ccccc5)cc4)c4ccc(-c5ccccc5)cc4)c4oc5ccccc5c43)c12 ; Cc1cccc2oc3c(cc(N(c4ccc(-c5ccccc5)cc4)c4cc5ccccc5c5ccccc45)c4ccc5ccccc5c43)c12 ; Cc1cccc2oc3c(cc(N(c4ccc(-c5ccccc5)cc4)c4ccc5c(c4)C(C)(C)c4ccccc4-5)c4sc5ccccc5c43)c12]

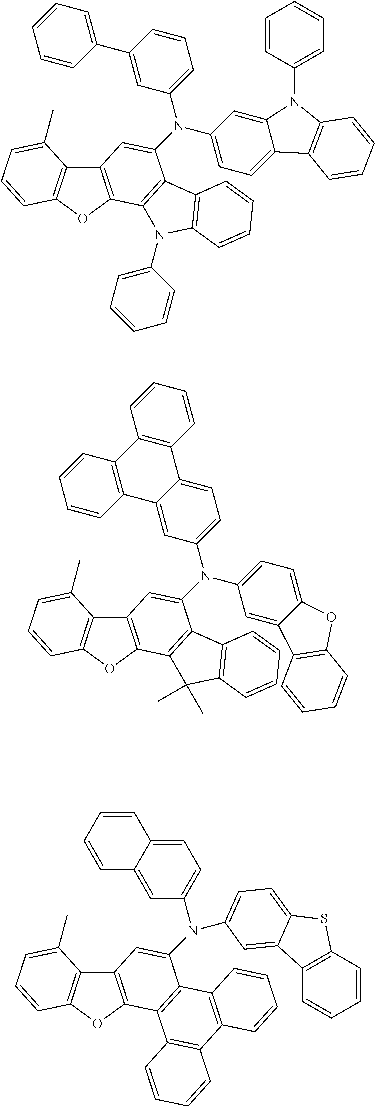 [SMILES: Cc1cccc2oc3c(cc(N(c4cccc(-c5ccccc5)c4)c4ccc5c6ccccc6n(-c6ccccc6)c5c4)c4c5ccccc5n(-c5ccccc5)c34)c12 ; Cc1cccc2oc3c4c(c(N(c5ccc6oc7ccccc7c6c5)c5ccc6c7ccccc7c7ccccc7c6c5)cc3c12)-c1ccccc1C4(C)C ; Cc1cccc2oc3c(cc(N(c4ccc5ccccc5c4)c4ccc5sc6ccccc6c5c4)c4c5ccccc5c5ccccc5c34)c12]

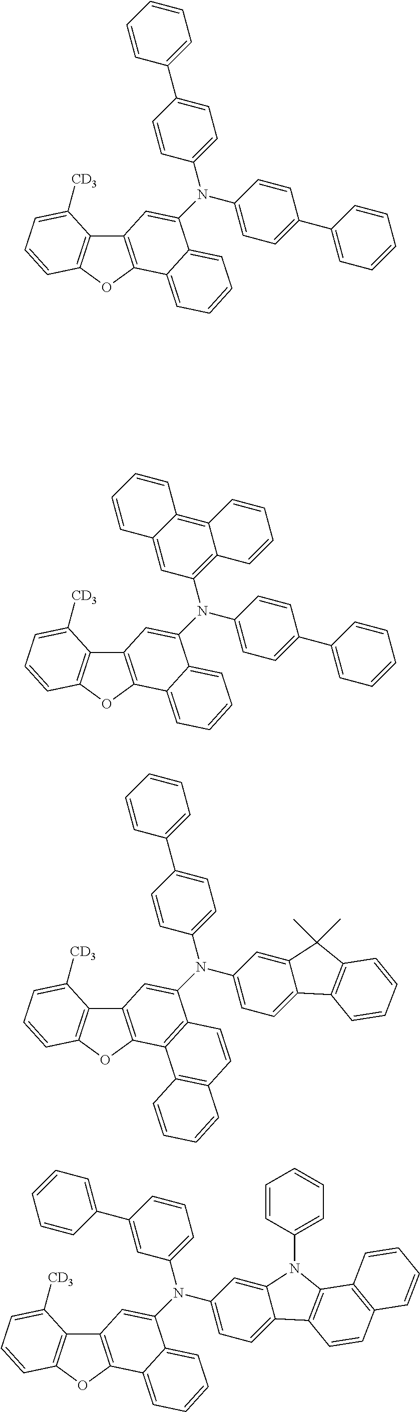 [SMILES: [2H]C([2H])([2H])c1cccc2oc3c4ccccc4c(N(c4ccc(-c5ccccc5)cc4)c4ccc(-c5ccccc5)cc4)cc3c12 ; [2H]C([2H])([2H])c1cccc2oc3c4ccccc4c(N(c4ccc(-c5ccccc5)cc4)c4cc5ccccc5c5ccccc45)cc3c12 ; [2H]C([2H])([2H])c1cccc2oc3c(cc(N(c4ccc(-c5ccccc5)cc4)c4ccc5c(c4)C(C)(C)c4ccccc4-5)c4ccc5ccccc5c43)c12 ; [2H]C([2H])([2H])c1cccc2oc3c4ccccc4c(N(c4cccc(-c5ccccc5)c4)c4ccc5c6ccc7ccccc7c6n(-c6ccccc6)c5c4)cc3c12]

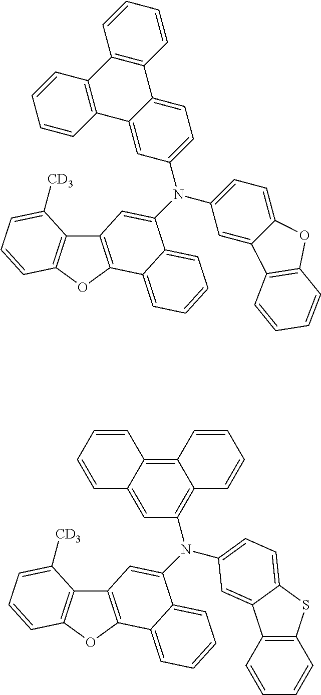 [SMILES: [2H]C([2H])([2H])c1cccc2oc3c4ccccc4c(N(c4ccc5oc6ccccc6c5c4)c4ccc5c6ccccc6c6ccccc6c5c4)cc3c12 ; [2H]C([2H])([2H])c1cccc2oc3c4ccccc4c(N(c4ccc5sc6ccccc6c5c4)c4cc5ccccc5c5ccccc45)cc3c12]

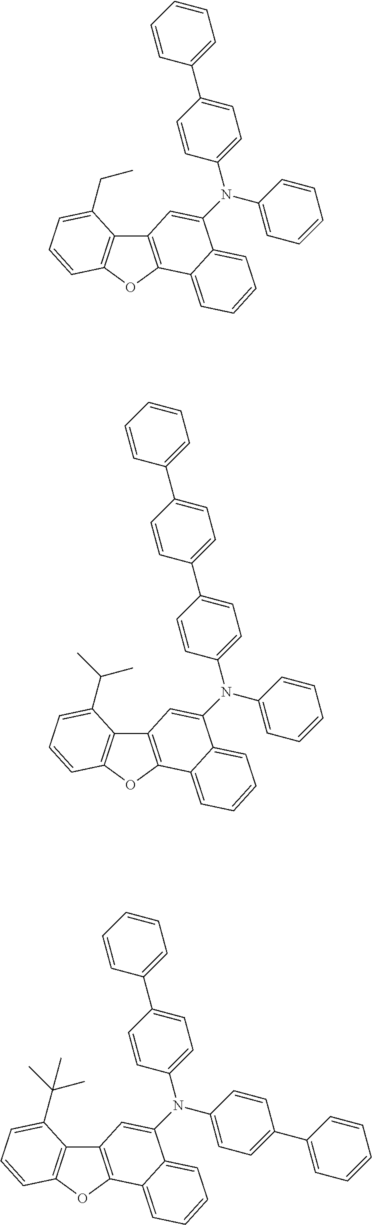 [SMILES: CCc1cccc2oc3c4ccccc4c(N(c4ccccc4)c4ccc(-c5ccccc5)cc4)cc3c12 ; CC(C)c1cccc2oc3c4ccccc4c(N(c4ccccc4)c4ccc(-c5ccc(-c6ccccc6)cc5)cc4)cc3c12 ; CC(C)(C)c1cccc2oc3c4ccccc4c(N(c4ccc(-c5ccccc5)cc4)c4ccc(-c5ccccc5)cc4)cc3c12]

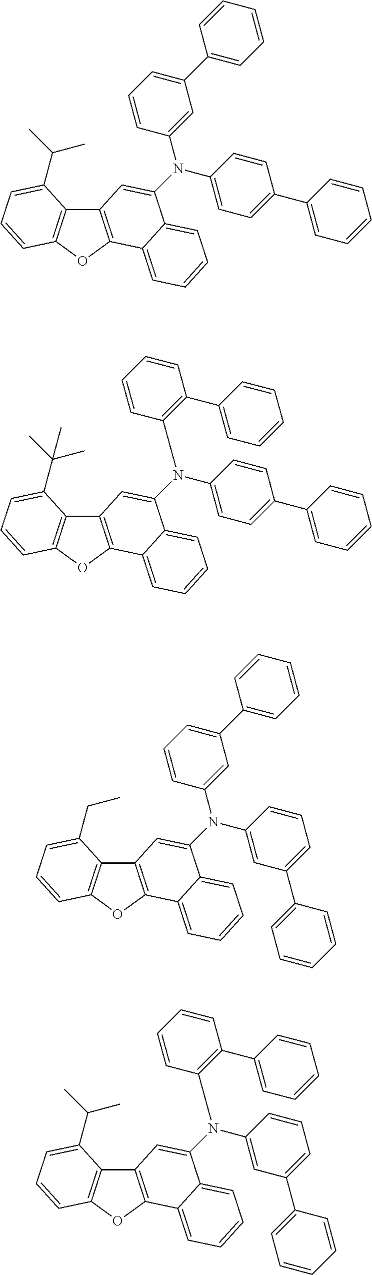 [SMILES: CC(C)c1cccc2oc3c4ccccc4c(N(c4ccc(-c5ccccc5)cc4)c4cccc(-c5ccccc5)c4)cc3c12 ; CC(C)(C)c1cccc2oc3c4ccccc4c(N(c4ccc(-c5ccccc5)cc4)c4ccccc4-c4ccccc4)cc3c12 ; CCc1cccc2oc3c4ccccc4c(N(c4cccc(-c5ccccc5)c4)c4cccc(-c5ccccc5)c4)cc3c12 ; CC(C)c1cccc2oc3c4ccccc4c(N(c4cccc(-c5ccccc5)c4)c4ccccc4-c4ccccc4)cc3c12]

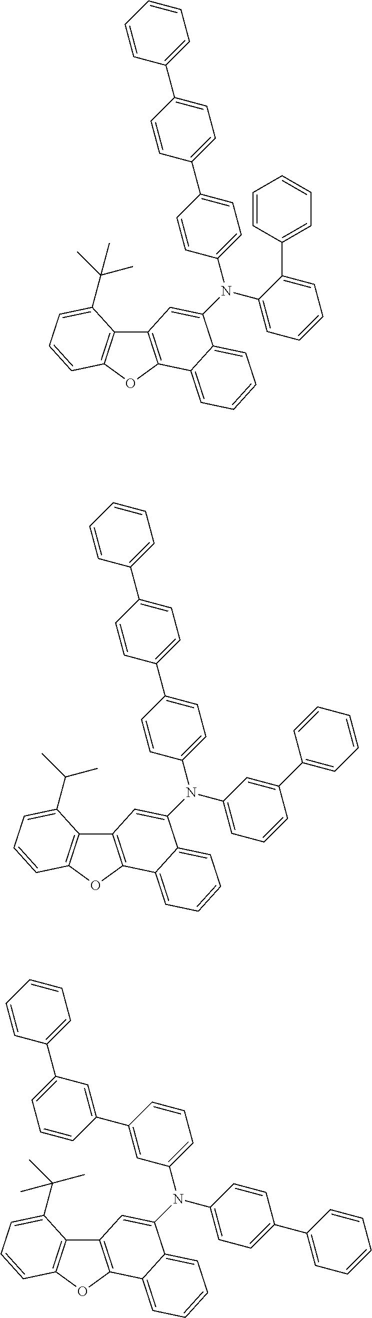 [SMILES: CC(C)(C)c1cccc2oc3c4ccccc4c(N(c4ccc(-c5ccc(-c6ccccc6)cc5)cc4)c4ccccc4-c4ccccc4)cc3c12 ; CC(C)c1cccc2oc3c4ccccc4c(N(c4ccc(-c5ccc(-c6ccccc6)cc5)cc4)c4cccc(-c5ccccc5)c4)cc3c12 ; CC(C)(C)c1cccc2oc3c4ccccc4c(N(c4ccc(-c5ccccc5)cc4)c4cccc(-c5cccc(-c6ccccc6)c5)c4)cc3c12]

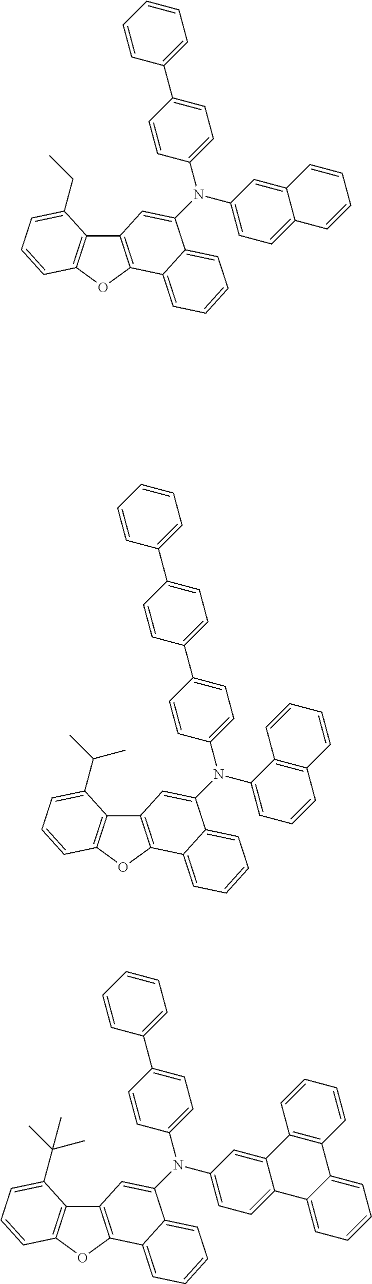 [SMILES: CCc1cccc2oc3c4ccccc4c(N(c4ccc(-c5ccccc5)cc4)c4ccc5ccccc5c4)cc3c12 ; CC(C)c1cccc2oc3c4ccccc4c(N(c4ccc(-c5ccc(-c6ccccc6)cc5)cc4)c4cccc5ccccc45)cc3c12 ; CC(C)(C)c1cccc2oc3c4ccccc4c(N(c4ccc(-c5ccccc5)cc4)c4ccc5c6ccccc6c6ccccc6c5c4)cc3c12]

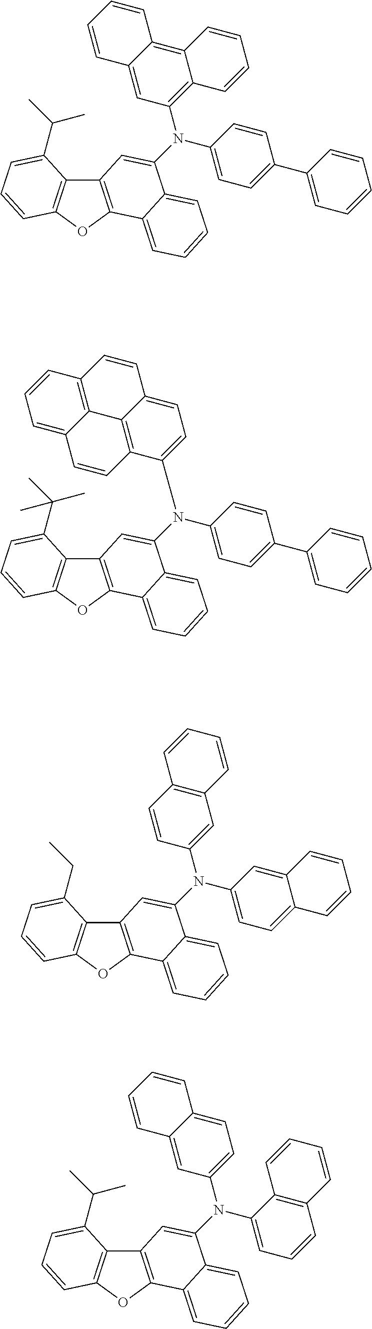 [SMILES: CC(C)c1cccc2oc3c4ccccc4c(N(c4ccc(-c5ccccc5)cc4)c4cc5ccccc5c5ccccc45)cc3c12 ; CC(C)(C)c1cccc2oc3c4ccccc4c(N(c4ccc(-c5ccccc5)cc4)c4ccc5ccc6cccc7ccc4c5c67)cc3c12 ; CCc1cccc2oc3c4ccccc4c(N(c4ccc5ccccc5c4)c4ccc5ccccc5c4)cc3c12 ; CC(C)c1cccc2oc3c4ccccc4c(N(c4ccc5ccccc5c4)c4cccc5ccccc45)cc3c12]

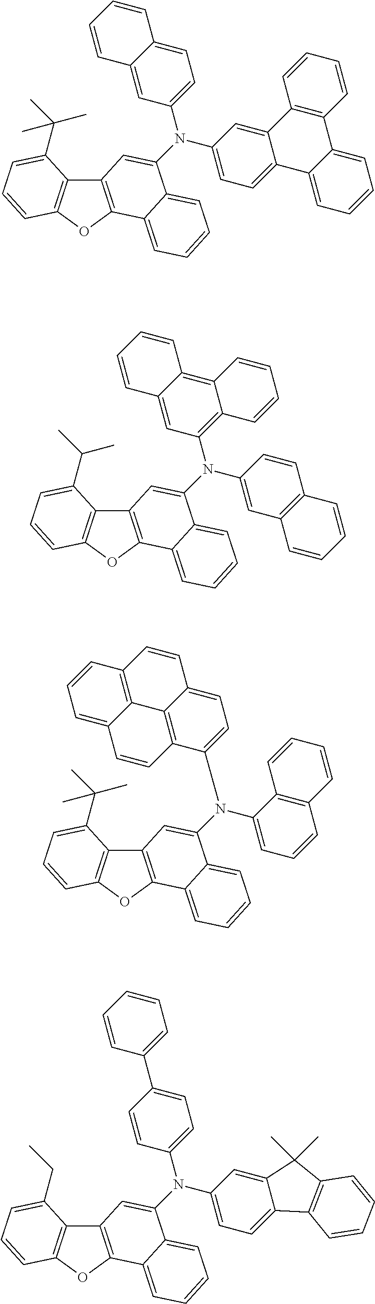 [SMILES: CC(C)(C)c1cccc2oc3c4ccccc4c(N(c4ccc5ccccc5c4)c4ccc5c6ccccc6c6ccccc6c5c4)cc3c12 ; CC(C)c1cccc2oc3c4ccccc4c(N(c4ccc5ccccc5c4)c4cc5ccccc5c5ccccc45)cc3c12 ; CC(C)(C)c1cccc2oc3c4ccccc4c(N(c4cccc5ccccc45)c4ccc5ccc6cccc7ccc4c5c67)cc3c12 ; CCc1cccc2oc3c4ccccc4c(N(c4ccc(-c5ccccc5)cc4)c4ccc5c(c4)C(C)(C)c4ccccc4-5)cc3c12]

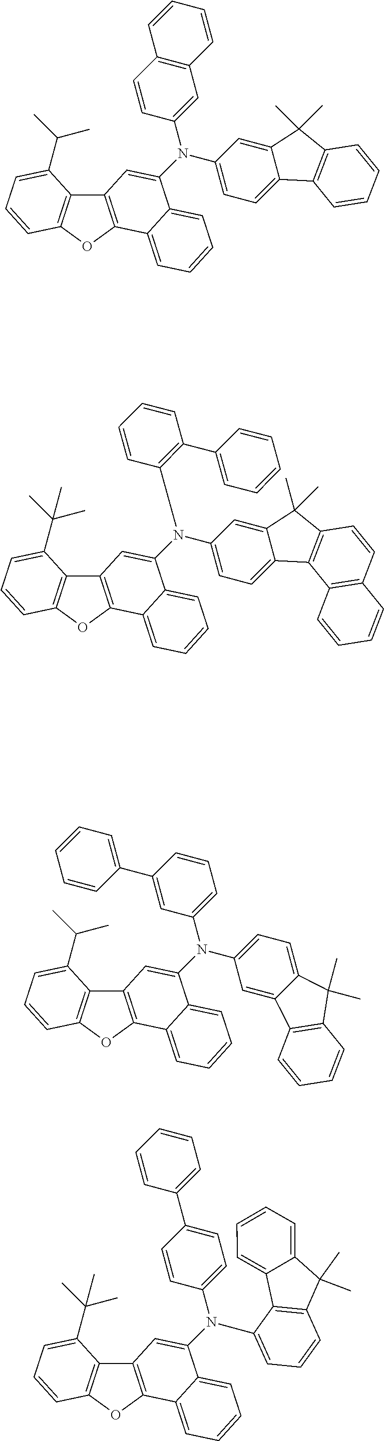 [SMILES: CC(C)c1cccc2oc3c4ccccc4c(N(c4ccc5c(c4)C(C)(C)c4ccccc4-5)c4ccc5ccccc5c4)cc3c12 ; CC(C)(C)c1cccc2oc3c4ccccc4c(N(c4ccc5c(c4)C(C)(C)c4ccc6ccccc6c4-5)c4ccccc4-c4ccccc4)cc3c12 ; CC(C)c1cccc2oc3c4ccccc4c(N(c4cccc(-c5ccccc5)c4)c4ccc5c(c4)-c4ccccc4C5(C)C)cc3c12 ; CC(C)(C)c1cccc2oc3c4ccccc4c(N(c4ccc(-c5ccccc5)cc4)c4cccc5c4-c4ccccc4C5(C)C)cc3c12]

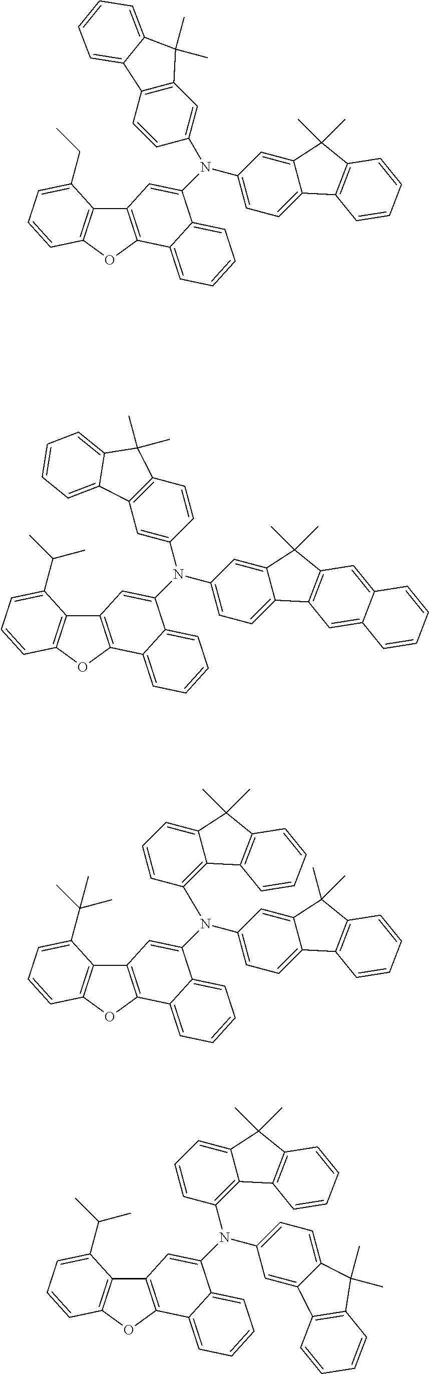 [SMILES: CCc1cccc2oc3c4ccccc4c(N(c4ccc5c(c4)C(C)(C)c4ccccc4-5)c4ccc5c(c4)C(C)(C)c4ccccc4-5)cc3c12 ; CC(C)c1cccc2oc3c4ccccc4c(N(c4ccc5c(c4)-c4ccccc4C5(C)C)c4ccc5c(c4)C(C)(C)c4cc6ccccc6cc4-5)cc3c12 ; CC(C)(C)c1cccc2oc3c4ccccc4c(N(c4ccc5c(c4)C(C)(C)c4ccccc4-5)c4cccc5c4-c4ccccc4C5(C)C)cc3c12 ; CC(C)c1cccc2oc3c4ccccc4c(N(c4ccc5c(c4)-c4ccccc4C5(C)C)c4cccc5c4-c4ccccc4C5(C)C)cc3c12]

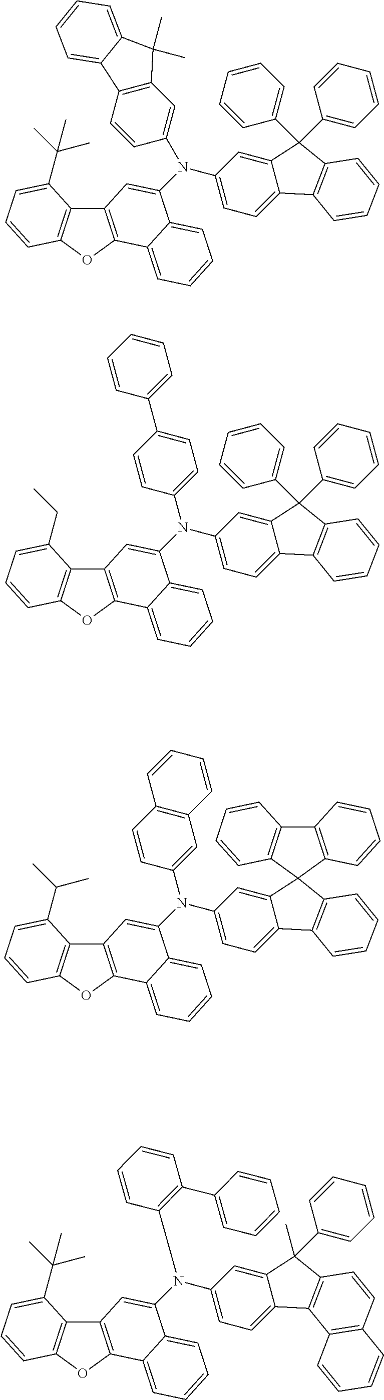 [SMILES: CC(C)(C)c1cccc2oc3c4ccccc4c(N(c4ccc5c(c4)C(C)(C)c4ccccc4-5)c4ccc5c(c4)C(c4ccccc4)(c4ccccc4)c4ccccc4-5)cc3c12 ; CCc1cccc2oc3c4ccccc4c(N(c4ccc(-c5ccccc5)cc4)c4ccc5c(c4)C(c4ccccc4)(c4ccccc4)c4ccccc4-5)cc3c12 ; CC(C)c1cccc2oc3c4ccccc4c(N(c4ccc5c(c4)C4(c6ccccc6-c6ccccc64)c4ccccc4-5)c4ccc5ccccc5c4)cc3c12 ; CC(C)(C)c1cccc2oc3c4ccccc4c(N(c4ccc5c(c4)C(C)(c4ccccc4)c4ccc6ccccc6c4-5)c4ccccc4-c4ccccc4)cc3c12]

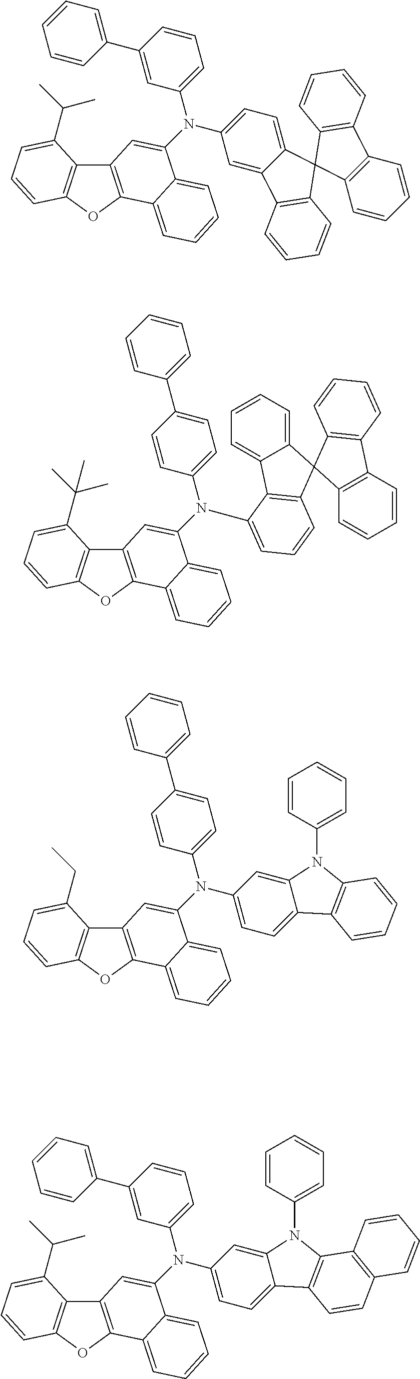 [SMILES: CC(C)c1cccc2oc3c4ccccc4c(N(c4cccc(-c5ccccc5)c4)c4ccc5c(c4)-c4ccccc4C54c5ccccc5-c5ccccc54)cc3c12 ; CC(C)(C)c1cccc2oc3c4ccccc4c(N(c4ccc(-c5ccccc5)cc4)c4cccc5c4-c4ccccc4C54c5ccccc5-c5ccccc54)cc3c12 ; CCc1cccc2oc3c4ccccc4c(N(c4ccc(-c5ccccc5)cc4)c4ccc5c6ccccc6n(-c6ccccc6)c5c4)cc3c12 ; CC(C)c1cccc2oc3c4ccccc4c(N(c4cccc(-c5ccccc5)c4)c4ccc5c6ccc7ccccc7c6n(-c6ccccc6)c5c4)cc3c12]

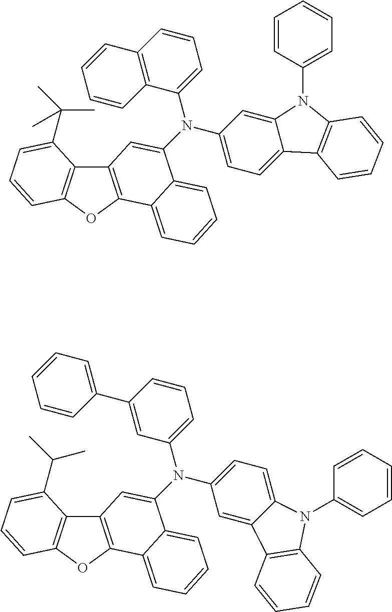 [SMILES: CC(C)(C)c1cccc2oc3c4ccccc4c(N(c4ccc5c6ccccc6n(-c6ccccc6)c5c4)c4cccc5ccccc45)cc3c12 ; CC(C)c1cccc2oc3c4ccccc4c(N(c4cccc(-c5ccccc5)c4)c4ccc5c(c4)c4ccccc4n5-c4ccccc4)cc3c12]

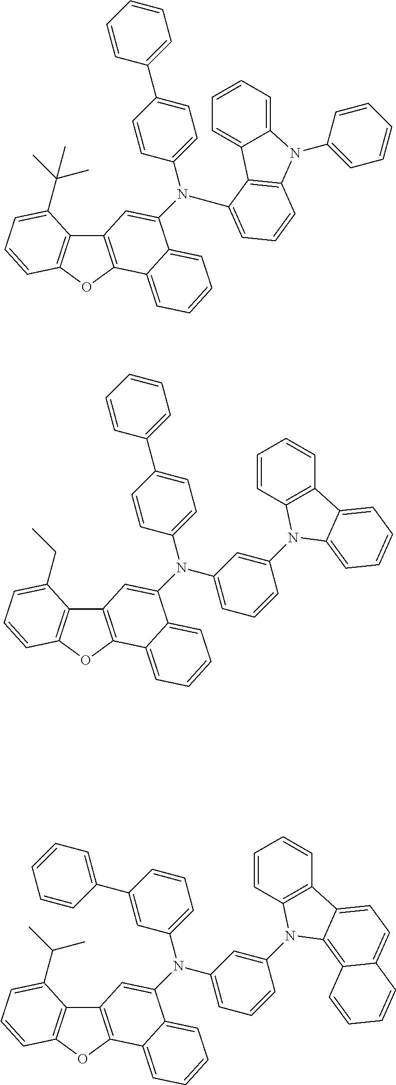 [SMILES: CC(C)(C)c1cccc2oc3c4ccccc4c(N(c4ccc(-c5ccccc5)cc4)c4cccc5c4c4ccccc4n5-c4ccccc4)cc3c12 ; CCc1cccc2oc3c4ccccc4c(N(c4ccc(-c5ccccc5)cc4)c4cccc(-n5c6ccccc6c6ccccc65)c4)cc3c12 ; CC(C)c1cccc2oc3c4ccccc4c(N(c4cccc(-c5ccccc5)c4)c4cccc(-n5c6ccccc6c6ccc7ccccc7c65)c4)cc3c12]

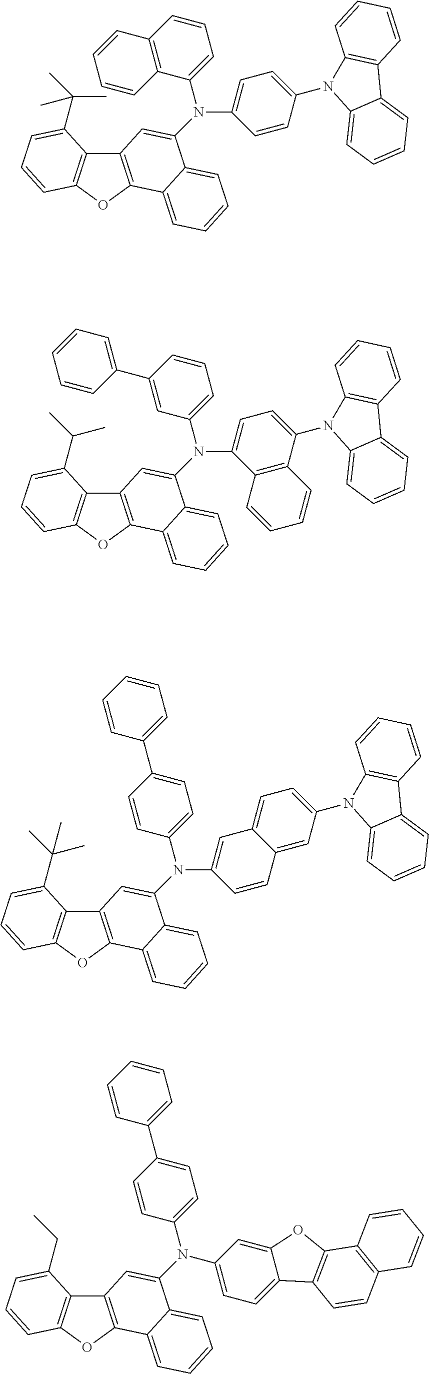 [SMILES: CC(C)(C)c1cccc2oc3c4ccccc4c(N(c4ccc(-n5c6ccccc6c6ccccc65)cc4)c4cccc5ccccc45)cc3c12 ; CC(C)c1cccc2oc3c4ccccc4c(N(c4cccc(-c5ccccc5)c4)c4ccc(-n5c6ccccc6c6ccccc65)c5ccccc45)cc3c12 ; CC(C)(C)c1cccc2oc3c4ccccc4c(N(c4ccc(-c5ccccc5)cc4)c4ccc5cc(-n6c7ccccc7c7ccccc76)ccc5c4)cc3c12 ; CCc1cccc2oc3c4ccccc4c(N(c4ccc(-c5ccccc5)cc4)c4ccc5c(c4)oc4c6ccccc6ccc54)cc3c12]

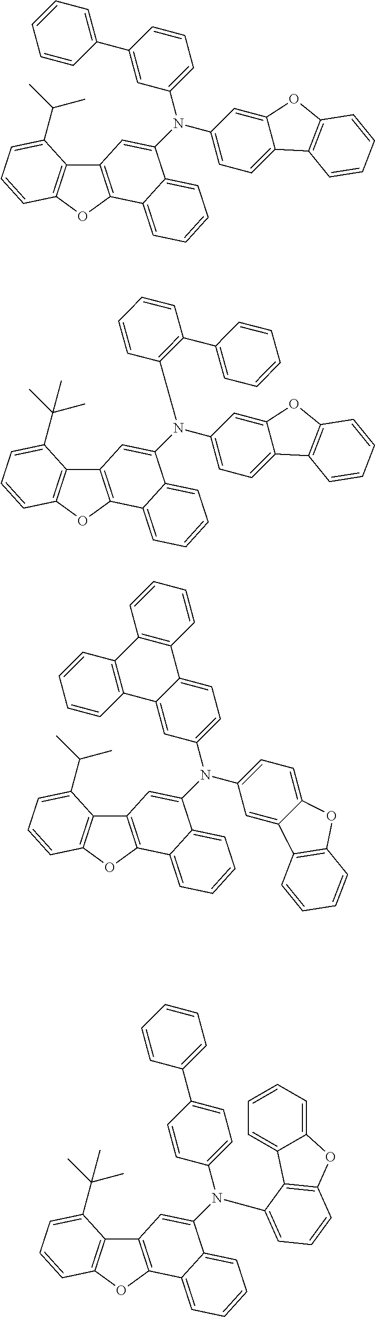

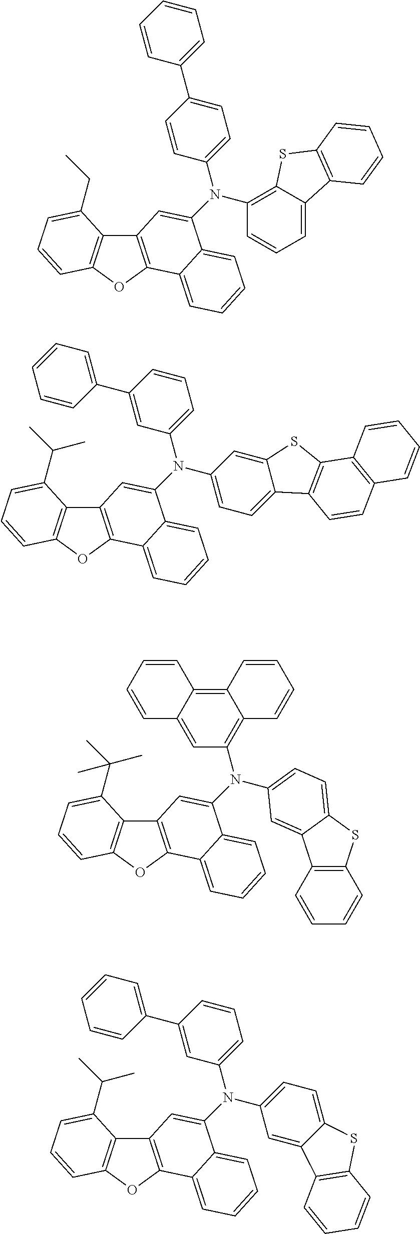 [SMILES: CCc1cccc2oc3c4ccccc4c(N(c4ccc(-c5ccccc5)cc4)c4cccc5c4sc4ccccc45)cc3c12 ; CC(C)c1cccc2oc3c4ccccc4c(N(c4cccc(-c5ccccc5)c4)c4ccc5c(c4)sc4c6ccccc6ccc54)cc3c12 ; CC(C)(C)c1cccc2oc3c4ccccc4c(N(c4ccc5sc6ccccc6c5c4)c4cc5ccccc5c5ccccc45)cc3c12 ; CC(C)c1cccc2oc3c4ccccc4c(N(c4cccc(-c5ccccc5)c4)c4ccc5sc6ccccc6c5c4)cc3c12]

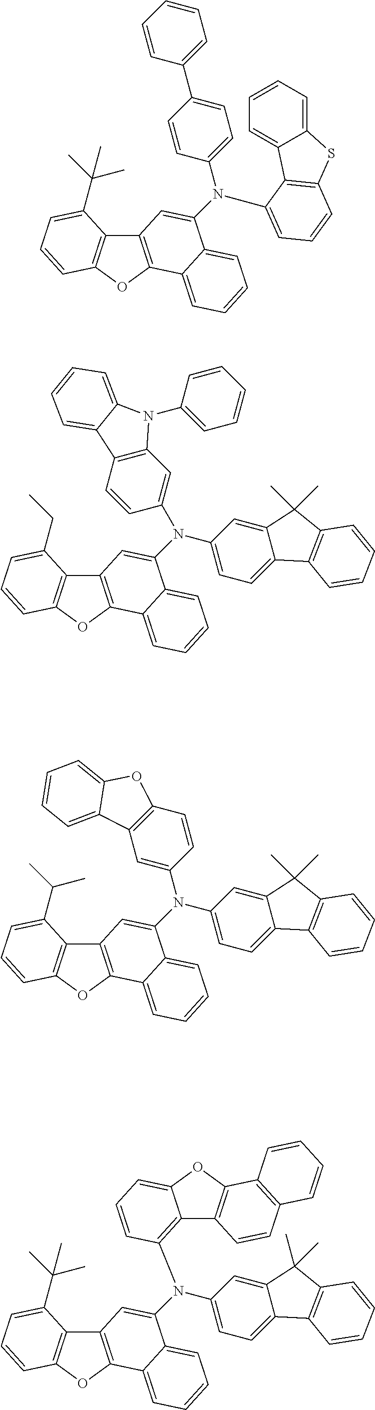 [SMILES: CC(C)(C)c1cccc2oc3c4ccccc4c(N(c4ccc(-c5ccccc5)cc4)c4cccc5sc6ccccc6c45)cc3c12 ; CCc1cccc2oc3c4ccccc4c(N(c4ccc5c(c4)C(C)(C)c4ccccc4-5)c4ccc5c6ccccc6n(-c6ccccc6)c5c4)cc3c12 ; CC(C)c1cccc2oc3c4ccccc4c(N(c4ccc5c(c4)C(C)(C)c4ccccc4-5)c4ccc5oc6ccccc6c5c4)cc3c12 ; CC(C)(C)c1cccc2oc3c4ccccc4c(N(c4ccc5c(c4)C(C)(C)c4ccccc4-5)c4cccc5oc6c7ccccc7ccc6c45)cc3c12]

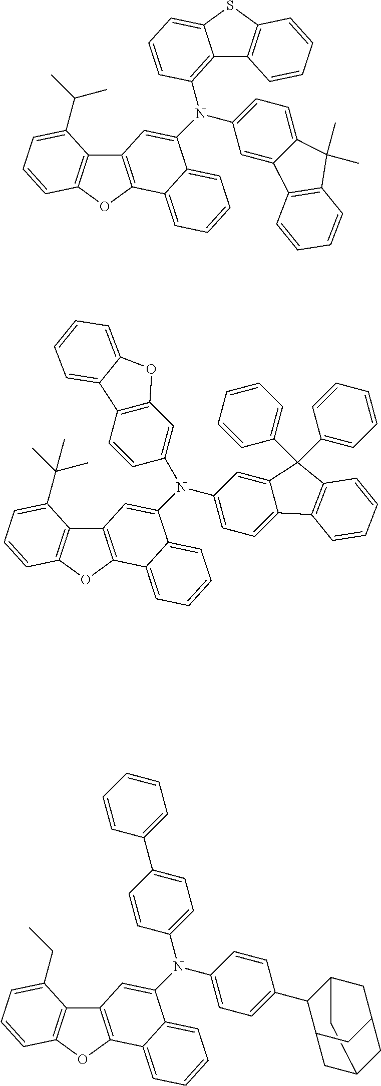 [SMILES: CC(C)c1cccc2oc3c4ccccc4c(N(c4ccc5c(c4)-c4ccccc4C5(C)C)c4cccc5sc6ccccc6c45)cc3c12 ; CC(C)(C)c1cccc2oc3c4ccccc4c(N(c4ccc5c(c4)C(c4ccccc4)(c4ccccc4)c4ccccc4-5)c4ccc5c(c4)oc4ccccc45)cc3c12 ; CCc1cccc2oc3c4ccccc4c(N(c4ccc(-c5ccccc5)cc4)c4ccc(C5C6CC7CC(C6)CC5C7)cc4)cc3c12]

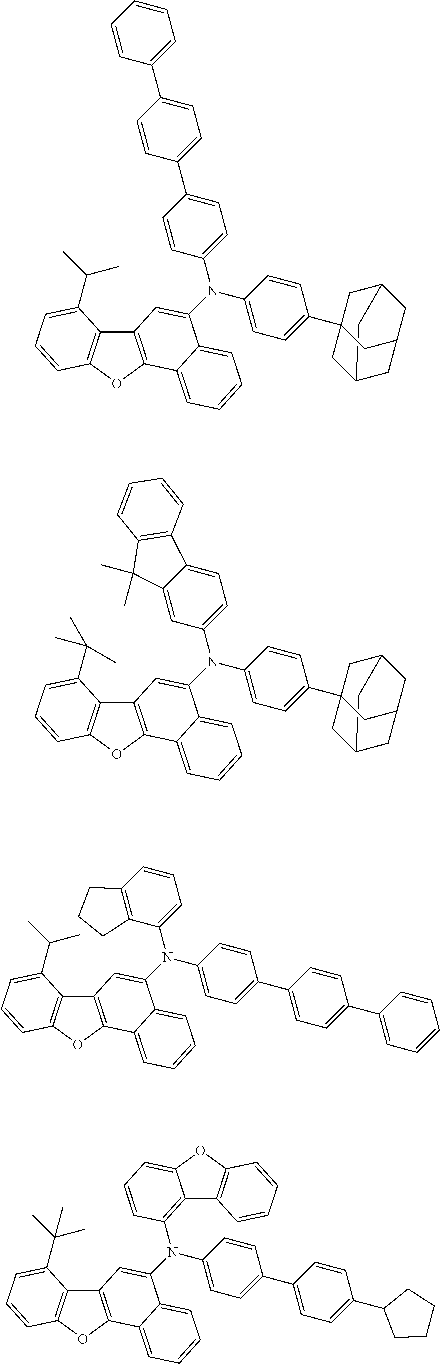 [SMILES: CC(C)c1cccc2oc3c4ccccc4c(N(c4ccc(-c5ccc(-c6ccccc6)cc5)cc4)c4ccc(C56CC7CC(CC(C7)C5)C6)cc4)cc3c12 ; CC(C)(C)c1cccc2oc3c4ccccc4c(N(c4ccc(C56CC7CC(CC(C7)C5)C6)cc4)c4ccc5c(c4)C(C)(C)c4ccccc4-5)cc3c12 ; CC(C)c1cccc2oc3c4ccccc4c(N(c4ccc(-c5ccc(-c6ccccc6)cc5)cc4)c4cccc5c4CCC5)cc3c12 ; CC(C)(C)c1cccc2oc3c4ccccc4c(N(c4ccc(-c5ccc(C6CCCC6)cc5)cc4)c4cccc5oc6ccccc6c45)cc3c12]

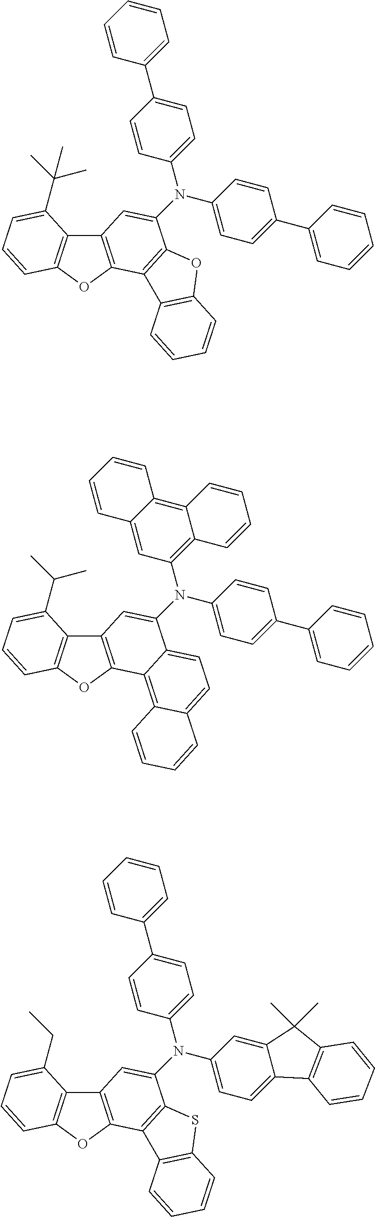 [SMILES: CC(C)(C)c1cccc2oc3c(cc(N(c4ccc(-c5ccccc5)cc4)c4ccc(-c5ccccc5)cc4)c4oc5ccccc5c43)c12 ; CC(C)c1cccc2oc3c(cc(N(c4ccc(-c5ccccc5)cc4)c4cc5ccccc5c5ccccc45)c4ccc5ccccc5c43)c12 ; CCc1cccc2oc3c(cc(N(c4ccc(-c5ccccc5)cc4)c4ccc5c(c4)C(C)(C)c4ccccc4-5)c4sc5ccccc5c43)c12]

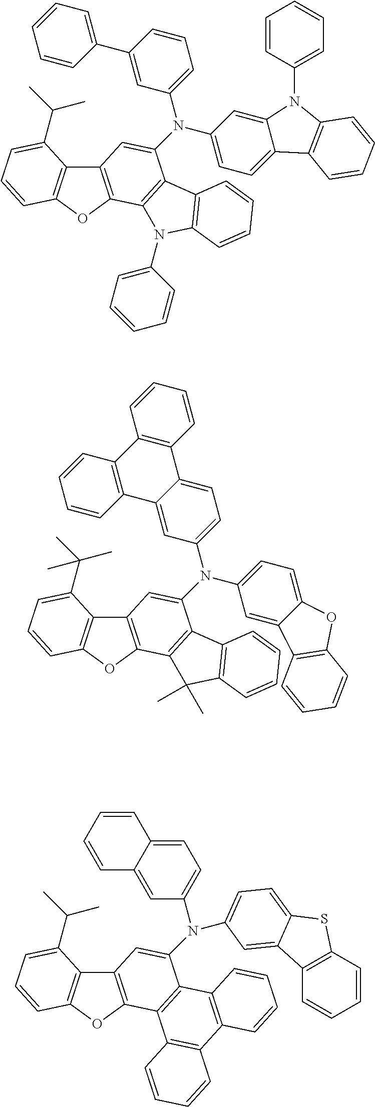 [SMILES: CC(C)c1cccc2oc3c(cc(N(c4cccc(-c5ccccc5)c4)c4ccc5c6ccccc6n(-c6ccccc6)c5c4)c4c5ccccc5n(-c5ccccc5)c34)c12 ; CC(C)(C)c1cccc2oc3c4c(c(N(c5ccc6oc7ccccc7c6c5)c5ccc6c7ccccc7c7ccccc7c6c5)cc3c12)-c1ccccc1C4(C)C ; CC(C)c1cccc2oc3c(cc(N(c4ccc5ccccc5c4)c4ccc5sc6ccccc6c5c4)c4c5ccccc5c5ccccc5c34)c12]

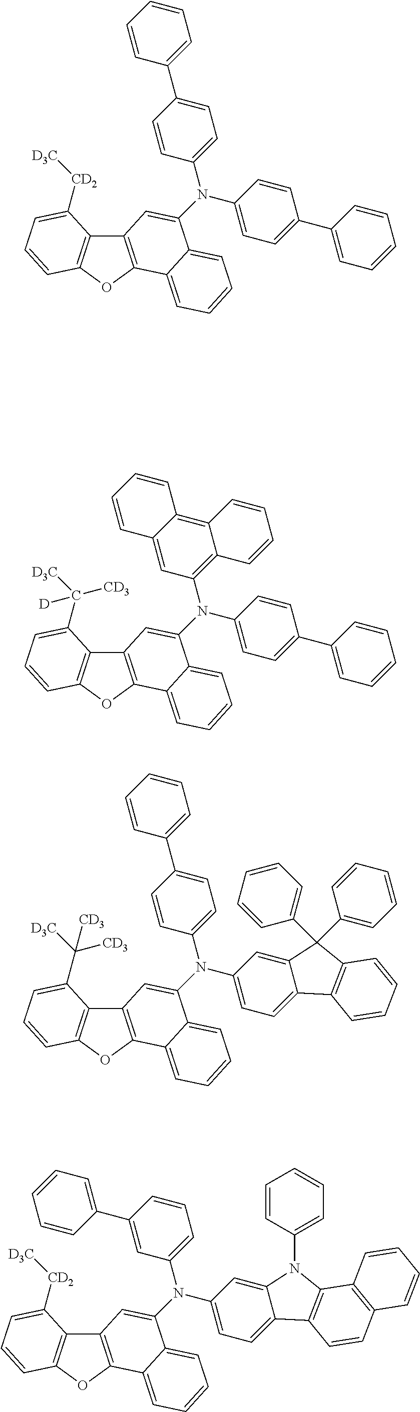

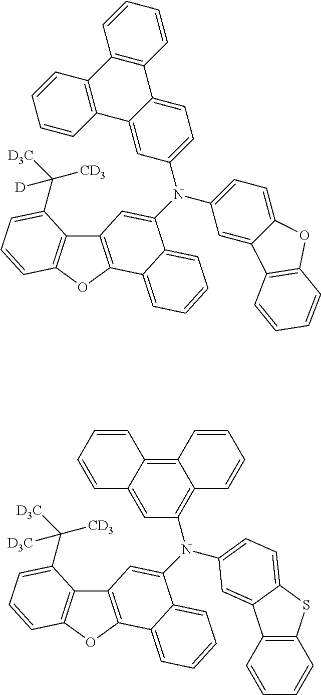 [SMILES: [2H]C([2H])([2H])C([2H])(c1cccc2oc3c4ccccc4c(N(c4ccc5oc6ccccc6c5c4)c4ccc5c6ccccc6c6ccccc6c5c4)cc3c12)C([2H])([2H])[2H] ; [2H]C([2H])([2H])C(c1cccc2oc3c4ccccc4c(N(c4ccc5sc6ccccc6c5c4)c4cc5ccccc5c5ccccc45)cc3c12)(C([2H])([2H])[2H])C([2H])([2H])[2H]]

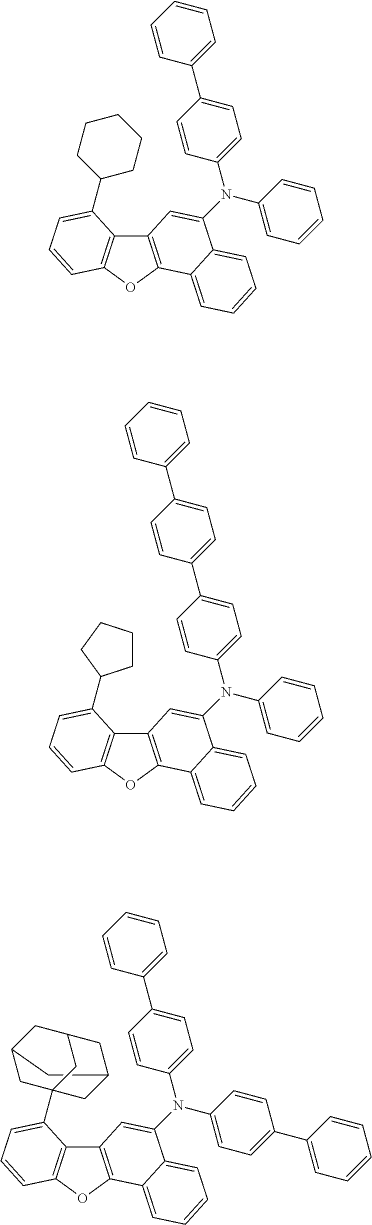 [SMILES: c1ccc(-c2ccc(N(c3ccccc3)c3cc4c(oc5cccc(C6CCCCC6)c54)c4ccccc34)cc2)cc1 ; c1ccc(-c2ccc(-c3ccc(N(c4ccccc4)c4cc5c(oc6cccc(C7CCCC7)c65)c5ccccc45)cc3)cc2)cc1 ; c1ccc(-c2ccc(N(c3ccc(-c4ccccc4)cc3)c3cc4c(oc5cccc(C67CC8CC(CC(C8)C6)C7)c54)c4ccccc34)cc2)cc1]

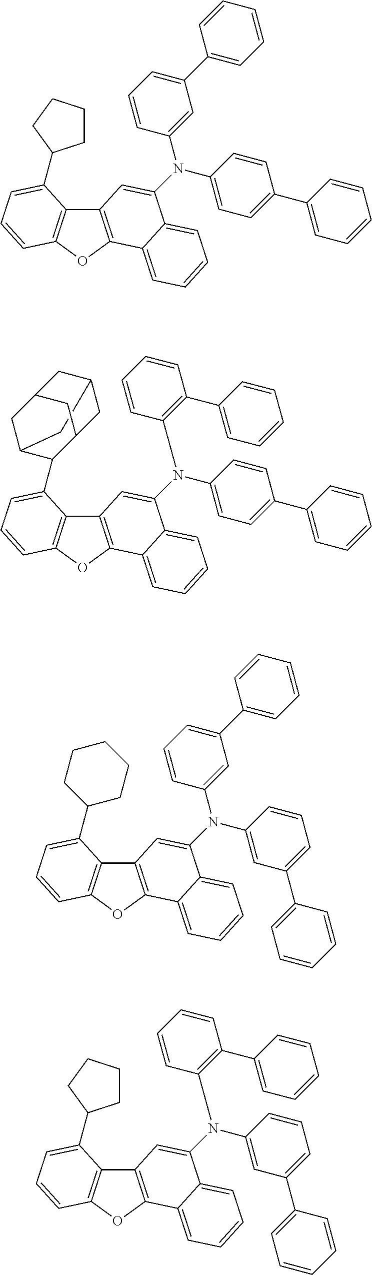 [SMILES: c1ccc(-c2ccc(N(c3cccc(-c4ccccc4)c3)c3cc4c(oc5cccc(C6CCCC6)c54)c4ccccc34)cc2)cc1 ; c1ccc(-c2ccc(N(c3ccccc3-c3ccccc3)c3cc4c(oc5cccc(C6C7CC8CC(C7)CC6C8)c54)c4ccccc34)cc2)cc1 ; c1ccc(-c2cccc(N(c3cccc(-c4ccccc4)c3)c3cc4c(oc5cccc(C6CCCCC6)c54)c4ccccc34)c2)cc1 ; c1ccc(-c2cccc(N(c3ccccc3-c3ccccc3)c3cc4c(oc5cccc(C6CCCC6)c54)c4ccccc34)c2)cc1]

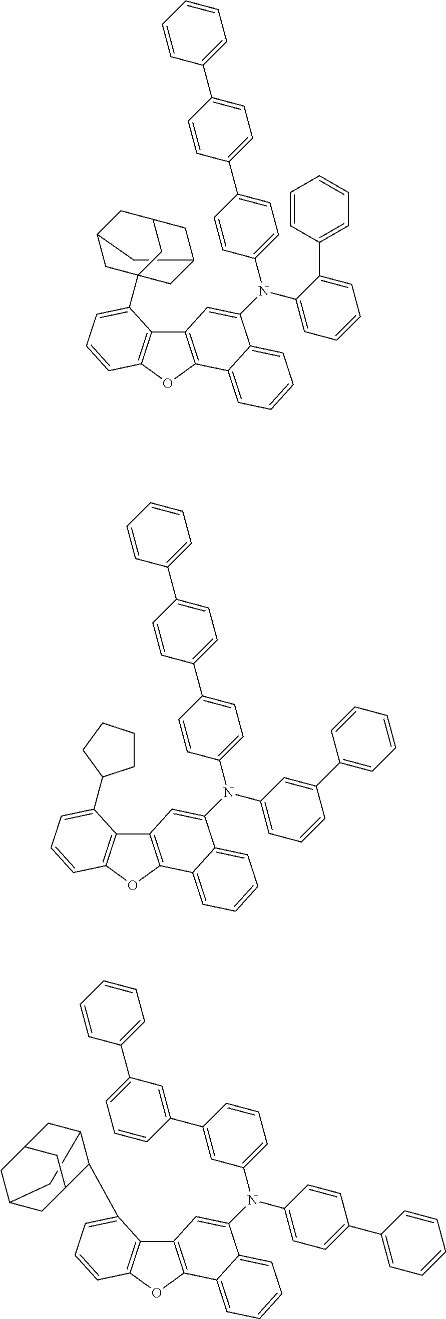 [SMILES: c1ccc(-c2ccc(-c3ccc(N(c4ccccc4-c4ccccc4)c4cc5c(oc6cccc(C78CC9CC(CC(C9)C7)C8)c65)c5ccccc45)cc3)cc2)cc1 ; c1ccc(-c2ccc(-c3ccc(N(c4cccc(-c5ccccc5)c4)c4cc5c(oc6cccc(C7CCCC7)c65)c5ccccc45)cc3)cc2)cc1 ; c1ccc(-c2ccc(N(c3cccc(-c4cccc(-c5ccccc5)c4)c3)c3cc4c(oc5cccc(C6C7CC8CC(C7)CC6C8)c54)c4ccccc34)cc2)cc1]

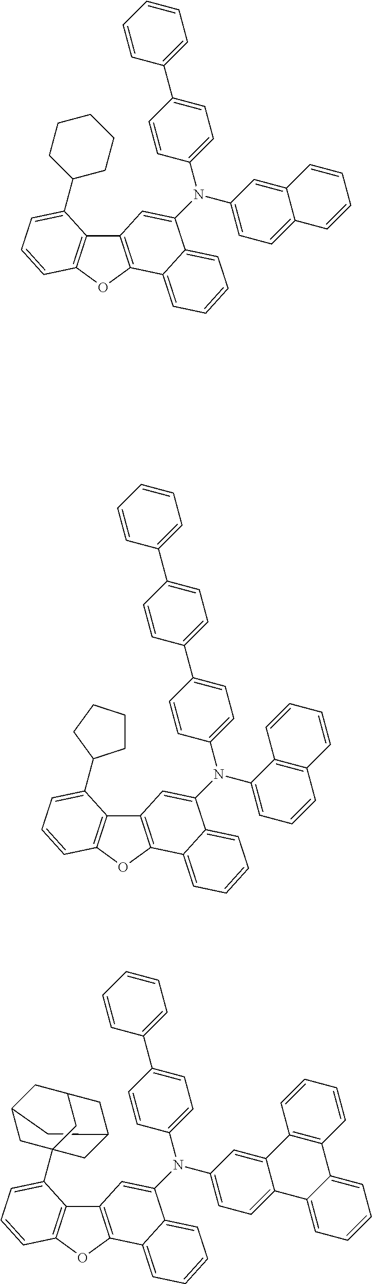 [SMILES: c1ccc(-c2ccc(N(c3ccc4ccccc4c3)c3cc4c(oc5cccc(C6CCCCC6)c54)c4ccccc34)cc2)cc1 ; c1ccc(-c2ccc(-c3ccc(N(c4cccc5ccccc45)c4cc5c(oc6cccc(C7CCCC7)c65)c5ccccc45)cc3)cc2)cc1 ; c1ccc(-c2ccc(N(c3ccc4c5ccccc5c5ccccc5c4c3)c3cc4c(oc5cccc(C67CC8CC(CC(C8)C6)C7)c54)c4ccccc34)cc2)cc1]

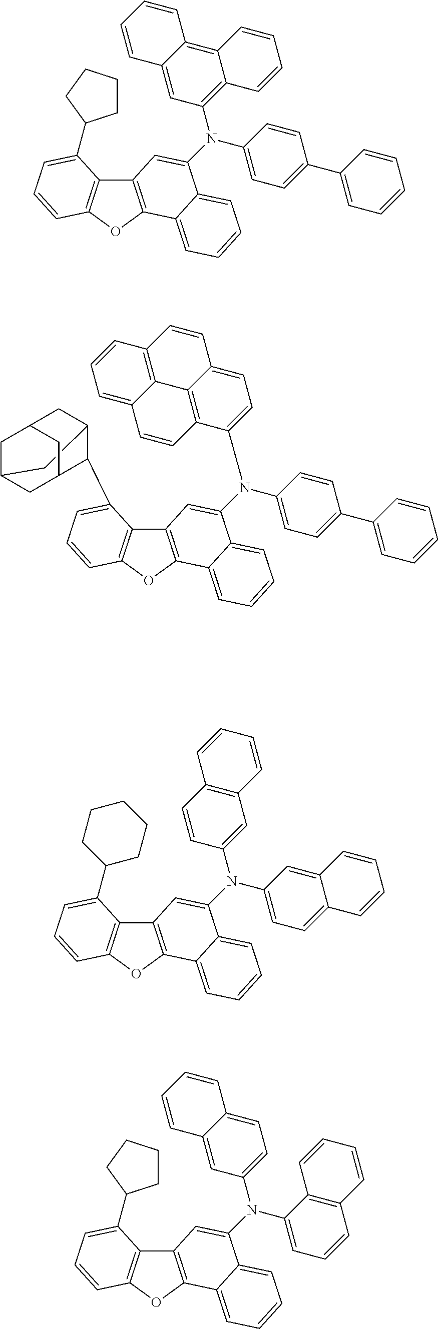 [SMILES: c1ccc(-c2ccc(N(c3cc4ccccc4c4ccccc34)c3cc4c(oc5cccc(C6CCCC6)c54)c4ccccc34)cc2)cc1 ; c1ccc(-c2ccc(N(c3cc4c(oc5cccc(C6C7CC8CC(C7)CC6C8)c54)c4ccccc34)c3ccc4ccc5cccc6ccc3c4c56)cc2)cc1 ; c1ccc2cc(N(c3ccc4ccccc4c3)c3cc4c(oc5cccc(C6CCCCC6)c54)c4ccccc34)ccc2c1 ; c1ccc2cc(N(c3cccc4ccccc34)c3cc4c(oc5cccc(C6CCCC6)c54)c4ccccc34)ccc2c1]

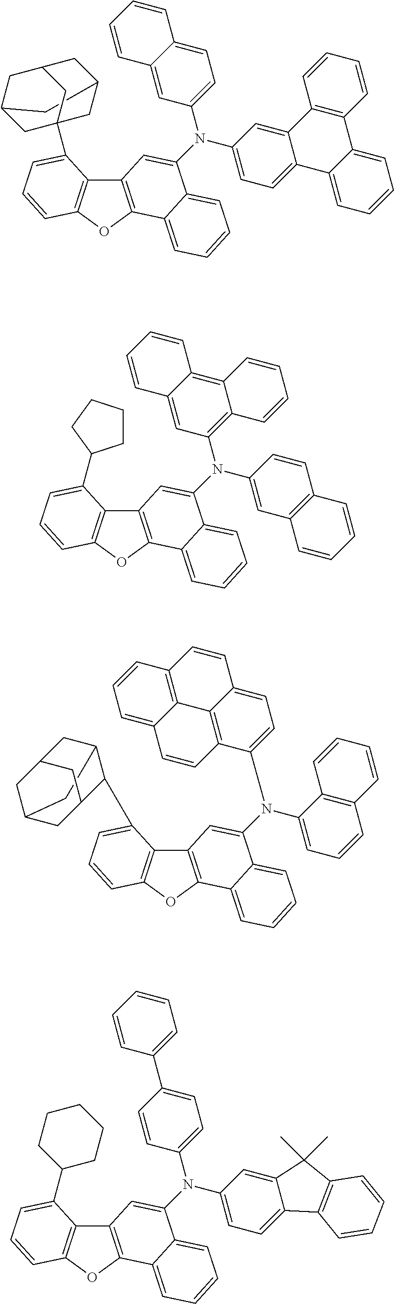 [SMILES: c1ccc2cc(N(c3ccc4c5ccccc5c5ccccc5c4c3)c3cc4c(oc5cccc(C67CC8CC(CC(C8)C6)C7)c54)c4ccccc34)ccc2c1 ; c1ccc2cc(N(c3cc4ccccc4c4ccccc34)c3cc4c(oc5cccc(C6CCCC6)c54)c4ccccc34)ccc2c1 ; c1ccc2c(N(c3cc4c(oc5cccc(C6C7CC8CC(C7)CC6C8)c54)c4ccccc34)c3ccc4ccc5cccc6ccc3c4c56)cccc2c1 ; CC1(C)c2ccccc2-c2ccc(N(c3ccc(-c4ccccc4)cc3)c3cc4c(oc5cccc(C6CCCCC6)c54)c4ccccc34)cc21]

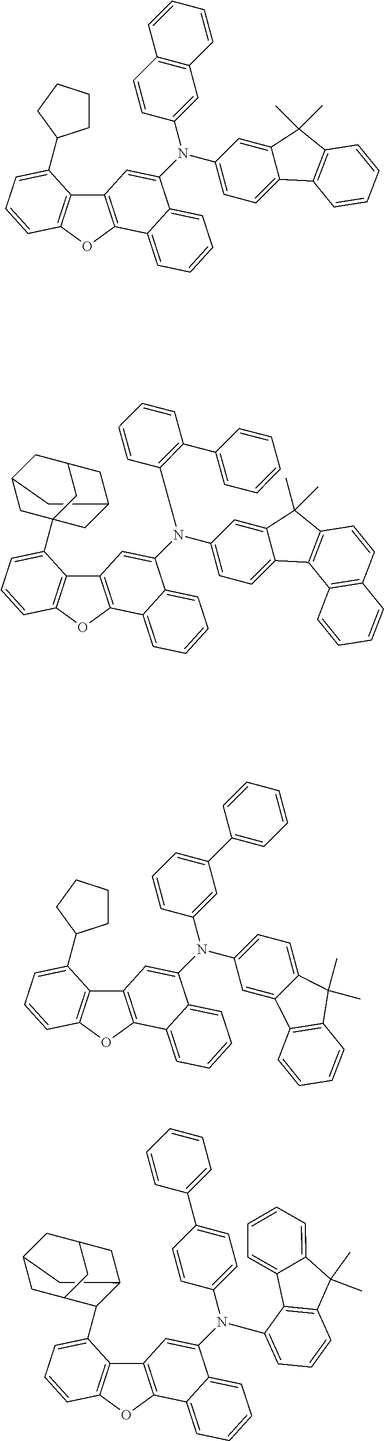 [SMILES: CC1(C)c2ccccc2-c2ccc(N(c3ccc4ccccc4c3)c3cc4c(oc5cccc(C6CCCC6)c54)c4ccccc34)cc21 ; CC1(C)c2cc(N(c3ccccc3-c3ccccc3)c3cc4c(oc5cccc(C67CC8CC(CC(C8)C6)C7)c54)c4ccccc34)ccc2-c2c1ccc1ccccc21 ; CC1(C)c2ccccc2-c2cc(N(c3cccc(-c4ccccc4)c3)c3cc4c(oc5cccc(C6CCCC6)c54)c4ccccc34)ccc21 ; CC1(C)c2ccccc2-c2c(N(c3ccc(-c4ccccc4)cc3)c3cc4c(oc5cccc(C6C7CC8CC(C7)CC6C8)c54)c4ccccc34)cccc21]

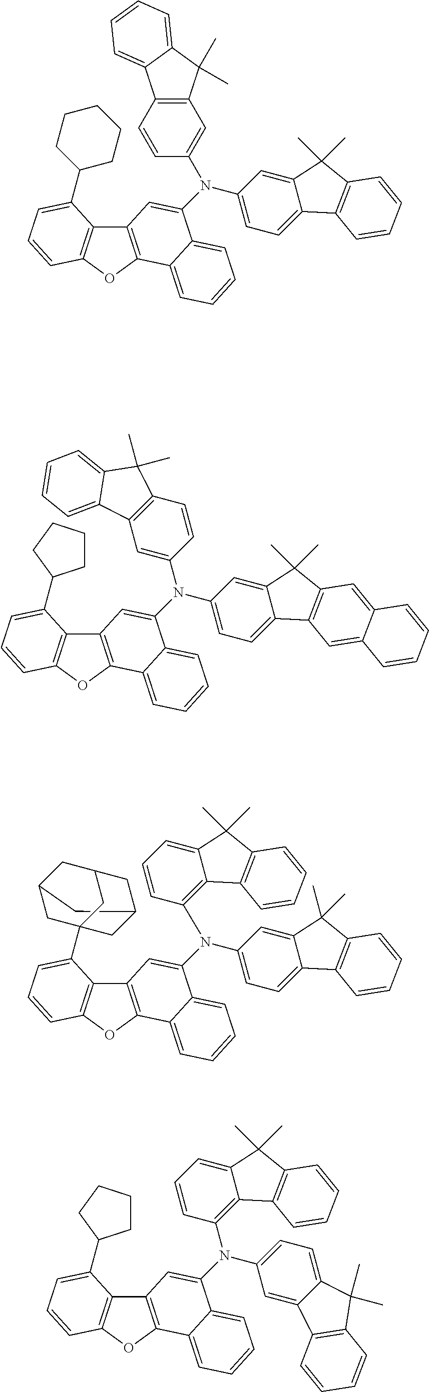 [SMILES: CC1(C)c2ccccc2-c2ccc(N(c3ccc4c(c3)C(C)(C)c3ccccc3-4)c3cc4c(oc5cccc(C6CCCCC6)c54)c4ccccc34)cc21 ; CC1(C)c2ccccc2-c2cc(N(c3ccc4c(c3)C(C)(C)c3cc5ccccc5cc3-4)c3cc4c(oc5cccc(C6CCCC6)c54)c4ccccc34)ccc21 ; CC1(C)c2ccccc2-c2ccc(N(c3cccc4c3-c3ccccc3C4(C)C)c3cc4c(oc5cccc(C67CC8CC(CC(C8)C6)C7)c54)c4ccccc34)cc21 ; CC1(C)c2ccccc2-c2cc(N(c3cccc4c3-c3ccccc3C4(C)C)c3cc4c(oc5cccc(C6CCCC6)c54)c4ccccc34)ccc21]

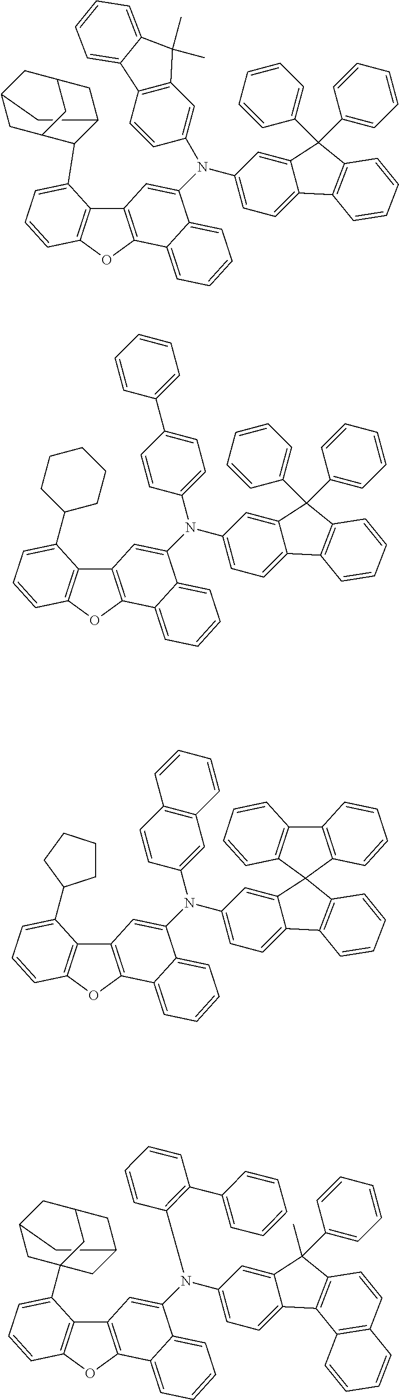 [SMILES: CC1(C)c2ccccc2-c2ccc(N(c3ccc4c(c3)C(c3ccccc3)(c3ccccc3)c3ccccc3-4)c3cc4c(oc5cccc(C6C7CC8CC(C7)CC6C8)c54)c4ccccc34)cc21 ; c1ccc(-c2ccc(N(c3ccc4c(c3)C(c3ccccc3)(c3ccccc3)c3ccccc3-4)c3cc4c(oc5cccc(C6CCCCC6)c54)c4ccccc34)cc2)cc1 ; c1ccc2c(c1)-c1ccccc1C21c2ccccc2-c2ccc(N(c3ccc4ccccc4c3)c3cc4c(oc5cccc(C6CCCC6)c54)c4ccccc34)cc21 ; CC1(c2ccccc2)c2cc(N(c3ccccc3-c3ccccc3)c3cc4c(oc5cccc(C67CC8CC(CC(C8)C6)C7)c54)c4ccccc34)ccc2-c2c1ccc1ccccc21]

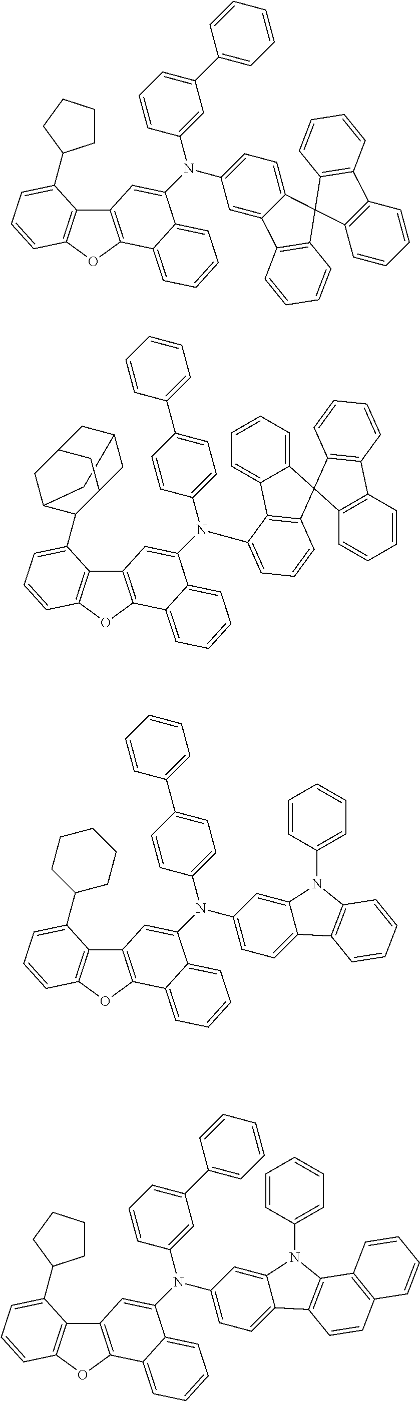 [SMILES: c1ccc(-c2cccc(N(c3ccc4c(c3)-c3ccccc3C43c4ccccc4-c4ccccc43)c3cc4c(oc5cccc(C6CCCC6)c54)c4ccccc34)c2)cc1 ; c1ccc(-c2ccc(N(c3cccc4c3-c3ccccc3C43c4ccccc4-c4ccccc43)c3cc4c(oc5cccc(C6C7CC8CC(C7)CC6C8)c54)c4ccccc34)cc2)cc1 ; c1ccc(-c2ccc(N(c3ccc4c5ccccc5n(-c5ccccc5)c4c3)c3cc4c(oc5cccc(C6CCCCC6)c54)c4ccccc34)cc2)cc1 ; c1ccc(-c2cccc(N(c3ccc4c5ccc6ccccc6c5n(-c5ccccc5)c4c3)c3cc4c(oc5cccc(C6CCCC6)c54)c4ccccc34)c2)cc1]

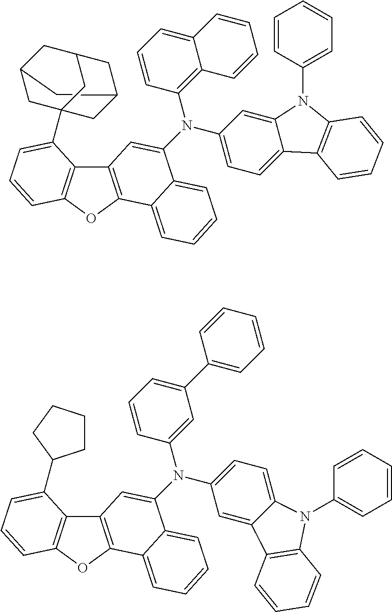 [SMILES: c1ccc(-n2c3ccccc3c3ccc(N(c4cccc5ccccc45)c4cc5c(oc6cccc(C78CC9CC(CC(C9)C7)C8)c65)c5ccccc45)cc32)cc1 ; c1ccc(-c2cccc(N(c3ccc4c(c3)c3ccccc3n4-c3ccccc3)c3cc4c(oc5cccc(C6CCCC6)c54)c4ccccc34)c2)cc1]

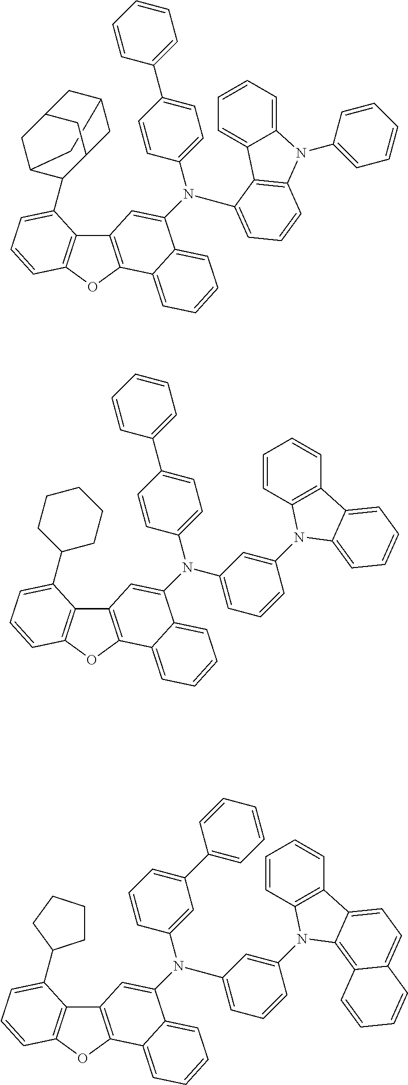 [SMILES: c1ccc(-c2ccc(N(c3cc4c(oc5cccc(C6C7CC8CC(C7)CC6C8)c54)c4ccccc34)c3cccc4c3c3ccccc3n4-c3ccccc3)cc2)cc1 ; c1ccc(-c2ccc(N(c3cccc(-n4c5ccccc5c5ccccc54)c3)c3cc4c(oc5cccc(C6CCCCC6)c54)c4ccccc34)cc2)cc1 ; c1ccc(-c2cccc(N(c3cccc(-n4c5ccccc5c5ccc6ccccc6c54)c3)c3cc4c(oc5cccc(C6CCCC6)c54)c4ccccc34)c2)cc1]

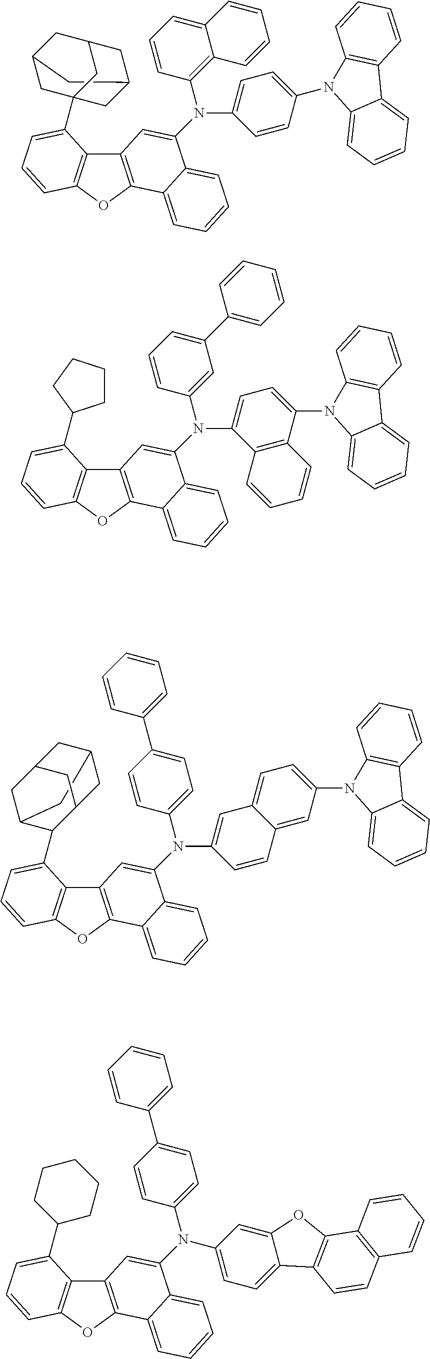 [SMILES: c1ccc2c(N(c3ccc(-n4c5ccccc5c5ccccc54)cc3)c3cc4c(oc5cccc(C67CC8CC(CC(C8)C6)C7)c54)c4ccccc34)cccc2c1 ; c1ccc(-c2cccc(N(c3ccc(-n4c5ccccc5c5ccccc54)c4ccccc34)c3cc4c(oc5cccc(C6CCCC6)c54)c4ccccc34)c2)cc1 ; c1ccc(-c2ccc(N(c3ccc4cc(-n5c6ccccc6c6ccccc65)ccc4c3)c3cc4c(oc5cccc(C6C7CC8CC(C7)CC6C8)c54)c4ccccc34)cc2)cc1 ; c1ccc(-c2ccc(N(c3ccc4c(c3)oc3c5ccccc5ccc43)c3cc4c(oc5cccc(C6CCCCC6)c54)c4ccccc34)cc2)cc1]

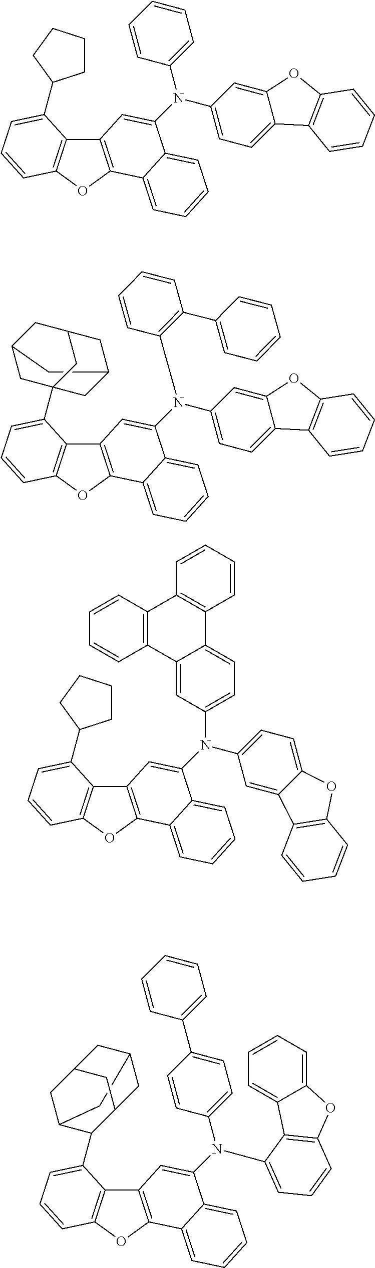

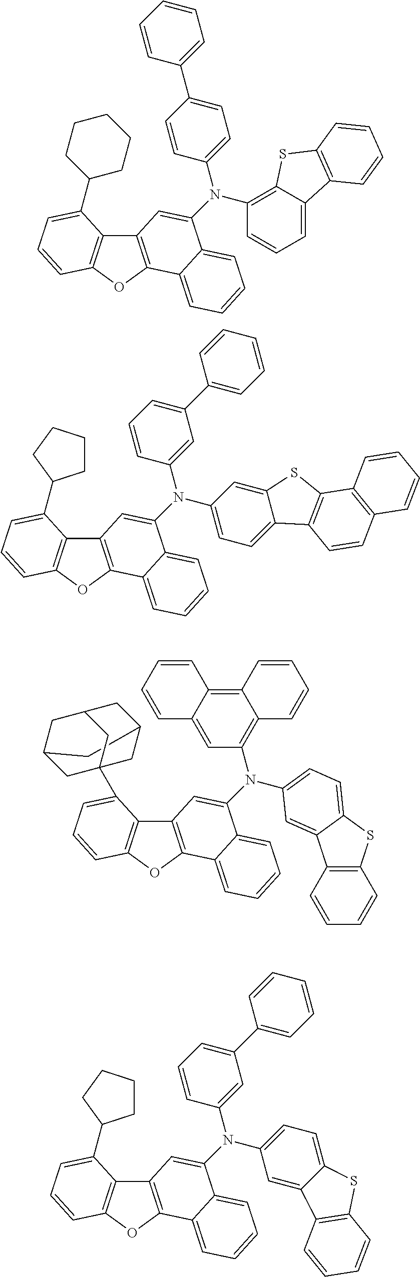

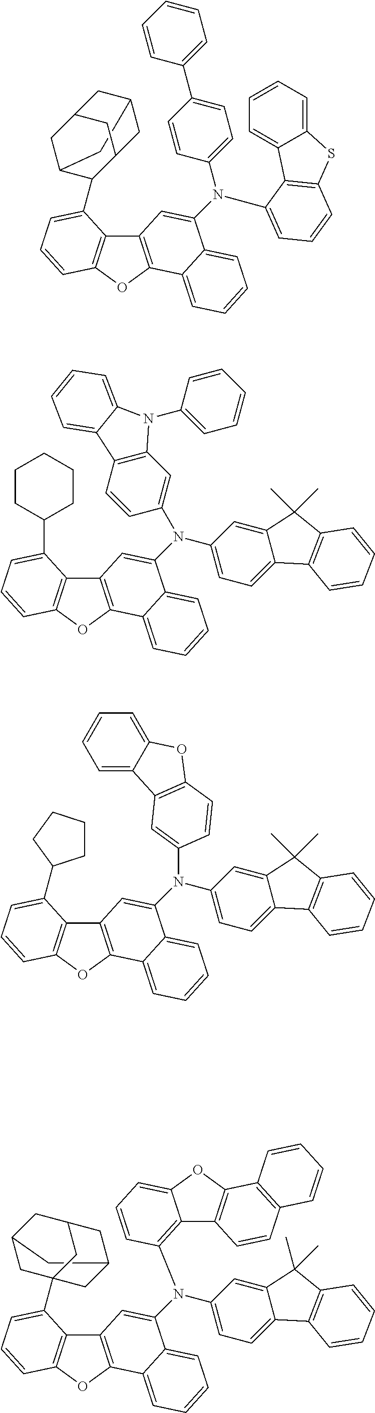 [SMILES: c1ccc(-c2ccc(N(c3cc4c(oc5cccc(C6C7CC8CC(C7)CC6C8)c54)c4ccccc34)c3cccc4sc5ccccc5c34)cc2)cc1 ; CC1(C)c2ccccc2-c2ccc(N(c3ccc4c5ccccc5n(-c5ccccc5)c4c3)c3cc4c(oc5cccc(C6CCCCC6)c54)c4ccccc34)cc21 ; CC1(C)c2ccccc2-c2ccc(N(c3ccc4oc5ccccc5c4c3)c3cc4c(oc5cccc(C6CCCC6)c54)c4ccccc34)cc21 ; CC1(C)c2ccccc2-c2ccc(N(c3cc4c(oc5cccc(C67CC8CC(CC(C8)C6)C7)c54)c4ccccc34)c3cccc4oc5c6ccccc6ccc5c34)cc21]

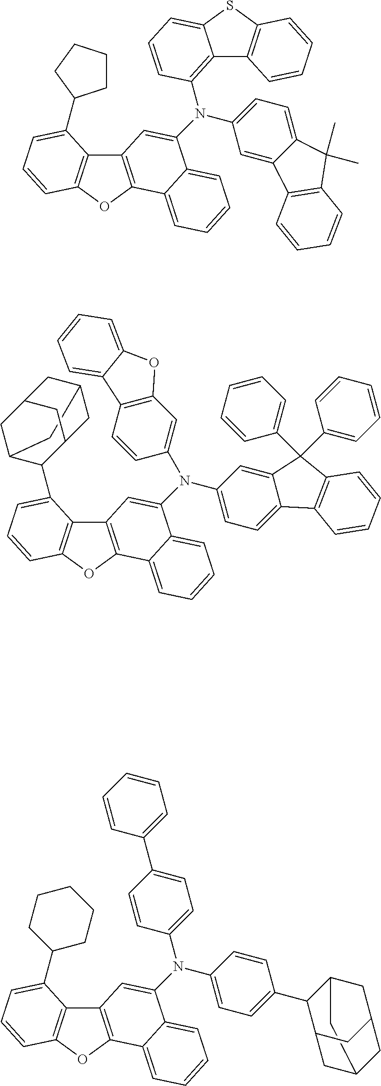 [SMILES: CC1(C)c2ccccc2-c2cc(N(c3cc4c(oc5cccc(C6CCCC6)c54)c4ccccc34)c3cccc4sc5ccccc5c34)ccc21 ; c1ccc(C2(c3ccccc3)c3ccccc3-c3ccc(N(c4ccc5c(c4)oc4ccccc45)c4cc5c(oc6cccc(C7C8CC9CC(C8)CC7C9)c65)c5ccccc45)cc32)cc1 ; c1ccc(-c2ccc(N(c3ccc(C4C5CC6CC(C5)CC4C6)cc3)c3cc4c(oc5cccc(C6CCCCC6)c54)c4ccccc34)cc2)cc1]

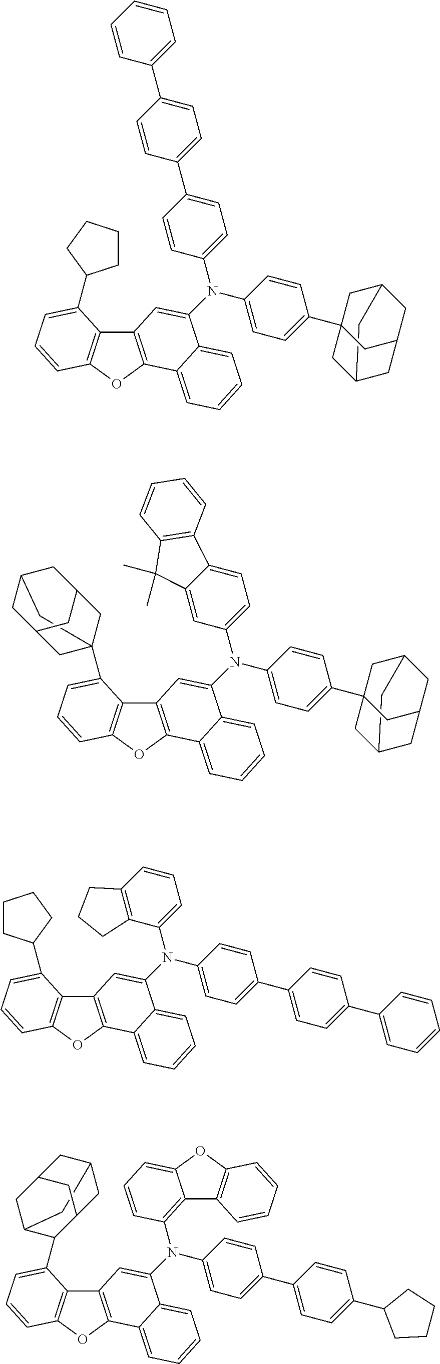 [SMILES: c1ccc(-c2ccc(-c3ccc(N(c4ccc(C56CC7CC(CC(C7)C5)C6)cc4)c4cc5c(oc6cccc(C7CCCC7)c65)c5ccccc45)cc3)cc2)cc1 ; CC1(C)c2ccccc2-c2ccc(N(c3ccc(C45CC6CC(CC(C6)C4)C5)cc3)c3cc4c(oc5cccc(C67CC8CC(CC(C8)C6)C7)c54)c4ccccc34)cc21 ; c1ccc(-c2ccc(-c3ccc(N(c4cccc5c4CCC5)c4cc5c(oc6cccc(C7CCCC7)c65)c5ccccc45)cc3)cc2)cc1 ; c1ccc2c(c1)oc1cccc(N(c3ccc(-c4ccc(C5CCCC5)cc4)cc3)c3cc4c(oc5cccc(C6C7CC8CC(C7)CC6C8)c54)c4ccccc34)c12]

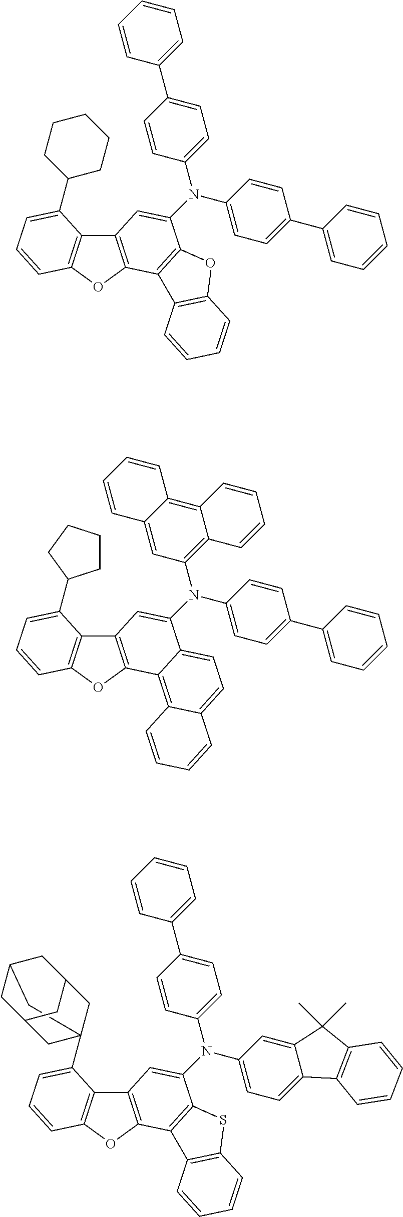 [SMILES: c1ccc(-c2ccc(N(c3ccc(-c4ccccc4)cc3)c3cc4c(oc5cccc(C6CCCCC6)c54)c4c3oc3ccccc34)cc2)cc1 ; c1ccc(-c2ccc(N(c3cc4ccccc4c4ccccc34)c3cc4c(oc5cccc(C6CCCC6)c54)c4c3ccc3ccccc34)cc2)cc1 ; CC1(C)c2ccccc2-c2ccc(N(c3ccc(-c4ccccc4)cc3)c3cc4c(oc5cccc(C67CC8CC(CC(C8)C6)C7)c54)c4c3sc3ccccc34)cc21]

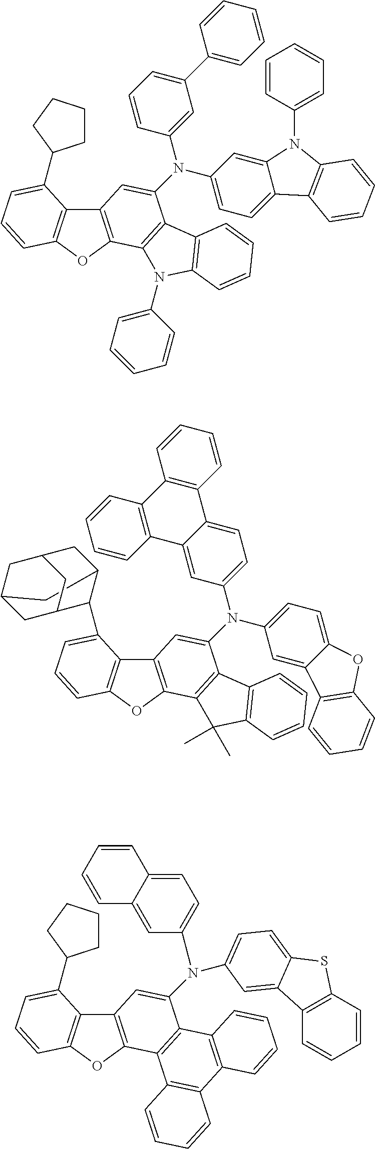 [SMILES: c1ccc(-c2cccc(N(c3ccc4c5ccccc5n(-c5ccccc5)c4c3)c3cc4c(oc5cccc(C6CCCC6)c54)c4c3c3ccccc3n4-c3ccccc3)c2)cc1 ; CC1(C)c2ccccc2-c2c(N(c3ccc4oc5ccccc5c4c3)c3ccc4c5ccccc5c5ccccc5c4c3)cc3c(oc4cccc(C5C6CC7CC(C6)CC5C7)c43)c21 ; c1ccc2cc(N(c3ccc4sc5ccccc5c4c3)c3cc4c(oc5cccc(C6CCCC6)c54)c4c5ccccc5c5ccccc5c34)ccc2c1]

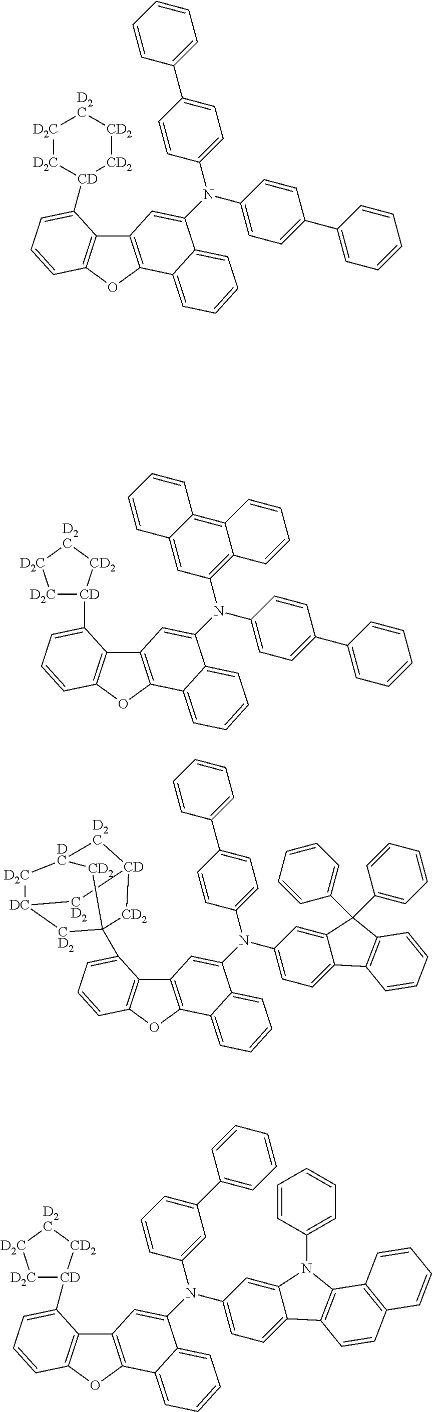

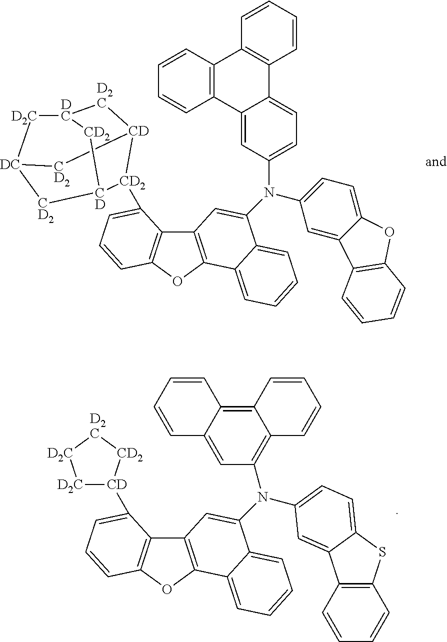

16. A composition, comprising an aromatic amine compound represented by formula (1) and an organic solvent; 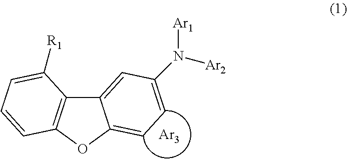
wherein Ar₁ and Ar₂ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof;
wherein Ar₃ is selected from a substituted or unsubstituted aromatic group with 6 to 30 ring atoms or a substituted or unsubstituted heteroaromatic group with 5 to 30 ring atoms; and
wherein R₁ is selected from substituted or unsubstituted methyl, substituted or unsubstituted ethyl, substituted or unsubstituted isopropyl, substituted or unsubstituted tertbutyl, substituted or unsubstituted cyclopentyl, substituted or unsubstituted cyclohexyl, or a substituted or unsubstituted adamantane group.

17. The composition of claim 16, wherein R₁ is selected from one of following substituted or unsubstituted groups: 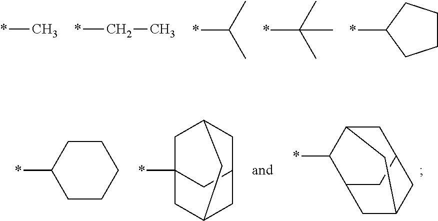
wherein * indicates a linking site.

18. The composition of claim 16, wherein Ar₁ and Ar₂ are each independently selected from one of following substituted or unsubstituted groups: 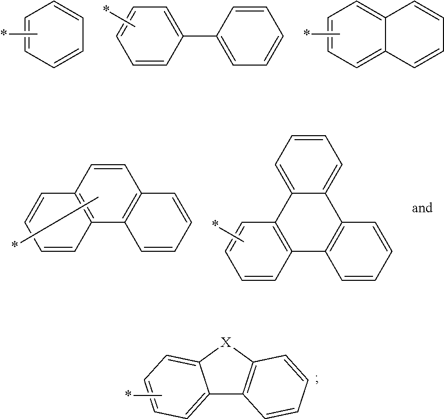
wherein X is selected from NR₂, CR₃R₄, O, S, SiR₃R₄, or PR₂; and wherein R₂, R₄, and R₄ are each independently selected from a substituted or unsubstituted C1-C20 alkyl group, a substituted or unsubstituted aromatic group with 6 to 60 ring atoms, a substituted or unsubstituted heteroaromatic group with 5 to 60 ring atoms, or combinations thereof; and
wherein * indicates a linking site.

19. The composition of claim 16, wherein Ar₃ is selected from one of following groups: 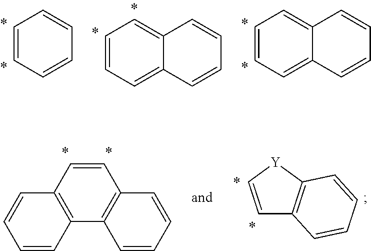
wherein Y is selected from NR₂, CR₃R₄, O, S, SiR₃R₄, or PR₂; and wherein R₂, R₃, and R₄ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof; and
wherein * * indicates a fused site.

20. An organic electronic device comprising a first electrode, a second electrode, and an organic functional layer disposed between the first electrode and the second electrode, wherein the organic functional layer comprises an aromatic amine compound represented by formula (1): 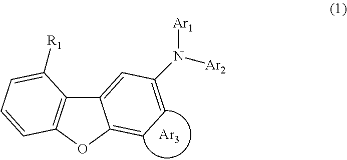
wherein Ar₁ and Ar₂ are each independently selected from a substituted or unsubstituted C₁-C₂₀ alkyl group, a substituted or unsubstituted aromatic group with 6 to 60 ring atoms, a substituted or unsubstituted heteroaromatic group with 5 to 60 ring atoms, or combinations thereof;
wherein Ar₃ is selected from a substituted or unsubstituted aromatic group with 6 to 30 ring atoms or a substituted or unsubstituted heteroaromatic group with 5 to 30 ring atoms; and
wherein R₁ is selected from substituted or unsubstituted methyl, substituted or unsubstituted ethyl, substituted or unsubstituted isopropyl, substituted or unsubstituted tertbutyl, substituted or unsubstituted cyclopentyl, substituted or unsubstituted cyclohexyl, or a substituted or unsubstituted adamantane group.
